(19) United States
(12) Patent Application Publication
(10) Pub. No.: US 2025/0215079 A1
(43) Pub. Date: Jul. 3, 2025

(54) ANTIBODIES SPECIFICALLY BINDING PD-1, TIM-3 OR PD-1 AND TIM-3 AND THEIR USES

(71) Applicant: Janssen Biotech, Inc., Horsham, PA (US)

(72) Inventors: Raluca Verona, Spring House, PA (US); Gordon Powers, Spring House, PA (US); Nina Chi Sabins, Spring House, PA (US); Nikki A. DeAngelis, Spring House, PA (US); Sandra Santulli-Marotto, Spring House, PA (US); Karla R. Wiehagen, Spring House, PA (US); Sheng-Jiun Wu, Spring House, PA (US); Catherine Ferrante, Spring House, PA (US); Enrique Zudaire Ubani, Spring House, PA (US)

(21) Appl. No.: 18/945,144

(22) Filed: Nov. 12, 2024

Related U.S. Application Data
(63) Continuation of application No. 17/090,961, filed on Nov. 6, 2020, now Pat. No. 12,173,064.
Application No. 17/090,961 is a division of application No. 15/340,109, filed on Nov. 1, 2016, now Pat. No. 10,894,830.
(60) Provisional application No. 62/250,095, filed on Nov. 3, 2015.

(51) Int. Cl. C07K 16/28; A61K 39/00; A61K 39/395; A61K 45/06; A61P 35/00; C07K 16/10; C07K 16/42

(52) U.S. Cl. CPC C07K 16/2803; A61K 39/3955; A61K 45/06; A61P 35/00; C07K 16/1063; C07K 16/2818; C07K 16/4258; A61K 2039/505; C07K 2317/21; C07K 2317/24; C07K 2317/31; C07K 2317/33; C07K 2317/34; C07K 2317/52; C07K 2317/55; C07K 2317/565; C07K 2317/71; C07K 2317/75; C07K 2317/76; C07K 2317/92

(57) ABSTRACT

The present invention relates to antibodies specifically binding PD-1, TIM-3 or PD-1 and TIM-3, polynucleotides encoding the antibodies or fragments, and methods of making and using the foregoing.

### CROSS-REFERENCE TO RELATED APPLICATIONS

This application is a continuation of U.S. application Ser. No. 17/090,961, filed Nov. 6, 2020, now U.S. Pat. No. 12,173,064, granted Dec. 24, 2024; which is a divisional of U.S. application Ser. No. 15/340,109, filed Nov. 1, 2016, now U.S. Pat. No. 10,894,830, granted Jan. 19, 2021, which claims the benefit of U.S. Provisional Application Ser. No. 62/250,095 filed Nov. 3, 2015, the entire contents of the aforementioned applications are incorporated herein by reference in their entireties.

### SEQUENCE LISTING

The instant application contains a Sequence Listing which has been submitted electronically in XML file format and is hereby incorporated by reference in its entirety. Said XML copy, created on Feb. 13, 2025, is named JBI5071USCNT1_SL.xml and is 428,069 bytes in size.

### FIELD OF THE INVENTION

The present invention relates antibodies specifically binding PD-1, TIM-3 or PD-1 and TIM-3, polynucleotides encoding the antibodies or fragments, and methods of making and using the foregoing.

### BACKGROUND OF THE INVENTION

The immune system is tightly controlled by a network of costimulatory and co-inhibitory ligands and receptors. These molecules provide secondary signals for T cell activation and provide a balanced network of positive and negative signals to maximize immune responses against infection and tumors, while limiting immunity to self (Wang et al., (Epub Mar. 7, 2011) J Exp Med 208(3):577-92; Lepenies et al., (2008) Endocr Metab Immune Disord Drug Targets 8:279-288).

Immune checkpoint therapy, targeting co-inhibitory pathways in T cells to promote antitumor immune responses, has led to advances in clinical care of cancer patients.

PD-1 is a negative immune checkpoint molecule that suppresses CD4⁺ and CD8⁺ T cell functions in the tumor microenvironment (TME). PD-1 engagement with its ligands (PD-L1 and PD-L2) drives T cell anergy and exhaustion in tumors by inhibiting multiple pathways downstream of the T cell receptor signaling, resulting in decreased T cell survival, growth and proliferation, compromised effector function, and altered metabolism. Preclinical studies have demonstrated that the PD-1 pathway blockade can reverse T cell exhaustion and stimulate anti-tumor immunity.

The PD-1 pathway hence contributes to downregulation of T cell functions in the (TME) and evasion of tumors via immune destruction. In the TME, exhausted T cells, in addition to expressing high levels of PD-1, express other inhibitory receptors including CTLA-4, TIM-3, LAG-3, CD244, TIGIT and CD160 (see e.g., Pauken & Wherry; 2015, Trends in Immunology 36(4): 265-276).

TIM-3 is a transmembrane receptor that is expressed on Th1 (T helper 1) CD4⁺ cells and cytotoxic CD8⁺ T cells that secrete IFN-γ. TIM-3 is generally not expressed on naïve T cells but rather upregulated on activated, effector T cells. TIM-3 has a role in regulating immunity and tolerance in vivo (see Hastings et al., (2009) Eur J Immunol 39(9):2492-501).

PD-1 antibodies have been described for example in: U.S. Pat. Nos. 5,897,862 and 7,488,802, and in Int. Patent Publ. Nos. WO2004/004771, WO2004/056875, WO2006/121168, WO2008/156712, WO2010/029435, WO2010/036959, WO2011/110604, WO2012/145493, WO2014/194302, WO2014/206107, WO2015/036394, WO2015/035606, WO2015/085847, WO2015/112900 and WO2015/112805.

TIM-3 antibodies have been described for example in: Monney et al., Nature (2002) 415(6871):536-41, and in Int. Patent Publ. Nos. WO2011/155607, WO2013/006490 and WO2015/117002.

Combinations with TIM-3 antibody and a PD-L1 antibody have been evaluated in for example in Int. Patent Publ. No. WO2011/159877.

While anti-PD-1/PD-L1 antibodies are demonstrating encouraging clinical responses in patients with multiple solid tumors, the response rates are still fairly low, about 15%-20% in pretreated patients (Swaika et al., (2015) Mol Immunol. doi: 10.1016/j.molimm.2015.02.009).

Therefore, there is a need for new therapeutics that inhibit the immunosuppressive activity of checkpoint inhibitors such as PD-1 and TIM-3, to be used for cancer immunotherapy and treatment of other conditions that would benefit from enhancement of an immune response, such as chronic infections.

### BRIEF SUMMARY OF THE INVENTION

The invention provides an isolated antagonistic antibody specifically binding PD-1, comprising a heavy chain complementarity determining region 1 (HCDR1), a HCDR2 and a HCDR3 of SEQ ID NOs: X₁YX₂IX₃ wherein X₁ is S or D; X₂ is V or A; and X₃ is H or S, 83 and 84, respectively, or SEQ ID NOs: X₁YX₂IX₃ wherein X₁ is S or D; X₂ is V or A; and X₃ is H or S, 83 and 85, respectively.

The invention also provides an isolated antagonistic antibody specifically binding PD-1, comprising a heavy chain complementarity determining region 1 (HCDR1), a HCDR2 and a HCDR3 of SEQ ID NOs: X₁YX₂IX₃ wherein X₁ is S or D; X₂ is V or A; and X₃ is H or S, 83 and 84, respectively, and a light chain complementarity determining region 1 (LCDR1), a LCDR2 and a LCDR3 of SEQ ID NOs: 86, 87 and 88, respectively.

The invention also provides an isolated antagonistic antibody specifically binding PD-1, comprising a heavy chain complementarity determining region 1 (HCDR1), a HCDR2 and a HCDR3 of SEQ ID NOs: X₁YX₂IX₃ wherein X₁ is S or D; X₂ is V or A; and X₃ is H or S, 83 and 85, respectively, and a light chain complementarity determining region 1 (LCDR1), a LCDR2 and a LCDR3 of SEQ ID NOs: 86, 87 and 88, respectively.

The invention also provides an isolated antagonistic antibody specifically binding PD-1, comprising certain HCDR1, HCDR2, HCDR3, LCDR1, LCDR2 and LCDR3 amino acid sequences as described herein.

The invention also provides an isolated antagonistic antibody specifically binding PD-1, comprising certain VH and VL amino acid sequences as described herein.

The invention also provides an isolated antagonistic antibody specifically binding TIM-3, comprising a heavy chain complementarity determining region 1 (HCDR1), a HCDR2 and a HCDR3 of SEQ ID NOs: X₁₇YX₁₈MX₁₉ wherein X₁₇ is N, S, G or D; X₁₈ is W or A; and X₁₉ is S or H, 165 and X₂₄X₂₅X₂₆X₂₇X₂₈X₂₉X₃₀X₃₁DY wherein X₂₄ is D, S, N, G or E; X₂₅ is H, P, E, T or L; X₂₆ is W, E, N or deleted; X₂₇ is D, P or deleted; X₂₈ is P, Y, D or deleted; X₂₉ is N, A, D, G or deleted; X₃₀ is F, P, R, W or V; and X₃₁ is L or F, respectively.

The invention also provides an isolated antagonistic antibody specifically binding TIM-3, comprising the HCDR1, the HCDR2 and the HCDR3 of SEQ ID NOs: X₁₇YX₁₈MX₁₉ wherein X₁₇ is N, S, G or D; X₁₈ is W or A; and X₁₉ is S or H, 165 and X₂₄X₂₅X₂₆X₂₇X₂₈X₂₉X₃₀X₃₁DY wherein X₂₄ is D, S, N, G or E; X₂₅ is H, P, E, T or L; X₂₆ is W, E, N or deleted; X₂₇ is D, P or deleted; X₂₈ is P, Y, D or deleted; X₂₉ is N, A, D, G or deleted; X₃₀ is F, P, R, W or V; and X₃₁ is L or F, respectively, and the LCDR1, the LCDR2 and the LCDR3 of SEQ ID NOs: 167, X₄₃ASX₄₄RX₄₅X₄₆ wherein X₄₃ is G, D, W or T; X₄₄ is S, N or T; X₄₅ is A or E; and X₄₆ is T or S and 169 respectively.

The invention also provides an isolated antagonistic antibody specifically binding TIM-3, comprising certain HCDR1, HCDR2, HCDR3, LCDR1, LCDR2 and LCDR3 amino acid sequences as described herein.

The invention also provides an isolated antagonistic antibody specifically binding TIM-3, comprising certain VH and VL amino acid sequences as described herein.

The invention also provides an isolated antagonistic bispecific PD1/TIM-3 antibody comprising a first domain specifically binding PD-1 and a second domain specifically binding TIM-3.

The invention also provides an isolated antagonistic bispecific PD1/TIM-3 antibody comprising a first domain specifically binding PD-1 and a second domain specifically binding TIM-3, comprising certain HCDR1, HCDR2, HCDR3, LCDR1, LCDR2, LCDR3, VH, VL, heavy chain or light chain amino acids sequences as described herein.

The invention also provides an isolated antagonistic bispecific PD1/TIM-3 antibody comprising a first domain specifically binding PD-1 and a second domain specifically binding TIM-3, wherein the first domain comprises the HCDR1, the HCDR2, the HCDR3, the LCDR1, the LCDR2 and the LCDR3 of SEQ ID NOs: 10, 14, 17, 23, 26 and 32, respectively, and the second domain comprises the HCDR1, the HCDR2, the HCDR3, the LCDR1, the LCDR2 and the LCDR3 of SEQ ID NOs: 97, 105, 115, 124, 133 and 143, respectively.

The invention also provides an isolated antagonistic bispecific PD1/TIM-3 antibody comprising a first domain specifically binding PD-1 and a second domain specifically binding TIM-3, wherein the first domain comprises the HCDR1, the HCDR2, the HCDR3, the LCDR1, the LCDR2 and the LCDR3 of SEQ ID NOs: 10, 14, 17, 23, 26 and 32, respectively, and the second domain comprises the HCDR1, the HCDR2, the HCDR3, the LCDR1, the LCDR2 and the LCDR3 of SEQ ID NOs: 91, 99, 108, 118, 127 and 136, respectively.

The invention also provides an isolated antagonistic bispecific PD1/TIM-3 antibody comprising a first domain specifically binding PD-1 and a second domain specifically binding TIM-3, wherein the first domain comprises the HCDR1, the HCDR2, the HCDR3, the LCDR1, the LCDR2 and the LCDR3 of SEQ ID NOs: 66, 67, 68, 69, 70 and 71, respectively, and the second domain comprises the HCDR1, the HCDR2, the HCDR3, the LCDR1, the LCDR2 and the LCDR3 of SEQ ID NOs: 97, 105, 115, 124, 133 and 143, respectively.

The invention also provides an isolated antagonistic bispecific PD1/TIM-3 antibody comprising a first domain specifically binding PD-1 and a second domain specifically binding TIM-3, wherein the first domain comprises the HCDR1, the HCDR2, the HCDR3, the LCDR1, the LCDR2 and the LCDR3 of SEQ ID NOs: 66, 67, 68, 69, 70 and 71, respectively, and the second domain comprises the HCDR1, the HCDR2, the HCDR3, the LCDR1, the LCDR2 and the LCDR3 of SEQ ID NOs: 91, 99, 108, 118, 127 and 136, respectively.

The invention also provides an isolated antagonistic bispecific PD1/TIM-3 antibody comprising a first domain specifically binding PD-1 and a second domain specifically binding TIM-3, wherein the first domain comprises the HCDR1, the HCDR2, the HCDR3, the LCDR1, the LCDR2 and the LCDR3 of SEQ ID NOs: 10, 14, 16, 723, 26 and 32, respectively, and the second domain comprises the HCDR1, the HCDR2, the HCDR3, the LCDR1, the LCDR2 and the LCDR3 of SEQ ID NOs: 97, 105, 115, 124, 133 and 143, respectively.

The invention also provides an isolated antagonistic bispecific PD1/TIM-3 antibody comprising a first domain specifically binding PD-1 and a second domain specifically binding TIM-3, wherein the first domain comprises a heavy chain variable region (VH) of SEQ ID NO: 48 and a light chain variable region (VL) of SEQ ID NO: 56, and the second domain comprises the VH of SEQ ID NO: 153 and the VL of SEQ ID NO: 162.

The invention also provides an isolated antagonistic bispecific PD1/TIM-3 antibody comprising a first domain specifically binding PD-1 and a second domain specifically binding TIM-3, wherein the first domain comprises the VH of SEQ ID NO: 48 and the VL of SEQ ID NO: 56, and the second domain comprises the VH of SEQ ID NO: 146 and the VL of SEQ ID NO: 156.

The invention also provides an isolated antagonistic bispecific PD1/TIM-3 antibody comprising a first domain specifically binding PD-1 and a second domain specifically binding TIM-3, wherein the first domain comprises the VH of SEQ ID NO: 64 and the VL of SEQ ID NO: 65, and the second domain comprises the VH of SEQ ID NO: 153 and the VL of SEQ ID NO: 162.

The invention also provides an isolated antagonistic bispecific PD1/TIM-3 antibody comprising a first domain specifically binding PD-1 and a second domain specifically binding TIM-3, wherein the first domain comprises the VH of SEQ ID NO: 64 and the VL of SEQ ID NO: 65, and the second domain comprises the VH of SEQ ID NO: 146 and the VL of SEQ ID NO: 156.

The invention also provides an isolated antagonistic bispecific PD1/TIM-3 antibody comprising a first domain specifically binding PD-1 and a second domain specifically binding TIM-3, wherein the first domain comprises the VH of SEQ ID NO: 48 and the VL of SEQ ID NO: 56, and the second domain comprises the VH of SEQ ID NO: 172 and the VL of SEQ ID NO: 173.

The invention also provides an immunoconjugate comprising the antibody or antigen-binding portion thereof of the invention linked to a therapeutic agent or to an imaging agent.

The invention also provides a pharmaceutical composition comprising the antibody of the invention and a pharmaceutically accepted carrier.

The invention also provides a polynucleotide encoding the antibody VH, the antibody VL or the antibody VH and the antibody VL of the invention.

The invention also provides a vector comprising the polynucleotide encoding the antibody VH, the antibody VL or the antibody VH and the VL of the invention.

The invention also provides a host cell comprising the vector of the invention.

The invention also provides a method of producing the antibody of the invention, comprising culturing the host cell of the invention in conditions that the antibody is expressed, and recovering the antibody produced by the host cell.

The invention also provides a method of treating a cancer in a subject, comprising administering a therapeutically effective amount of the isolated antibody of the invention to the subject in need thereof for a time sufficient to treat the cancer.

The invention also provides a method of enhancing an immune response in a subject, comprising administering a therapeutically effective amount of the isolated antibody of the invention to the subject in need thereof for a time sufficient to enhance the immune response.

The invention also provides an anti-idiotypic antibody binding to the antibody of the invention.

The invention also provides a kit comprising the antibody of the invention.

### BRIEF DESCRIPTION OF THE DRAWINGS

FIG. 1A shows that TIM-3 surface expression is elevated in tumors after treatment with anti-PD-1 antibodies. Balb/c mice with established CT26 colon carcinoma tumors were treated biweekly with anti-PD-1 antibody or vehicle. Tumors were harvested at day 22 and TIM-3 expression was evaluated on tumor-infiltrating T cells using flow cytometry. MFJ: mean fluorescent intensity. PBS: control

FIG. 1B shows that TIM-3 surface expression is elevated on tumor infiltrated lymphocytes (TIL) after treatment with anti-PD-1 antibodies. Balb/c mice with established MC38 colon carcinoma tumors were treated biweekly with anti-PD-1 antibody or vehicle. Geometric mean fluorescent intensity (gMFI) of TIM-3 expression on total CD8 TIL population is shown in vehicle treated (PBS) or anti-PD-1 antibody treated (PD-1) animals. p=0.003 vehicle vs anti-PD-1 antibody treated groups.

FIG. 1C shows the relative frequency of TIM-3Y CD8 cells of total CD8⁺ TILs in MC38 tumors harvested from mice treated with vehicle (PBS) or anti-PD-1 antibody (PD-1). p=0.045 vehicle vs anti-PD-1 antibody treated groups.

FIG. 2A shows that CD137 surface expression (gMFI) is elevated on TILs in MC38 colon carcinoma tumors in animals treated with anti-PD-1 antibodies (PD-1 group) when compared to vehicle treated (PBS) group. p=0.005 vehicle vs anti-PD-1 antibody treated groups. Each point represents one mouse. Data are representative of at least 2 independent experiments.

FIG. 2B shows that the relative frequency of CD137T CD8 cells of total CD8⁺ TILs in is elevated in MC38 colon carcinoma tumors in animals treated with anti-PD-1 antibodies (PD-1 group) when compared to vehicle treated (PBS) group. p=0.0475 vehicle vs anti-PD-1 antibody treated groups. Each point represents one mouse. Data are representative of at least 2 independent experiments.

FIG. 3A shows that OX40 surface expression (gMFI) is elevated on TILs in MC38 colon carcinoma tumors in animals treated with anti-PD-1 antibodies (PD-1 group) when compared to vehicle treated (PBS) group. p=0.0013 vehicle vs anti-PD-1 antibody treated groups. Each point represents one mouse. Data are representative of at least 2 independent experiments.

FIG. 3B shows that the relative frequency of OX40⁺ CD8 cells of total CD8⁺ TILs in is elevated in MC38 colon carcinoma tumors in animals treated with anti-PD-1 antibodies (PD-1 group) when compared to vehicle treated (PBS) group. p=0.03 vehicle vs anti-PD-1 antibody treated groups. Each point represents one mouse. Data are representative of at least 2 independent experiments.

FIG. 4A shows that GITR surface expression (gMFI) is elevated on TILs in MC38 colon carcinoma tumors in animals treated with anti-PD-1 antibodies (PD-1 group) when compared to vehicle treated (PBS) group. p=0.0004 vehicle vs anti-PD-1 antibody treated groups. Each point represents one mouse. Data are representative of at least 2 independent experiments.

FIG. 4B shows that the relative frequency of GITR CD8 cells of total CD8⁺ TILs in is elevated in MC38 colon carcinoma tumors in animals treated with anti-PD-1 antibodies (PD-1 group) when compared to vehicle treated (PBS) group. p=0.0015 vehicle vs anti-PD-1 antibody treated groups. Each point represents one mouse. Data are representative of at least 2 independent experiments.

FIG. 5 shows that treatment with anti-TIM-3 antibodies after anti-PD-1 antibody treatment further induces antigen-specific immune response. The antibodies were tested in the CMV assay using PBMCs from CMV positive donors, in which antigen-specific immune responses were induced with pp65 peptide pools. The cells were treated for 5 days with anti-PD-1 antibody PD1B244, re-stimulated, and treated for 24 hours with anti-TIM-3 antibody TM3B105. Immune response was determined by measuring increases in IFN-γ secretion. IgG2s Iso: IgG2sigma isotype control. CMV: sample treated with cytomegalovirus p65 peptides in the absence of antibodies.

FIG. 6 shows the HCDR1 sequences of select anti-PD-1 antibodies and the HCDR1 genus sequence.

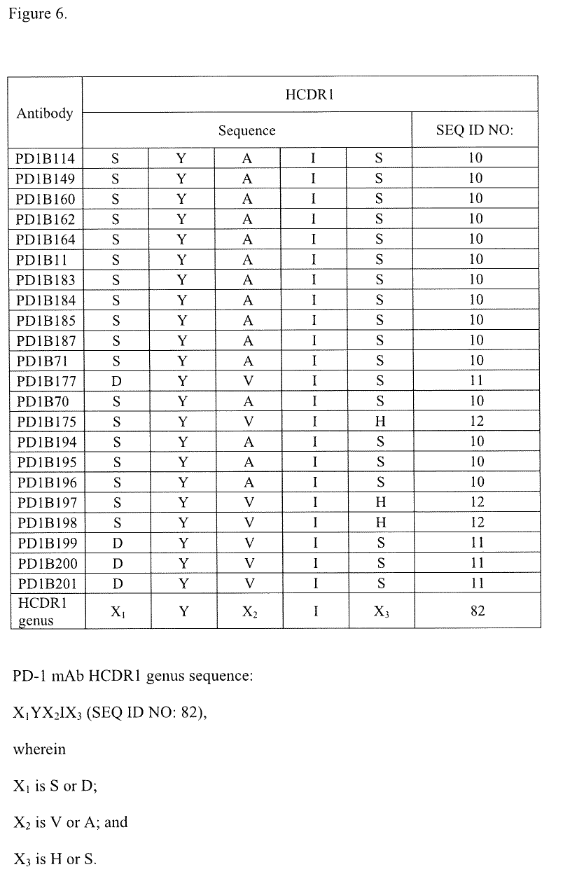

FIG. 7 shows the HCDR2 sequences of select anti-PD-1 antibodies and the HCDR2 genus sequence.

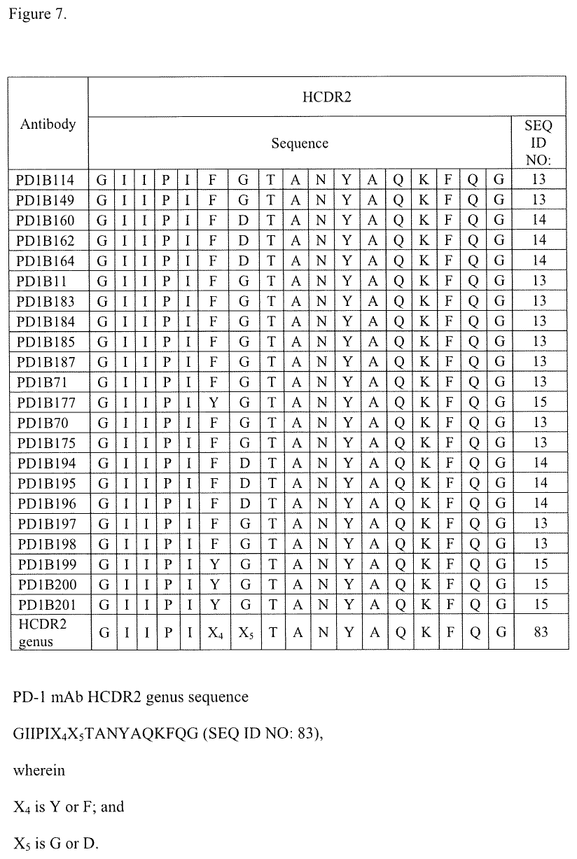

FIG. 8 shows the HCDR3 sequences of select anti-PD-1 antibodies and the first HCDR3 genus sequence.

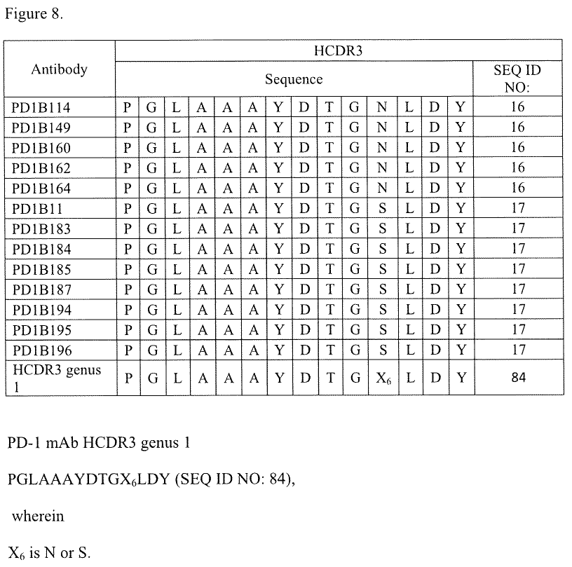

FIG. 9 shows the HCDR3 sequences of select anti-PD-1 antibodies and the second HCDR3 genus sequence.

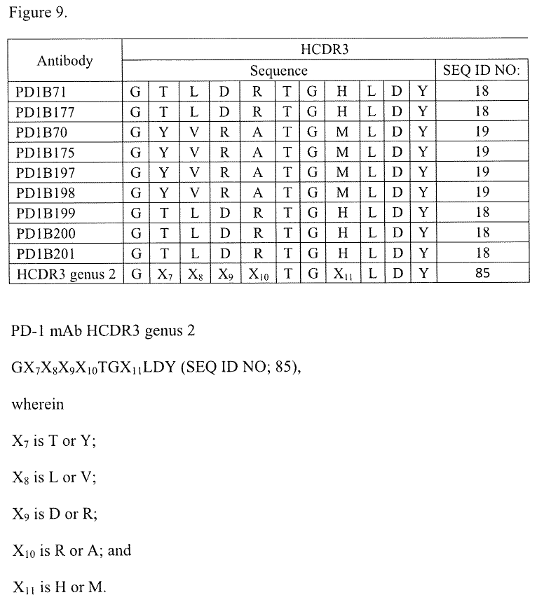

FIG. 10 shows the LCDR1 sequences of select anti-PD-1 antibodies and the LCDR1 genus sequence.

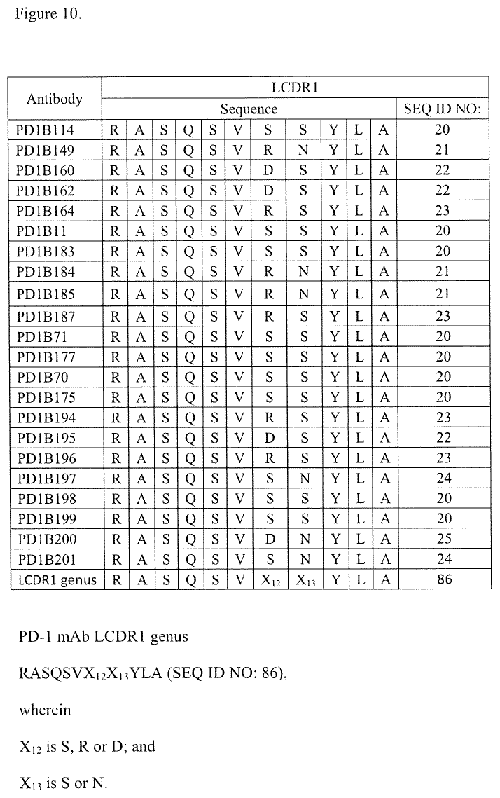

FIG. 11 shows the LCDR2 sequences of select anti-PD-1 antibodies and the LCDR2 genus sequence.

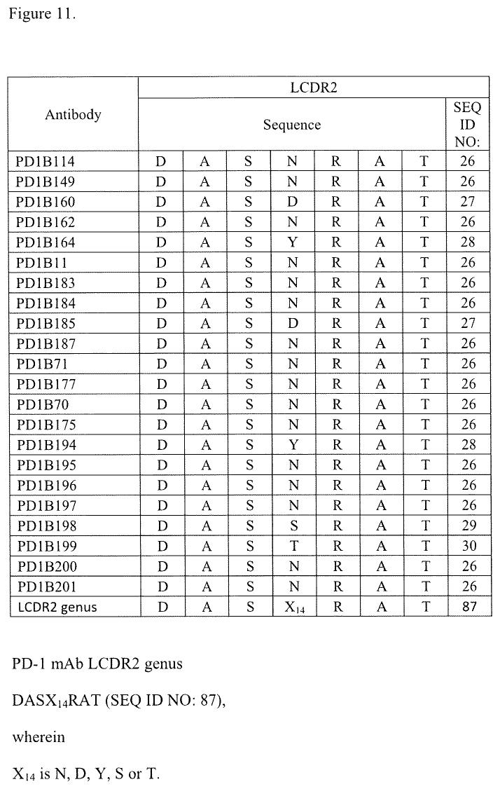

FIG. 12 shows the LCDR3 sequences of select anti-PD-1 antibodies and the LCDR3 genus sequence.

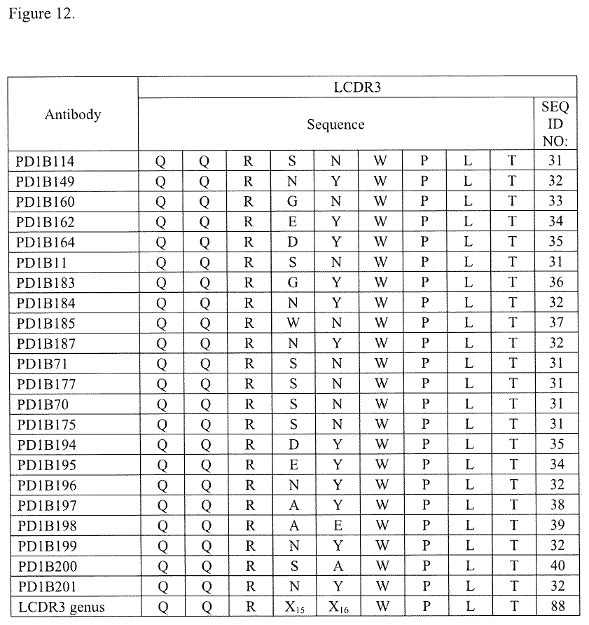

FIG. 13 shows the HCDR1 sequences of select anti-TIM-3 antibodies and the HCDR1 genus sequence. The genus sequence was determined by generating molecular models for all Fv (VH/VL pairs) in MOE (CCG, Montreal) using a default protocol for antibody modeling. For CDRs that have different lengths, these structural models were aligned based upon the structurally conserved regions and the structurally equivalent CDRs positions were identified.

FIG. 14 shows the HCDR2 sequences of select anti-TIM-3 antibodies and the HCDR2 genus sequence. The HCDR2 genus sequence was generated as described for FIG. 10.

FIG. 15 shows the HCDR3 sequences of select anti-TIM-3 antibodies and the first HCDR3 genus sequence. The HCDR3 genus sequence was generated as described for FIG. 10.

FIG. 16 shows the LCDR1 sequences of select anti-TIM-3 antibodies and the LCDR1 genus sequence. The LCDR1 genus sequence was generated as described for FIG. 10.

FIG. 17 shows the LCDR2 sequences of select anti-TIM-3 antibodies and the LCDR2 genus sequence. The LCDR2 genus sequence was generated as described for FIG. 10.

FIG. 18 shows the LCDR3 sequences of select anti-TIM-3 antibodies and the LCDR3 genus sequence. The LCDR3 genus sequence was generated as described for FIG. 10.

FIG. 19A shows that TIGIT surface expression (gMFI) is elevated on TILs in MC38 colon carcinoma tumors in animals treated with anti-TIM-3 antibodies (TIM-3 group) when compared to vehicle treated (PBS) group. p=0.0181 vehicle vs anti-TIM-3 antibody treated groups. Each point represents one mouse. Data are representative of at least 2 independent experiments.

FIG. 19B shows that the relative frequency of TIGIT+ CD8 cells of total CD8⁺ TILs in is elevated in MC38 colon carcinoma tumors in animals treated with anti-TIM-3 antibodies (TIM-3 group) when compared to vehicle treated (PBS) group. p=0.0475 vehicle vs anti-TIM-3 antibody treated groups. Each point represents one mouse. Data are representative of at least 2 independent experiments.

FIG. 20A shows that TIGIT surface expression (gMFI) is elevated on TILs in CT26 colon carcinoma tumors in animals treated with anti-TIM-3 antibodies (TIM-3 group) when compared to vehicle treated (PBS) group. p<0.001 vehicle vs anti-TIM-3 antibody treated groups. Each point represents one mouse. Data are representative of at least 2 independent experiments.

FIG. 20B shows that the relative frequency of TIGIT+ CD8 cells of total CD8⁺ TILs in is elevated in CT26 colon carcinoma tumors in animals treated with anti-TIM-3 antibodies (TIM-3 group) when compared to vehicle treated (PBS) group. p=0.0105 vehicle vs anti-TIM-3 antibody treated groups. Each point represents one mouse. Data are representative of at least 2 independent experiments.

FIG. 21 shows upregulation of TIM-3 expression on peripheral T cells in melanoma patients PBMCs from treatment naïve melanoma patients stimulated with melanoma antigen peptide pools (NY-ESO, gp100, MART-1) in the presence or absence of anti-PD-1 or anti-TIM-3 function blocking antibodies. Expression of TIM-3 was determined by flow cytometry on restimulated cells on day 6.

FIG. 22A shows that TM3B403 treatment increases frequency of activated NK cells in IL-2 stimulated human PBMCs. IgG2s: Isotype control. NK cell activation was assessed as percentage (%) of CD69 expressing cells in the stimulated PBMCs.

FIG. 22B shows that TM3B403 treatment increases frequency of activated NK cells in IL-2 stimulated human PBMCs. IgG2s: Isotype control. NK cell activation was assessed as percentage (%) of CD25 expressing cells in the stimulated PBMCs.

### DETAILED DESCRIPTION OF THE INVENTION

All publications, including but not limited to patents and patent applications, cited in this specification are herein incorporated by reference as though fully set forth.

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

As used in this specification and the appended claims, the singular forms “a,” “an,” and “the” include plural referents unless the content clearly dictates otherwise.

Thus, for example, reference to “a cell” includes a combination of two or more cells, and the like.

“Specific binding” or “specifically binds” or “binds” refers to an antibody binding to an antigen or an epitope within the antigen with greater affinity than for other antigens. Typically, the antibody binds to the antigen or the epitope within the antigen with an equilibrium dissociation constant (K_{D}) of about 1×10⁻⁸ M or less, for example about 1×10⁻⁹ M or less, about 1×10⁻¹⁰ M or less, about 1×10⁻¹¹ M or less, or about 1×10⁻² M or less, typically with the K_{D} that is at least one hundred fold less than its K_{D} for binding to a non-specific antigen (e.g., BSA, casein). The dissociation constant may be measured using standard procedures. Antibodies that specifically bind to the antigen or the epitope within the antigen may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset). While a monospecific antibody specifically binds one antigen or one epitope, a bispecific antibody specifically binds two distinct antigens or two distinct epitopes.

“Antibodies” is meant in a broad sense and includes immunoglobulin molecules including monoclonal antibodies including murine, human, humanized and chimeric monoclonal antibodies, antigen-binding fragments, bispecific or multispecific antibodies, dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy (H) chains and two light (L) chains inter-connected by disulfide bonds as well as multimers thereof (for example IgM). Each heavy chain is comprised of a heavy chain variable region (VH) and a heavy chain constant region (comprised of domains CH1, hinge CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-terminus to carboxy-terminus in the following order: FR1, CDR1, FR2, CDR2, FR3, CDR3, and FR4.

“Complementarity determining regions (CDR)” are “antigen binding sites” in an antibody. CDRs may be defined using various terms: (i) Complementarity Determining Regions (CDRs), three in the VH (HCDR1, HCDR2, HCDR3) and three in the VL (LCDR1, LCDR2, LCDR3) are based on sequence variability (Wu and Kabat, (1970) J Exp Med 132:211-50; Kabat et al., Sequences of Proteins of Immunological Interest, 5th Ed. Public Health Service, National Institutes of Health, Bethesda, Md., 1991). (ii) “Hypervariable regions”, “HVR”, or “HV”, three in the VH (H1, H2, H3) and three in the VL (L1, L2, L3) refer to the regions of an antibody variable domains which are hypervariable in structure as defined by Chothia and Lesk (Chothia and Lesk, (1987) Mol Biol 196:901-17). The International ImMunoGeneTics (IMGT) database (http://www_imgt_org) provides a standardized numbering and definition of antigen-binding sites. The correspondence between CDRs, HVs and IMGT delineations is described in Lefranc et al., (2003) Dev Comparat Immunol 27:55-77. The term “CDR”, “HCDR1”, “HCDR2”, “HCDR3”, “LCDR1”, “LCDR2” and “LCDR3” as used herein includes CDRs defined by any of the methods described supra, Kabat, Chothia or IMGT, unless otherwise explicitly stated in the specification.

Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may assigned to one of two clearly distinct types, namely kappa (x) and lambda (a), based on the amino acid sequences of their constant domains.

“Antibody fragments” or “antigen-binding portion” refers to a portion of an immunoglobulin molecule that retains the antigen binding properties of the parental full length antibody. Exemplary antigen-binding portions are heavy chain complementarity determining regions (HCDR) 1, 2 and 3, light chain complementarity determining regions (LCDR) 1, 2 and 3, a heavy chain variable region (VH), a light chain variable region (VL), Fab, F(ab′)2, Fd and Fv fragments as well as domain antibodies (dAb) consisting of either one VH or VL domain. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“Monoclonal antibody” refers to an antibody population with single amino acid composition in each heavy and each light chain, except for possible well known alterations such as removal of C-terminal lysine from the antibody heavy chain. Monoclonal antibodies typically bind one antigenic epitope, except that multispecific monoclonal antibodies bind two or more distinct antigens or epitopes. Bispecific monoclonal antibodies bind two distinct antigenic epitopes. Monoclonal antibodies may have heterogeneous glycosylation within the antibody population. Monoclonal antibodies may be monospecific or multispecific, or monovalent, bivalent or multivalent. A multispecific antibody, such as a bispecific antibody or a trispecific antibody is included in the term monoclonal antibody.

“Isolated antibody” refers to an antibody or antibody fragment that is substantially free of other antibodies having different antigenic specificities (e.g., an isolated antibody specifically binding PD-1 is substantially free of antibodies that specifically bind antigens other than PD-1). An isolated antibody specifically binding TIM-3 is substantially free of antibodies that specifically bind antigens other than TIM-3. In case of bispecific PD-1/TIM-3 antibodies, the bispecific antibody specifically binds both PD-1 and TIM-3, and is substantially free of antibodies that specifically bind antigens other that PD-1 and TIM-3. “Isolated antibody” encompasses antibodies that are isolated to a higher purity, such as antibodies that are 80%, 81%, 82%, 83%, 84%, 85%, 86%, 87%, 88%, 89%, 90%, 91%, 92%, 93%, 94%, 95%, 96%, 97%, 98%, 99% or 100% pure.

“Humanized antibodies” refers to antibodies in which at least one CDR is derived from non-human species and the variable region frameworks are derived from human immunoglobulin sequences. Humanized antibodies may include intentionally introduced mutations in the framework regions so that the framework may not be an exact copy of expressed human immunoglobulin or germline gene sequences.

“Human antibody” refers to an antibody having heavy and light chain variable regions in which both the framework and all 6 CDRs are derived from sequences of human origin. If the antibody contains a constant region or a portion of the constant region, the constant region also is derived from sequences of human origin.

Human antibody comprises heavy or light chain variable regions that are “derived from” sequences of human origin if the variable regions of the antibody are obtained from a system that uses human germline immunoglobulin or rearranged immunoglobulin genes.

Such exemplary systems are human immunoglobulin gene libraries displayed on phage, and transgenic non-human animals such as mice or rats carrying human immunoglobulin loci as described herein. “Human antibody” may contain amino acid differences when compared to the human germline immunoglobulin or rearranged immunoglobulin genes due to for example naturally occurring somatic mutations or intentional introduction of substitutions into the framework or antigen binding site, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462.

Human antibodies derived from human immunoglobulin sequences may be generated using systems such as phage display incorporating synthetic CDRs and/or synthetic frameworks, or may be subjected to in vitro mutagenesis to improve antibody properties, resulting in antibodies that are not expressed by the human antibody germline repertoire in vivo.

“Recombinant” refers to antibodies and other proteins that are prepared, expressed, created or isolated by recombinant means.

“Epitope” refers to a portion of an antigen to which an antibody specifically binds. Epitopes typically consist of chemically active (such as polar, non-polar or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule. Antibody “epitope” depends on the methodology used to identify the epitope.

“Multispecific” refers to an antibody that specifically binds at least two distinct antigens or two distinct epitopes within the antigens, for example three, four or five distinct antigens or epitopes.

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset), or may bind an epitope that is shared between two or more distinct antigens.

“Variant” refers to a polypeptide or a polynucleotide that differs from a reference polypeptide or a reference polynucleotide by one or more modifications for example, substitutions, insertions or deletions.

“Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system. Examples of such biological systems may include a cell, virus, animal, plant, and reconstituted biological systems utilizing biological components capable of duplicating a vector. The polynucleotide comprising a vector may be DNA or RNA molecules or a hybrid of these.

“Expression vector” refers to a vector that can be utilized in a biological system or in a reconstituted biological system to direct the translation of a polypeptide encoded by a polynucleotide sequence present in the expression vector.

“Polynucleotide” refers to a synthetic molecule comprising a chain of nucleotides covalently linked by a sugar-phosphate backbone or other equivalent covalent chemistry. cDNA is a typical example of a polynucleotide.

“Polypeptide” or “protein” refers to a molecule that comprises at least two amino acid residues linked by a peptide bond to form a polypeptide. Small polypeptides of less than 50 amino acids may be referred to as “peptides”.

PD-1 refers to human programmed cell death protein 1, PD-1. PD-1 is also known as CD279 or PDCD1. The amino acid sequence of the mature human PD-1 (without signal sequence) is shown in SEQ ID NO: 1. The extracellular domain spans residues 1-150, the transmembrane domain spans residues 151-171 and the cytoplasmic domain spans residues 172-268 of SEQ ID NO: 1. Throughout the specification, “the extracellular domain of human PD-1 “huPD1-ECD” refers to protein having amino acid sequence of residues 1-149 of SEQ ID NO: 1, and shown in SEQ ID NO:2. “PD-1” in the specification refers to human mature PD-1, unless explicitly stated to the contrary.

TIM-3 refers to human hepatitis A virus cellular receptor 2, also called HAVCR2. The amino acid sequence of the mature human TIM-3 (without signal sequence) is shown in SEQ ID NO: 138. The extracellular domain spans residues 1-181, the transmembrane domain spans residues 182-202 and the cytoplasmic domain spans residues 203-280 of SEQ ID NO: 138. Throughout the specification, “the extracellular domain of human TIM-3 “huTIM-3-ECD” refers to protein having amino acid sequence of residues 1-179 of SEQ ID NO: 138, and shown in SEQ ID NO: 89. TIM-3 in the specification refers to human mature TIM-3, unless explicitly stated to the contrary.

“In combination with” means that two or more therapeutics are administered to a subject together in a mixture, concurrently as single agents or sequentially as single agents in any order.

“Overexpress”, “overexpressed” and “overexpressing” is used interchangeably and refers to a sample such as a cancer cell, malignant cell or cancer tissue that has measurably higher levels of PD-1, TIM-3, PD-L1, PD-L2 or TIM-3 ligand when compared to a reference sample. The overexpression may be caused by gene amplification or by increased transcription or translation. Expression and overexpression of protein in the sample may be measured using well know assays using for example ELISA, immunofluorescence, flow cytometry or radioimmunoassay on live or lysed cells. Expression and overexpression of a polynucleotide in the sample may be measured for example using fluorescent in situ hybridization, Southern blotting, or PCR techniques. A protein or a polynucleotide is overexpressed when the level of the protein or the polynucleotide in the sample at least 1.5-fold higher or statistically significant when compared to the reference sample. Selection of the reference sample is known.

“Sample” refers to a collection of similar fluids, cells, or tissues isolated from a subject, as well as fluids, cells, or tissues present within a subject. Exemplary samples are biological fluids such as blood, serum and serosal fluids, plasma, lymph, urine, saliva, cystic fluid, tear drops, feces, sputum, mucosal secretions of the secretory tissues and organs, vaginal secretions, ascites fluids such as those associated with non-solid tumors, fluids of the pleural, pericardial, peritoneal, abdominal and other body cavities, fluids collected by bronchial lavage, liquid solutions contacted with a subject or biological source, for example, cell and organ culture medium including cell or organ conditioned medium, lavage fluids and the like, tissue biopsies, fine needle aspirations or surgically resected tumor tissue.

A “cancer cell” or a “tumor cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo (Freshney, Culture of Animal Cells: A Manual of Basic Technique (3rd ed. 1994)).

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Bispecific PD-1/TIM-3 antibody”, “PD-1/TIM-3 antibody”, “bispecific anti-PD-1/TIM-3 antibody” or “anti-PD-1/TIM-3 antibody” refers to a molecule comprising at least one binding domain specifically binding PD-1 and at least one binding domain specifically binding TIM-3. The domains specifically binding PD-1 and TIM-3 are typically VH/VL pairs. The bispecific anti-PD-1/TIM-3 antibody may be monovalent in terms of its binding to either PD-1 or TIM-3.

“Valent” refers to the presence of a specified number of binding sites specific for an antigen in a molecule. As such, the terms “monovalent”, “bivalent”, “tetravalent”, and “hexavalent” refer to the presence of one, two, four and six binding sites, respectively, specific for an antigen in a molecule.

“An antigen specific CD4⁺ or CD8⁺ T cell” refers to a CD4⁺ or CD8⁺ T cell activated by a specific antigen, or immunostimulatory epitope thereof.

“CD137” (also called tumor necrosis factor receptor superfamily member 9, TNFRSF9, 4-1BBL) refers to a human CD137 molecule having the amino acid sequence shown in SEQ ID NO: 281.

SEQ ID NO: 281
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

“TIGIT” (also called T-cell immunoreceptor with Ig and ITIM domains) refers to human TIGIT molecule having the amino acid sequence shown in SEQ ID NO: 301.

SEQ ID NO: 301
MMTGTIETTGNISAEKGGSIILQCHLSSTTAQVTQVNWEQQDQLLAICNA
DLGWHISPSFKDRVAPGPGLGLTLQSLTVNDTGEYFCIYHTYPDGTYTGR
IFLEVLESSVAEHGARFQIPLLGAMAATLVVICTAVIVVVALTRKKKALR
IHSVEGDLRRKSAGQEEWSPSAPSPPGSCVQAEAAPAGLCGEQRGEDCAE
LHDYFNVLSYRSLGNCSFFTETG

“Agonist” refers to a molecule that, when bound to a cellular protein, induces at least one reaction or activity that is induced by a natural ligand of the protein. The molecule is an agonist when the at least one reaction or activity is induced by at least about 30%, 40%, 45%, 50%, 55%, 60%, 65%, 70%, 75%, 80%, 85%, 90%, 95%, or 100% greater than the at least one reaction or activity induced in the absence of the agonist (e.g., negative control), or when the induction is statistically significant when compared to the induction in the absence of the agonist. Agonist may be an antibody, a soluble ligand, or a small molecule. An exemplary agonist is an agonistic antibody that specifically binds a T cell activating molecule.

“Antagonist” refers to a molecule that, when bound to a cellular protein, suppresses at least one reaction or activity that is induced by a natural ligand of the protein. A molecule is an antagonist when the at least one reaction or activity is suppressed by at least about 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist. Antagonist may be an antibody, a soluble ligand, a small molecule, a DNA or RNA such as siRNA. Exemplary antagonists are an antagonistic antibody specifically binding PD-1, an antagonistic antibody specifically binding TIM-3, an antagonistic bispecific PD-1/TIM-3 antibody or an antagonistic antibody specifically binding a T cell inhibitory molecule. A typical reaction or activity that is induced by PD-1 binding to its receptor PD-L1 or PD-L2 may be reduced antigen-specific CD4⁺ or CD8⁺ cell proliferation or reduced interferon-y (IFN-γ) production by T cells, resulting in suppression of immune responses against for example tumor. A typical reaction or activity that is induced by TIM-3 binding to its receptor, such as galectin-9, may be reduced antigen specific CD4⁺ or CD8⁺ cell proliferation, reduced IFN-γ production by T cells, or reduced CD137 surface expression on CD4⁺ or CD8⁺ cells, resulting in suppression of immune responses against for example tumor. Similarly, a typical reaction or activity that is induced by a T cell inhibitory molecule is immunosuppression. Hence, an antagonistic PD-1 antibody specifically binding PD-1, an antagonistic antibody specifically binding TIM-3, an antagonistic bispecific PD-1/TIM-3 antibody, or an antagonistic antibody specifically binding a T cell inhibitory molecule induces immune responses by inhibiting the inhibitory pathways.

“Subject” includes any human or nonhuman animal. “Nonhuman animal” includes all vertebrates, e.g., mammals and non-mammals, such as nonhuman primates, sheep, dogs, cats, horses, cows chickens, amphibians, reptiles, etc. Except when noted, the terms “patient” or “subject” are used interchangeably.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated.

Conventional one and three-letter amino acid codes are used herein as shown in Table 1.

TABLE 1

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### Compositions of Matter

The present invention provides antagonistic antibodies specifically binding PD-1, antagonistic antibodies specifically binding TIM-3, and antagonistic bispecific PD-1/TIM-3 antibodies. The present invention provides polypeptides and polynucleotides encoding the antibodies of the invention or complementary nucleic acids thereof, vectors, host cells, and methods of making and using them.

### Antagonistic Antibodies Specifically Binding PD-1

PD-1, upon ligand engagement, suppresses T cell functions through multiple mechanisms (Pauken & Wherry (2015) Trends in Immunology 36(4): 265-276). PD-1 engagement directly inhibits T cell receptor (TCR) signaling through co-localization with the TCR and subsequent induction of dephosphorylation of TCR proximal signaling molecules, inhibition of Ras/MEK/ERK pathway leading to inhibition of the cell cycle progression and T cell proliferation, inhibition of cell growth and survival and reprogramming of T cell metabolism through suppression of PI3K/AKT pathway, leading to the upregulation of the BATF transcription factor, and modulation of development, maintenance and function of regulatory T cells. PD-1 has also been proposed to increase T cell motility and to limit duration of interaction between T cells and target cells, thereby reducing the extent of T cell activation (Honda et al., (2014) Immunity 40(2):235-47).

Tumors have co-opted the PD-1 pathway to downregulate T cell function in the tumor microenvironment (TME) and to evade immune destruction. In the TME, under conditions of persistent antigen and inflammation, T cells become exhausted, or dysfunctional, and progressively lose their effector function and proliferative capacity. Exhausted T cells express high levels of PD-1, often together with other inhibitory receptors such as TIM-3 or LAG-3 (Pauken & Wherry (2015) Trends in Immunology 36(4): 265-276). One of the PD-I ligands, PD-LI, is also upregulated in various tumors. PD-L1 expression occurs on the cancer cells themselves and/or infiltrating immune cells, including tumor associated macrophages, dendritic cells, fibroblasts and activated T cells (Chen et al., 2012 Clin Cancer Res 18(24):6580-7). In this setting, PD-I engagement is hypothesized to limit anti-tumor T cell responses and lead to immune evasion. Recent studies have shown that a higher frequency and level of PD-1 expression occurs on tumor infiltrating lymphocytes (TILs) in multiple solid tumors. Importantly, these PD-I TILs are functionally impaired, as evidenced by lower proliferation and effector functions (Pauken & Wherry; 2015, Trends in Immunology 36(4): 265-276) These data support the hypothesis that PD-1 mediates immune suppression in the TME.

T cell exhaustion in tumors is reversible, at least partially, by PD-I pathway blockade. Anti-PD-1/PD-L1 antibodies have been shown to enhance T cell function and lead to improved anti-tumor immunity in a number of preclinical tumor models. PD-1/PD-L1 antibodies have also shown encouraging clinical responses in multiple solid tumors, with 20-40% overall response rate (ORR) in melanoma, 10-24% in non-small cell lung cancer (NSCLC), 12-31% in renal cell carcinoma (RCC), 24-52% in bladder cancer, and 20% in head and neck cancer (Swaika et al., (2015) Mol Immunol 67(2 Pt A):4-17).

The invention provides an isolated antagonistic antibody specifically binding PD-1 or an antigen-binding portion thereof comprising a heavy chain complementarity determining region I (HCDR1), a HCDR2 and a HCDR3 of SEQ ID NOs: X₁YX₂IX₃ wherein X₁ is S or D; X₂ is V or A; and X₃ is H or S, 83 and 84, respectively, or SEQ ID NOs: X₁YX₂IX₃ wherein X₁ is S or D; X₂ is V or A; and X₃ is H or S, 83 and 85, respectively.

The invention also provides an isolated antagonistic antibody specifically binding PD-1 or an antigen-binding portion thereof comprising a light chain complementarity determining region 1 (LCDR1), a LCDR2 and a LCDR3 of SEQ ID NOs: 86, 87 and 88, respectively.

The invention also provides an isolated antagonistic antibody specifically binding PD-1 or an antigen-binding portion thereof comprising the HCDR1, the HCDR2 and the HCDR3 of SEQ ID NOs: X₁YX₂IX₃ wherein X₁ is S or D; X₂ is V or A; and X₃ is H or S, 83 and 84, respectively, and the LCDR1, the LCDR2 and the LCDR3 of SEQ ID NOs: 86, 87 and 88, respectively.

The invention also provides an isolated antagonistic antibody specifically binding PD-1 or an antigen-binding portion thereof comprising the HCDR1, the HCDR2 and the HCDR3 of SEQ ID NOs: X₁YX₂IX₃ wherein X₁ is S or D; X₂ is V or A; and X₃ is H or S, 83 and 85, respectively, and the LCDR1, the LCDR2 and the LCDR3 of SEQ ID NOs: 86, 87 and 88, respectively.

SEQ ID NOs: X₁YX₂IX₃ wherein X₁ is S or D; X₂ is V or A; and X₃ is H or S, 83, 84, 85, 86, 87 and 88 represent the HCDR1, the HCDR2, the HCDR3, the LCDR1, the LCDR2 and the LCDR3 genus sequences of affinity-matured variants of antagonistic antibodies specifically binding PD-1 having similar HCDR1, HCDR2, LCDR1, LCDR2 and LCDR3 sequences, and two similar HCDR3 groups of sequences. Antibodies within the genus bind PD-1 with the K_{D} of less than about 1×10⁻⁷ M, such as less than about 1×10-8 M, for example less than about 1×10⁻⁹ M, or for example less than about 1×10⁻¹⁰ M.

Exemplary such antibodies are antibodies having the HCDR1, the HCDR2, the HCDR3, the LCDR1, the LCDR2 and the LCDR3 amino acid sequences of antibodies PD1B114, PD1B149, PD1B160, PD1B162, PD1B164, PD1B11, PD1B183, PD1B184, PD1B185, PD1B187, PD1B71, PD1B177, PD1B70, PD1B175, PD1B194, PD1B195, PD1B196, PD1B197, PD1B198, PD1B199, PD1B200, PD1B201 and PD1B244 as described herein.
X₁YX₂IX₃,
wherein
X₁ is S or D;
X₂ is V or A; and
X₃ is H or S.

SEQ ID NO: 83
GIIPIX₄X₅TANYAQKFQG,

wherein
X₄ is Y or F; and
X₅ is G or D.

SEQ ID NO: 84
PGLAAAYDTGX₆LDY,

wherein
X₆ is N or S.

SEQ ID NO: 85
GX₇X₈X₉X₁₀TGX₁₁LDY,

wherein
X₇ is T or Y; X₈ is L or V;
X₉ is D or R;
X₁₀ is R or A; and
X₁₁ is H or M.

SEQ ID NO: 86
RASQSVX₁₂X₁₃YLA,

wherein
X₁₂ is S, R or D; and
X₁₃ is S or N.

SEQ ID NO: 87
DASX₁₄RAT,

wherein
X₁₄ is N, D, Y, S or T.

SEQ ID NO: 88
QQRX₁₅X₁₆WPLT,

wherein
X₁₅ is S, N, G, E, D, W or A; and
X₁₆ is N, Y, E or A.

In some embodiments, the isolated antagonistic antibody specifically binding PD-1 or the antigen-binding portion thereof has one, two, three, four or five of the following properties:
a) enhances an activation of antigen specific CD4⁺ or CD8⁺ T cells in a dose dependent manner, wherein the activation is measured using a cytomegalovirus antigen recall assay (CMV assay) as described in Example 1;
b) binds human PD-1 with an equilibrium dissociation constant (K_{D}) of less than about 100 nM, wherein the K_{D} is measured using ProteOn XPR36 system at +25° C.;
c) binds human PD-1 with the K_{D} of less than about 1 nM, wherein the K_{D} is measured using ProteOn XPR36 system at +25° C.;
d) binds cynomolgus PD-1 with the K_{D} of less than about 100 nM, wherein the K_{D} is measured using ProteOn XPR36 system at +25° C., or
e) binds cynomolgus PD-1 with the K_{D} of less than about 1 nM, wherein the K_{D} is measured using ProteOn XPR36 system at +25° C.

Exemplary such antibodies are PD-1 antibodies PD1B114, PD1B149, PD1B160, PD1B162, PD1B164, PD1B11, PD1B183, PD1B184, PD1B185, PD1B187, PD1B71, PD1B177, PD1B70, PD1B175, PD1B194, PD1B195, PD1B196, PD1B197, PD1B198, PD1B199, PD1B200, PD1B201 and PD1B244 as described herein.

In some embodiments, the isolated antagonistic antibody specifically binding PD-1 or the antigen-binding portion thereof enhances an activation of antigen specific CD4⁺ or CD8⁺ T cells in a dose dependent manner, wherein the activation is measured using a cytomegalovirus antigen recall assay (CMV assay) as described in Example 1, and binds human PD-1 with an equilibrium dissociation constant (K_{D}) of less than about 100 nM, wherein the K_{D} is measured using ProteOn XPR36 system at +25° C.

In some embodiments, the isolated antagonistic antibody specifically binding PD-1 or the antigen-binding portion thereof enhances an activation of antigen specific CD4⁺ or CD8⁺ T cells in dose dependent manner, wherein the activation is measured using a cytomegalovirus antigen recall assay (CMV assay) as described in Example 1, and binds human PD-1 with an equilibrium dissociation constant (K_{D}) of less than about 10 nM, wherein the K_{D} is measured using ProteOn XPR36 system at +25° C.

In some embodiments, the isolated antagonistic antibody specifically binding PD-1 or the antigen-binding portion thereof enhances an activation of antigen specific CD4 or CD8⁺ T cells in dose dependent manner, wherein the activation is measured using a cytomegalovirus antigen recall assay (CMV assay) as described in Example 1, and binds cynomolgus PD-1 with an equilibrium dissociation constant (K_{D}) of less than about 100 nM, wherein the K_{D} is measured using ProteOn XPR36 system at +25° C.

In some embodiments, the isolated antagonistic antibody specifically binding PD-1 or the antigen-binding portion thereof enhances an activation of antigen specific CD4⁺ or CD8⁺ T cells in dose dependent manner, wherein the activation is measured using a cytomegalovirus antigen recall assay (CMV assay) as described in Example 1, and binds cynomolgus PD-1 with an equilibrium dissociation constant (K_{D}) of less than about 10 nM, wherein the K_{D} is measured using ProteOn XPR36 system at +25° C.

Activation of antigen specific CD4⁺ or CD8⁺ T cells may be assessed by measuring increased T cell proliferation in a Mixed Lymphocyte Reaction (MLR) assay, increased interferon-y (IFN-γ) secretion in the MLR assay, increased TNF-α secretion in the MLR assay, increased IFN-γ secretion in a cytomegalovirus antigen assay (CMV assay) or increased TNF-α secretion in the CMV assay using known protocols and those described in Example 1. Antibodies of the invention enhance the activation of antigen specific CD4⁺ or CD8⁺ T when the measured T cell functionality is increased by the antibodies of the invention in a dose-dependent manner.

The affinity of an antibody to human or cynomolgus PD-1 may be determined experimentally using any suitable method. Such methods may utilize ProteOn XPR36, Biacore 3000 or KinExA instrumentation, ELISA or competitive binding assays known to those skilled in the art. The measured affinity of a particular antibody/PD-1 interaction may vary if measured under different conditions (e.g., osmolarity, pH). Thus, measurements of affinity and other binding parameters (e.g., K_{D}, Kₒₙ, K_{off}) are typically made with standardized conditions and a standardized buffer, such as the buffer described herein. Skilled in the art will appreciate that the internal error for affinity measurements for example using Biacore 3000 or ProteOn (measured as standard deviation, SD) may typically be within 5-33% for measurements within the typical limits of detection. Therefore the term “about” in the context of K_{D} reflects the typical standard deviation in the assay. For example, the typical SD for a K_{D} of 1×10⁻⁹ M is up to ±0.33×10⁻⁹ M.

In some embodiments, the antagonistic antibody specifically binding PD-1 or the antigen-binding portion thereof comprises the HCDR1, the HCDR2 and the HCDR3 contained within a heavy chain variable region (VH) of SEQ ID NOs: 41, 42, 43, 44, 45, 46, 47 or 48, wherein the HCDR1, the HCDR2 and the HCDR3 are defined by Chothia, Kabat, or IMGT.

In some embodiments, the antagonistic antibody specifically binding PD-1 or the antigen-binding portion thereof of the invention comprises the LCDR1, the LCDR2 and the LCDR3 contained within a light chain variable region (VL) of SEQ ID NOs: 49, 50, 51, 52, 53, 54, 55, 56, 57, 58, 59, 60, 61 or 62, wherein the LCDR1, the LCDR2 and the LCDR are defined by Chothia, Kabat, or IMGT.

In some embodiments, the antagonistic antibody specifically binding PD-1 or the antigen-binding portion thereof of the invention comprises
the HCDR1 of SEQ ID NOs: 10, 11 or 12;
the HCDR2 of SEQ ID NOs: 13, 14 or 15; and
the HCDR3 of SEQ ID NOs: 16, 17, 18 or 19.

In some embodiments, the antagonistic antibody specifically binding PD-1 or the antigen-binding portion thereof of the invention comprises
the LCDR1 of SEQ ID NOs: 20, 21, 22, 23, 24 or 25;
the LCDR2 of SEQ ID NOs: 26, 27, 28, 29 or 30; and
the LCDR3 of SEQ ID NOs: 31, 32, 33, 34, 35, 36, 37, 38, 39, or 40.

In some embodiments, the antagonistic antibody specifically binding PD-1 or the antigen-binding portion thereof of the invention comprises
the HCDR1 of SEQ ID NOs: 10, 11 or 12;
the HCDR2 of SEQ ID NOs: 13, 14 or 15;
the HCDR3 of SEQ ID NOs: 16, 17, 18 or 19;
the LCDR1 of SEQ ID NOs: 20, 21, 22, 23, 24 or 25;
the LCDR2 of SEQ ID NOs: 26, 27, 28, 29 or 30; and
the LCDR3 of SEQ ID NOs: 31, 32, 33, 34, 35, 36, 37, 38, 39 or 40.

In some embodiments, the antagonistic antibody specifically binding PD-1 or the antigen-binding portion thereof of the invention comprises the HCDR1, the HCDR2 and the HCDR3 of
SEQ ID NOs: 10, 13 and 16, respectively;
SEQ ID NOs: 10, 14 and 16, respectively;
SEQ ID NOs: 10, 13 and 17, respectively;
SEQ ID NOs: 10, 13 and 18, respectively;
SEQ ID NOs: 11, 15 and 18, respectively;
SEQ ID NOs: 10, 13 and 19, respectively;
SEQ ID NOs: 10, 14 and 17, respectively; or
SEQ ID NOs: 12, 13 and 19, respectively.

In some embodiments, the antagonistic antibody specifically binding PD-1 or the antigen-binding portion thereof of the invention comprises the LCDR1, the LCDR2 and the LCDR3 of
SEQ ID NOs: 20, 26 and 31, respectively;
SEQ ID NOs: 21, 26 and 32, respectively;
SEQ ID NOs: 22, 27 and 33, respectively;
SEQ ID NOs: 22, 26 and 34, respectively;
SEQ ID NOs: 23, 28 and 35, respectively;
SEQ ID NOs: 20, 26 and 36, respectively;
SEQ ID NOs: 21, 27 and 37, respectively;
SEQ ID NOs: 23, 26 and 32, respectively;
SEQ ID NOs: 22, 26 and 32, respectively;
SEQ ID NOs: 24, 26 and 38, respectively;
SEQ ID NOs: 20, 29 and 39, respectively;
SEQ ID NOs: 20, 30 and 32, respectively;
SEQ ID NOs: 25, 26 and 40, respectively; or
SEQ ID NOs: 24, 26 and 32, respectively.

The invention also provides an antagonistic antibody specifically binding PD-1 or an antigen-binding portion thereof comprising the HCDR1, the HCDR2, the HCDR3, the LCDR1, the LCDR2 and the LCDR3 of SEQ ID NOs: 10, 14, 17, 23, 26 and 32, respectively.

In some embodiments, the antibody or the antigen-binding portion thereof binds human PD-1 with an equilibrium dissociation constant (K_{D}) of less than about 100 nM, optionally less than about 10 nM, for example less than about 1 nM such as less than about 500 pM, wherein the K_{D} is measured using ProteOn XPR36 system at +25° C.

In some embodiments, the antibody or the antigen-binding portion thereof binds cynomolgous PD-1 with an equilibrium dissociation constant (K_{D}) of less than about 100 nM, optionally less than about 10 nM, for example less than about 1 nM such as less than about 500 pM, wherein the K_{D} is measured using ProteOn XPR36 system at +25° C.

In some embodiments, the antibody or the antigen-binding portion thereof comprises the VH of SEQ ID NO: 48 and the VL of SEQ ID NO: 56.

In some embodiments, the VH and the VL are encoded by polynucleotide sequences of SEQ ID NOs: 196 and 197, respectively.

In some embodiments, the antibody is an IgG4 isotype, optionally comprising a S228P substitution when compared to the wild type IgG4.

In some embodiments, the antibody is an IgG4/x isotype, optionally comprising the S228P substitution when compared to the wild type IgG4.

In some embodiments, the antibody comprises the VH of SEQ ID NO: 48 and the VL of SEQ ID NO: 56 and is an IgG4 isotype, optionally comprising the S228P substitution when compared to the wild type IgG4.

In some embodiments, the antibody comprises the VH of SEQ ID NO: 48 and the VL of SEQ ID NO: 56 and is an IgG4/x isotype comprising the S228P substitution when compared to the wild type IgG4.

In some embodiments, the antibody comprises a heavy chain (HC) of SEQ ID NO: 72 and a light chain (LC) of SEQ ID NO: 73.

In some embodiments, the antibody is an IgG2 isotype, optionally comprising V234A, G237A, P238S, H268A, V309L, A330S and P331S substitutions when compared to the wild type IgG2.

In some embodiments, the antibody is an IgG2/κ isotype, optionally comprising V234A, G237A, P238S, H268A, V309L, A330S and P331S substitutions when compared to the wild type IgG2.

In some embodiments, the antibody comprises the VH of SEQ ID NO: 48 and the VL of SEQ ID NO: 56 and is an IgG2/κ isotype, optionally comprising V234A, G237A, P238S, H268A, V309L, A330S and P331S substitutions when compared to the wild type IgG2.

In some embodiments, the antibody comprises the VH of SEQ ID NO: 48 and the VL of SEQ ID NO: 56 and is an IgG2/κ isotype comprising V234A, G237A, P238S, H268A, V309L, A330S and P331S substitutions when compared to the wild type IgG2.

In some embodiments, the antibody is an IgG1 isotype.

In some embodiments, the antibody is an IgG3 isotype.

In some embodiments, the antibody is a bispecific antibody, such as a bispecific PD-1/TIM-3 antibody.

The antibody is suitable for use in therapy, for example in treating cancer.

The antibody is suitable for use in therapy, for example in treating a solid tumor.

The antibody is suitable for use in therapy, for example in treating a melanoma.

The antibody is suitable for use in therapy, for example in treating a lung cancer.

The antibody is suitable for use in therapy, for example in treating non-small cell lung cancer (NSCLC).

The antibody is suitable for use in therapy, for example in treating a squamous NSCLC.

The antibody is suitable for use in therapy, for example in treating a non-squamous NSCLC.

The antibody is suitable for use in therapy, for example in treating a lung adenocarcinoma.

The antibody is suitable for use in therapy, for example in treating a renal cell carcinoma (RCC).

The antibody is suitable for use in therapy, for example in treating a mesothelioma.

The antibody is suitable for use in therapy, for example in treating a nasopharyngeal carcinoma (NPC).

The antibody is suitable for use in therapy, for example in treating a colorectal cancer.

The antibody is suitable for use in therapy, for example in treating a prostate cancer.

The antibody is suitable for use in therapy, for example in treating a castration-resistant prostate cancer.

The antibody is suitable for use in therapy, for example in treating a stomach cancer.

The antibody is suitable for use in therapy, for example in treating an ovarian cancer.

The antibody is suitable for use in therapy, for example in treating a gastric cancer.

The antibody is suitable for use in therapy, for example in treating a liver cancer.

The antibody is suitable for use in therapy, for example in treating a pancreatic cancer.

The antibody is suitable for use in therapy, for example in treating a thyroid cancer.

The antibody is suitable for use in therapy, for example in treating a squamous cell carcinoma of the head and neck.

The antibody is suitable for use in therapy, for example in treating a carcinomas of the esophagus or gastrointestinal tract.

The antibody is suitable for use in therapy, for example in treating a breast cancer. The antibody is suitable for use in therapy, for example in treating a fallopian tube cancer.

The antibody is suitable for use in therapy, for example in treating a brain cancer.

The antibody is suitable for use in therapy, for example in treating an urethral cancer.

The antibody is suitable for use in therapy, for example in treating an endometriosis.

The antibody is suitable for use in therapy, for example in treating a cervical cancer.

The antibody is suitable for use in therapy, for example in treating a metastatic lesion of the cancer.

The antibody is suitable for use in therapy, for example in treating a hematological malignancy.

The antibody is suitable for use in therapy, for example in treating a non-Hodgkin's lymphoma.

The antibody is suitable for use in therapy, for example in treating a chronic lymphocytic leukemia.

The antibody is suitable for use in therapy, for example in treating a cancer, in combination with an antagonistic antibody specifically binding TIM-3.

The antibody is suitable for use in therapy, for example in treating a cancer, in combination with an antagonistic antibody specifically binding TIM-3 comprising the VH of SEQ ID NO: 146 and the VL of SEQ ID NO: 156.

The antibody is suitable for use in therapy, for example in treating a cancer, in combination with an antagonistic antibody specifically binding TIM-3 comprising the VH of SEQ ID NO: 145 and the VL of SEQ ID NO: 155.

The antibody is suitable for use in therapy, for example in treating a cancer, in combination with an antagonistic antibody specifically binding TIM-3 comprising the VH of SEQ ID NO: 172 and the VL of SEQ ID NO: 173.

The antibody is suitable for use in therapy, for example in treating cancer, such as a solid tumor, in combination with a FGFR inhibitor.

The antibody is suitable for use in therapy, for example in treating cancer, such as a solid tumor, in combination with a vaccine.

The antibody is suitable for use in therapy, for example in treating cancer, such as a solid tumor, in combination with an agonistic antibody specifically binding GITR (SEQ ID NO: 271).

The antibody is suitable for use in therapy, for example in treating cancer, such as a solid tumor, in combination with an agonistic antibody specifically binding CD137 (SEQ ID NO: 281).

The antibody is suitable for use in therapy, for example in treating cancer, such as a solid tumor, in combination with an agonistic antibody specifically binding OX-40 (SEQ ID NO: 279).

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 66, 67, 68, 69, 70 and 71, respectively.

In some embodiments, the antibody comprises the VH of SEQ ID NO: 64 and the VL of SEQ ID NO: 65.

In some embodiments, the VH and the VL are encoded by polynucleotide sequences of SEQ ID NOs: 198 and 199, respectively.

In some embodiments, the antibody comprises the VH of SEQ ID NO: 63 and the VL of SEQ ID NO: 65.

In some embodiments, the antibody or the antigen-binding portion thereof binds human PD-1 with an equilibrium dissociation constant (K_{D}) of less than about 100 nM, optionally less than about 10 nM, for example less than about 1 nM such as less than about 100 pM, wherein the K_{D} is measured using ProteOn XPR36 system at +25° C.

In some embodiments, the antibody is an IgG4 isotype, optionally comprising a S228P substitution when compared to the wild type IgG4.

In some embodiments, the antibody is an IgG4/x isotype, optionally comprising the S228P substitution when compared to the wild type IgG4.

In some embodiments, the antibody comprises the VH of SEQ ID NO: 64 and the VL of SEQ ID NO: 65 and is an IgG4 isotype, optionally comprising the S228P substitution when compared to the wild type IgG4.

In some embodiments, the antibody comprises the VH of SEQ ID NO: 64 and the VL of SEQ ID NO: 65 and is an IgG4x isotype, comprising the S228P substitution when compared to the wild type IgG4.

In some embodiments, the antibody comprises the HC of SEQ ID NO: 74 and the LC of SEQ ID NO: 75.

In some embodiments, the antibody is an IgG2 isotype, optionally comprising V234A, G237A, P238S, H268A, V309L, A330S and P331S substitutions when compared to the wild type IgG2.

In some embodiments, the antibody is an IgG2/κ isotype, optionally comprising V234A, G237A, P238S, H268A, V309L, A330S and P331S substitution when compared to the wild type IgG2.

In some embodiments, the antibody comprises the VH of SEQ ID NO: 64 and the VL of SEQ ID NO: 65 and is an IgG2/κ isotype, optionally comprising V234A, G237A, P238S, H268A, V309L, A330S and P331S substitution when compared to the wild type IgG2.

In some embodiments, the antibody comprises the VH of SEQ ID NO: 64 and the VL of SEQ ID NO: 65 and is an IgG2/κ isotype comprising V234A, G237A, P238S, H268A, V309L, A330S and P331S substitution when compared to the wild type IgG2.

In some embodiments, the antibody is an IgG1 isotype.

In some embodiments, the antibody is an IgG3 isotype.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The antibody is suitable for use in therapy, for example in treating a melanoma.

The antibody is suitable for use in therapy, for example in treating a lung cancer.

The antibody is suitable for use in therapy, for example in treating non-small cell lung cancer (NSCLC).

The antibody is suitable for use in therapy, for example in treating a squamous NSCLC.

The antibody is suitable for use in therapy, for example in treating a non-squamous NSCLC.

The antibody is suitable for use in therapy, for example in treating a lung adenocarcinoma.

The antibody is suitable for use in therapy, for example in treating a renal cell carcinoma (RCC).

The antibody is suitable for use in therapy, for example in treating a mesothelioma.

The antibody is suitable for use in therapy, for example in treating a nasopharyngeal carcinoma (NPC).

The antibody is suitable for use in therapy, for example in treating a colorectal cancer.

The antibody is suitable for use in therapy, for example in treating a prostate cancer.

The antibody is suitable for use in therapy, for example in treating a castration-resistant prostate cancer.

The antibody is suitable for use in therapy, for example in treating a stomach cancer.

The antibody is suitable for use in therapy, for example in treating an ovarian cancer.

The antibody is suitable for use in therapy, for example in treating a gastric cancer.

The antibody is suitable for use in therapy, for example in treating a liver cancer.

The antibody is suitable for use in therapy, for example in treating a pancreatic cancer.

The antibody is suitable for use in therapy, for example in treating a thyroid cancer.

The antibody is suitable for use in therapy, for example in treating a squamous cell carcinoma of the head and neck.

The antibody is suitable for use in therapy, for example in treating a carcinomas of the esophagus or gastrointestinal tract.

The antibody is suitable for use in therapy, for example in treating a breast cancer.

The antibody is suitable for use in therapy, for example in treating a fallopian tube cancer.

The antibody is suitable for use in therapy, for example in treating a brain cancer.

The antibody is suitable for use in therapy, for example in treating an urethral cancer.

The antibody is suitable for use in therapy, for example in treating an endometriosis.

The antibody is suitable for use in therapy, for example in treating a cervical cancer.

The antibody is suitable for use in therapy, for example in treating a metastatic lesion of the cancer.

The antibody is suitable for use in therapy, for example in treating a hematological malignancy.

The antibody is suitable for use in therapy, for example in treating a non-Hodgkin's lymphoma.

The antibody is suitable for use in therapy, for example in treating a chronic lymphocytic leukemia.

The antibody is suitable for use in therapy, for example in treating a cancer, in combination with an antagonistic antibody specifically binding TIM-3.

The antibody is suitable for use in therapy, for example in treating a cancer, in combination with an antagonistic antibody specifically binding TIM-3 comprising the VH of SEQ ID NO: 146 and the VL of SEQ ID NO: 156.

The antibody is suitable for use in therapy, for example in treating a cancer, in combination with an antagonistic antibody specifically binding TIM-3 comprising the VH of SEQ ID NO: 145 and the VL of SEQ ID NO: 155.

The antibody is suitable for use in therapy, for example in treating a cancer, in combination with an antagonistic antibody specifically binding TIM-3 comprising the VH of SEQ ID NO: 172 and the VL of SEQ ID NO: 173.

The antibody is suitable for use in therapy, for example in treating cancer, such as a solid tumor, in combination with a FGFR inhibitor.

The antibody is suitable for use in therapy, for example in treating cancer, such as a solid tumor, in combination with a vaccine.

The antibody is suitable for use in therapy, for example in treating cancer, such as a solid tumor, in combination with an agonistic antibody specifically binding GITR (SEQ ID NO: 271).

The antibody is suitable for use in therapy, for example in treating cancer, such as a solid tumor, in combination with an agonistic antibody specifically binding CD137 (SEQ ID NO: 281).

The antibody is suitable for use in therapy, for example in treating cancer, such as a solid tumor, in combination with an agonistic antibody specifically binding OX-40 (SEQ ID NO: 279).

The invention also provides an antagonistic antibody specifically binding PD-1 or an antigen-biding portion thereof, comprising the HCDR1, the HCDR2, the HCDR3, the LCDR1, the LCDR2 and the LCDR3 of SEQ ID NOs: 12, 13, 19, 24, 26 and 38, respectively.

In some embodiments, the antibody or the antigen-biding portion thereof comprises the VH of SEQ ID NO: 47 and the VL of SEQ ID NO: 58.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-biding portion thereof comprising the HCDR1, the HCDR2, the HCDR3, the LCDR1, the LCDR2 and the LCDR3 of SEQ ID NOs: 11, 15, 18, 20, 30 and 32, respectively.

In some embodiments, the antibody or the antigen-binding portion thereof comprises the VH of SEQ ID NO: 45 and the VL of SEQ ID NO: 60.

In some embodiments, the VH and the VL are encoded by polynucleotide sequences of SEQ ID NOs: 202 and 203, respectively.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody comprises the HC of SEQ ID NO: 76 and the LC of SEQ ID NO: 77.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor. The invention also provides an antagonistic antibody specifically binding PD-1 or an antigen-biding portion thereof, comprising the HCDR1, the HCDR2, the HCDR3, the LCDR1, the LCDR2 and the LCDR3 of SEQ ID NOs: 10, 13, 16, 20, 26 and 31, respectively.

In some embodiments, the antibody or the antigen-biding portion thereof comprises the VH of SEQ ID NO: 41 and the VL of SEQ ID NO: 49.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody comprises the HC of SEQ ID NO: 212 and the LC of SEQ ID NO: 213.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-biding portion thereof, comprising the HCDR1, the HCDR2, the HCDR3, the LCDR1, the LCDR2 and the LCDR3 of SEQ ID NOs: 10, 13, 16, 21, 26 and 32, respectively.

In some embodiments, the antibody or the antigen-biding portion thereof comprises the VH of SEQ ID NO: 41 and the VL of SEQ ID NO: 50.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody comprises the HC of SEQ ID NO: 214 and the LC of SEQ ID NO: 215.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-biding portion thereof, comprising the HCDR1, the HCDR2, the HCDR3, the LCDR1, the LCDR2 and the LCDR3 of SEQ ID NOs: 10, 14, 16, 22, 27 and 33, respectively.

In some embodiments, the antibody or the antigen-binding portion thereof comprises the VH of SEQ ID NO: 42 and the VL of SEQ ID NO: 51.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody comprises the HC of SEQ ID NO: 216 and the LC of SEQ ID NO: 217.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-biding portion thereof, comprising the HCDR1, the HCDR2, the HCDR3, the LCDR1, the LCDR2 and the LCDR3 of SEQ ID NOs: 10, 14, 16, 22, 26 and 34, respectively.

In some embodiments, the antibody or the antigen-biding portion thereof comprises the VH of SEQ ID NO: 42 and the VL of SEQ ID NO: 52.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody comprises the HC of SEQ ID NO: 218 and the LC of SEQ ID NO: 219.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 10, 14, 16, 23, 28 and 35, respectively.

In some embodiments, the antibody or the antigen-binding portion thereof comprises the VH of SEQ ID NO: 42 and the VL of SEQ ID NO: 53.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody comprises the HC of SEQ ID NO: 220 and the LC of SEQ ID NO: 221.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 10, 13, 17, 20, 26 and 31, respectively.

In some embodiments, the antibody or the antigen-binding portion thereof comprises the VH of SEQ ID NO: 43 and the VL of SEQ ID NO: 49.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 10, 13, 17, 20, 26 and 36, respectively.

In some embodiments, the antibody or the antigen-binding portion thereof comprises the VH of SEQ ID NO: 43 and the VL of SEQ ID NO: 54.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody comprises the HC of SEQ ID NO: 222 and the LC of SEQ ID NO: 223.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 10, 13, 17, 21, 26 and 32, respectively.

In some embodiments, the antibody or the antigen-binding portion thereof comprises the VH of SEQ ID NO: 43 and the VL of SEQ ID NO: 50.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody comprises the HC of SEQ ID NO: 224 and the LC of SEQ ID NO: 225.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR1, the HCDR2, the HCDR3, the LCDR1, the LCDR2 and the LCDR3 of SEQ ID NOs: 10, 13, 17, 21, 27 and 37, respectively.

In some embodiments, the antibody or the antigen-binding portion thereof comprises the VH of SEQ ID NO: 43 and the VL of SEQ ID NO: 55.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody comprises the HC of SEQ ID NO: 226 and the LC of SEQ ID NO: 227.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 10, 13, 17, 23, 26 and 32, respectively.

In some embodiments, the antibody or the antigen-binding portion thereof comprises the VH of SEQ ID NO: 43 and the VL of SEQ ID NO: 56.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 10, 13, 17, 22, 26 and 32, respectively.

In some embodiments, the antibody comprises the VH of SEQ ID NO: 43 and the VL of SEQ ID NO: 57.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody comprises the HC of SEQ ID NO: 228 and the LC of SEQ ID NO: 229.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 10, 13, 18, 20, 26 and 31, respectively.

In some embodiments, the antibody or the antigen-binding portion thereof comprises the VH of SEQ ID NO: 44 and the VL of SEQ ID NO: 49.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 11, 15, 18, 20, 26 and 31, respectively.

In some embodiments, the antibody or the antigen-binding portion thereof comprises the VH of SEQ ID NO: 45 and the VL of SEQ ID NO: 49.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 10, 13, 19, 20, 26 and 31, respectively.

In some embodiments, the antibody or the antigen-binding portion thereof comprises the VH of SEQ ID NO: 46 and the VL of SEQ ID NO: 49.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 12, 13, 19, 20, 26 and 31, respectively.

In some embodiments, the antibody or the antigen-binding portion thereof comprises the VH of SEQ ID NO: 47 and the VL of SEQ ID NO: 49.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 10, 14, 17, 23, 28 and 35, respectively.

In some embodiments, the antibody comprises the VH of SEQ ID NO: 48 and the VL of SEQ ID NO: 53.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 10, 14, 17, 22, 26 and 34, respectively.

In some embodiments, the antibody or the antigen-binding portion thereof comprises the VH of SEQ ID NO: 48 and the VL of SEQ ID NO: 52.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 12, 13, 19, 20, 29 and 39, respectively.

In some embodiments, the antibody or the antigen-binding portion thereof comprises the VH of SEQ ID NO: 47 and the VL of SEQ ID NO: 59.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 11, 15, 18, 25, 26 and 40, respectively.

In some embodiments, the antibody or the antigen-binding portion thereof comprises the VH of SEQ ID NO: 45 and the VL of SEQ ID NO: 61.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 11, 15, 18, 24, 26 and 32, respectively.

In some embodiments, the antibody of the antigen-binding portion thereof comprises the VH of SEQ ID NO: 45 and the VL of SEQ ID NO: 62.

In some embodiments, the antibody is an IgG1 isotype.

In some embodiments, the antibody is an IgG2 isotype.

In some embodiments, the antibody is an IgG2 isotype comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype.

In some embodiments, the antibody is an IgG4 isotype comprising the S228P substitution when compared to the wild type IgG4.

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The invention also provides an antagonistic antibody specifically binding PD-1 or an antigen-binding portion thereof, comprising the VH of SEQ ID NOs: 41, 42, 43, 44, 45, 46, 47, 48, 63 or 64.

The invention also provides an antagonistic antibody specifically binding PD-1 or an antigen-binding portion thereof, comprising the VL of SEQ ID NOs: 49, 50, 51, 52, 53, 54, 55, 56, 57, 58, 59, 60, 61, 62 or 65.

The invention also provides an antagonistic antibody specifically binding PD-1 or an antigen-binding portion thereof, comprising the VH of SEQ ID NOs: 41, 42, 43, 44, 45, 46, 47, 48, 63 or 64 and the VL of SEQ ID NOs: 49, 50, 51, 52, 53, 54, 55, 56, 57, 58, 59, 60, 61, 62 or 65.

The VH, the VL, the HCDR and the LCDR sequences of exemplary antagonistic antibodies specifically binding PD-1 of the invention are shown in Table 2.

Although the embodiments illustrated in the Examples comprise pairs of variable regions, one from a heavy chain and one from a light chain, a skilled artisan will recognize that alternative embodiments may comprise single heavy or light chain variable regions. The single variable region may be used to screen for variable domains capable of forming a two-domain specific antigen-binding fragment capable of, for example, binding to human PD-1. The screening may be accomplished by phage display screening methods using for example hierarchical dual combinatorial approach disclosed in Int. Patent Publ. No. WO1992/01047. In this approach, an individual colony containing either a VH or a VL chain clone is used to infect a complete library of clones encoding the other chain (VL or VH), and the resulting two-chain specific antigen-binding domain is selected in accordance with phage display techniques using known methods and those described herein. Therefore, the individual VH and VL polypeptide chains are useful in identifying additional antibodies specifically binding to human PD-1 using the methods disclosed in Int. Patent Publ. No. WO1992/01047.

In some embodiments, the antagonistic antibody specifically binding PD-1 is a multispecific antibody.

In some embodiments, the antagonistic antibody specifically binding PD-1 is a bispecific antibody.

In some embodiments, antagonistic bispecific antibody specifically binding PD-1 binds PD-L1 (SEQ ID NO: 5), PD-L2 (SEQ ID NO: 8), LAG-3 (SEQ ID NO: 293), TIM-3 (SEQ ID NO: 138), CEACAM-1 (SEQ ID NO: 296), CEACAM-5 (SEQ ID NO: 307), OX-40 (SEQ ID NO: 279), GITR (SEQ ID NO: 271), CD27 (SEQ ID NO: 280), VISTA (SEQ ID NO: 286), CD137 (SEQ ID NO: 281), TIGIT (SEQ ID NO: 301) or CTLA-4 (SEQ ID NO: 292). Bispecific and multispecific antibodies may be generated using methods described herein.

TABLE 2

| | SEQ ID NO: | | | | | | | |
|---|---|---|---|---|---|---|---|---|
| Antibody | HCDR1 | HCDR2 | HCDR3 | LCDR1 | LCDR2 | LCDR3 | VH | VL |
| PD1B114 | 10 | 13 | 16 | 20 | 26 | 31 | 41 | 49 |
| PD1B149 | 10 | 13 | 16 | 21 | 26 | 32 | 41 | 50 |
| PD1B160 | 10 | 14 | 16 | 22 | 27 | 33 | 42 | 51 |
| PD1B162 | 10 | 14 | 16 | 22 | 26 | 34 | 42 | 52 |
| PD1B164 | 10 | 14 | 16 | 23 | 28 | 35 | 42 | 53 |
| PD1B11 | 10 | 13 | 17 | 20 | 26 | 31 | 43 | 49 |
| PD1B183 | 10 | 13 | 17 | 20 | 26 | 36 | 43 | 54 |
| PD1B184 | 10 | 13 | 17 | 21 | 26 | 32 | 43 | 50 |
| PD1B185 | 10 | 13 | 17 | 21 | 27 | 37 | 43 | 55 |
| PD1B187 | 10 | 13 | 17 | 23 | 26 | 32 | 43 | 56 |
| PD1B192 | 10 | 13 | 17 | 22 | 26 | 32 | 43 | 57 |
| PD1B71 | 10 | 13 | 18 | 20 | 26 | 31 | 44 | 49 |
| PD1B177 | 11 | 15 | 18 | 20 | 26 | 31 | 45 | 49 |
| PD1B70 | 10 | 13 | 19 | 20 | 26 | 31 | 46 | 49 |
| PD1B175 | 12 | 13 | 19 | 20 | 26 | 31 | 47 | 49 |
| PD1B194 | 10 | 14 | 17 | 23 | 28 | 35 | 48 | 53 |
| PD1B195 | 10 | 14 | 17 | 22 | 26 | 34 | 48 | 52 |
| PD1B196 | 10 | 14 | 17 | 23 | 26 | 32 | 48 | 56 |
| PD1B197 | 12 | 13 | 19 | 24 | 26 | 38 | 47 | 58 |
| PD1B198 | 12 | 13 | 19 | 20 | 29 | 39 | 47 | 59 |
| PD1B199 | 11 | 15 | 18 | 20 | 30 | 32 | 45 | 60 |
| PD1B200 | 11 | 15 | 18 | 25 | 26 | 40 | 45 | 61 |
| PD1B201 | 11 | 15 | 18 | 24 | 26 | 32 | 45 | 62 |
| PD1B131 | 66 | 67 | 68 | 69 | 70 | 71 | 63 | 65 |
| PD1B132 | 66 | 67 | 68 | 69 | 70 | 71 | 64 | 65 |

### Homologous Antibodies

Variants of the antagonistic antibodies specifically binding PD-1 or the antigen-binding portion thereof of the invention comprising the VH, the VL or the VH and the VL amino acid sequences shown in Table 2, Table 21 and Table 22 are within the scope of the invention. For example, variants may comprise one, two, three, four, five, six, seven, eight, nine, ten, eleven, twelve, thirteen, fourteen or fifteen amino acid substitutions in the VH and/or the VL as long as the homologous antibodies retain or have improved functional properties when compared to the parental antibodies. In some embodiments, the sequence identity may be about 90%, 91%, 92%, 93%, 94%, 95%, 96%, 97%, 98% or 99% to a VH or the VL amino acid sequence of the invention. Optionally, any variation of the variant compared to the parental antibody is not within the CDRs of the variant.

The invention also provides an antagonistic antibody specifically binding PD-1 or an antigen-binding portion thereof, comprising the VH of SEQ ID NOs: 41, 42, 43, 44, 45, 46, 47, 48, 63 or 64, the VH optionally having one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding PD-1 or an antigen-binding portion thereof, comprising the VL of SEQ ID NOs: 49, 50, 51, 52, 53, 54, 55, 56, 57, 58, 59, 60, 61, 62 or 65, the VL optionally having one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding PD-1 or an antigen-binding portion thereof, comprising the VH of SEQ ID NO: 48 and the VL of SEQ ID NO: 56, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding PD-1 or an antigen-binding portion thereof, comprising the VH of SEQ ID NO: 64 and the VL of SEQ ID NO: 65, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding PD-1 or an antigen-binding portion thereof, comprising
the VH of SEQ ID NO: 41 and the VL of SEQ ID NO: 49;
the VH of SEQ ID NO: 41 and the VL of SEQ ID NO: 50;
the VH of SEQ ID NO: 42 and the VL of SEQ ID NO: 51;
the VH of SEQ ID NO: 42 and the VL of SEQ ID NO: 52;
the VH of SEQ ID NO: 42 and the VL of SEQ ID NO: 53;
the VH of SEQ ID NO: 43 and the VL of SEQ ID NO: 49;
the VH of SEQ ID NO: 43 and the VL of SEQ ID NO: 54;
the VH of SEQ ID NO: 43 and the VL of SEQ ID NO: 50;
the VH of SEQ ID NO: 43 and the VL of SEQ ID NO: 55;
the VH of SEQ ID NO: 43 and the VL of SEQ ID NO: 56;
the VH of SEQ ID NO: 43 and the VL of SEQ ID NO: 57;
the VH of SEQ ID NO: 44 and the VL of SEQ ID NO: 49;
the VH of SEQ ID NO: 45 and the VL of SEQ ID NO: 49;
the VH of SEQ ID NO: 46 and the VL of SEQ ID NO: 49;
the VH of SEQ ID NO: 47 and the VL of SEQ ID NO: 49;
the VH of SEQ ID NO: 48 and the VL of SEQ ID NO: 53;
the VH of SEQ ID NO: 48 and the VL of SEQ ID NO: 52;
the VH of SEQ ID NO: 47 and the VL of SEQ ID NO: 58;
the VH of SEQ ID NO: 47 and the VL of SEQ ID NO: 59;
the VH of SEQ ID NO: 45 and the VL of SEQ ID NO: 60;
the VH of SEQ ID NO: 45 and the VL of SEQ ID NO: 61;
the VH of SEQ ID NO: 45 and the VL of SEQ ID NO: 62; or
the VH of SEQ ID NO: 63 and the VL of SEQ ID NO: 65, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding PD-1 or an antigen-binding portion thereof, comprising the VH having the amino acid sequence at least 90%, 91%, 92%, 93%, 94%, 95%, 96%, 97%, 98% or 99% identical to the VH of SEQ ID NOs: 41, 42, 43, 44, 45, 46, 47, 48, 64 or 65. Optionally, any variation from the sequences of the SEQ ID NOs is not within the CDRs.

The invention also provides an antagonistic antibody specifically binding PD-1 or an antigen-binding portion thereof, comprising the VL having the amino acid sequence at least 90%, 91%, 92%, 93%, 94%, 95%, 96%, 97%, 98% or 99% identical to the VL of SEQ ID NOs: 49, 50, 51, 52, 53, 54, 55, 56, 57, 58, 59, 60, 61, 62 or 65. Optionally, any variation from the sequences of the SEQ ID NOs is not within the CDRs.

The invention also provides an antagonistic antibody specifically binding PD-1 or an antigen-binding portion thereof, comprising the VH having the amino acid sequence at least 90%, 91%, 92%, 93%, 94%, 95%, 96%, 97%, 98% or 99% identical to the VH of SEQ ID NOs: 41, 42, 43, 44, 45, 46, 47, 48, 63 or 64 and the VL having the amino acid sequence at least 90%, 91%, 92%, 93%, 94%, 95%, 96%, 97%, 98% or 99% identical to the VL of SEQ ID NOs: 49, 50, 51, 52, 53, 54, 55, 56, 57, 58, 59, 60, 61, 62 or 65. Optionally, any variation from the sequences of the SEQ ID NOs is not within the CDRs.

The invention also provides an antagonistic antibody specifically binding PD-1 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 48 and the VL of SEQ ID NO: 56. Optionally, any variation from the sequences of the SEQ ID NOs is not within the CDRs.

The invention also provides an antagonistic antibody specifically binding PD-1 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 64 and the VL of SEQ ID NO: 65. Optionally, any variation from the sequences of the SEQ ID NOs is not within the CDRs.

The invention also provides an antagonistic antibody specifically binding PD-1 or an antigen-binding portion thereof, comprising the VH and the VL having the amino acid sequences at least 90%, 91%, 92%, 93%, 94%, 95%, 96%, 97%, 98% or 99% identical to the VH and the VL of SEQ ID NOs:
41 and 49, respectively;
41 and 50, respectively;
42 and 51, respectively;
42 and 52, respectively;
42 and 53, respectively;
43 and 49, respectively;
43 and 54, respectively;
43 and 50, respectively;
43 and 55, respectively;
43 and 56, respectively;
43 and 57, respectively;
44 and 49, respectively;
45 and 49, respectively;
46 and 49, respectively;
47 and 49, respectively;
48 and 53, respectively;
48 and 52, respectively;
47 and 58, respectively;
47 and 59, respectively;
45 and 60, respectively;
45 and 61, respectively;
45 and 62, respectively; or
63 and 65, respectively. Optionally, any variation from the sequences of the SEQ ID NOs is not within the CDRs.

The homologous antagonistic antibodies specifically binding PD-1 or the antigen-binding portions thereof of the invention have one, two, three, four or five of the following properties:
a) enhance an activation of antigen specific CD4⁺ or CD8⁺ T cells in a dose dependent manner, wherein the activation is measured using a cytomegalovirus antigen recall assay (CMV assay) as described in Example 1;
b) bind human PD-1 with an equilibrium dissociation constant (K_{D}) of less than about 100 nM, wherein the K_{D} is measured using ProteOn XPR36 system at +25° C.;
c) bind human PD-1 with the K_{D} of less than about 1 nM, wherein the K_{D} is measured using ProteOn XPR36 system at +25° C.;
d) bind cynomolgus PD-1 of SEQ ID NO: 3 with the K_{D} of less than about 100 nM, wherein the K_{D} is measured using ProteOn XPR36 system at +25° C., or e) bind cynomolgus PD-1 of SEQ ID NO: 3 with the K_{D} of less than about 1 nM, wherein the K_{D} is measured using ProteOn XPR36 system at +25° C.

In some embodiments, the antibody enhances activation of antigen specific CD4⁺ or CD8⁺ T cells in a dose dependent manner, wherein activation is measured using a cytomegalovirus antigen recall assay (CMV assay) as described in Example 1, and binds human PD-1 with an equilibrium dissociation constant (K_{D}) of less than about 100 nM, wherein the K_{D} is measured using ProteOn XPR36 system at +25° C.

In some embodiments, the antibody enhances activation of antigen specific CD4⁺ or CD8⁺ T cells in dose dependent manner, wherein activation is measured using a cytomegalovirus antigen recall assay (CMV assay) as described in Example 1, and binds human PD-1 with an equilibrium dissociation constant (K_{D}) of less than about 10 nM, wherein the K_{D} is measured using ProteOn XPR36 system at +25° C.

The percent identity between the two sequences is a function of the number of identical positions shared by the sequences (i.e., % identity=number of identical positions/total number of positions x 100), taking into account the number of gaps, and the length of each gap, which need to be introduced for optimal alignment of the two sequences.

The percent identity between two amino acid sequences may be determined using the algorithm of E. Meyers and W. Miller (Comput Appl Biosci 4:11-17 (1988)) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch (J Mol Biol 48:444-453 (1970)) algorithm which has been incorporated into the GAP program in the GCG software package (available at http://_www_gcg_com), using either a Blossum 62 matrix or a PAM250 matrix, and a gap weight of 16, 14, 12, 10, 8, 6, or 4 and a length weight of 1, 2, 3, 4, 5, or 6.

Antibodies with Conservative Modifications

The invention also provides antagonistic antibodies specifically binding PD-1 or antigen-binding portions thereof comprising the VH comprising the HCDR1, the HCDR2 and the HCDR3 sequences and the VL comprising the LCDR1, the LCDR2 and the LCDR3 sequences, wherein one or more of the CDR sequences comprise specified amino acid sequences based on the antibodies described herein (e.g., antibodies shown in Table 2, Table 21 and Table 22, or conservative modifications thereof, and wherein the antibodies retain the desired functional properties of the parental antagonistic antibodies specifically binding PD-1 of the invention.

The antibodies with conservative modifications have one, two, three, four or five of the following properties:
a) enhance an activation of antigen specific CD4⁺ or CD8⁺ T cells in dose dependent manner, wherein the activation is measured using a cytomegalovirus antigen recall assay (CMV assay) as described in Example 1;
b) bind human PD-1 with an equilibrium dissociation constant (K_{D}) of less than about 100 nM, wherein the K_{D} is measured using ProteOn XPR36 system at +25° C.;
c) bind human PD-1 with the K_{D} of less than about 1 nM, wherein the K_{D} is measured using ProteOn XPR36 system at +25° C.;
d) bind cynomolgus PD-1 of SEQ ID NO: 3 with the K_{D} of less than about 100 nM, wherein the K_{D} is measured using ProteOn XPR36 system at +25° C., or
e) bind cynomolgus PD-1 of SEQ ID NO: 3 with the K_{D} of less than about 1 nM, wherein the K_{D} is measured using ProteOn XPR36 system at +25° C.

In some embodiments, the antibody enhances activation of antigen specific CD4⁺ or CD8⁺ T cells in dose dependent manner, wherein activation is measured using a cytomegalovirus antigen recall assay (CMV assay) as described in Example 1, and binds human PD-1 with an equilibrium dissociation constant (K_{D}) of less than about 100 nM, wherein the K_{D} is measured using ProteOn XPR36 system at +25° C.

In some embodiments, the antibody enhances activation of antigen specific CD4⁺ or CD8⁺ T cells in dose dependent manner, wherein activation is measured using a cytomegalovirus antigen recall assay (CMV assay) as described in Example 1, and binds human PD-1 with an equilibrium dissociation constant (K_{D}) of less than about 10 nM, wherein the K_{D} is measured using ProteOn XPR36 system at +25° C.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 10, 14, 17, 23, 26 and 32, respectively, and conservative modifications thereof.

The invention also provides an antagonistic antibody specifically binding PD-1 or an antigen-binding portion thereof, comprising the HCDR1, the HCDR2, the HCDR3, the LCDR1, the LCDR2 and the LCDR3 of SEQ ID NOs: 66, 67, 68, 69, 70 and 71, respectively, and conservative modifications thereof.

The invention also provides an antagonistic antibody specifically binding PD-1 or an antigen-binding portion thereof, comprising the HCDR1, the HCDR2, the HCDR3, the LCDR1, the LCDR2 and the LCDR3 of
SEQ ID NOs: 10, 13, 16, 20, 26 and 31, respectively;
SEQ ID NOs: 10, 13, 16, 21, 26 and 32, respectively;
SEQ ID NOs: 10, 14, 16, 22, 27 and 33, respectively;
SEQ ID NOs: 10, 14, 16, 22, 26 and 34, respectively;
SEQ ID NOs: 10, 14, 16, 23, 28 and 35, respectively;
SEQ ID NOs: 10, 13, 17, 20, 26 and 31, respectively;
SEQ ID NOs: 10, 13, 17, 20, 26 and 36, respectively;
SEQ ID NOs: 10, 13, 17, 21, 26 and 32, respectively;
SEQ ID NOs: 10, 13, 17, 21, 27 and 37, respectively;
SEQ ID NOs: 10, 13, 17, 23, 26 and 32, respectively;
SEQ ID NOs: 10, 13, 17, 22, 26 and 32, respectively;
SEQ ID NOs: 10, 13, 18, 20, 26 and 31, respectively;
SEQ ID NOs: 11, 15, 18, 20, 26 and 31, respectively;
SEQ ID NOs: 10, 13, 19, 20, 26 and 31, respectively;
SEQ ID NOs: 12, 13, 19, 20, 26 and 31, respectively;
SEQ ID NOs: 10, 14, 17, 23, 28 and 35, respectively;
SEQ ID NOs: 10, 14, 17, 22, 26 and 34, respectively;
SEQ ID NOs: 12, 13, 19, 24, 26 and 38, respectively;
SEQ ID NOs: 12, 13, 19, 20, 29 and 39, respectively;
SEQ ID NOs: 11, 15, 18, 20, 30 and 32, respectively;
SEQ ID NOs: 11, 15, 18, 25, 26 and 40, respectively;
SEQ ID NOs: 11, 15, 18, 24, 26 and 32, respectively, and conservative modifications thereof.

“Conservative modification” refers to amino acid modifications that do not significantly affect or alter the binding characteristics of the antibody containing the amino acid sequences. Conservative modifications include amino acid substitutions, additions and deletions. Conservative substitutions are those in which the amino acid is replaced with an amino acid residue having a similar side chain. The families of amino acid residues having similar side chains are well defined and include amino acids with acidic side chains (for example, aspartic acid, glutamic acid), basic side chains (for example, lysine, arginine, histidine), nonpolar side chains (for example, alanine, valine, leucine, isoleucine, proline, phenylalanine, methionine), uncharged polar side chains (for example, glycine, asparagine, glutamine, cysteine, serine, threonine, tyrosine, tryptophan), aromatic side chains (for example, phenylalanine, tryptophan, histidine, tyrosine), aliphatic side chains (for example, glycine, alanine, valine, leucine, isoleucine, serine, threonine), amide (for example, asparagine, glutamine), beta-branched side chains (for example, threonine, valine, isoleucine) and sulfur-containing side chains (cysteine, methionine). Furthermore, any native residue in the polypeptide may also be substituted with alanine, as has been previously described for alanine scanning mutagenesis (MacLennan et al., Acta Physiol. Scand. Suppl. 643:55-67, 1998; Sasaki et al., Adv. Biophys. 35:1-24, 1998). Amino acid substitutions to the antibodies of the invention may be made by well-known methods for example by PCR mutagenesis (U.S. Pat. No. 4,683,195). Alternatively, libraries of variants may be generated using known methods, for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be tested for their characteristics using assays described herein.

### Antagonistic Antibodies Specifically Binding TIM-3

T-cell immunoglobulin domain and mucin domain 3 (TIM-3, also known as Hepatitis A virus cellular receptor 2 (HAVCR2)) is a co-inhibitory immune checkpoint receptor that has been proposed to negatively regulate both adaptive and innate immune responses. TIM-3 is expressed on specific subsets of CD4 and CD8⁺ T cells and functions to limit the duration and magnitude of T cell responses.

Multiple lines of evidence support the inhibitory role of TIM-3 in regulating T cell responses. Tim-3-deficient mice exhibit defects in the induction of both antigen-specific and transplantation tolerance, consistent with TIM-3 inhibiting effector T cells during normal immune responses (Sabatos et al., (2003) Nat Immunol 4(11):1102-1110, Sanchez-Fueyo et al., (2003) Nat Immunol 4(11):1093-1101). Anti-TIM-3 antibodies exacerbate experimental autoimmune encephalomyelitis (EAE) in animal models (Monney et al., (2002) Nature 415(6871):536-541). TIM-3 has been shown to be a critical driver of the dysfunctional or exhausted T cell state that occurs in chronic infection and cancer (Sakuishi, K. and A. C. Anderson (2014). Tim-3 Regulation of Cancer Immunity. Tumor-Induced Immune Suppression. D. I. Gabrilovich and A. A. Hurwitz, Springer New York: 239-261).

Blockade of TIM-3 has been shown to restore activity in effector cells, such as cytokine secretion and proliferation. In virally exhausted cell populations, e.g., cells infected with HCV, TIM-3-expressing cells (TIM-3Y cells) express less TNF-α and IFN-γ cytokines than TIM-3 negative cells in both effector cell populations, CD4 and CD8⁺ T cells (Golden-Mason et al., (2009) J Virol 83:9122). Blockade of TIM-3 restored proliferation in CD8⁺ T cells from an HIV patient, or in cells that recapitulated viral exhaustion (Jones et al., (2008) J Exp Med 205:2763), or proliferation and IFN-γ and/or TNF-α secretion in NY-ESO-1 specific T cells from PBMCs from metastatic patients (Fourcade et al., (2010) J Exp Med 207:2175). TIM-3⁺ T cells have been found to be concentrated in tumors, and contribute to the immunosuppressive tumor environment (Sakuishi et al., (2013) Oncoimmunology, 2:e23849).

Blockade of TIM-3 (partially alone and additively or synergistically in combination with PD-1 pathway blockade) has shown anti-tumor efficacy in several preclinical cancer models, including CT26 colon carcinoma (Sakuishi et al., (2010) J Exp Med 207(10):2187-94), WT3 sarcoma and TRAMP-C1 prostate carcinoma (Ngiow et al., (2011) Cancer Res 71(10):3540-3551).

The mechanisms through which TIM-3 inhibits T cell responses are not fully understood. The cytoplasmic tail of TIM-3 contains multiple tyrosine residues (Ferris et al., (2014) J Immunol 193(4): 1525-1530) but lacks inhibitory signaling motifs such as ITIMs or ITSMs that are found in the PD-1 intracellular tail. The Src family tyrosine kinases Fyn and Lek have been shown to bind to TIM-3, although the exact consequences of these interactions remain to be confirmed in vivo. Two opposing models have been proposed for how TIM-3 regulates T cell signaling. On one hand, TIM-3 has been postulated to negatively regulate TCR signaling by recruiting a phosphatase to the immunological synapse, and de-phosphorylating Lek (Clayton, et al., (2014) J Immunol 192(2):782-791). In contrast, TIM-3 has also been proposed to enhance TCR signaling and paradoxically drive T cells towards a more exhausted state, through increased activation of NFAT activity and NFKB signaling.

In addition to expression on effector T cells, TIM-3 is also expressed on regulatory T cells (T-regs) and has been shown to mark a suppressive T-reg subset in tumors. Analyses using both primary human cells and mouse preclinical models have shown that TIM-3⁺ T-regs are more effective at inhibiting T helper1 (Th1) and T helper 17 (Th17) T cell responses than TIM-3⁻ T-regs (Gautron et al., (2014) Eur J Immunol 44(9): 2703-2711; Sakuishi et al., (2013) Oncoimmunology, 2:e23849). Since TIM-3 is expressed on highly suppressive Tregs, it can directly inhibit CD4⁺ and CD8⁺ T cell responses. In addition, TIM-3⁺ Tregs express high levels of IL-10, which has been proposed to drive exhaustion of effector T cells in the TME as an additional indirect mechanism of suppressing anti-tumor immune responses (Sakuishi et al., (2013) Oncoimmunology, 2:e23849).

TIM-3 is expressed on several innate immune cell types, including monocytes/macrophages, dendritic cells, and NK cells. Existing data are consistent with a suppressive role for TIM-3 in these different cell types.

TIM-3 is constitutively expressed by circulating CD14⁺ monocytes in healthy donors, and its expression on peripheral monocytes is significantly increased in patients with chronic inflammation and cancer (Rong et al., (2014) Tissue Antigens 83(2):76-81). TIM-3 levels are also upregulated on macrophages that infiltrate hepatocellular carcinoma (HCC) tumors, compared to macrophages from adjacent tissues, and is proposed to play a role in driving the polarization of macrophages to an M2 tumor-promoting phenotype.

Recently, TIM-3 was reported to be expressed on dendritic cells that infiltrate mouse tumors. In this setting, interaction of TIM-3 with HMBG1 was proposed to suppress innate immunity by interfering with the recognition of and response to immunostimulatory nucleic acid (Chiba et al., (2012) Immunol 13(9): 832-842). TIM-3 is also constitutively expressed on NK cells in peripheral blood. A recent study showed that NK cells from advanced melanoma patients express high levels of TIM-3 on peripheral NK cells. Importantly, TIM-3Y NK cells were functionally exhausted and anti-TIM-3 blockade was able to reverse the exhaustion and enhance NK cell functionality (da Silva et al., (2014) Cancer Immunol Res 2(5): 410-422).

TIM-3 binds ligands galectin-9 (Gal-9), phosphatidylserine (PtdSer), HMGB1 and CEACAM-1. S-type lectin galectin-9 can inhibit TIM-3-associated Th1 effector function and induce apoptosis on TIM-3-expressing T cells in murine models. PtdSer usually resides on the intracellular side of the plasma membrane, but is flipped to the extracellular side during apoptosis. PtdSer binds a preserved cleft in all three human TIM family members (TIM-1, 3, 4). Inhibition of PtdSer binding to TIM-3 may activate T-cell response. Galectin-9 is secreted by tumor cells and can contribute to evasion from anti-tumor immunity. DNA alarmin HMGB1, for which TIM-3 may act as a “sink,” can prevent the HMGB1/RAGE interactions that stimulate innate immunity. CEACAM-1 can interact with TIM-3 both in cis as a heterodimer on T cells and in trans as a ligand. Interaction between CEACAM-1 and TIM-3 may help mediate block immune response signaling. Co-blockade of TIM-3 and CEACAM-1 in CT26 colon carcinoma showed similar efficacy to that seen for co-blockade of PD-L1 and TIM-3.

Thus, blockade of TIM-3 using the antibodies of the invention described herein that inhibit TIM-3 function may improve the immune response against infection and anti-tumor immunity.

The invention also provides an isolated antagonistic antibody specifically binding TIM-3 or an antigen-binding portion thereof, wherein the antibody inhibits binding of TIM-3 to galectin-9.

Inhibition of binding of TIM-3 to galectin-9 by the antibodies of the invention may be assessed using competition ELISA. In an exemplary assay, 1 μg/ml recombinant human Fc-TIM-3 is bound on wells of microtiter plates, the wells are washed and blocked, and 10 μg/ml of the test antibody is added. Without washing, 7.5 μg/ml galectin-9 is added into the wells and incubated for 30 min, after which 0.5 μg/ml anti-galectin-9-biotin antibody is added and incubated for 30 min. The plates are washed and 0.5 μg/mL neutravidin-HRP conjugate polyclonal antibody is added and incubated for 30 minutes. The plates are washed and POD Chemiluminescence substrate added immediately prior to reading the luminescence signal. Antibodies of the invention inhibit binding of TIM-3 to galectin-9 when the binding of galectin-9 is reduced by at least about 50%, 55%, 60%, 65%, 70%, 75%, 80%, 85%, 90%, 95% or 100% using an assay described herein and in Example 1. Exemplary antibodies that inhibit TIM-3 binding to galectin-9 are antibodies TM3B103, TM3B105, TM3B107, TM3B108, TM3B109, TM3B113, TM3B189, TM3B190 and TM3B196.

In some embodiments, the antagonistic antibody specifically binding TIM-3 or the antigen-binding portion thereof enhances activation of antigen specific CD4⁺ or CD8⁺ T cells.

In some embodiments, the antagonistic antibody specifically binding TIM-3 or the antigen-binding portion thereof enhances an activation of antigen specific CD4⁺ or CD8⁺ T cells, wherein the activation of antigen-specific CD4⁺ or CD8⁺ T cells is assessed by measuring a statistically significant enhancement of CD137 surface expression on antigen specific CD4⁺ or CD8⁺ T cells according to methods described in Example 14.

Use of CD137 as a marker of antigen specific CD8⁺ and CD4⁺ T cells that expand in response to CMV antigen stimulation allowed the detection of the functional effects of the antagonistic TIM-3 antibodies of the invention.

In some embodiments, the antagonistic antibody specifically binding TIM-3 or the antigen-binding portion thereof binds TIM-3 within TIM-3 residues 32-47 (WGKGACPVFECGNVVL) (SEQ ID NO: 261).

In some embodiments, the antagonistic antibody specifically binding TIM-3 or the antigen-binding portion thereof binds TIM-3 within TIM-3 residues 32-47 (WGKGACPVFECGNVVL) (SEQ ID NO: 261) and residues 50-56 (DERDVNY) (SEQ ID NO: 262).

In some embodiments, the antagonistic antibody specifically binding TIM-3 or the antigen-binding portion thereof binds TIM-3 within TIM-3 residues 90-102 (RIQIPGIMNDEKF) (SEQ ID NO: 263).

In some embodiments, the antagonistic antibody specifically binding TIM-3 or the antigen-binding portion thereof binds TIM-3 within TIM-3 residues 90-102 (RIQIPGIMNDEKF) (SEQ ID NO: 263) and residues 50-56 (DERDVNY) SEQ ID NO: 262.

“Within” means that 80% or more of the epitope residues the antibody binds to reside within the recited amino acid stretches, and that up to 20% of the epitope residues the antibody binds to reside outside of the recited amino acid stretches.

The Tim-3 epitope the antibody binds to may be resolved for example using hydrogen/deuterium exchange (H/D exchange) or by analyzing a crystal structure of the antibody in complex with TIM-3. The epitope residues are those which are protected by the antibody by at least 5% difference in deuteration levels through H/D exchange or those surface exposed amino acid residues determined to bind the antibody in a crystal structure of a complex of the antibody and TIM-3. In the crystal structure of a complex of the antibody and TIM-3, the epitope residues are those TIM-3 residues that reside within 4 A distance or less from any of the antibody CDR residues.

In an H/D exchange assay, TIM-3 protein is incubated in the presence or absence of the antibody in deuterated water for predetermined times resulting in deuterium incorporation at exchangeable hydrogen atoms which are unprotected by the antibody, followed by protease digestion of the protein and analyses of the peptide fragments using LC-MS. In an exemplary assay, 5 μL of the test antibody (10 μg) or 5 μL of the complex of TIM-3 and the test antibody (10 and 7.35 μg, respectively) is incubated with 120 μL deuterium oxide labeling buffer (50 mM phosphate, 100 mM sodium chloride at pH 7.4) for 0 sec, 60 sec, 300 sec, 1800 sec, 7200 sec, and 14400 sec. Deuterium exchange is quenched by adding 63 μL of 5 M guanidine hydrochloride and final pH is 2.5. The quenched sample is subjected to on-column pepsin/protease type XIII digestion and LC-MS analysis. For pepsin/protease type XIII digestion, 5 μg of the samples in 125 μL control buffer (50 mM phosphate, 100 mM sodium chloride at pH 7.4) are denatured by adding 63 μL of 5 M guanidine hydrochloride (final pH is 2.5) and incubating the mixture for 3 min. Then, the mixture is subjected to on-column pepsin/protease type XIII digestion and the resultant peptides analyzed using an UPLC-MS system comprised of a Waters Acquity UPLC coupled to a Q Exactive™ Hybrid Quadrupole-Orbitrap Mass Spectrometer (Thermo). Raw MS data is processed using HDX WorkBench, software for the analysis of H/D exchange MS data. The deuterium levels are calculated using the average mass difference between the deuteriated peptide and its native form (t₀). Peptide identification is done through searching MS/MS data against the TIM-3 sequence with Mascot. The mass tolerance for the precursor and product ions is 20 ppm and 0.05 Da, respectively.

For X-ray crystallography, TIM-3 and the test antibody are expressed and purified using standard protocols. The TIM-3/test antibody complex is incubated overnight at 4° C., concentrated, and separated from the uncomplexed species using size-exclusion chromatography. The complex is crystallized by the vapor-diffusion method from various known test solutions for example solutions containing PEG3350, ammonium citrate and 2-(N-Morpholino)ethanesulfonic acid (MES).

Antibodies binding within Tim-3 residues 32-47 (WGKGACPVFECGNVVL) (SEQ ID NO: 261), 90-102 (RIQIPGIMNDEKF) (SEQ ID NO: 263) and/or 50-56 (DERDVNY) (SEQ ID NO: 262) may be generated by isolating antibodies binding TIM-3 using phage display libraries, selecting those antibodies that compete with the reference antibody TM3B105 (VH of SEQ ID NO: 146 and VL of SEQ ID NO: 156) or TM3B291 (VH of SEQ ID NO: 172 and VL of SEQ ID NO: 173) for binding to TIM-3 by 100%, and confirming the epitope of the generated antibodies by solving the crystal structure of the antibody/TIM-3 complex. Alternatively, mice, rats or rabbits may be immunized using peptides encompassing residues 32-47, 90-102 and/or 50-56 of TIM-3 and the generated antibodies may be evaluated for their binding within the recited region.

The invention also provides an antagonistic antibody specifically binding TIM-3 or an antigen-binding portion thereof comprising a heavy chain complementarity determining region 1 (HCDR1), a HCDR2 and a HCDR3 of SEQ ID NOs: X₁₇YX₁₈MX₁₉ wherein X₁₇ is N, S, G or D; X₁₈ is W or A; and X₁₉ is S or H, 165 and X₂₄X₂₅X₂₆X₂₇X₂₈X₂₉X₃₀X₃₁DY wherein X₂₄ is D, S, N, G or E; X₂₅ is H, P, E, T or L; X₂₆ is W, E, N or deleted; X₂₇ is D, P or deleted; X₂₈ is P, Y, D or deleted; X₂₉ is N, A, D, G or deleted; X₃₀ is F, P, R, W or V; and X₃₁ is L or F, respectively.

The invention also provides an antagonistic antibody specifically binding TIM-3 or an antigen-binding portion thereof comprising a light chain complementarity determining region 1 (LCDR1), LCDR2 and LCDR3 of SEQ ID NOs: 167, X₄₃ASX₄₄RX₄₅X₄₆ wherein X₄₃ is G, D, W or T; X₄₄ is S, N or T; X₄₅ is A or E; and X₄₆ is T or S and 169 respectively.

The invention also provides an antagonistic antibody specifically binding TIM-3 or an antigen-binding portion thereof comprising the HCDR1, the HCDR2 and the HCDR3 of SEQ ID NOs: X₁₇YX₁₈MX₁₉ wherein X₁₇ is N, S, G or D; X₁₈ is W or A; and X₁₉ is S or H, 165 and X₂₄X₂₅X₂₆X₂₇X₂₈X₂₉X₃₀X₃₁DY wherein X₂₄ is D, S, N, G or E; X₂₅ is H, P, E, T or L; X₂₆ is W, E, N or deleted; X₂₇ is D, P or deleted; X₂₈ is P, Y, D or deleted; X₂₉ is N, A, D, G or deleted; X₃₀ is F, P, R, W or V; and X₃₁ is L or F, respectively, and the LCDR1, the LCDR2 and the LCDR3 of SEQ ID NOs: 167, X₄₃ASX₄₄RX₄₅X₄₆ wherein X₄₃ is G, D, W or T; X₄₄ is S, N or T; X₄₅ is A or E; and X₄₆ is T or S and 169 respectively.

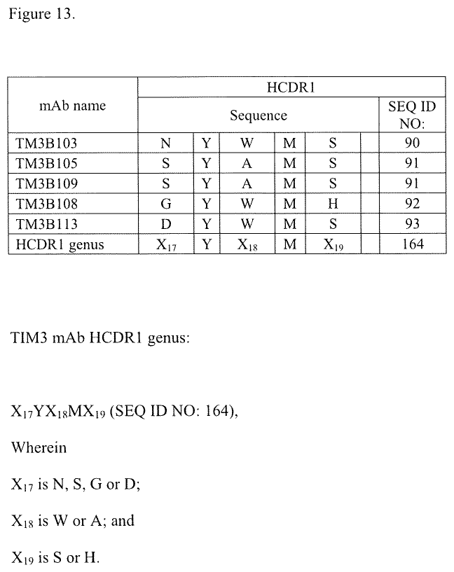

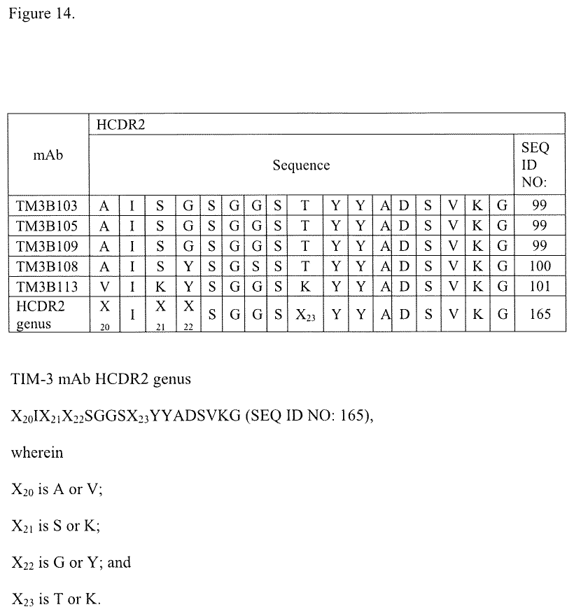

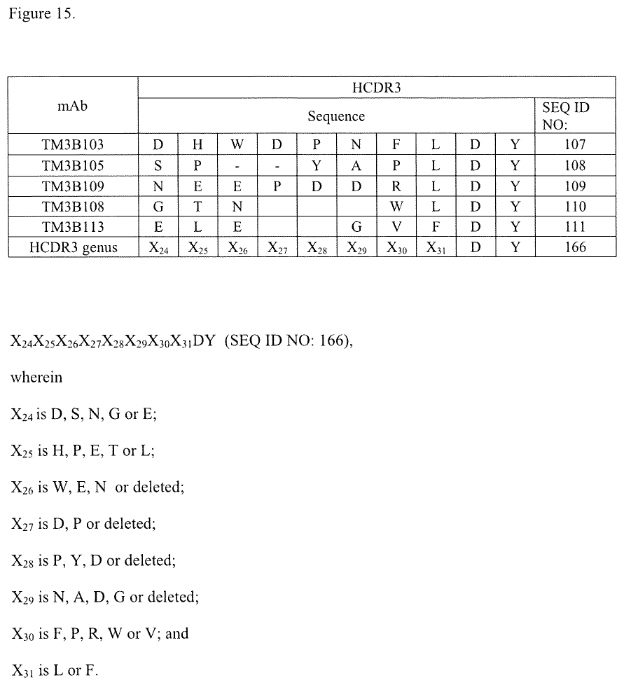

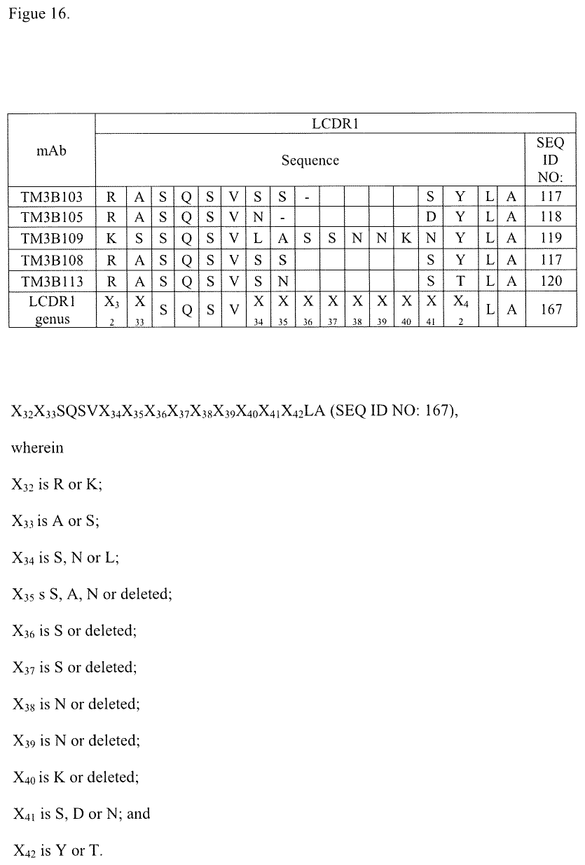

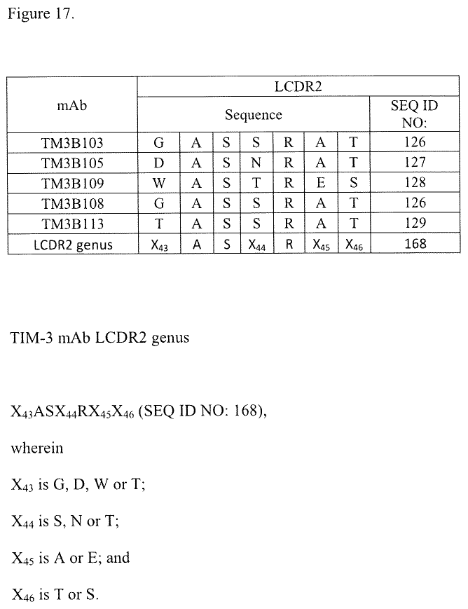

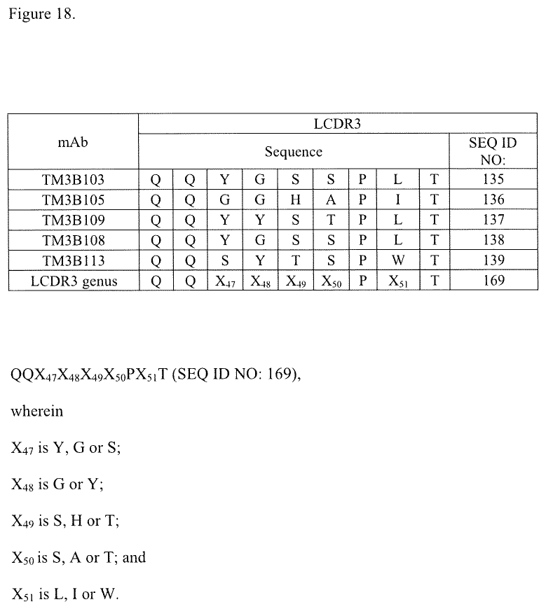

SEQ ID NOs: X₁₇YX₁₈MX₁₉ wherein X₁₇ is N, S, G or D; X₁₈ is W or A; and X₁₉ is S or H, 165, X₂₄X₂₅X₂₆X₂₇X₂₈X₂₉X₃₀X₃₁DY wherein X₂₄ is D, S, N, G or E; X₂₅ is H, P, E, T or L; X₂₆ is W, E, N or deleted; X₂₇ is D, P or deleted; X₂₈ is P, Y, D or deleted; X₂₉ is N, A, D, G or deleted; X₃₀ is F, P, R, W or V; and X₃₁ is L or F, 167, X₄₃ASX₄₄RX₄₅X₄₆ wherein X₄₃ is G, D, W or T; X₄₄ is S, N or T; X₄₅ is A or E; and X₄₆ is T or S and 169 represent the HCDR1, the HCDR2, the HCDR3, the LCDR1, the LCDR2 and the LCDR3 genus sequences of TIM-3 antagonists derived from phage display libraries. The genus sequences were generated based on structural models that resulted in the sequence alignments given in FIG. 13, FIG. 14, FIG. 15, FIG. 16, FIG. 17 and FIG. 18 and summarized herein.
X₁₇YX₁₈MX₁₉,
wherein
X₁₇ is N, S, G or D;
X₁₈ is W or A; and
X₁₉ is S or H.

SEQ ID NO: 165
X₂₀IX₂₁X₂₂SGGSX₂₃YYADSVKG,

wherein
X₂₀ is A or V;
X₂₁ is S or K;
X₂₂ is G or Y; and
X₂₃ is T or K.

X₂₄X₂₅X₂₆X₂₇X₂₈X₂₉X₃₀X₃₁DY,
wherein
X₂₄ is D, S, N, G or E;
X₂₅ is H, P, E, T or L;
X₂₆ is W, E, N or deleted;
X₂₇ is D, P or deleted;
X₂₈ is P, Y, D or deleted;
X₂₉ is N, A, D, G or deleted;
X₃₀ is F, P, R, W or V; and
X₃₁ is L or F.

SEQ ID NO: 167
X₃₂X₃₃SQSVX₃₄X₃₅X₃₆X₃₇X₃₈X₃₉X₄₀X₄₁X₄₂LA,

wherein
X₃₂ is R or K;
X₃₃ is A or S;
X₃₄ is S, N or L;
X₃₅ is S, A, N or deleted;
X₃₆ is S or deleted;
X₃₇ is S or deleted;
X₃₈ is N or deleted;
X₃₉ is N or deleted;
X₄₀ is K or deleted;
X₄₁ is S, D or N; and
X₄₂ is Y or T.

X₄₃ASX₄₄RX₄₅X₄₆,
wherein
X₄₃ is G, D, W or T;
X₄₄ is S, N or T;
X₄₅ is A or E; and
X₄₆ is T or S.

SEQ ID NO: 169
(SEQ ID NO: 169)
QQX₄₇X₄₈X₄₉X₅₀PX₅₁T,

wherein
X₄₇ is Y, G or S;
X₄₈ is G or Y;
X₄₉ is S, H or T;
X₅o is S, A or T; and
X₅₁ is L, I or W.

The invention also provides an antagonistic antibody specifically binding TIM-3 or an antigen-binding portion thereof comprising the HCDR1, the HCDR2 and the HCDR3 contained within a heavy chain variable region (VH) of SEQ ID NOs: 145, 146, 147, 148 or 149, wherein the HCDR1, the HCDR2 and the HCDR3 are defined by Chothia, Kabat, or IMGT.

The invention also provides an antagonistic antibody specifically binding TIM-3 or an antigen-binding portion thereof comprising the LCDR1, the LCDR2 and the LCDR3 contained within a light chain variable region (VL) of SEQ ID NOs: 155, 156, 157 or 158, wherein the LCDR1, the LCDR2 and the LCDR3 are defined by Chothia, Kabat, or IMGT.

In some embodiments, the antagonistic antibody specifically binding TIM-3 or the antigen-binding portion thereof comprises
the HCDR1 of SEQ ID NOs: 90, 91, 92 or 93;
the HCDR2 of SEQ ID NOs: 99, 100 or 101; and
the HCDR3 of SEQ ID NOs: 107, 108, 109, 110 or 111.

In some embodiments, the antagonistic antibody specifically binding TIM-3 or the antigen-binding portion thereof of the invention comprises
the LCDR1 of SEQ ID NOs: 117, 118, 119 or 120;
the LCDR2 of SEQ ID NOs: 126, 127, 128 or 129; and
the LCDR3 of SEQ ID NOs: 135, 136, 137 or 139.

In some embodiments, the antagonistic antibody specifically binding TIM-3 or the antigen-binding portion thereof comprises
the HCDR1 of SEQ ID NOs: 90, 91, 92 or 93;
the HCDR2 of SEQ ID NOs: 99, 100 or 101;
the HCDR3 of SEQ ID NOs: 107, 108, 109, 110 or 111;
the LCDR1 of SEQ ID NOs: 117, 118, 119 or 120;
the LCDR2 of SEQ ID NOs: 126, 127, 128 or 129; or
the LCDR3 of SEQ ID NOs: 135, 136, 137 or 139.

In some embodiments, the antagonistic antibody specifically binding TIM-3 or the antigen-binding portion thereof comprises the HCDR1, the HCDR2 and the HCDR3 of
SEQ ID NOs: 90, 99 and 107, respectively;
SEQ ID NOs: 91, 99 and 108, respectively;
SEQ ID NOs: 91, 99 and 109, respectively;
SEQ ID NOs: 92, 100 and 110, respectively; or
SEQ ID NOs: 93, 101 and 111, respectively;

In some embodiments, the antagonistic antibody specifically binding TIM-3 or the antigen-binding portion thereof comprises the LCDR1, the LCDR2 and the LCDR3 of
SEQ ID NOs: 117, 126 and 135, respectively;
SEQ ID NOs: 118, 127 and 136, respectively;
SEQ ID NOs: 119, 128 and 137, respectively; or
SEQ ID NOs: 120, 129 and 139, respectively.

In some embodiments, the antagonistic antibody specifically binding TIM-3 or the antigen-binding portion thereof comprises the HCDR1, the HCDR2, the HCDR3, the LCDR1, the LCDR2 and the LCDR3 of
SEQ ID NOs: 90, 99, 107, 117, 126 and 135, respectively;
SEQ ID NOs: 91, 99, 108, 118, 127 and 136, respectively;
SEQ ID NOs: 91, 99, 109, 119, 128 and 137, respectively;
SEQ ID NOs: 92, 100, 110, 117, 126 and 135, respectively; or
SEQ ID NOs: 93, 101, 111, 120, 129 and 139, respectively.

The invention also provides an isolated antagonistic antibody specifically binding TIM-3 or an antigen-binding portion thereof, comprising the HCDR1, the HCDR2 and, the HCDR3 of SEQ ID NOs: X₁₇YX₁₈MX₁₉ wherein X₁₇ is N, S, G or D; X₁₈ is W or A; and X₁₉ is S or H, 165 and 108, respectively, and the LCDR1, the LCDR2 and the LCDR3 of SEQ ID NOs: 118, X₄₃ASX₄₄RX₄₅X₄₆ wherein X₄₃ is G, D, W or T; X₄₄ is S, N or T; X₄₅ is A or E; and X₄₆ is T or S and 169 respectively.

The invent ion also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 91, 99, 108, 118, 127 and 136, respectively.

In some embodiments, the antibody specifically binding TIM-3 or the antigen-binding portion thereof binds TIM-3 within TIM-3 residues 32-47 (WGKGACPVFECGNVVL) (SEQ ID NO: 261).

In some embodiments, the antibody specifically binding TIM-3 or the antigen-binding portion thereof binds TIM-3 within TIM-3 residues 32-47 (WGKGACPVFECGNVVL) (SEQ ID NO: 261) and residues 50-56 (DERDVNY) SEQ ID NO: 262.

In some embodiments, the antibody specifically binding TIM-3 or the antigen-binding portion thereof inhibits binding of TIM-3 to galectin-9.

In some embodiments, the antibody or the antigen-binding portion thereof comprises a heavy chain framework derived from IGHV3-23 (SEQ ID NO: 174) and a light chain framework derived from IGKV3-11 (SEQ ID NO: 171).

In some embodiments, the antibody or the antigen-binding portion thereof comprises the VH of SEQ ID NO: 146 and the VL of SEQ ID NO: 156.

In some embodiments, the VH and the VL are encoded by polynucleotide sequences of SEQ ID NOs: 204 and 205, respectively.

In some embodiments, the antibody or the antigen-binding portion thereof enhances activation of antigen specific CD4⁺ or CD8⁺ T cells, wherein activation of antigen-specific CD4⁺ or CD8⁺ T cells is assessed by measuring a statistically significant enhancement of CD137 surface expression on antigen specific CD4⁺ or CD8⁺ T cells according to methods described in Example 14.

In some embodiments, the antibody is an IgG1 isotype.

In some embodiments, the antibody is an IgG3 isotype.

In some embodiments, the antibody is an IgG4 isotype, optionally comprising a S228P substitution when compared to the wild type IgG4.

In some embodiments, the antibody is an IgG4/x isotype, optionally comprising the S228P substitution when compared to the wild type IgG4.

In some embodiments, the antibody comprises the VH of SEQ ID NO: 146 and the VL of SEQ ID NO: 156 and is an IgG4 isotype, optionally comprising the S228P substitution when compared to the wild type IgG4.

In some embodiments, the antibody comprises the VH of SEQ ID NO: 146 and the VL of SEQ ID NO: 156 and is an IgG4K isotype comprising the S228P substitution when compared to the wild type IgG4.

In some embodiments, the antibody is an IgG2 isotype, optionally comprising V234A, G237A, P238S, H268A, V309L, A330S and P33IS substitutions when compared to the wild type IgG2.

In some embodiments, the antibody is an IgG2/K isotype, optionally comprising V234A, G237A, P238S, H268A, V309L, A330S and P33IS substitutions when compared to the wild type IgG2.

In some embodiments, the antibody comprises the VH of SEQ ID NO: 146 and the VL of SEQ ID NO: 156 and is an IgG2/K isotype, optionally comprising V234A, G237A, P238S, H268A, V309L, A330S and P33IS substitutions when compared to the wild type IgG2.

In some embodiments, the antibody comprises the VH of SEQ ID NO: 146 and the VL of SEQ ID NO: 156 and is an IgG2/K isotype comprising V234A, G237A, P238S, H268A, V309L, A330S and P331S substitutions when compared to the wild type IgG2.

In some embodiments, the antibody comprises the HC of SEQ ID NO: 78 and the LC of SEQ ID NO: 79.

In some embodiments, the antibody comprises the HC of SEQ ID NO: 240 and the LC of SEQ ID NO: 79.

SEQ ID NO: 78
EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKSP
YAPLDYWGQGTLVTVSSASTKGPSVFPLAPCSRSTSESTAALGCLVKDYF
PEPVTVSWNSGALTSGVHTFPAVLQSSGLYSLSSVVTVPSSNFGTQTYTC
NVDHKPSNTKVDKTVERKCCVECPPCPAPPAAASSVFLFPPKPKDTLMIS
RTPEVTCVVVDVSAEDPEVQFNWYVDGVEVHNAKTKPREEQFNSTFRVVS
VLTVLHQDWLNGKEYKCKVSNKGLPSSIEKTISKTKGQPREPQVYTLPPS
REEMTKNQVSLTCLVKGFYPSDIAVEWESNGQPENNYKTTPPMLDSDGSF
FLYSKLTVDKSRWQQGNVFSCSVMHEALHNHYTQKSLSLSPGK
SEQ ID NO: 79
EIVLTQSPATLSLSPGERATLSCRASQSVNDYLAWYQQKPGQAPRLLIYD
ASNRATGIPARFSGSGSGTDFTLTISSLEPEDFAVYYCQQGGHAPITFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 240
EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKSP
YAPLDYWGQGTLVTVSSASTKGPSVFPLAPCSRSTSESTAALGCLVKDYF
PEPVTVSWNSGALTSGVHTFPAVLQSSGLYSLSSVVTVTSSNFGTQTYTC
NVDHKPSNTKVDKTVERKCCVECPPCPAPPAAASSVFLFPPKPKDTLMIS
RTPEVTCVVVDVSAEDPEVQFNWYVDGVEVHNAKTKPREEQFNSTFRVVS
VLTVLHQDWLNGKEYKCKVSNKGLPSSIEKTISKTKGQPREPQVYTLPPS
REEMTKNQVSLTCLVKGFYPSDIAVEWESNGQPENNYKTTPPMLDSDGSF
FLYSKLTVDKSRWQQGNVFSCSVMHEALHNHYTQKSLSLSPGK

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The antibody is suitable for use in therapy, for example in treating a melanoma.

The antibody is suitable for use in therapy, for example in treating a lung cancer.

The antibody is suitable for use in therapy, for example in treating non-small cell lung cancer (NSCLC).

The antibody is suitable for use in therapy, for example in treating a squamous NSCLC.

The antibody is suitable for use in therapy, for example in treating a non-squamous NSCLC.

The antibody is suitable for use in therapy, for example in treating a lung adenocarcinoma.

The antibody is suitable for use in therapy, for example in treating a renal cell carcinoma (RCC).

The antibody is suitable for use in therapy, for example in treating a mesothelioma.

The antibody is suitable for use in therapy, for example in treating a nasopharyngeal carcinoma (NPC).

The antibody is suitable for use in therapy, for example in treating a colorectal cancer.

The antibody is suitable for use in therapy, for example in treating a prostate cancer.

The antibody is suitable for use in therapy, for example in treating a castration-resistant prostate cancer.

The antibody is suitable for use in therapy, for example in treating a stomach cancer.

The antibody is suitable for use in therapy, for example in treating an ovarian cancer.

The antibody is suitable for use in therapy, for example in treating a gastric cancer.

The antibody is suitable for use in therapy, for example in treating a liver cancer.

The antibody is suitable for use in therapy, for example in treating a pancreatic cancer.

The antibody is suitable for use in therapy, for example in treating a thyroid cancer.

The antibody is suitable for use in therapy, for example in treating a squamous cell carcinoma of the head and neck.

The antibody is suitable for use in therapy, for example in treating a carcinomas of the esophagus or gastrointestinal tract.

The antibody is suitable for use in therapy, for example in treating a breast cancer.

The antibody is suitable for use in therapy, for example in treating a fallopian tube cancer.

The antibody is suitable for use in therapy, for example in treating a brain cancer.

The antibody is suitable for use in therapy, for example in treating an urethral cancer.

The antibody is suitable for use in therapy, for example in treating an endometriosis.

The antibody is suitable for use in therapy, for example in treating a cervical cancer.

The antibody is suitable for use in therapy, for example in treating a metastatic lesion of the cancer.

The antibody is suitable for use in therapy, for example in treating a cancer, in combination with an antagonistic antibody that specifically binds PD-1.

The antibody is suitable for use in therapy, for example in treating a cancer, in combination with the antagonistic antibody that specifically binds PD-1 comprising the VH of SEQ ID NO: 48 and the VL of SEQ ID NO: 56.

The antibody is suitable for use in therapy, for example in treating a cancer, in combination with the antagonistic antibody that specifically binds PD-1 comprising the VH of SEQ ID NO: 47 and the VL of SEQ ID NO: 58.

The antibody is suitable for use in therapy, for example in treating a cancer, in combination with the antagonistic antibody that specifically binds PD-1 comprising the VH of SEQ ID NO: 45 and the VL of SEQ ID NO: 60.

The antibody is suitable for use in therapy, for example in treating a cancer, in combination with the antagonistic antibody that specifically binds PD-1 comprising the VH of SEQ ID NO: 64 and the VL of SEQ ID NO: 65.

The antibody is suitable for use in therapy, for example in treating cancer, such as a solid tumor, in combination with an antagonistic antibody specifically binding TIGIT (SEQ ID NO: 301).

The antibody is suitable for use in therapy, for example in treating cancer, such as a solid tumor, in combination with a FGFR inhibitor.

The antibody is suitable for use in therapy, for example in treating cancer, such as a solid tumor, in combination with a vaccine.

The antibody is suitable for use in therapy, for example in treating cancer, such as a solid tumor, in combination with an agonistic antibody specifically binding GITR (SEQ ID NO: 271).

The antibody is suitable for use in therapy, for example in treating cancer, such as a solid tumor, in combination with an agonistic antibody specifically binding CD137 (SEQ ID NO: 281).

The antibody is suitable for use in therapy, for example in treating cancer, such as a solid tumor, in combination with an agonistic antibody specifically binding OX-40 (SEQ ID NO: 279).

The antibody is suitable for use in therapy in a subject who is being treated or who has been treated with an antagonistic antibody specifically binding PD-1 comprising the VH of SEQ ID NO: 230 and the VL of SEQ ID NO: 231. (e.g. KEYTRUDA® (pembrolizumab)).

The antibody is suitable for use in therapy in a subject who is being treated or who has been treated with an antagonistic antibody specifically binding PD-1 comprising the VH of SEQ ID NO: 232 and the VL of SEQ ID NO: 233. (e.g. OPDIVO® (nivolumab)).

The antibody is suitable for use in therapy in a subject who is refractory to treatment with the antagonistic antibody specifically binding PD-1 comprising the VH of SEQ ID NO: 230 and the VL of SEQ ID NO: 231. (e.g. KEYTRUDA® (pembrolizumab)).

The antibody is suitable for use in therapy in a subject who is refractory to treatment with the antagonistic antibody specifically binding PD-1 comprising the VH of SEQ ID NO: 232 and the VL of SEQ ID NO: 233. (e.g. OPDIVO® (nivolumab)).

The antibody is suitable for use in therapy in a subject who has a relapsed tumor after treatment with the antagonistic antibody specifically binding PD-1 comprising the VH of SEQ ID NO: 230 and the VL of SEQ ID NO: 231. (e.g.

KEYTRUDA® (pembrolizumab).

The antibody is suitable for use in therapy in a subject who has a relapsed tumor after treatment with the antagonistic antibody specifically binding PD-1 comprising the VH of SEQ ID NO: 232 and the VL of SEQ ID NO: 233. (e.g. OPDIVO® (nivolumab)).

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 97, 105, 115, 124, 133 and 143, respectively.

In some embodiments, the antibody or the antigen-binding portion thereof comprises a heavy chain framework derived from IGHV5-51 (SEQ ID NO: 179) and a light chain framework derived from IGKV1-39 (SEQ ID NO: 182).

In some embodiments, the antibody comprises the VH of SEQ ID NO: 172 and the VL of SEQ ID NO: 173.

In some embodiments, the VH and the VL are encoded by polynucleotide sequences of SEQ ID NOs: 206 and 207, respectively.

In some embodiments, the antibody or the antigen-binding portion thereof enhances activation of antigen specific CD4⁺ or CD8⁺ T cells, wherein the activation of antigen-specific CD4⁺ or CD8⁺ T cells is assessed by measuring a statistically significant enhancement of CD137 surface expression on antigen specific CD4⁺ or CD8⁺ T cells according to methods described in Example 14.

In some embodiments, the antibody specifically binding TIM-3 or the antigen-binding portion thereof binds TIM-3 within TIM-3 residues 90-102 (RIQIPGIMNDEKF) (SEQ ID NO: 263).

In some embodiments, the antibody specifically binding TIM-3 or the antigen-binding portion thereof binds TIM-3 within TIM-3 residues 90-102 (RIQIPGIMNDEKF) (SEQ ID NO: 263) and residues 50-56 (DERDVNY) SEQ ID NO: 262.

In some embodiments, the antibody specifically binding TIM-3 or the antigen-binding portion thereof inhibits binding of TIM-3 to galectin-9.

In some embodiments, the antibody is an IgG1 isotype.

In some embodiments, the antibody is an IgG3 isotype.

In some embodiments, the antibody is an IgG4 isotype, optionally comprising a S228P substitution when compared to the wild type IgG4.

In some embodiments, the antibody is an IgG4/K isotype, optionally comprising the S228P substitution when compared to the wild type IgG4.

In some embodiments, the antibody comprises the VH of SEQ ID NO: 172 and the VL of SEQ ID NO: 173 and is an IgG4 isotype, optionally comprising the S228P substitution when compared to the wild type IgG4.

In some embodiments, the antibody comprises the VH of SEQ ID NO: 172 and the VL of SEQ ID NO: 173 and is an IgG4K isotype comprising the S228P substitution when compared to the wild type IgG4.

In some embodiments, the antibody is an IgG2 isotype, optionally comprising V234A, G237A, P238S, H268A, V309L, A330S and P331S substitutions when compared to the wild type IgG2.

In some embodiments, the antibody is an IgG2/K isotype, optionally comprising V234A, G237A, P238S, H268A, V309L, A330S and P331S substitutions when compared to the wild type IgG2.

In some embodiments, the antibody comprises the VH of SEQ ID NO: 172 and the VL of SEQ ID NO: 173 and is an IgG2/K isotype, optionally comprising V234A, G237A, P238S, H268A, V309L, A330S and P331S substitutions when compared to the wild type IgG2.

In some embodiments, the antibody comprises the VH of SEQ ID NO: 172 and the VL of SEQ ID NO: 173 and is an IgG2/K isotype comprising V234A, G237A, P238S, H268A, V309L, A330S and P331S substitutions when compared to the wild type IgG2.

In some embodiments, the antibody comprises the HC of SEQ ID NO: 80 and the LC of SEQ ID NO: 81.

SEQ ID NO: 80
EVQLVQSGAEVKKPGESLKISCKGSGYSFTSYWMQWVRQMPGKGLEWMGA
IYPGDGDIRYTQNFKGQVTISADKSISTAYLQWSSLKASDTAMYYCARWE
KSTTVVQRNYFDYWGQGTTVTVSSASTKGPSVFPLAPCSRSTSESTAALG
CLVKDYFPEPVTVSWNSGALTSGVHTFPAVLQSSGLYSLSSVVTVPSSNF
GTQTYTCNVDHKPSNTKVDKTVERKCCVECPPCPAPPAAASSVFLFPPKP
KDTLMISRTPEVTCVVVDVSAEDPEVQFNWYVDGVEVHNAKTKPREEQFN
STFRVVSVLTVLHQDWLNGKEYKCKVSNKGLPSSIEKTISKTKGQPREPQ
VYTLPPSREEMTKNQVSLTCLVKGFYPSDIAVEWESNGQPENNYKTTPPM
LDSDGSFFLYSRLTVDKSRWQQGNVFSCSVMHEALHNHYTQKSLSLSPGK
SEQ IN NO: 81
DIQMTQSPSSLSASVGDRVTITCKASENVGTFVSWYQQKPGKAPKLLIYG
ASNRYTGVPSRFSGSGSGTDFTLTISSLQPEDFATYYCGQSYSYPTFGQG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC

In some embodiments, the antibody is a bispecific antibody, such as a bispecific PD-1/TIM-3 antibody.

The antibody is suitable for use in therapy, for example in treating a cancer.

The antibody is suitable for use in therapy, for example in treating a solid tumor.

The antibody is suitable for use in therapy, for example in treating a melanoma.

The antibody is suitable for use in therapy, for example in treating a lung cancer.

The antibody is suitable for use in therapy, for example in treating non-small cell lung cancer (NSCLC).

The antibody is suitable for use in therapy, for example in treating a squamous NSCLC.

The antibody is suitable for use in therapy, for example in treating a non-squamous NSCLC.

The antibody is suitable for use in therapy, for example in treating a lung adenocarcinoma.

The antibody is suitable for use in therapy, for example in treating a renal cell carcinoma (RCC).

The antibody is suitable for use in therapy, for example in treating a mesothelioma.

The antibody is suitable for use in therapy, for example in treating a nasopharyngeal carcinoma (NPC).

The antibody is suitable for use in therapy, for example in treating a colorectal cancer.

The antibody is suitable for use in therapy, for example in treating a prostate cancer.

The antibody is suitable for use in therapy, for example in treating a castration-resistant prostate cancer.

The antibody is suitable for use in therapy, for example in treating a stomach cancer.

The antibody is suitable for use in therapy, for example in treating an ovarian cancer.

The antibody is suitable for use in therapy, for example in treating a gastric cancer.

The antibody is suitable for use in therapy, for example in treating a liver cancer.

The antibody is suitable for use in therapy, for example in treating a pancreatic cancer.

The antibody is suitable for use in therapy, for example in treating a thyroid cancer.

The antibody is suitable for use in therapy, for example in treating a squamous cell carcinoma of the head and neck.

The antibody is suitable for use in therapy, for example in treating a carcinomas of the esophagus or gastrointestinal tract.

The antibody is suitable for use in therapy, for example in treating a breast cancer.

The antibody is suitable for use in therapy, for example in treating a fallopian tube cancer.

The antibody is suitable for use in therapy, for example in treating a brain cancer.

The antibody is suitable for use in therapy, for example in treating an urethral cancer.

The antibody is suitable for use in therapy, for example in treating an endometriosis.

The antibody is suitable for use in therapy, for example in treating a cervical cancer.

The antibody is suitable for use in therapy, for example in treating a metastatic lesion of the cancer.

The antibody is suitable for use in therapy, for example in treating a hematological malignancy.

The antibody is suitable for use in therapy, for example in treating an acute lymphoblastic leukemia (ALL).

The antibody is suitable for use in therapy, for example in treating a cancer, in combination with an antagonistic antibody that specifically binds PD-1.

The antibody is suitable for use in therapy, for example in treating a cancer, in combination with the antagonistic antibody that specifically binds PD-1 comprising the VH of SEQ ID NO: 48 and the VL of SEQ ID NO: 56.

The antibody is suitable for use in therapy, for example in treating a cancer, in combination with the antagonistic antibody that specifically binds PD-1 comprising the VH of SEQ ID NO: 47 and the VL of SEQ ID NO: 58.

The antibody is suitable for use in therapy, for example in treating a cancer, in combination with the antagonistic antibody that specifically binds PD-1 comprising the VH of SEQ ID NO: 45 and the VL of SEQ ID NO: 60.

The antibody is suitable for use in therapy, for example in treating a cancer, in combination with the antagonistic antibody that specifically binds PD-1 comprising the VH of SEQ ID NO: 65 and the VL of SEQ ID NO: 65.

The antibody is suitable for use in therapy, for example in treating cancer, such as a solid tumor, in combination with an antagonistic antibody specifically binding TIGIT (SEQ ID NO: 301).

The antibody is suitable for use in therapy, for example in treating cancer, such as a solid tumor, in combination with a FGFR inhibitor.

The antibody is suitable for use in therapy, for example in treating cancer, such as a solid tumor, in combination with a vaccine.

The antibody is suitable for use in therapy, for example in treating cancer, such as a solid tumor, in combination with an agonistic antibody specifically binding GITR (SEQ ID NO: 271).

The antibody is suitable for use in therapy, for example in treating cancer, such as a solid tumor, in combination with an agonistic antibody specifically binding CD137 (SEQ ID NO: 281).

The antibody is suitable for use in therapy, for example in treating cancer, such as a solid tumor, in combination with an agonistic antibody specifically binding OX-40 (SEQ ID NO: 279).

The antibody is suitable for use in therapy in a subject who is being treated or who has been treated with an antagonistic antibody specifically binding PD-1 comprising the VH of SEQ ID NO: 230 and the VL of SEQ ID NO: 231. (e.g. KEYTRUDA® (pembrolizumab)).

The antibody is suitable for use in therapy in a subject who is being treated or who has been treated with an antagonistic antibody specifically binding PD-1 comprising the VH of SEQ ID NO: 232 and the VL of SEQ ID NO: 233. (e.g. OPDIVO® (nivolumab)).

The antibody is suitable for use in therapy in a subject who is refractory to treatment with the antagonistic antibody specifically binding PD-1 comprising the VH of SEQ ID NO: 230 and the VL of SEQ ID NO: 231. (e.g. KEYTRUDA® (pembrolizumab)).

The antibody is suitable for use in therapy in a subject who is refractory to treatment with the antagonistic antibody specifically binding PD-1 comprising the VH of SEQ ID NO: 232 and the VL of SEQ ID NO: 233. (e.g. OPDIVO® (nivolumab)).

The antibody is suitable for use in therapy in a subject who has a relapsed tumor after treatment with the antagonistic antibody specifically binding PD-1 comprising the VH of SEQ ID NO: 230 and the VL of SEQ ID NO: 231. (e.g. KEYTRUDA® (pembrolizumab).

The antibody is suitable for use in therapy in a subject who has a relapsed tumor after treatment with the antagonistic antibody specifically binding PD-1 comprising the VH of SEQ ID NO: 232 and the VL of SEQ ID NO: 233. (e.g. OPDIVO® (nivolumab)).

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 90, 99, 107, 117, 126 and 135, respectively.

In some embodiments, the antibody or the antigen-binding portion thereof comprises a heavy chain framework derived from IGHV3-23 (SEQ ID NO: 174) and a light chain framework derived from IGKV3-20 (SEQ ID NO: 180).

In some embodiments, the antibody or the antigen-binding portion thereof comprises the VH of SEQ ID NO: 145 and the VL of SEQ ID NO: 155.

In some embodiments, the VH and the VL are encoded by polynucleotide sequences of SEQ ID NOs: 208 and 209, respectively.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 91, 99, 109, 119, 128 and 137, In some embodiments, the antibody or the antigen-binding portion thereof comprises a heavy chain framework derived from IGHV3-23 (SEQ ID NO: 174) and a light chain framework derived from IGKV4-1 (SEQ ID NO: 181).

In some embodiments, the antibody or the antigen-binding portion thereof comprises the VH of SEQ ID NO: 148 and the VL of SEQ ID NO: 157.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 92, 100, 110, 117, 126 and 135, respectively.

In some embodiments, the antibody or the antigen-binding portion thereof comprises a heavy chain framework derived from IGHV3-23 (SEQ ID NO: 174) and a light chain framework derived from IGKV3-20 (SEQ ID NO: 180).

In some embodiments, the antibody or the antigen-binding portion thereof comprises the VH of SEQ ID NO: 147 and the VL of SEQ ID NO: 155.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 93, 101, 111, 120, 129 and 139, respectively.

In some embodiments, the antibody or the antigen-binding portion thereof comprises a heavy chain framework derived from IGHV3-23 (SEQ ID NO: 174) and a light chain framework derived from IGKV3-20 (SEQ ID NO: 180).

In some embodiments, the antibody or the antigen-binding portion thereof comprises the VH of SEQ ID NO: 149 and the VL of SEQ ID NO: 158.

In some embodiments, the VH and the VL are encoded by polynucleotide sequences of SEQ ID NOs: 201 and 211, respectively.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 94, 102, 112, 121, 130 and 140, respectively.

In some embodiments, the antibody or the antigen-binding portion thereof comprises a heavy chain framework derived from IGHV1-02 (SEQ ID NO: 175) and a light chain framework derived from IGKV4-1 (SEQ ID NO: 181).

In some embodiments, the antibody or the antigen-binding portion thereof comprises the VH of SEQ ID NO: 150 and the VL of SEQ ID NO: 159.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 95, 103, 113, 122, 131 and 141, respectively.

In some embodiments, the antibody or the antigen-binding portion thereof comprises a heavy chain framework derived from IGHV4-30-4 (SEQ ID NO: 176) and a light chain framework derived from IGKV1-39 (SEQ ID NO: 182).

In some embodiments, the antibody or the antigen-binding portion thereof comprises the VH of SEQ ID NO: 151 and the VL of SEQ ID NO: 160.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 96, 104, 114, 123, 132 and 142, respectively.

In some embodiments, the antibody or the antigen-binding portion thereof comprises a heavy chain framework derived from IGHV1-03 (SEQ ID NO: 177) and a light chain framework derived from IGKV1-33 (SEQ ID NO: 183).

In some embodiments, the antibody or the antigen-binding portion thereof comprises the VH of SEQ ID NO: 152 and the VL of SEQ ID NO: 161.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 97, 105, 115, 124, 133 and 143, respectively.

In some embodiments, the antibody or the antigen-binding portion thereof comprises a heavy chain framework derived from IGHV1-03 (SEQ ID NO: 177) and a light chain framework derived from IGKV1-39 (SEQ ID NO: 182).

In some embodiments, the antibody or the antigen-binding portion thereof comprises the VH of SEQ ID NO: 153 and the VL of SEQ ID NO: 162.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 98, 106, 116, 125, 134 and 144, respectively.

In some embodiments, the antibody or the antigen-binding portion thereof comprises a heavy chain framework derived from IGHV2-26 (SEQ ID NO: 178) and a light chain framework derived from IGKV4-1 (SEQ ID NO: 181).

In some embodiments, the antibody or the antigen-binding portion thereof comprises the VH of SEQ ID NO: 154 and the VL of SEQ ID NO: 163.

In some embodiments, the antibody or the antigen-binding portion thereof enhances activation of antigen specific CD4⁺ or CD8⁺ T cells, wherein activation of antigen-specific CD4⁺ or CD8⁺ T cells is assessed by measuring a statistically significant enhancement of CD137 surface expression on antigen specific CD4⁺ or CD8⁺ T cells according to methods described in Example 14.

In some embodiments, the antibody is an IgG1 isotype.

In some embodiments, the antibody is an IgG3 isotype.

In some embodiments, the antibody is an IgG4 isotype, optionally comprising a S228P substitution when compared to the wild type IgG4.

In some embodiments, the antibody is an IgG2 isotype, optionally comprising V234A, G237A, P238S, H268A, V309L, A330S and P331S substitutions when compared to the wild type IgG2.

The VH, the VL, the HCDR and the LCDR sequences of exemplary antagonistic antibodies specifically binding TIM-3 of the invention are shown in Table 3.

Although the embodiments illustrated in the Examples comprise pairs of variable regions, one from a heavy chain and one from a light chain, a skilled artisan will recognize that alternative embodiments may comprise single heavy or light chain variable regions.

The single variable region may be used to screen for variable domains capable of forming a two-domain specific antigen-binding fragment capable of, for example, binding to human TIM-3. The screening may be accomplished by phage display screening methods similarly as described herein.

In some embodiments, the antagonistic antibody specifically binding TIM-3 is a multispecific antibody.

In some embodiments, the antagonistic antibody specifically binding TIM-3 is a bispecific antibody.

In some embodiments, the bispecific or the multispecific antibody binds PD-1 (SEQ ID NO: 1), PD-L1 (SEQ ID NO: 5), PD-L2 (SEQ ID NO: 8), LAG-3 (SEQ ID NO: 293), CEACAM-1 (SEQ ID NO: 296), CEACAM-5 (SEQ ID NO: 307), NKG2D (SEQ ID NO: 282), or TIGIT (SEQ ID NO: 301). Bispecific and multispecific antibodies may be generated using methods described herein.

TABLE 3

| mAb | SEQ ID NO: | | | | | | | |
|---|---|---|---|---|---|---|---|---|
| name | HCDR1 | HCDR2 | HCDR3 | LCDR1 | LCDR2 | LCDR3 | VH | VL |
| TM3B103 | 90 | 99 | 107 | 117 | 126 | 135 | 145 | 155 |
| TM3B105 | 91 | 99 | 108 | 118 | 127 | 136 | 146 | 156 |
| TM3B109 | 91 | 99 | 109 | 119 | 128 | 137 | 148 | 157 |
| TM3B108 | 92 | 100 | 110 | 117 | 126 | 135 | 147 | 155 |
| TM3B113 | 93 | 101 | 111 | 120 | 129 | 139 | 149 | 158 |
| TM3B189 | 94 | 102 | 112 | 121 | 130 | 140 | 150 | 159 |
| TM3B190 | 95 | 103 | 113 | 122 | 131 | 141 | 151 | 160 |
| TM3B193 | 96 | 104 | 114 | 123 | 132 | 142 | 152 | 161 |
| TM3B195 | 97 | 105 | 115 | 124 | 133 | 143 | 153 | 162 |
| TM3B196 | 98 | 106 | 116 | 125 | 134 | 144 | 154 | 163 |
| TM3B291 | 97 | 105 | 115 | 124 | 133 | 143 | 172 | 173 |

### Homologous Antibodies

Variants of the antagonistic antibodies specifically binding TIM-3 of the invention comprising VH or VL amino acid sequences shown in Table 3, Table 36 and Table 37 are within the scope of the invention. For example, variants may comprise one, two, three, four, five, six, seven, eight, nine, ten, eleven, twelve, thirteen, fourteen or fifteen amino acid substitutions in the VH and/or the VL as long as the homologous antibodies retain or have improved functional properties when compared to the parental antibodies. In some embodiments, the sequence identity may be about 90%, 91%, 92%, 93%, 94%, 95%, 96%, 97%, 98% or 99% to a VH or the VL amino acid sequence of the invention.

The invention also provides an antagonistic antibody specifically binding TIM-3 or an antigen-binding portion thereof, comprising the VH of SEQ ID NO: 145 and the VL of SEQ ID NO: 155, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding TIM-3 or an antigen-binding portion thereof, comprising the VH of SEQ ID NO: 146 and the VL of SEQ ID NO: 156, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding TIM-3 or an antigen-binding portion thereof, comprising the VH of SEQ ID NO: 148 and the VL of SEQ ID NO: 157, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding TIM-3 or an antigen-binding portion thereof, comprising the VH of SEQ ID NO: 147 and the VL of SEQ ID NO: 155, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding TIM-3 or an antigen-binding portion thereof, comprising the VH of SEQ ID NO: 149 and the VL of SEQ ID NO: 158, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding TIM-3 or an antigen-binding portion thereof, comprising the VH of SEQ ID NO: 150 and the VL of SEQ ID NO: 159, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding TIM-3 or an antigen-binding portion thereof, comprising the VH of SEQ ID NO: 151 and the VL of SEQ ID NO: 160, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding TIM-3 or an antigen-binding portion thereof, comprising the VH of SEQ ID NO: 152 and the VL of SEQ ID NO: 161, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding TIM-3 or an antigen-binding portion thereof, comprising the VH of SEQ ID NO: 153 and the VL of SEQ ID NO: 162, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding TIM-3 or an antigen-binding portion thereof, comprising the VH of SEQ ID NO: 154 and the VL of SEQ ID NO: 163, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding TIM-3 or an antigen-binding portion thereof, comprising the VH of SEQ ID NO: 172 and the VL of SEQ ID NO: 173, wherein the VH, the VL or both the VH and the VL optionally comprise one, two, three, four, five, six, seven, eight, nine, ten, eleven, twelve, thirteen, fourteen or fifteen amino acid substitutions. Optionally, any substitutions are not within the CDRs.

The invention also provides an antagonistic antibody specifically binding TIM-3 or an antigen-binding portion thereof, comprising the VH having the amino acid sequence at least 90%, 91%, 92%, 93%, 94%, 95%, 96%, 97%, 98% or 99% identical to the VH of SEQ ID NOs: 145, 146, 147, 148, 149, 150, 151, 152, 153, 154 or 172. Optionally, any variation from the sequences of the SEQ ID NOs is not within the CDRs.

The invention also provides an antagonistic antibody specifically binding TIM-3 or an antigen-binding portion thereof, comprising the VL having the amino acid sequence at least 90%, 91%, 92%, 93%, 94%, 95%, 96%, 97%, 98% or 99% identical to the VL of SEQ IS NOs: 155, 156, 157, 158, 159, 160, 161, 162, 163 or 173. Optionally, any variation from the sequences of the SEQ ID NOs is not within the CDRs.

The invention also provides an antagonistic antibody specifically binding TIM-3 or an antigen-binding portion thereof, comprising the VH having the amino acid sequence at least 90%, 91%, 92%, 93%, 94%, 95%, 96%, 97%, 98% or 99% identical to the VH of SEQ ID NOs: 145, 146, 147, 148, 149, 150, 151, 152, 153, 154 or 172 and the VL having the amino acid sequence at least 90%, 91%, 92%, 93%, 94%, 95%, 96%, 97%, 98% or 99% identical to the VL of SEQ ID NOs: 155, 156, 157, 158, 159, 160, 161, 162, 163 or 173. Optionally, any variation from the sequences of the SEQ ID NOs is not within the CDRs.

The invention also provides an antagonistic antibody specifically binding TIM-3 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 145 and the VL of SEQ ID NO: 155. Optionally, any variation from the sequences of the SEQ ID NOs is not within the CDRs.

The invention also provides an antagonistic antibody specifically binding TIM-3 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 146 and the VL of SEQ ID NO: 156. Optionally, any variation from the sequences of the SEQ ID NOs is not within the CDRs.

The invention also provides an antagonistic antibody specifically binding TIM-3 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 148 and the VL of SEQ ID NO: 157. Optionally, any variation from the sequences of the SEQ ID NOs is not within the CDRs.

The invention also provides an antagonistic antibody specifically binding TIM-3 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 147 and the VL of SEQ ID NO: 155. Optionally, any variation from the sequences of the SEQ ID NOs is not within the CDRs.

The invention also provides an antagonistic antibody specifically binding TIM-3 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 149 and the VL of SEQ ID NO: 158. Optionally, any variation from the sequences of the SEQ ID NOs is not within the CDRs.

The invention also provides an antagonistic antibody specifically binding TIM-3 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 150 and the VL of SEQ ID NO: 159. Optionally, any variation from the sequences of the SEQ ID NOs is not within the CDRs.

The invention also provides an antagonistic antibody specifically binding TIM-3 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 151 and the VL of SEQ ID NO: 160. Optionally, any variation from the sequences of the SEQ ID NOs is not within the CDRs.

The invention also provides an antagonistic antibody specifically binding TIM-3 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 152 and the VL of SEQ ID NO: 161. Optionally, any variation from the sequences of the SEQ ID NOs is not within the CDRs.

The invention also provides an antagonistic antibody specifically binding TIM-3 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 153 and the VL of SEQ ID NO: 162. Optionally, any variation from the sequences of the SEQ ID NOs is not within the CDRs.

The invention also provides an antagonistic antibody specifically binding TIM-3 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 154 and the VL of SEQ ID NO: 163. Optionally, any variation from the sequences of the SEQ ID NOs is not within the CDRs.

The invention also provides an antagonistic antibody specifically binding TIM-3 or an antigen-binding portion thereof, comprising the VH and the VL having the amino acid sequences at least 90%, 91%, 92%, 93%, 94%, 95%, 96%, 97%, 98% or 99% identical to the VH of SEQ ID NO: 172 and the VL of SEQ ID NO: 173. Optionally, any variation from the sequences of the SEQ ID NOs is not within the CDRs.

The homologous antibodies of the invention described herein have substantially similar functionality when compared to the parental TIM-3 antibodies.

Antagonistic antibodies specifically binding TIM-3 of the invention with conservative modifications The invention also provides an antagonistic antibody specifically binding TIM-3 or an antigen-binding portion thereof, comprising the VH comprising the HCDR1, the HCDR2 and the HCDR3 sequences and the VL comprising the LCDR1, the LCDR2 and the LCDR3 sequences, wherein one or more of the CDR sequences comprise specified amino acid sequences based on the antibodies described herein (e.g., antibodies shown in Table 3, Table 36 or Table 37 or conservative modifications thereof, and wherein the antibodies retain the desired functional properties of the parental antagonistic antibodies specifically binding TIM-3 of the invention.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 90, 99, 107, 117, 126 and 135, respectively, and conservative modifications thereof.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 91, 99, 108, 118, 127 and 136, respectively, and conservative modifications thereof.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 91, 99, 109, 119, 128 and 137, respectively, and conservative modifications thereof.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 92, 100, 110, 117, 126 and 135, respectively, and conservative modifications thereof.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 93, 101, 111, 120, 129 and 139, respectively, and conservative modifications thereof.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 94, 102, 112, 121, 130 and 140, respectively, and conservative modifications thereof.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 95, 103, 113, 122, 131 and 141, respectively, and conservative modifications thereof.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 96, 104, 114, 123, 132 and 142, respectively, and conservative modifications thereof.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 97, 105, 115, 124, 133 and 143, respectively, and conservative modifications thereof.

The invention also provides an antagonistic antibody specifically binding TIM-3 or an antigen-binding portion thereof, comprising the HCDR1, the HCDR2, the HCDR3, the LCDR1, the LCDR2 and the LCDR3 of SEQ ID NOs: 98, 106, 116, 125, 134 and 144, respectively, and conservative modifications thereof.

“Conservative modification” refers to modifications as described herein.

Antagonistic Antibodies Specifically Binding TIM-3 of the Invention with Specific Framework Sequences

The invention also provides an antagonistic antibody specifically binding TIM-3 or an antigen-binding portion thereof, comprising the VH and the VL derived from particular human germline immunoglobulin sequences.

The invention also provides an antagonistic antibody specifically binding TIM-3 or an antigen-binding portion thereof, comprising the VH framework derived from IGHV3-23 (SEQ ID NO: 174), IGHV1-02 (SEQ ID NO: 175), IGHV4-30-4 (SEQ ID NO: 176), IGHV1-03 (SEQ ID NO: 177), IGHV2-26 (SEQ ID NO: 178) or IGHV5-51 (SEQ ID NO: 179).

The invention also provides an antagonistic antibody specifically binding TIM-3 or an antigen-binding portion thereof, comprising the VL framework derived from IGKV3-20 (A27) (SEQ ID NO: 180), IGKV3-11 (L6) (SEQ ID NO: 171), IGKV4-1 (B3) (SEQ ID NO: 181), IGKV1-39) (012) (SEQ ID NO: 182) or IGKV1-33 (018) (SEQ ID NO: 183).

The invention also provides an antagonistic antibody specifically binding TIM-3 or an antigen-binding portion thereof, comprising the VH framework derived from IGHV3-23 (SEQ ID NO: 174) and the VL framework derived from IGKV3-20 (SEQ ID NO: 180).

The invention also provides an antagonistic antibody specifically binding TIM-3 or an antigen-binding portion thereof, comprising the VH framework derived from IGHV3-23 (SEQ ID NO: 174) and the VL framework derived from IGKV3-11 (SEQ ID NO: 171).

The invention also provides an antagonistic antibody specifically binding TIM-3 or an antigen-binding portion thereof, comprising the VH framework derived from IGHV3-23 (SEQ ID NO: 174) and the VL framework derived from IGKV4-1 (SEQ ID NO: 181).

The invention also provides an antagonistic antibody specifically binding TIM-3 or an antigen-binding portion thereof, comprising the VH framework derived from IGHV1-02 (SEQ ID NO: 175) and the VL framework derived from IGKV4-1 (SEQ ID NO: 181).

The invention also provides an antagonistic antibody specifically binding TIM-3 or an antigen-binding portion thereof, comprising the VH framework derived from IGHV4-30-4 (SEQ ID NO: 176) and the VL framework derived from IGKV1-39 (SEQ ID NO: 182).

The invention also provides an antagonistic antibody specifically binding TIM-3 or an antigen-binding portion thereof, comprising the VH framework derived from IGHV1-03 (SEQ ID NO: 177) and the VL framework derived from IGKV1-33 (SEQ ID NO: 183).

The invention also provides an antagonistic antibody specifically binding TIM-3 or an antigen-binding portion thereof, comprising the VH framework derived from IGHV1-03 (SEQ ID NO: 177) and the VL framework derived from IGKV1-39 (SEQ ID NO: 182).

The invention also provides an antagonistic antibody specifically binding TIM-3 or an antigen-binding portion thereof, comprising the VH framework derived from IGHV2-26 (SEQ ID NO: 178) and the VL framework derived from IGKV4-1 (SEQ ID NO: 181).

The invention also provides an antagonistic antibody specifically binding TIM-3 or an antigen-binding portion thereof, comprising the VH framework derived from IGHV5-51 (SEQ ID NO: 179) and the VL framework derived from IGKV1-39 (SEQ ID NO: 182).

The antibodies of the invention comprising heavy or light chain variable regions “derived from” a particular framework or germline sequence refer to antibodies obtained from a system that uses human germline immunoglobulin genes, such as from transgenic mice or from phage display libraries as discussed herein. An antibody that is “derived from” a particular framework or germline sequence may contain amino acid differences as compared to the sequence it was derived from, due to, for example, naturally-occurring somatic mutations or intentional substitutions.

Exemplary antagonistic antibodies specifically binding TIM-3 having certain VH and VL framework sequences are shown in Table 38.

### Bispecific Anti-PD-1/TIM-3 Antibodies

The invention also provides antagonistic bispecific PD-I/TIM-3 antibodies.

The invention also provides an isolated antagonistic bispecific PD-I/TIM-3 antibody comprising a first domain specifically binding PD-1 and a second domain specifically binding TIM-3.

In some embodiments, the antagonistic bispecific PD-1/TIM-3 antibody of the invention enhances activation of antigen-specific CD4⁺ or CD8⁺ T cells.

In some embodiments, the antagonistic bispecific PD-1/TIM-3 antibody of the invention enhances activation of antigen-specific CD4⁺ or CD8⁺ T cells, wherein enhanced activation of antigen-specific CD4⁺ or CD8⁺ T cells is assessed by measuring a statistically significant increase of CD137 surface expression on antigen-specific CD4⁺ or CD8⁺ T cells.

In some embodiments, the antagonistic bispecific PD-1/TIM-3 antibody of the invention inhibits TIM-3 binding to galectin-9.

In some embodiments, the antagonistic bispecific PD-1/TIM-3 antibody of the invention
binds human PD-I with an equilibrium dissociation constant (K_{D}) of less than about 100 nM, wherein the K_{D} is measured using ProteOn XPR36 system at +25° C.;
binds human PD-1 with the K_{D} of less than about 1 nM, wherein the K_{D} is measured using ProteOn XPR36 system at +25° C.;
binds cynomolgus PD-1 with the K_{D} of less than about 100 nM, wherein the K_{D} is measured using ProteOn XPR36 system at +25° C.; or binds cynomolgus PD-1 with the K_{D} of less than about 1 nM;
wherein the K_{D} is measured using ProteOn XPR36 system at +25° C.

In some embodiments, the antagonistic bispecific PD-1/TIM-3 antibody of the invention enhances an activation of antigen-specific CD4⁺ or CD8⁺ T cells, wherein the activation of antigen-specific CD4⁺ or CD8⁺ T cells is assessed by measuring a statistically significant increase of CD137 surface expression on antigen-specific CD4⁺ or CD8⁺ T cells and binds human PD-I with an equilibrium dissociation constant (K_{D}) of less than about 100 nM, wherein the K_{D} is measured using ProteOn XPR36 system at +25° C.

In some embodiments, the antagonistic bispecific PD-1/TIM-3 antibody of the invention enhances the activation of antigen-specific CD4⁺ or CD8⁺ T cells, wherein the activation of antigen-specific CD4⁺ or CD8⁺ T cells is assessed by measuring a statistically significant increase of CD137 surface expression on antigen-specific CD4⁺ or CD8⁺ T cells, and binds human PD-1 with an equilibrium dissociation constant (K_{D}) of less than about 1 nM, wherein the K_{D} is measured using ProteOn XPR36 system at +25° C.

In some embodiments, the antagonistic bispecific PD-1/TIM-3 antibody of the invention enhances the activation of antigen-specific CD4⁺ or CD8⁺ T cells, wherein the activation of antigen-specific CD4⁺ or CD8⁺ T cells is assessed by measuring a statistically significant increase of CD137 surface expression on antigen-specific CD4⁺ or CD8⁺ T cells and binds cynomolgus PD-1 with an equilibrium dissociation constant (K_{D}) of less than about 100 nM, wherein the K_{D} is measured using ProteOn XPR36 system at +25° C.

In some embodiments, the antagonistic bispecific PD-1/TIM-3 antibody of the invention enhances the activation of antigen-specific CD4⁺ or CD8⁺ T cells, wherein the activation of antigen-specific CD4⁺ or CD8⁺ T cells is assessed by measuring a statistically significant increase of CD137 surface expression on antigen-specific CD4⁺ or CD8⁺ T cells, and binds cynomolgus PD-1 with an equilibrium dissociation constant (K_{D}) of less than about 1 nM, wherein the K_{D} is measured using ProteOn XPR36 system at +25° C.

The antagonistic bispecific PD-1/TIM-3 antibodies of the invention described herein may be evaluated for their ability to enhance antigen specific CD4⁺ or CD8⁺ T cell activation, to inhibit TIM-3 binding to galectin-9, and binding kinetics to human or cynomolgus PD-1 or TIM-3 may be assessed using methods described herein.

For example, CD137 may be used as a marker for activation of antigen specific CD4⁺ or CD8⁺ T cells. CD137 surface expression may be measured on T cells cultured in the presence or in the absence of a test antibody, such as the bispecific PD-1/TIM-3 antibody, using anti-CD137 antibody and a secondary antibody conjugated for example to a fluorescent dye. The statistically significant difference in the obtained signal on T cells cultured in the presence or in the absence of the test antibody is evaluated.

In some embodiments, the antagonistic bispecific PD-1/TIM-3 antibody of the invention binds TIM-3 within TIM-3 residues 32-47 (WGKGACPVFECGNVVL) (SEQ ID NO: 261).

In some embodiments, the antagonistic bispecific PD-1/TIM-3 antibody of the invention binds TIM-3 within TIM-3 residues 32-47 (WGKGACPVFECGNVVL) (SEQ ID NO: 261) and residues 50-56 (DERDVNY) SEQ ID NO: 262.

In some embodiments, the antagonistic bispecific PD-1/TIM-3 antibody of the invention binds TIM-3 within TIM-3 residues 90-102 (RIQIPGIMNDEKF) (SEQ ID NO: 263).

In some embodiments, the antagonistic bispecific PD-1/TIM-3 antibody of the invention binds TIM-3 within TIM-3 residues 90-102 (RIQIPGIMNDEKF) (SEQ ID NO: 263) and residues 50-56 (DERDVNY) SEQ ID NO: 262.

In some embodiments, the first domain comprises a heavy chain complementarity determining region (HCDR) 1 a HCDR2 and a HCDR3 of SEQ ID NOs: X₁YX₂IX₃ wherein X₁ is S or D; X₂ is V or A; and X₃ is H or S, 83 and 84, respectively.

In some embodiments, the first domain comprises the HCDR1, the HCDR2 and the HCDR3 of SEQ ID NOs: X₁YX₂IX₃ wherein X₁ is S or D; X₂ is V or A; and X₃ is H or S, 83 and 85, respectively.

In some embodiments, the first domain comprises a light chain complementarity determining regions (LCDR) 1, a LCDR2 and a LCDR3 of SEQ ID NOs: 86, 87 and 88, respectively.

In some embodiments, the first domain comprises the HCDR1, the HCDR2 and the HCDR3 of SEQ ID NOs: X₁YX₂IX₃ wherein X₁ is S or D; X₂ is V or A; and X₃ is H or S, 83 and 84, respectively, and the LCDR1, the LCDR2 and the LCDR3 of SEQ ID NOs: 86, 87 and 88, respectively.

In some embodiments, the first domain comprises the HCDR1, the HCDR2 and the HCDR3 of SEQ ID NOs: X₁YX₂IX₃ wherein X₁ is S or D; X₂ is V or A; and X₃ is H or S, 83 and 85, respectively, and the LCDR1, the LCDR2 and the LCDR3 of SEQ ID NOs: 86, 87 and 88, respectively.

In some embodiments, the second domain comprises the HCDR1, the HCDR2 and the HCDR3 amino acid sequences of SEQ ID NOs: X₁₇YX₁₈MX₁₉ wherein X₁₇ is N, S, G or D; X₁₈ is W or A; and X₁₉ is S or H, 165 and X₂₄X₂₅X₂₆X₂₇X₂₈X₂₉X₃₀X₃₁DY wherein X₂₄ is D, S, N, G or E; X₂₅ is H, P, E, T or L; X₂₆ is W, E, N or deleted; X₂₇ is D, P or deleted; X₂₈ is P, Y, D or deleted; X₂₉ is N, A, D, G or deleted; X₃₀ is F, P, R, W or V; and X₃₁ is L or F, respectively.

In some embodiments, the second domain comprises the LCDR1, the LCDR2 and the LCDR3 amino acid sequences of SEQ ID NOs: 167, X₄₃ASX₄₄RX₄₅X₄₆ wherein X43 is G, D, W or T; X44 is S, N or T; X45 is A or E; and X46 is T or S and 169, respectively.

In some embodiments, the second domain comprises the HCDR1, the HCDR2 and the HCDR3 amino acid sequences of SEQ ID NOs: X₁₇YX₁₈MX₁₉ wherein X₁₇ is N, S, G or D; X₁₈ is W or A; and X₁₉ is S or H, 165 and X₂₄X₂₅X₂₆X₂₇X₂₈X₂₉X₃₀X₃₁DY wherein X₂₄ is D, S, N, G or E; X₂₅ is H, P, E, T or L; X₂₆ is W, E, N or deleted; X₂₇ is D, P or deleted; X₂₈ is P, Y, D or deleted; X₂₉ is N, A, D, G or deleted; X₃₀ is F, P, R, W or V; and X₃₁ is L or F, respectively, and the LCDR1, the LCDR2 and the LCDR3 amino acid sequences of SEQ ID NOs: 167, X₄₃ASX₄₄RX₄₅X₄₆ wherein X₄₃ is G, D, W or T;
X₄₄ is S, N or T; X₄₅ is A or E; and X₄₆ is T or S and 169 respectively.

In some embodiments, the first domain comprises the HCDR1, the HCDR2, the HCDR3, the LCDR1, the LCDR2 and the LCDR3 of
SEQ ID NOs: 10, 13, 16, 20, 26 and 31, respectively;
SEQ ID NOs: 10, 13, 16, 21, 26 and 32, respectively;
SEQ ID NOs: 10, 14, 16, 22, 27 and 33, respectively;
SEQ ID NOs: 10, 14, 16, 22, 26 and 34, respectively;
SEQ ID NOs: 10, 14, 16, 23, 28 and 35, respectively;
SEQ ID NOs: 10, 13, 17, 20, 26 and 31, respectively;
SEQ ID NOs: 10, 13, 17, 20, 26 and 36, respectively;
SEQ ID NOs: 10, 13, 17, 21, 26 and 32, respectively;
SEQ ID NOs: 10, 13, 17, 21, 27 and 37, respectively;
SEQ ID NOs: 10, 13, 17, 23, 26 and 32, respectively;
SEQ ID NOs: 10, 13, 17, 22, 26 and 32, respectively;
SEQ ID NOs: 10, 13, 18, 20, 26 and 31, respectively;
SEQ ID NOs: 11, 15, 18, 20, 26 and 31, respectively;
SEQ ID NOs: 10, 13, 19, 20, 26 and 31, respectively;
SEQ ID NOs: 12, 13, 19, 20, 26 and 31, respectively;
SEQ ID NOs: 10, 14, 17, 23, 28 and 35, respectively;
SEQ ID NOs: 10, 14, 17, 22, 26 and 34, respectively;
SEQ ID NOs: 10, 14, 17, 23, 26 and 32, respectively;
SEQ ID NOs: 12, 13, 19, 24, 26 and 38, respectively;
SEQ ID NOs: 12, 13, 19, 20, 29 and 39, respectively;
SEQ ID NOs: 11, 15, 18, 20, 30 and 32, respectively;
SEQ ID NOs: 11, 15, 18, 25, 26 and 40, respectively;
SEQ ID NOs: 11, 15, 18, 24, 26 and 32, respectively; or
SEQ ID NOs: 66, 67, 68, 69, 70 and 71, respectively.

In some embodiments, the second domain comprises the HCDR1, the HCDR2, the HCDR3, the LCDR1, the LCDR2 and the LCDR3 of
SEQ ID NOs: 90, 99, 107, 117, 126 and 135, respectively;
SEQ ID NOs: 91, 99, 108, 118, 127 and 136, respectively;
SEQ ID NOs: 91, 99, 109, 119, 128 and 137, respectively;
SEQ ID NOs: 92, 100, 110, 117, 126 and 135, respectively;
SEQ ID NOs: 93, 101, 111, 120, 129 and 139, respectively;
SEQ ID NOs: 94, 102, 112, 121, 130 and 140, respectively;
SEQ ID NOs: 95, 103, 113, 122, 131 and 141, respectively;
SEQ ID NOs: 96, 104, 114, 123, 132 and 142, respectively;
SEQ ID NOs: 97, 105, 115, 124, 133 and 143, respectively; or
SEQ ID NOs: 98, 106, 116, 125, 134 and 144, respectively.

In some embodiments, the first domain comprises the VH of SEQ ID NOs: 41, 42, 43, 44, 45, 46, 47, 48, 63 or 64, the VH optionally having one, two, three, four, five, six, seven, eight, nine, ten, eleven, twelve, thirteen, fourteen or fifteen conservative amino acid substitutions. Optionally, any substitutions are not within the CDRs.

In some embodiments, the first domain comprises the VL of SEQ ID NOs: 49, 50, 51, 52, 53, 54, 55, 56, 57, 58, 59, 60, 61, 62 or 65, the VL optionally having one, two, three, four, five, six, seven, eight, nine, ten, eleven, twelve, thirteen, fourteen or fifteen conservative amino acid substitutions. Optionally, any substitutions are not within the CDRs.

In some embodiments, the first domain comprises the VH of SEQ ID NOs: 41, 42, 43, 44, 45, 46, 47, 48, 63 or 64 and the VL of SEQ ID NOs: 49, 50, 51, 52, 53, 54, 55, 56, 57, 58, 59, 60, 61, 62 and 65, the VH, the VL, or the VH and the VL optionally having one, two, three, four, five, six, seven, eight, nine, ten, eleven, twelve, thirteen, fourteen or fifteen conservative amino acid substitutions. Optionally, any substitutions are not within the CDRs.

In some embodiments, the second domain comprises the VH of SEQ ID NOs: 145, 146, 147, 148, 149, 150, 151, 152, 153, 154 or 172, the VH optionally having one, two, three, four, five, six, seven, eight, nine, ten, eleven, twelve, thirteen, fourteen or fifteen conservative amino acid substitutions. Optionally, any substitutions are not within the CDRs.

In some embodiments, the second domain comprises the VL of SEQ IS NOs: 155, 156, 157, 158, 159, 160, 161, 162, 163 or 173, the VL optionally having one, two, three, four, five, six, seven, eight, nine, ten, eleven, twelve, thirteen, fourteen or fifteen conservative amino acid substitutions. Optionally, any substitutions are not within the CDRs.

In some embodiments, the second domain comprises the VH of SEQ ID NOs: 145, 146, 147, 148, 149, 150, 151, 152, 153, 154 or 172 and the VL of SEQ ID NOs: 155, 156, 157, 158, 159, 160, 161, 162, 163 or 173, the VH and the VL optionally having one, two, three, four, five, six, seven, eight, nine, ten, eleven, twelve, thirteen, fourteen or fifteen conservative amino acid substitutions. Optionally, any substitutions are not within the CDRs.

In some embodiments, the first domain comprises the VH of SEQ ID NO: 41 and the VL of SEQ ID NO: 49.

In some embodiments, the first domain comprises the VH of SEQ ID NO: 41 and the VL of SEQ ID NO: 50.

In some embodiments, the first domain comprises the VH of SEQ ID NO: 42 and the VL of SEQ ID NO: 51.

In some embodiments, the first domain comprises the VH of SEQ ID NO: 42 and the VL of SEQ ID NO: 52.

In some embodiments, the first domain comprises the VH of SEQ ID NO: 42 and the VL of SEQ ID NO: 53.

In some embodiments, the first domain comprises the VH of SEQ ID NO: 43 and the VL of SEQ ID NO: 49.

In some embodiments, the first domain comprises the VH of SEQ ID NO: 43 and the VL of SEQ ID NO: 54.

In some embodiments, the first domain comprises the VH of SEQ ID NO: 43 and the VL of SEQ ID NO: 50.

In some embodiments, the first domain comprises the VH of SEQ ID NO: 43 and the VL of SEQ ID NO: 55.

In some embodiments, the first domain comprises the VH of SEQ ID NO: 43 and the VL of SEQ ID NO: 56.

In some embodiments, the first domain comprises the VH of SEQ ID NO: 43 and the VL of SEQ ID NO: 57.

In some embodiments, the first domain comprises the VH of SEQ ID NO: 44 and the VL of SEQ ID NO: 49.

In some embodiments, the first domain comprises the VH of SEQ ID NO: 45 and the VL of SEQ ID NO: 49.

In some embodiments, the first domain comprises the VH of SEQ ID NO: 46 and the VL of SEQ ID NO: 49.

In some embodiments, the first domain comprises the VH of SEQ ID NO: 47 and the VL of SEQ ID NO: 49.

In some embodiments, the first domain comprises the VH of SEQ ID NO: 48 and the VL of SEQ ID NO: 53.

In some embodiments, the first domain comprises the VH of SEQ ID NO: 48 and the VL of SEQ ID NO: 52.

In some embodiments, the first domain comprises the VH of SEQ ID NO: 48 and the VL of SEQ ID NO: 56.

In some embodiments, the first domain comprises the VH of SEQ ID NO: 47 and the VL of SEQ ID NO: 58.

In some embodiments, the first domain comprises the VH of SEQ ID NO: 47 and the VL of SEQ ID NO: 59.

In some embodiments, the first domain comprises the VH of SEQ ID NO: 45 and the VL of SEQ ID NO: 60.

In some embodiments, the first domain comprises the VH of SEQ ID NO: 45 and the VL of SEQ ID NO: 61.

In some embodiments, the first domain comprises the VH of SEQ ID NO: 45 and the VL of SEQ ID NO: 62.

In some embodiments, the first domain comprises the VH of SEQ ID NO: 63 and the VL of SEQ ID NO: 65.

In some embodiments, the first domain comprises the VH of SEQ ID NO: 64 and the VL of SEQ ID NO: 65.

In some embodiments, the second domain comprises the VH of SEQ ID NO: 145 and the VL of SEQ ID NO: 155.

In some embodiments, the second domain comprises the VH of SEQ ID NO: 146 and the VL of SEQ ID NO: 156.

In some embodiments, the second domain comprises the VH of SEQ ID NO: 148 and the VL of SEQ ID NO: 157.

In some embodiments, the second domain comprises the VH of SEQ ID NO: 147 and the VL of SEQ ID NO: 155.

In some embodiments, the second domain comprises the VH of SEQ ID NO: 149 and the VL of SEQ ID NO: 158.

In some embodiments, the second domain comprises the VH of SEQ ID NO: 150 and the VL of SEQ ID NO: 159.

In some embodiments, the second domain comprises the VH of SEQ ID NO: 151 and the VL of SEQ ID NO: 160.

In some embodiments, the second domain comprises the VH of SEQ ID NO: 152 and the VL of SEQ ID NO: 161.

In some embodiments, the second domain comprises the VH of SEQ ID NO: 153 and the VL of SEQ ID NO: 162.

In some embodiments, the second domain comprises the VH of SEQ ID NO: 154 and the VL of SEQ ID NO: 163.

In some embodiments, the second domain comprises the VH of SEQ ID NO: 172 and the VL of SEQ ID NO: 173.

The invention also provides an isolated antagonistic bispecific PD-1/TIM-3 antibody comprising a first domain specifically binding PD-1 and a second domain specifically binding TIM-3, wherein the first domain comprises the HCDR1, the HCDR2, the HCDR3, the LCDR1, the LCDR2 and the LCDR3 of SEQ ID NOs: 10, 14, 17, 23, 26 and 32, respectively, and the second domain comprises the HCDR1, the HCDR2, the HCDR3, the LCDR1, the LCDR2 and the LCDR3 of SEQ ID NOs: 91, 99, 108, 118, 127 and 136, respectively.

In some embodiments, the antagonistic bispecific PD-1/TIM-3 antibody binds TIM-3 within TIM-3 residues 32-47 (WGKGACPVFECGNVVL) (SEQ ID NO: 261).

In some embodiments, the antagonistic bispecific PD-1/TIM-3 antibody binds TIM-3 within TIM-3 residues 32-47 (WGKGACPVFECGNVVL) (SEQ ID NO: 261) and residues 50-56 (DERDVNY) SEQ ID NO: 262.

In some embodiments, the antagonistic bispecific PD-1/TIM-3 antibody inhibits TIM-3 binding to galectin-9.

In some embodiments, the first domain comprises the VH of SEQ ID NO: 48 and the VL of SEQ ID NO: 56 and the second domain comprises the VH of SEQ ID NO: 146 and the VL of SEQ ID NO: 156.

In some embodiments, the antibody is an IgG1 isotype.

In some embodiments, the antibody is an IgG2 isotype.

In some embodiments, the antibody is an IgG2 isotype comprising a F405L and/or a K409R substitution.

In some embodiments, the antibody is an IgG2 isotype, optionally comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype, optionally comprising a S228P substitution when compared to the wild type IgG4.

In some embodiments, the antibody is an IgG4 isotype comprising a F405L and a K409R substitution.

In some embodiments, the antibody is an IgG4 isotype comprising a heavy chain substitution S228P when compared to the wild type IgG4.

In some embodiments, the isolated antagonistic bispecific PD-1/TIM-3 antibody comprises a first heavy chain (HC1) a first light chain (LC1), a second heavy chain (HC2) and a second light chain (LC2) of SEQ ID NOs: 241, 188, 245 or 194, respectively.

In some embodiments, the isolated antagonistic bispecific PD-1/TIM-3 antibody comprises the HC1, the LC1, the HC2 and the LC2 of SEQ ID NOs: 186, 188, 191 or 194, respectively.

In some embodiments, the isolated antagonistic bispecific PD-1/TIM-3 antibody comprises the HC1, the LC1, the HC2 and the LC2 of SEQ ID NOs: 186, 188, 248 or 194, respectively.

In some embodiments, the isolated antagonistic bispecific PD-1/TIM-3 antibody comprises the HC1, the LC1, the HC2 and the LC2 of SEQ ID NOs: 243, 188, 246 or 194, respectively.

The antibody is suitable for use in therapy, for example in treating a cancer.

The antibody is suitable for use in therapy, for example in treating a solid tumor.

The antibody is suitable for use in therapy, for example in treating a melanoma.

The antibody is suitable for use in therapy, for example in treating a lung cancer.

The antibody is suitable for use in therapy, for example in treating a non-small cell lung cancer (NSCLC) The antibody is suitable for use in therapy, for example in treating a squamous NSCLC.

The antibody is suitable for use in therapy, for example in treating a non-squamous NSCLC.

The antibody is suitable for use in therapy, for example in treating a lung adenocarcinoma.

The antibody is suitable for use in therapy, for example in treating a renal cell carcinoma (RCC).

The antibody is suitable for use in therapy, for example in treating a mesothelioma.

The antibody is suitable for use in therapy, for example in treating a nasopharyngeal carcinoma (NPC).

The antibody is suitable for use in therapy, for example in treating a colorectal cancer.

The antibody is suitable for use in therapy, for example in treating a prostate cancer.

The antibody is suitable for use in therapy, for example in treating a castration-resistant prostate cancer.

The antibody is suitable for use in therapy, for example in treating a stomach cancer.

The antibody is suitable for use in therapy, for example in treating an ovarian cancer.

The antibody is suitable for use in therapy, for example in treating a gastric cancer.

The antibody is suitable for use in therapy, for example in treating a liver cancer.

The antibody is suitable for use in therapy, for example in treating pancreatic cancer.

The antibody is suitable for use in therapy, for example in treating a thyroid cancer.

The antibody is suitable for use in therapy, for example in treating a squamous cell carcinoma of the head and neck.

The antibody is suitable for use in therapy, for example in treating a carcinomas of the esophagus or gastrointestinal tract.

The antibody is suitable for use in therapy, for example in treating a breast cancer.

The antibody is suitable for use in therapy, for example in treating a fallopian tube cancer.

The antibody is suitable for use in therapy, for example in treating a brain cancer.

The antibody is suitable for use in therapy, for example in treating an urethral cancer.

The antibody is suitable for use in therapy, for example in treating an endometriosis.

The antibody is suitable for use in therapy, for example in treating a cervical cancer.

The antibody is suitable for use in therapy, for example in treating a metastatic lesion of the cancer.

The antibody is suitable for use in therapy in a subject who is being treated or who has been treated with anti-PD-1 antibody comprising the VH of SEQ ID NO: 230 and the VL of SEQ ID NO: 231. (e.g. KEYTRUDA® (pembrolizumab)).

The antibody is suitable for use in therapy in a subject who is being treated or who has been treated with anti-PD-1 antibody comprising the VH of SEQ ID NO: 232 and the VL of SEQ ID NO: 233. (e.g. OPDIVO® (nivolumab)).

The antibody is suitable for use in therapy in a subject who is refractory to treatment with the anti-PD-1 antibody comprising the VH of SEQ ID NO: 230 and the VL of SEQ ID NO: 231. (e.g. KEYTRUDA® (pembrolizumab)).

The antibody is suitable for use in therapy in a subject who is refractory to treatment with the anti-PD-1 antibody comprising the VH of SEQ ID NO: 232 and the VL of SEQ ID NO: 233. (e.g. OPDIVO® (nivolumab)).

The antibody is suitable for use in therapy in a subject who has a relapsed tumor after treatment with the anti-PD-1 antibody comprising the VH of SEQ ID NO: 230 and the VL of SEQ ID NO: 231. (e.g. KEYTRUDA® (pembrolizumab).

The antibody is suitable for use in therapy in a subject who has a relapsed tumor after treatment with the anti-PD-1 antibody comprising the VH of SEQ ID NO: 232 and the VL of SEQ ID NO: 233. (e.g. OPDIVO® (nivolumab)).

The invention also provides an isolated antagonistic bispecific PD-1/TIM-3 antibody comprising a first domain specifically binding PD-1 and a second domain specifically binding TIM-3, wherein the first domain comprises the HCDR1, the HCDR2, the HCDR3, the LCDR1, the LCDR2 and the LCDR3 of SEQ ID NOs: 66, 67, 68, 69, 70 and 71, respectively, and the second domain comprises the HCDR1, the HCDR2, the HCDR3, the LCDR1, the LCDR2 and the LCDR3 of SEQ ID NOs 97, 105, 115, 124, 133 and 143, respectively.

In some embodiments, the antagonistic bispecific PD-1/TIM-3 antibody binds TIM-3 within TIM-3 residues 90-102 (RIQIPGIMNDEKF) (SEQ ID NO: 263).

In some embodiments, the bispecific PD-1/TIM-3 antibody binds TIM-3 within TIM-3 residues 90-102 (RIQIPGIMNDEKF) (SEQ ID NO: 263) and residues 50-56 (DERDVNY) SEQ ID NO: 262.

In some embodiments, the bispecific PD-1/TIM-3 antibody inhibits binding of TIM-3 to galectin-9.

In some embodiments, the first domain comprises the VH of SEQ ID NO: 64 and the VL of SEQ ID NO: 65 and the second domain comprises the VH of SEQ ID NO: 153 and the VL of SEQ ID NO: 162.

In some embodiments, the antibody is an IgG1 isotype.

In some embodiments, the antibody is an IgG2 isotype.

In some embodiments, the antibody is an IgG2 isotype comprising a F405L and/or a K409R substitution.

In some embodiments, the antibody is an IgG2 isotype, optionally comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype, optionally comprising a S228P substitution when compared to the wild type IgG4.

In some embodiments, the antibody is an IgG4 isotype comprising a F405L and a K409R substitution.

In some embodiments, the antibody is an IgG4 isotype comprising a heavy chain substitution S228P when compared to the wild type IgG4.

In some embodiments, the isolated bispecific PD-1/TIM-3 antibody comprises the HC1, the LC1, the HC2 and the LC2 of SEQ ID NOs: 187, 189, 190 and 193, respectively.

The invention also provides an isolated antagonistic bispecific PD-1/TIM-3 antibody comprising a first domain specifically binding PD-1 and a second domain specifically binding TIM-3, wherein the first domain comprises the HCDR1, the HCDR2, the HCDR3, the LCDR1, the LCDR2 and the LCDR3 of SEQ ID NOs: 66, 67, 68, 69, 70 and 71, respectively, and the second domain comprises the HCDR1, the HCDR2, the HCDR3, the LCDR1, the LCDR2 and the LCDR3 of SEQ ID NOs: 91, 99, 108, 118, 127 and 136, respectively.

In some embodiments, the first domain comprises the VH of SEQ ID NO: 64 and the VL of SEQ ID NO: 65 and the second domain comprises the VH of SEQ ID NO: 146 and the VL of SEQ ID NO: 156.

In some embodiments, the antibody is an IgG1 isotype.

In some embodiments, the antibody is an IgG2 isotype.

In some embodiments, the antibody is an IgG2 isotype comprising a F405L and/or a K409R substitution.

In some embodiments, the antibody is an IgG2 isotype, optionally comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype, optionally comprising a S228P substitution when compared to the wild type IgG4.

In some embodiments, the antibody is an IgG4 isotype comprising a F405L and a K409R substitution.

In some embodiments, the antibody is an IgG4 isotype comprising a heavy chain substitution S228P when compared to the wild type IgG4.

In some embodiments, the isolated antagonistic bispecific PD-1/TIM-3 antibody comprises the HC1, the LC1, the HC2 and the LC2 of SEQ ID NOs: 187, 189, 191 and 194, respectively.

In some embodiments, the isolated bispecific PD-1/TIM-3 antibody comprises the HC1, the LC1, the HC2 and the LC2 of SEQ ID NOs: 242, 189, 246 and 194, respectively.

The invention also provides an isolated antagonistic bispecific PD-1/TIM-3 antibody comprising a first domain specifically binding PD-1 and a second domain specifically binding TIM-3, wherein the first domain comprises the HCDR1, the HCDR2, the HCDR3, the LCDR1, the LCDR2 and the LCDR3 of SEQ ID NOs: 10, 14, 17, 23, 26 and 32, respectively, and the second domain comprises the HCDR1, the HCDR2, the HCDR3, the LCDR1, the LCDR2 and the LCDR3 of SEQ ID NOs: 97, 105, 115, 124, 133 and 143, respectively.

In some embodiments, the first domain comprises the VH of SEQ ID NO: 48 and the VL of SEQ ID NO: 56 and the second domain comprises the VH of SEQ ID NO: 172 and the VL of SEQ ID NO: 173.

In some embodiments, the antibody is an IgG1 isotype.

In some embodiments, the antibody is an IgG2 isotype.

In some embodiments, the antibody is an IgG2 isotype comprising a F405L and/or a K409R substitution.

In some embodiments, the antibody is an IgG2 isotype, optionally comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype, optionally comprising a S228P substitution when compared to the wild type IgG4.

In some embodiments, the antibody is an IgG4 isotype comprising a F405L and a K409R substitution.

In some embodiments, the antibody is an IgG4 isotype comprising a heavy chain substitution S228P when compared to the wild type IgG4.

In some embodiments, the isolated antagonistic bispecific PD-1/TIM-3 antibody comprises the HC1, the LC1, the HC2 and the LC2 of SEQ ID NOs: 186, 188, 192 and 195, respectively.

In some embodiments, the isolated antagonistic bispecific PD-1/TIM-3 antibody comprises the HC1, the LC1, the HC2 and the LC2 of SEQ ID NOs: 241, 188, 244 and 195, respectively.

In some embodiments, the isolated antagonistic bispecific PD-1/TIM-3 antibody comprises the HC1, the LC1, the HC2 and the LC2 of SEQ ID NOs: 243, 188, 247 and 195, respectively.

In some embodiments, the antibody enhances activation of antigen specific CD4⁺ or CD8⁺ T cells, wherein activation of antigen-specific CD4⁺ or CD8⁺ T cells is assessed by measuring a statistically significant enhancement of CD137 surface expression on antigen specific CD4⁺ or CD8⁺ T cells according to methods described in Example 14.

The invention also provides an isolated antagonistic bispecific PD-1/TIM-3 antibody comprising a first domain specifically binding PD-1 and a second domain specifically binding TIM-3, wherein the first domain comprises the HCDR1, the HCDR2, the HCDR3, the LCDR1, the LCDR2 and the LCDR3 of SEQ ID NOs: 10, 14, 17, 23, 26 and 32, respectively, and the second domain comprises the HCDR1, the HCDR2, the HCDR3, the LCDR1, the LCDR2 and the LCDR3 of SEQ ID NOs: 97, 105, 115, 124, 133 and 143, respectively.

In some embodiments, the first domain comprises the VH of SEQ ID NO: 48 and the VL of SEQ ID NO: 56 and the second domain comprises the VH of SEQ ID NO: 153 and the VL of SEQ ID NO: 156.

In some embodiments, the antibody is an IgG1 isotype.

In some embodiments, the antibody is an IgG2 isotype.

In some embodiments, the antibody is an IgG2 isotype comprising a F405L and/or a K409R substitution.

In some embodiments, the antibody is an IgG2 isotype, optionally comprising V234A, G237A, P238S, H268A, V309L, A330S and P331S substitutions when compared to the wild type IgG2.

In some embodiments, the antibody is an IgG3 isotype.

In some embodiments, the antibody is an IgG4 isotype, optionally comprising a S228P substitution when compared to the wild type IgG4.

In some embodiments, the antibody is an IgG4 isotype comprising a F405L and a K409R substitution.

In some embodiments, the antibody is an IgG4 isotype comprising a heavy chain substitution S228P when compared to the wild type IgG4.

In some embodiments, the isolated bispecific PD-1/TIM-3 antibody comprises the HC1I, the LC1I, the HC2 and the LC2 of SEQ ID NOs: 186, 188, 190 and 193, respectively.

Exemplary antagonistic bispecific PD-1/TIM-3 antibodies of the invention having certain VH, VL, HCDR and LCDR sequences as shown in Table 4 and Table 5.

TABLE 4

| | PD-1 binding arm SEQ ID NOs: | | | | | | | |
|---|---|---|---|---|---|---|---|---|
| | | | HCDRs | | | LCDRs | | |
| mAb | VH | VL | 1 | 2 | 3 | 1 | 2 | 3 |
| PTBB14 | 48 | 56 | 10 | 14 | 17 | 23 | 26 | 32 |
| PTBB15 | 48 | 56 | 10 | 14 | 17 | 23 | 26 | 32 |
| PTBB16 | 64 | 65 | 66 | 67 | 68 | 69 | 70 | 71 |
| PTBB17 | 64 | 65 | 66 | 67 | 68 | 69 | 70 | 71 |
| PTBB24 | 48 | 56 | 10 | 14 | 17 | 23 | 26 | 32 |
| PTBB30 | 48 | 56 | 10 | 14 | 17 | 23 | 26 | 32 |
| PTBB27 | 48 | 56 | 10 | 14 | 17 | 23 | 26 | 32 |
| PTBB28 | 48 | 56 | 10 | 14 | 17 | 23 | 26 | 32 |
| PTBB18 | 64 | 65 | 66 | 67 | 68 | 69 | 70 | 71 |
| PTBB20 | 48 | 56 | 10 | 14 | 17 | 23 | 26 | 32 |
| PTBB21 | 48 | 56 | 10 | 14 | 17 | 23 | 26 | 32 |

TABLE 5

| | TIM-3 binding arm SEQ ID NOs: | | | | | | | |
|---|---|---|---|---|---|---|---|---|
| | | | HCDRs | | | LCDR2 | | |
| mAb | VH | VL | 1 | 2 | 3 | 1 | 2 | 3 |
| PTBB14 | 153 | 162 | 97 | 105 | 115 | 124 | 133 | 143 |
| PTBB15 | 146 | 156 | 91 | 99 | 108 | 118 | 127 | 136 |
| PTBB16 | 153 | 162 | 97 | 105 | 115 | 124 | 133 | 143 |
| PTBB17 | 146 | 156 | 91 | 99 | 108 | 118 | 127 | 136 |
| PTBB24 | 172 | 173 | 97 | 105 | 115 | 124 | 133 | 143 |
| PTBB30 | 146 | 156 | 91 | 99 | 108 | 118 | 127 | 136 |
| PTBB27 | 172 | 173 | 97 | 105 | 115 | 124 | 133 | 143 |
| PTBB28 | 146 | 156 | 91 | 99 | 108 | 118 | 127 | 136 |
| PTBB18 | 146 | 156 | 91 | 99 | 108 | 118 | 127 | 136 |
| PTBB20 | 146 | 156 | 91 | 99 | 108 | 118 | 127 | 136 |
| PTBB21 | 172 | 173 | 97 | 105 | 115 | 124 | 133 | 143 |

### Engineered and Modified Antibodies

The antibodies of the invention may further be engineered to generate modified antibodies with similar or altered properties when compared to the parental antibodies. The VH, the VL, the VH and the VL, the constant regions, VH framework, VL framework, or any or all of the six CDRs may be engineered in the antibodies of the invention.

“The antibodies of the invention” as used herein refers to the antagonistic antibodies specifically binding PD-1, the antagonistic antibodies specifically binding TIM-3, and the antagonistic bispecific PD-1/TIM-3 antibodies comprising a first domain specifically binding PD-1 and a second domain specifically binding TIM-3 (e.g. bispecific PD-1/TIM-3 antibodies) as described herein.

The antibodies of the invention may be engineered by CDR grafting. One or more CDR sequences of the antibodies of the invention described herein may be grafted to a different framework sequence. CDR grafting may be done using known methods and methods described herein.

In some embodiments, the antagonistic antibodies specifically binding PD-1 or the bispecific PD-1/TIM-3 antibodies of the invention comprise the VH that comprises the HDCR1 of SEQ ID NOs: 10, 11 or 12, the HCDR2 of SEQ ID NOs: 13, 14 or 15, the HCDR3 of SEQ ID NOs: 16, 17, 18 or 19, and the VL that comprises the LCDR1 of SEQ ID NOs: 20, 21, 22, 23, 24 or 25, the LCDR2 of SEQ ID NOs: 26, 27, 28, 29 or 30, and/or the LCDR3 of SEQ ID NOs: 31, 32, 33, 34, 35, 36, 37, 38, 39 or 40, wherein the VH framework is derived from the VH framework other than VH1-69 (SEQ ID NO: 170) and the VL framework is derived from the VL framework other than IGKV3-11 (SEQ ID NO: 171).

In some embodiments, the antagonistic antibodies specifically binding TIM-3 or the bispecific PD-1/TIM-3 antibodies of the invention comprise the HDCR1 of SEQ ID NOs: 90, 91, 92, 93, 94, 95, 96, 97 or 98, the HCDR2 of SEQ ID NOs: 99, 100, 101, 102, 10, 104, 105 or 106, the HCDR3 of SEQ ID NOs: 107, 108, 109, 110, 111, 112, 113, 114, 115 or 116, and the VL that comprises the LCDR1 of SEQ ID NOs: 117, 118, 119, 120, 121, 122, 123, 124 or 125, the LCDR2 of SEQ ID NOs: 126, 127, 128, 129, 130, 131, 132, 133 or 134, and/or the LCDR3 of SEQ ID NOs: 135, 136, 137, 139, 140, 141, 142, 143 or 144, wherein the VH framework is derived from the human VH germline gene sequences other than those of IGHV3-23 (SEQ ID NO: 174), IGHV1-02 (SEQ ID NO: 175), IGHV4-30-4 (SEQ ID NO: 176), IGHV1-03 (SEQ ID NO: 177), IGHV2-26 (SEQ ID NO: 178) or IGHV5-51 (SEQ ID NO: 179), and the VL framework is derived from the human VL germline gene sequences other than those of IGKV3-20 (A27) (SEQ ID NO: 180), IGKV3-11 (L6) (SEQ ID NO: 171), IGKV4-1 (B3) (SEQ ID NO: 181), IGKV1-39 (012) (SEQ ID NO: 182) or IGKV1-33 (018) (SEQ ID NO: 183).

The framework sequences to be used may be obtained from public DNA databases or published references that include germline antibody gene sequences. For example, germline DNA and the encoded protein sequences of human heavy and light chain variable region genes may be found at IMGT®, the international ImMunoGeneTics information System® (http://_www-imgt_org). Framework sequences that may be used to replace the existing framework sequences in the antibodies of the invention may be those that show the highest percent identity to the parental frameworks over the entire length of the VH or the VL, or over the length of the FR1, FR2, FR3 and FR4. In addition, suitable frameworks may further be selected based on the VH and the VL CDR1 and CDR2 lengths or identical LCDR1, LCDR2, LCDR3, HCDR1 and HCDR2 canonical structure. Suitable frameworks may be selected using known methods, such as human framework adaptation described in U.S. Pat. No. 8,748,356 or superhumanization described in U.S. Pat. No. 7,709,226.

The framework sequences of the parental and engineered antibodies may further be modified, for example by backmutations to restore and/or improve binding of the generated antibody to the antigen as described for example in U.S. Pat. No. 6,180,370. The framework sequences of the parental or engineered antibodies may further be modified by mutating one or more residues within the framework region, or within one or more CDR regions, to remove T-cell epitopes to thereby reduce the potential immunogenicity of the antibody. This approach is also referred to as “deimmunization” and described in further detail in U.S. Patent Publ. No. US20070014796.

The CDR residues of the antibodies of the invention may be mutated to improve affinity of the antibodies to PD-1, TIM-3, or PD-1 and TIM-3.

The CDR residues of the antibodies of the invention may be mutated for example to minimize risk of post-translational modifications. Amino acid residues of putative motifs for deamination (NS), acid-catalyzed hydrolysis (DP), isomerization (DS), or oxidation (W) may be substituted with any of the naturally occurring amino acids to mutagenize the motifs, and the resulting antibodies may be tested for their functionality and stability using methods described herein.

Fc substitutions may be made to the antibodies of the invention to modulate antibody effector functions and pharmacokinetic properties. In traditional immune function, the interaction of antibody-antigen complexes with cells of the immune system results in a wide array of responses, ranging from effector functions such as antibody-dependent cytotoxicity, mast cell degranulation, and phagocytosis to immunomodulatory signals such as regulating lymphocyte proliferation and antibody secretion. All of these interactions are initiated through the binding of the Fc domain of antibodies or immune complexes to specialized cell surface receptors on hematopoietic cells. The diversity of cellular responses triggered by antibodies and immune complexes results from the structural heterogeneity of the three Fc receptors: FcγRI (CD64), FcγRII (CD32), and FcγRIII (CD16). FcγRI (CD64), FcγRIIA (CD32A) and FcγRIII (CD16) are “activating Fcγ receptors” (i e, immune system enhancing); FcγRIIB (CD32B) is an inhibiting Fcγ receptor” (i.e., immune system dampening). Binding to the FcRn receptor modulates antibody half-life.

In some embodiments, the antagonistic antibodies of the invention comprise at least one substitution in an Fc region

In some embodiments, the antagonistic antibodies of the invention comprise one, two, three, four, five, six, seven, eight, nine, ten, eleven, twelve, thirteen, fourteen or Fc positions that may be substituted to modulate antibody half-life are those described for example in Dall'Acqua et al., (2006) J Biol Chem 281:23514-240, Zalevsky et al., (2010) Nat Biotechnol 28:157-159, Hinton et al., (2004) J Biol Chem 279(8):6213-6216, Hinton et al., (2006) J Immunol 176:346-356, Shields et al. (2001) J Biol Chem 276:6591-6607, Petkova et al., (2006). Int Immunol 18:1759-1769, Datta-Mannan et al., (2007) Drug Metab Dispos, 35:86-94, 2007, Vaccaro et al., (2005) Nat Biotechnol 23:1283-1288, Yeung et al., (2010) Cancer Res, 70:3269-3277 and Kim et al., (1999) Eur J Immunol 29: 2819, and include positions 250, 252, 253, 254, 256, 257, 307, 376, 380, 428, 434 and 435. Exemplary substitutions that may be made singularly or in combination are substitutions T250Q, M252Y, I253A, S254T, T256E, P2571, T307A, D376V, E380A, M428L, H433K, N434S, N434A, N434H, N434F, H435A and H435R. Exemplary singular or combination substitutions that may be made to increase the half-life of the antibody are substitutions M428L/N434S, M252Y/S254T/T256E, T250Q/M428L, N434A and T307A/E380A/N434A. Exemplary singular or combination substitutions that may be made to reduce the half-life of the antibody are substitutions H435A, P2571/N434H, D376V/N434H, M252Y/S254T/T256E/H433K/N434F, T308P/N434A and H435R.

In some embodiments, the antibodies of the invention comprise at least one substitution in the antibody Fe at amino acid position 250, 252, 253, 254, 256, 257, 307, 376, 380, 428, 434 or 435.

In some embodiments, the antibodies of the invention comprise at least one substitution in the antibody Fc selected from the group consisting of T250Q, M252Y, I253A, S254T, T256E, P2571, T307A, D376V, E380A, M428L, H433K, N434S, N434A, N434H, N434F, H435A and H435R.

In some embodiments, the antibodies of the invention comprise at least one substitution in the antibody Fc selected from the group consisting of M428L/N434S, M252Y/S254T/T256E, T250Q/M428L, N434A, T307A/E380A/N434A, H435A, P2571/N434H, D376V/N434H, M252Y/S254T/T256E/H433K/N434F, T308P/N434A and H435R.

In some embodiments, the antibodies of the invention comprise at least one substitution in the antibody Fe that reduces binding of the antibody to an activating Fcγ receptor (FcγR) and/or reduces Fe effector functions such as C1q binding, complement dependent cytotoxicity (CDC), antibody-dependent cell-mediated cytotoxicity (ADCC) or phagocytosis (ADCP).

Fc positions that may be substituted to reduce binding of the antibody to the activating FcγR and subsequently to reduce effector function are those described for example in Shields et al., (2001) J Biol Chem 276:6591-6604, Intl. Patent Publ. No.

WO2011/066501, U.S. Pat. Nos. 6,737,056 and 5,624,821, Xu et al., (2000) Cell Immunol, 200:16-26, Alegre et al., (1994) Transplantation 57:1537-1543, Bolt et al., (1993) Eur J Immunol 23:403-411, Cole et al., (1999) Transplantation, 68:563-571, Rother et al., (2007) Nat Biotechnol 25:1256-1264, Ghevaert et al., (2008) J Clin Invest 118:2929-2938, An et al., (2009) mAbs, 1:572-579) and include positions 214, 233, 234, 235, 236, 237, 238, 265, 267, 268, 270, 295, 297, 309, 327, 328, 329, 330, 331 and 365. Exemplary substitutions that may be made singularly or in combination are substitutions K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S in IgG1, IgG2, IgG3 or IgG4. Exemplary combination substitutions that result in antibodies with reduced ADCC are substitutions L234A/L235A on IgG1, V234A,/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P33IS on IgG1, S228P/F234A/L235A/G237A/P238S on IgG4, and S228P/F234A/L235A/G236-deleted/G237A/P238S on IgG4. Hybrid IgG2/4 Fe domains may also be used, such as Fc with residues 117-260 from IgG2 and residues 261-447 from IgG4.

Well-known S228P substitution may be made in IgG4 antibodies to enhance IgG4 stability.

In some embodiments, the antibodies of the invention comprise a substitution in at least one residue position 214, 233, 234, 235, 236, 237, 238, 265, 267, 268, 270, 295, 297, 309, 327, 328, 329, 330, 331 or 365, wherein residue numbering is according to the EU Index.

In some embodiments, the antibodies of the invention comprise at least one substitution selected from the group consisting of K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S, wherein residue numbering is according to the EU Index.

In some embodiments, the antibodies of the invention comprise a substitution in at least one residue position 228, 234, 235, 237, 238, 268, 330 or 331, wherein residue numbering is according to the EU Index.

In some embodiments, the antibodies of the invention comprise a S228P substitution, wherein residue numbering is according to the EU Index.

In some embodiments, the antibodies of the invention comprise a V234A substitution, wherein residue numbering is according to the EU Index.

In some embodiments, the antibodies of the invention comprise a F234A substitution, wherein residue numbering is according to the EU Index.

In some embodiments, the antibodies of the invention comprise a G237A substitution, wherein residue numbering is according to the EU Index.

In some embodiments, the antibodies of the invention comprise a P238S substitution, wherein residue numbering is according to the EU Index.

In some embodiments, the antibodies of the invention comprise a H268A substitution, wherein residue numbering is according to the EU Index.

In some embodiments, the antibodies of the invention comprise a Q268A substitution, wherein residue numbering is according to the EU Index.

In some embodiments, the antibodies of the invention comprise an A330S substitution, wherein residue numbering is according to the EU Index.

In some embodiments, the antibodies of the invention comprise a P331S substitution, wherein residue numbering is according to the EU Index.

In some embodiments, the antibodies of the invention comprise L234A, L235A, G237A, P238S, H268A, A330S and P331S substitutions, wherein residue numbering is according to the EU Index.

In some embodiments, the antibodies of the invention comprise V234A, G237A, P238S, H268A, V309L, A330S and P331S substitutions, wherein residue numbering is according to the EU Index.

In some embodiments, the antibodies of the invention comprise F234A, L235A, G237A, P238S and Q268A substitutions, wherein residue numbering is according to the EU Index.

In some embodiments, the antibodies of the invention comprise L234A, L235A or L234A and L235A substitutions, wherein residue numbering is according to the EU Index.

In some embodiments, the antibodies of the invention comprise F234A, L235A or F234A and L235A substitutions, wherein residue numbering is according to the EU Index.

In some embodiments, the antibodies of the invention comprise S228P, F234A and L235A substitutions, wherein residue numbering is according to the EU Index.

In some embodiments, the antibodies of the invention comprise at least one substitution in an antibody Fe that enhances binding of the antibody to an Fcγ receptor (FcγR) and/or enhances Fe effector functions such as C1q binding, complement dependent cytotoxicity (CDC), antibody-dependent cell-mediated cytotoxicity (ADCC) or phagocytosis (ADCP).

In addition to their immunomodulatory activity, the PD-1 or the TIM-3 antibodies of the invention may kill tumor cells expressing PD-1 and/or TIM-3 directly via antibody-mediated effector functions, for example by ADCC, ADCP or CDC.

Fc positions that may be substituted to increase binding of the antibody to the activating Fcγ and/or enhance antibody effector functions are those described for example in U.S. Pat. No. 6,737,056, U.S. Patent Publ. No. 2015/0259434, Shields et al., (2001) J Biol Chem 276:6591-6604, Lazar et al., (2006) Proc Natal Acad Sci, 103:4005-4010, Stavenhagen et al., (2007) Cancer Res 67:8882-8890, Richards et al., (2008) Mol Cancer Ther 7:2517-2527, Diebolder et al., Science; published online Mar. 13, 2014; doi:10.1126/science.1248943, and include positions 236, 239, 243, 256, 290, 292, 298, 300, 305, 312, 326, 330, 332, 333, 334, 345, 360, 339, 378, 396 or 430 (residue numbering according to the EU index). Exemplary substitutions that may be made singularly or in combination are G236A, S239D, F243L, T256A, K290A, R292P, S298A, Y300L, V305L, K326A, A330K, 1332E, E333A, K334A, A339T and P396L. Exemplary combination substitutions that result in antibodies with increased ADCC or ADCP are substitutions S239D/1332E, S298A/E333A/K334A, F243L/R292P/Y300L, F243L/R292P/Y300L/P396L, F243L/R292P/Y300L/V305I/P396L and G236A/S239D/1332E on IgG1.

Fc positions that may be substituted to enhance CDC of the antibody are those described for example in Int. Patent Appl. WO2014/108198, Idusogie et al., (2001) J Immunol 166:2571-2575 and Moore et al., (2010) Mabs, 2:181-189, and include positions 267, 268, 324, 326, 333, 345 and 430. Exemplary substitutions that may be made singularly or in combination are substitutions S267E, H268F, S324T, K326A, K326W, E333A, E345K, E345Q, E345R, E345Y, E430S, E430F and E430T. Exemplary combination substitutions that result in antibodies with increased CDC are substitutions K326A/E333A, K326W/E333A, H268F/S324T, S267E/H268F, S267E/S324T and S267E/H268F/S324T on IgG1.

“Antibody-dependent cellular cytotoxicity”, “antibody-dependent cell-mediated cytotoxicity” or “ADCC” is a mechanism for inducing cell death that depends upon the interaction of antibody-coated target cells with effector cells possessing lytic activity, such as natural killer cells, monocytes, macrophages and neutrophils via Fe gamma receptors (FcγR) expressed on effector cells. For example, NK cells express FcγRIIIa, whereas monocytes express FcγRI, FcγRII and FcγRIIIa. Death of the antibody-coated target cell, such as PD-1 or TIM-3 expressing cells, occurs as a result of effector cell activity through the secretion of membrane pore-forming proteins and proteases. To assess ADCC activity of the antibody of the invention described herein, the antibody may be added to TIM-3 or PD-1 expressing cells in combination with immune effector cells, which may be activated by the antigen antibody complexes resulting in cytolysis of the target cell. Cytolysis may be detected by the release of label (e.g. radioactive substrates, fluorescent dyes or natural intracellular proteins) from the lysed cells. Exemplary effector cells for such assays include peripheral blood mononuclear cells (PBMC) and NK cells. Exemplary target cells include cells expressing TIM-3 or PD-1 either endogenously or recombinantly. In an exemplary assay, target cells are used with a ratio of 1 target cell to 50 effector cells. Target cells are pre-labeled with BATDA (PerkinElmer) for 20 minutes at 37° C., washed twice and resuspended in DMEM, 10% heat-inactivated FBS, 2 mM L-glutamine (all from Invitrogen). Target (1×10⁴ cells) and effector cells (0.5×10⁶ cells) are combined and 100 μl of cells are added to the wells of 96-well U-bottom plates. An additional 100 l is added with or without the test antibodies. The plates are centrifuged at 200 g for 3 minutes, incubated at 37° C. for 2 hours, and then centrifuged again at 200 g for 3 minutes. A total of 20 μl of supernatant is removed per well and cell lysis is measured by the addition of 200 μl of the DELPHIA Europium-based reagent (PerkinElmer). Data is normalized to maximal cytotoxicity with 0.67% Triton X-100 (Sigma Aldrich) and minimal control determined by spontaneous release of BATDA from target cells in the absence of any antibody. The antibody of the invention may induce ADCC by about 20%, 25%, 30%, 35%, 40%, 45%, 50%, 55%, 60%, 65%, 70%, 75%, 80%, 85%, 90%, 95% or 100%.

“Antibody-dependent cellular phagocytosis” (“ADCP”) refers to a mechanism of elimination of antibody-coated target cells by internalization by phagocytic cells, such as macrophages or dendritic cells. ADCP may be evaluated by using monocyte-derived macrophages as effector cells and Daudi cells (ATCC® CCL-213™) or B cell leukemia or lymphoma or tumor cells expressing TIM-3 or PD-1 as target cells engineered to express GFP or other labeled molecule. Effector:target cell ratio may be for example 4:1. Effector cells may be incubated with target cells for 4 hours with or without the antibody of the invention. After incubation, cells may be detached using accutase. Macrophages may be identified with anti-CD11b and anti-CD14 antibodies coupled to a fluorescent label, and percent phagocytosis may be determined based on % GFP fluorescence in the CD11⁺CD14⁺ macrophages using standard methods. The antibody of the invention may induce ADCP by about 20%, 25%, 30%, 35%, 40%, 45%, 50%, 55%, 60%, 65%, 70%, 75%, 80%, 85%, 90%, 95% or 100%.

“Complement-dependent cytotoxicity”, or “CDC”, refers to a mechanism for inducing cell death in which the Fe effector domain of a target-bound antibody binds and activates complement component C1q which in turn activates the complement cascade leading to target cell death. Activation of complement may also result in deposition of complement components on the target cell surface that facilitate ADCC by binding complement receptors (e.g., CR3) on leukocytes. CDC of TIM-3 or PD-1 expressing cells may be measured for example by plating Daudi cells at 1×10⁵ cells/well (50 1l/well) in RPMI-B (RPMI supplemented with 1% BSA), adding 50 μl of test antibodies to the wells at final concentration between 0-100 g/ml, incubating the reaction for 15 min at room temperature, adding 11 μl of pooled human serum to the wells, and incubation the reaction for 45 min at 37° C. Percentage (%) lysed cells may be detected as % propidium iodide stained cells in FACS assay using standard methods. Antibodies of the invention may induce CDC by about 20%, 25%, 30%, 35%, 40%, 45%, 50%, 55%, 60%, 65%, 70%, 75%, 80%, 85%, 90%, 95% or 100%.

The ability of antibodies of the invention described herein to induce ADCC may be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen binding or CDC activity. Such mAbs may be achieved using different methods reported to lead to the successful expression of relatively high defucosylated antibodies bearing the biantennary complex-type of Fe oligosaccharides such as control of culture osmolality (Konno et al., (2012) Cytotechnology 64:249-65), application of a variant CHO line Lec13 as the host cell line (Shields et al., (2002) J Biol Chem 277:26733-26740), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., (2003) J Biol Chem 278:3466-3473), introduction of small interfering RNA specifically against the a 1,6-fucosyltrasferase (FUT8) gene (Mori et al., (2004) Biotechnol Bioeng 88:901-908), or coexpression of β-1,4-N-acetylglucosaminyltransferase III and Golgi a-mannosidase II or a potent alpha-mannosidase I inhibitor, kifunensine (Ferrara et al., (2006) J Biol Chem 281:5032-5036, Ferrara et al., (2006) Biotechnol Bioeng 93:851-861; Xhou et al., (2008) Biotechnol Bioeng 99:652-65).

In some embodiments, the antibodies of the invention comprise at least one substitution in the antibody Fe that enhances effector function of the antibody.

In some embodiments, the antibodies of the invention comprise at least one substitution in the antibody Fe at amino acid position 236, 239, 243, 256, 267, 268, 290, 292, 298, 300, 305, 312, 324, 326, 330, 332, 333, 334, 345, 360, 339, 378, 396 or 430.

In some embodiments, the antibodies of the invention comprise at least one substitution in the antibody Fc selected from the group consisting of G236A, S239D, F243L, T256A, K290A, R292P, S298A, Y300L, V305L, K326A, A330K, 1332E, E333A, K334A, A339T, P396L, S267E, H268F, S324T, K326A, K326W, E333A, E345K, E345Q, E345R, E345Y, E430S, E430F and E430T.

In some embodiments, the antibodies of the invention comprise at least one substitution in the antibody Fc selected from the group consisting of S239D/I332E, S298A/E333A/K334A, F243L/R292P/Y300L, F243L/R292P/Y300L/P396L, F243L/R292P/Y300L/V305I/P396L, G236A/S239D/I332E, K326A/E333A, K326W/E333A, H268F/S324T, S267E/H268F, S267E/S324T and S267E/H268F/S324T.

In some embodiments, the antibodies of the invention have a biantennary glycan structure with fucose content of about between 0% to about 15%, for example 15%, 14%, 13%, 12%, 11% 10%, 9%, 8%, 7%, 6%, 5%, 4%, 3%, 2%, 1% or 0%.

In some embodiments, the antibodies of the invention have a biantennary glycan structure with fucose content of about 50%, 40%, 45%, 40%, 35%, 30%, 25%, 20%, 15%, 14%, 13%, 12%, 11% 10%, 9%, 8%, 7%, 6%, 5%, 4%, 3%, 2%, 1% or 0%.

Substitutions in the Fe and reduced fucose content may enhance the ADCC activity of the antagonistic antibodies specifically binding TIM-3 or PD-1 of the invention. TIM-3 or PD-1 antibodies with enhanced ADCC, ADCP and/or CDC activity may be useful in the treatment of patients with TIM-3 and/or PD-1 expressing tumors, including heme malignancies.

“Fucose content” means the amount of the fucose monosaccharide within the sugar chain at Asn297. The relative amount of fucose is the percentage of fucose-containing structures related to all glycostructures. These may be characterized and quantified by multiple methods, for example: 1) using MALDI-TOF of N-glycosidase F treated sample (e.g. complex, hybrid and oligo- and high-mannose structures) as described in Intl. Patent Publ. No. WO2008/077546; 2) by enzymatic release of the Asn297 glycans with subsequent derivatization and detection/quantitation by HPLC (UPLC) with fluorescence detection and/or HPLC-MS (UPLC-MS); 3) intact protein analysis of the native or reduced mAb, with or without treatment of the Asn297 glycans with Endo S or other enzyme that cleaves between the first and the second GlcNAc monosaccharides, leaving the fucose attached to the first GlcNAc; 4) digestion of the mAb to constituent peptides by enzymatic digestion (e.g., trypsin or endopeptidase Lys-C), and subsequent separation, detection and quantitation by HPLC-MS (UPLC-MS) or 5) separation of the mAb oligosaccharides from the mAb protein by specific enzymatic deglycosylation with PNGase F at Asn 297. The oligosaccharides released may be labeled with a fluorophore, separated and identified by various complementary techniques which allow fine characterization of the glycan structures by matrix-assisted laser desorption ionization (MALDI) mass spectrometry by comparison of the experimental masses with the theoretical masses, determination of the degree of sialylation by ion exchange HPLC (GlycoSep C), separation and quantification of the oligosaccharide forms according to hydrophilicity criteria by normal-phase HPLC (GlycoSep N), and separation and quantification of the oligosaccharides by high performance capillary electrophoresis-laser induced fluorescence (HPCE-LIF).

“Low fucose” or “low fucose content” refers to antibodies with fucose content of about 0%-15%.

“Normal fucose” or ‘normal fucose content” refers to antibodies with fucose content of about over 50%, typically about over 60%, 70%, 80% or over 85%.

The antibodies of the invention may be post-translationally modified by processes such as glycosylation, isomerization, deglycosylation or non-naturally occurring covalent modification such as the addition of polyethylene glycol moieties (pegylation) and lipidation. Such modifications may occur in vivo or in vitro. For example, the antibodies of the invention described herein may be conjugated to polyethylene glycol (PEGylated) to improve their pharmacokinetic profiles. Conjugation may be carried out by techniques known to those skilled in the art. Conjugation of therapeutic antibodies with PEG has been shown to enhance pharmacodynamics while not interfering with function (Knigh et al., (2004) Platelets 15:409-18; Leong et al., (2001) Cytokine 16:106-19; Yang et al., (2003) Protein Eng 16:761-70).

Antibodies of the invention may be modified to improve stability, selectivity, cross-reactivity, affinity, immunogenicity or other desirable biological or biophysical property are within the scope of the invention. Stability of an antibody is influenced by a number of factors, including (1) core packing of individual domains that affects their intrinsic stability, (2) protein/protein interface interactions that have impact upon the HC and LC pairing, (3) burial of polar and charged residues, (4) H-bonding network for polar and charged residues; and (5) surface charge and polar residue distribution among other intra- and inter-molecular forces (Worn et al., (2001) J Mol Biol 305:989-1010). Potential structure destabilizing residues may be identified based upon the crystal structure of the antibody or by molecular modeling in certain cases, and the effect of the residues on antibody stability may be tested by generating and evaluating variants harboring mutations in the identified residues. One of the ways to increase antibody stability is to raise the thermal transition midpoint (Tₘ) as measured by differential scanning calorimetry (DSC). In general, the protein Tₘ is correlated with its stability and inversely correlated with its susceptibility to unfolding and denaturation in solution and the degradation processes that depend on the tendency of the protein to unfold (Remmele et al., (2000) Biopharm 13:36-46). A number of studies have found correlation between the ranking of the physical stability of formulations measured as thermal stability by DSC and physical stability measured by other methods (Gupta et al., (2003) AAPS PharmSci 5E8; Zhang et al., (2004) J Pharm Sci 93:3076-89; Maa et al., (1996) Int J Pharm 140:155-68; Bedu-Addo et al., (2004) Pharm Res 21:1353-61; Remmele et al., (1997) Pharm Res 15:200-8). Formulation studies suggest that a Fab Tₘ has implication for long-term physical stability of a corresponding mAb.

C-terminal lysine (CTL) may be removed from injected antibodies by endogenous circulating carboxypeptidases in the blood stream (Cai et al., (2011) Biotechnol Bioeng 108:404-412). During manufacturing, CTL removal may be controlled to less than the maximum level by control of concentration of extracellular Zn²⁺, EDTA or EDTA—Fe³⁺ as described in U.S. Patent Publ. No. US20140273092. CTL content in antibodies can be measured using known methods.

In some embodiments, the antibodies of the invention have a C-terminal lysine content of about 10% to about 90%, about 20% to about 80%, about 40% to about 70%, about 55% to about 70%, or about 60%.

In some embodiments, the antibodies of the invention have a C-terminal lysine content of about 0%, 5%, 10%, 20%, 30%, 40%, 50%, 60%, 70%, 80%, 90% or 100%.

Methods of Generating Homologous Antibodies, Antibodies with Conservative Modifications, and Engineered and Modified Antibodies

The antibodies of the invention that have altered amino acid sequences when compared to the parental antibodies may be generated using standard cloning and expression technologies. For example, site-directed mutagenesis or PCR-mediated mutagenesis may be performed to introduce the mutation(s) and the effect on antibody binding or other property of interest, may be evaluated using well known methods and the methods described herein in the Examples.

### Antibody Allotypes

The antibody of the invention may be an IgG1, IgG2, IgG3 or IgG4 isotype.

In some embodiments, the antibody of the invention is an IgG1 isotype.

In some embodiments, the antibody of the invention is an IgG2 isotype.

In some embodiments, the antibody of the invention is an IgG3 isotype.

In some embodiments, the antibody of the invention is an IgG4 isotype.

Immunogenicity of therapeutic antibodies is associated with increased risk of infusion reactions and decreased duration of therapeutic response (Baert et al., (2003) N Engl J Med 348:602-08). The extent to which therapeutic antibodies induce an immune response in the host may be determined in part by the allotype of the antibody (Stickler et al., (2011) Genes and Immunity 12:213-21). Antibody allotype is related to amino acid sequence variations at specific locations in the constant region sequences of the antibody.

Table 6 shows select IgG1, IgG2 and IgG4 allotypes.

In some embodiments, the antagonistic antibodies specifically binding PD-1 of the invention are of G2m(n), G2m(n-), G2m(n)/(n-), nG4m(a), G1m(17) or G1m(17,1) allotype.

In some embodiments, the antagonistic antibodies specifically binding TIM-3 of the invention are of G2m(n), G2m(n-), G2m(n)/(n-), nG4m(a), G1m(17) or G1m(17,1) allotype.

In some embodiments, the bispecific PD-1/TIM-3 antibodies of the invention are of G2m(n), G2m(n-), G2m(n)/(n-), nG4m(a), G1m(17) or G1m(17,1) allotype.

TABLE 6

| | Amino acid residue at position of diversity | | | | | | | |
|---|---|---|---|---|---|---|---|---|
| | (residue numbering: EU Index) | | | | | | | |
| | IgG2 | | IgG4 | | IgG1 | | | |
| Allotype | 189 | 282 | 309 | 422 | 214 | 356 | 358 | 431 |
| G2m(n) | T | M | | | | | | |
| G2m(n-) | P | V | | | | | | |
| G2m(n)/(n-) | T | V | | | | | | |
| nG4m(a) | | | L | R | | | | |
| G1m(17) | | | | | K | E | M | A |
| Glm(17,1) | | | | | K | D | L | A |

### Anti-Idiotypic Antibodies

The present invention provides an anti-idiotypic antibody binding to the antibody of the invention.

The invention also provides an anti-idiotypic antibody specifically binding to the anti-PD-1 antibody of the invention.

The invention also provides an anti-idiotypic antibody specifically binding the antibody comprising the VH of SEQ ID NO: 41 and the VL of SEQ ID NO: 49.

The invention also provides an anti-idiotypic antibody specifically binding the antibody comprising the VH of SEQ ID NO: 41 and the VL of SEQ ID NO: 50.

The invention also provides an anti-idiotypic antibody specifically binding the antibody comprising the VH of SEQ ID NO: 42 and the VL of SEQ ID NO: 51.

The invention also provides an anti-idiotypic antibody specifically binding the antibody comprising the VH of SEQ ID NO: 42 and the VL of SEQ ID NO: 52.

The invention also provides an anti-idiotypic antibody specifically binding the antibody comprising the VH of SEQ ID NO: 42 and the VL of SEQ ID NO: 53.

The invention also provides an anti-idiotypic antibody specifically binding the antibody comprising the VH of SEQ ID NO: 43 and the VL of SEQ ID NO: 49.

The invention also provides an anti-idiotypic antibody specifically binding the antibody comprising the VH of SEQ ID NO: 43 and the VL of SEQ ID NO: 54.

The invention also provides an anti-idiotypic antibody specifically binding the antibody comprising the VH of SEQ ID NO: 43 and the VL of SEQ ID NO: 50.

The invention also provides an anti-idiotypic antibody specifically binding the antibody comprising the VH of SEQ ID NO: 43 and the VL of SEQ ID NO: 55.

The invention also provides an anti-idiotypic antibody specifically binding the antibody comprising the VH of SEQ ID NO: 43 and the VL of SEQ ID NO: 56.

The invention also provides an anti-idiotypic antibody specifically binding the antibody comprising the VH of SEQ ID NO: 43 and the VL of SEQ ID NO: 57.

The invention also provides an anti-idiotypic antibody specifically binding the antibody comprising the VH of SEQ ID NO: 44 and the VL of SEQ ID NO: 49.

The invention also provides an anti-idiotypic antibody specifically binding the antibody comprising the VH of SEQ ID NO: 45 and the VL of SEQ ID NO: 49.

The invention also provides an anti-idiotypic antibody specifically binding the antibody comprising the VH of SEQ ID NO: 46 and the VL of SEQ ID NO: 49.

The invention also provides an anti-idiotypic antibody specifically binding the antibody comprising the VH of SEQ ID NO: 47 and the VL of SEQ ID NO: 49. The invention also provides an anti-idiotypic antibody specifically binding the antibody comprising the VH of SEQ ID NO: 48 and the VL of SEQ ID NO: 53.

The invention also provides an anti-idiotypic antibody specifically binding the antibody comprising the VH of SEQ ID NO: 48 and the VL of SEQ ID NO: 52.

The invention also provides an anti-idiotypic antibody specifically binding the antibody comprising the VH of SEQ ID NO: 48 and the VL of SEQ ID NO: 56.

The invention also provides an anti-idiotypic antibody specifically binding the antibody comprising the VH of SEQ ID NO: 47 and the VL of SEQ ID NO: 58.

The invention also provides an anti-idiotypic antibody specifically binding the antibody comprising the VH of SEQ ID NO: 47 and the VL of SEQ ID NO: 59.

The invention also provides an anti-idiotypic antibody specifically binding the antibody comprising the VH of SEQ ID NO: 45 and the VL of SEQ ID NO: 60.

The invention also provides an anti-idiotypic antibody specifically binding the antibody comprising the VH of SEQ ID NO: 45 and the VL of SEQ ID NO: 61.

The invention also provides an anti-idiotypic antibody specifically binding the antibody comprising the VH of SEQ ID NO: 45 and the VL of SEQ ID NO: 62.

The invention also provides an anti-idiotypic antibody specifically binding the antibody comprising the VH of SEQ ID NO: 63 and the VL of SEQ ID NO: 65.

The invention also provides an anti-idiotypic antibody specifically binding the antibody comprising the VH of SEQ ID NO: 64 and the VL of SEQ ID NO: 65.

The invention also provides an anti-idiotypic antibody specifically binding the antagonistic antibody specifically binding TIM-3 of the invention.

The invention also provides an anti-idiotypic antibody specifically binding the antibody comprising the VH of SEQ ID NO: 145 and the VL of SEQ ID NO: 155.

The invention also provides an anti-idiotypic antibody specifically binding the antibody comprising the VH of SEQ ID NO: 146 and the VL of SEQ ID NO: 156.

The invention also provides an anti-idiotypic antibody specifically binding the antibody comprising the VH of SEQ ID NO: 148 and the VL of SEQ ID NO: 157.

The invention also provides an anti-idiotypic antibody specifically binding the antibody comprising the VH of SEQ ID NO: 147 and the VL of SEQ ID NO: 155.

The invention also provides an anti-idiotypic antibody specifically binding the antibody comprising the VH of SEQ ID NO: 149 and the VL of SEQ ID NO: 158.

The invention also provides an anti-idiotypic antibody specifically binding the antibody comprising the VH of SEQ ID NO: 150 and the VL of SEQ ID NO: 159.

The invention also provides an anti-idiotypic antibody specifically binding the antibody comprising the VH of SEQ ID NO: 151 and the VL of SEQ ID NO: 160.

The invention also provides an anti-idiotypic antibody specifically binding the antibody comprising the VH of SEQ ID NO: 152 and the VL of SEQ ID NO: 161.

The invention also provides an anti-idiotypic antibody specifically binding the antibody comprising the VH of SEQ ID NO: 153 and the VL of SEQ ID NO: 162.

The invention also provides an anti-idiotypic antibody specifically binding the antibody comprising the VH of SEQ ID NO: 154 and the VL of SEQ ID NO: 163.

In some embodiments, the kit comprises the antagonistic antibody specifically binding PD-I comprising the VH of SEQ ID NO: 48 and the VL of SEQ ID NO: 56.

In some embodiments, the kit comprises the antagonistic antibody specifically binding PD-I comprising the VH of SEQ ID NO: 64 and the VL of SEQ ID NO: 65.

The invention also provides an anti-idiotypic antibody specifically binding the antibody comprising the VH of SEQ ID NO: 172 and the VL of SEQ ID NO: 173.

In some embodiments, the anti-idiotypic antibody is used for detecting the level of the therapeutic antibodies (e.g. anti-PD-1, anti-TIM-3 or the bispecific PD-I/TIM-3 antibodies of the invention described herein) in a sample.

An anti-idiotypic (Id) antibody is an antibody which recognizes the antigenic determinants (e.g. the paratope or CDRs) of the antibody. The Id antibody may be antigen-blocking or non-blocking. The antigen-blocking Id may be used to detect the free antibody in a sample (e.g. anti-PD-1, anti-TIM-3 or the bispecific PD-I/TIM-3 antibody of the invention described herein). The non-blocking Id may be used to detect the total antibody (free, partially bond to antigen, or fully bound to antigen) in a sample. An Id antibody may be prepared by immunizing an animal with the antibody to which an anti-Id is being prepared.

An anti-Id antibody may also be used as an immunogen to induce an immune response in yet another animal, producing a so-called anti-anti-Id antibody. An anti-anti-Id may be epitopically identical to the original mAb, which induced the anti-Id. Thus, by using antibodies to the idiotypic determinants of a mAb, it is possible to identify other clones expressing antibodies of identical specificity. Anti-Id antibodies may be varied (thereby producing anti-Id antibody variants) and/or derivatized by any suitable technique, such as those described elsewhere herein with respect to the antibodies specifically binding PD-I or TIM-3, or the bispecific PD-I/TIM-3 antibodies.

### Immunoconjugates

An “immunoconjugate” refers to the antibody of the invention conjugated to one or more heterologous molecule(s).

In some embodiments, the antibody of the invention is conjugated to one or more cytotoxic agents or an imaging agent.

Exemplary cytotoxic agents include chemotherapeutic agents or drugs, growth inhibitory agents, toxins (e.g., protein toxins, enzymatically active toxins of bacterial, fungal, plant, or animal origin, or fragments thereof), and radionuclides.

The cytotoxic agent may be one or more drugs, such as to a mayatansinoid (see, e.g., U.S. Pat. No. 5,208,020, 5,416,06), an auristatin such as monomethylauristatin drug moieties DE and DF (MMAE and MMAF) (see, e.g., U.S. Pat. Nos. 5,635,483 and 5,780,588, and 7,498,298), a dolastatin, a calicheamicin or derivative thereof (see, e.g., U.S. Pat. Nos. 5,712,374, 5,714,586, 5,739, 116, 5,767,285, 5,770,701, 5,770,710, 5,773,001, and 5,877,296; Hinman et al., (1993) Cancer Res 53:3336-3342; and Lode et al.,(1998) Cancer Res 58:2925-2928); an anthracycline such as daunomycin or doxorubicin (see, e.g., Kratz et al., (2006) Current Med. Chem 13:477-523; Jeffrey et al., (2006) Bioorganic & Med Chem Letters 16:358-362; Torgov et al., (2005) Bioconj Chem 16:717-721; Nagy et al., (2000) Proc Natl Acad Sci USA 97:829-834; Dubowchik et al, Bioorg. & Med. Chem. Letters 12: 1529-1532 (2002); King et al., (2002) J Med Chem 45:4336-4343; and U.S. Pat. No. 6,630,579), methotrexate, vindesine, a taxane such as docetaxel, paclitaxel, larotaxel, tesetaxel, and ortataxel.

The cytotoxic agent may also be an enzymatically active toxin or fragment thereof, such as diphtheria A chain, nonbinding active fragments of diphtheria toxin, exotoxin A chain (from Pseudomonas aeruginosa), ricin A chain, modeccin A chain, alpha-sarcin, Aleurites fordii proteins, dianthins, Phytolacca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

The cytotoxic agent or an imaging agent may also be a radionuclide. Exemplary radionuclides include Ac-225, At-211, 1-131, 1-125, Y-90, Re-186, Re-188, Sm-153, Bi-212, P-32, Pb-212 and radioactive isotopes of Lu. When the radioconjugate is used for detection, it may comprise a radioactive atom for scintigraphic studies, for example Tc-99m or I-123, or a spin label for nuclear magnetic resonance (NMR) imaging (also known as magnetic resonance imaging, mri), such as 1-123, 1-131, In-111, F-19, C-13, N-15 or 0-17.

Conjugates of the antibodies of the invention and the heterologous molecule may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HQ), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin may be prepared as described in Vitetta et al., (1987) Science 238: 1098. Carbon-14-labeled 1-isothiocyanatobenzyl-3-methyldiethylene triaminepentaacetic acid (MX-DTPA) is an exemplary chelating agent for conjugation of radionucleotide to the antibody. See, e.g., WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., (1992) Cancer Res 52: 127-131; U.S. Pat. No. 5,208,020) may be used.

Conjugates of the antibodies of the invention and the heterologous molecule may be prepared with cross-linker reagents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

The invention also provides an immunoconjugate comprising the antagonistic antibody specifically binding PD-1 of the invention linked to a therapeutic agent or an imaging agent.

The invention also provides an immunoconjugate comprising the antagonistic antibody specifically binding TIM-3 of the invention linked to a therapeutic agent or an imaging agent.

The invention also provides an immunoconjugate comprising the bispecific PD-1/TIM-3 antibody of the invention linked to a therapeutic agent or an imaging agent.

### Generation of Monospecific Antibodies of the Invention

In some embodiments, the antibodies of the invention are human.

In some embodiments, the antibodies of the invention are humanized.

Monospecific antibodies of the invention described herein (e.g. antibodies specifically binding PD-1 or TIM-3) may be generated using various technologies. For example, the hybridoma method of Kohler and Milstein, Nature 256:495, 1975 may be used to generate monoclonal antibodies. In the hybridoma method, a mouse or other host animal, such as a hamster, rat or monkey, is immunized with human or cyno PD-1 or TIM-3 or fragments of PD-1 or TIM-3, such as the extracellular domain of PD-1 or TIM-3, followed by fusion of spleen cells from immunized animals with myeloma cells using standard methods to form hybridoma cells (Goding, Monoclonal Antibodies: Principles and Practice, pp. 59-103 (Academic Press, 1986)). Colonies arising from single immortalized hybridoma cells are screened for production of antibodies with desired properties, such as specificity of binding, cross-reactivity or lack thereof, and affinity for the antigen.

Various host animals may be used to produce the antibodies of the invention. For example, Balb/c mice may be used to generate mouse anti-human PD-1 or TIM-3 antibodies. The antibodies made in Balb/c mice and other non-human animals may be humanized using various technologies to generate more human-like sequences.

Exemplary humanization techniques including selection of human acceptor frameworks are known and include CDR grafting (U.S. Pat. No. 5,225,539), SDR grafting (U.S. Pat. No. 6,818,749), Resurfacing (Padlan, (1991) Mol Immunol 28:489-499), Specificity Determining Residues Resurfacing (U.S. Patent Publ. No. 2010/0261620), human framework adaptation (U.S. Pat. No. 8,748,356) or superhumanization (U.S. Pat. No. 7,709,226). In these methods, CDRs of parental antibodies are transferred onto human frameworks that may be selected based on their overall homology to the parental frameworks, based on similarity in CDR length, or canonical structure identity, or a combination thereof.

Humanized antibodies may be further optimized to improve their selectivity or affinity to a desired antigen by incorporating altered framework support residues to preserve binding affinity (backmutations) by techniques such as those described in Int. Patent Publ. Nos. WO1090/007861 and WO1992/22653, or by introducing variation at any of the CDRs for example to improve affinity of the antibody.

Transgenic animals, such as mice or rats carrying human immunoglobulin (Ig) loci in their genome may be used to generate human antibodies against a target protein, and are described in for example U.S. Pat. No. 6,150,584, Int. Patent Publ. No. WO99/45962, Int. Patent Publ. Nos. WO2002/066630, WO2002/43478, WO2002/043478 and WO1990/04036, Lonberg et al (1994) Nature 368:856-9; Green et al (1994) Nature Genet. 7:13-21; Green & Jakobovits (1998) Exp. Med. 188:483-95; Lonberg and Huszar (1995) Int Rev Immunol 13:65-93; Bruggemann et al., (1991) Eur J Immunol 21:1323-1326; Fishwild et al., (1996) Nat Biotechnol 14:845-851; Mendez et al., (1997) Nat Genet 15:146-156; Green (1999) J Immunol Methods 231:11-23; Yang et al., (1999) Cancer Res 59:1236-1243; Brnggemann and Taussig (1997) Curr Opin Biotechnol 8:455-458. The endogenous immunoglobulin loci in such animal may be disrupted or deleted, and at least one complete or partial human immunoglobulin locus may be inserted into the genome of the antimal using homologous or non-homologous recombination, using transchromosomes, or using minigenes. Companies such as Regeneron (http://_www_regeneron_com), Harbour Antibodies (http://_www_harbourantibodies_com), Open Monoclonal Technology, Inc. (OMT) (http://_www_omtinc net), KyMab (http://_www_kymab_com), Trianni (http://_www.trianni_com) and Ablexis (http://_www_ablexis_com) may be engaged to provide human antibodies directed against a selected antigen using technologies as described above.

Human antibodies may be selected from a phage display library, where the phage is engineered to express human immunoglobulins or portions thereof such as Fabs, single chain antibodies (scFv), or unpaired or paired antibody variable regions (Knappik et al., (2000) J Mol Biol 296:57-86; Krebs et al., (2001) J Immunol Meth 254:67-84; Vaughan et al., (1996) Nature Biotechnology 14:309-314; Sheets et al., (1998) PITAS (USA) 95:6157-6162; Hoogenboom and Winter (1991) J Mol Biol 227:381; Marks et al., (1991) J Mol Biol 222:581). The antibodies of the invention may be isolated for example from phage display library expressing antibody heavy and light chain variable regions as fusion proteins with bacteriophage pIX coat protein as described in Shi et al., (2010) J Mol Biol 397:385-96, and Int. Patent Publ. No. WO09/085462). The libraries may be screened for phage binding to human and/or cyno PD-1 or TIM-3 and the obtained positive clones may be further characterized, the Fabs isolated from the clone lysates, and expressed as full length IgGs. Such phage display methods for isolating human antibodies are described in for example: U.S. Pat. Nos. 5,223,409, 5,403,484, 5,571,698, 5,427,908, 5,580,717, 5,969,108, 6,172,197, 5,885,793; 6,521,404; 6,544,731; 6,555,313; 6,582,915 and 6,593,081.

Preparation of immunogenic antigens and monoclonal antibody production may be performed using any suitable technique, such as recombinant protein production. The immunogenic antigens may be administered to an animal in the form of purified protein, or protein mixtures including whole cells or cell or tissue extracts, or the antigen may be formed de novo in the animal's body from nucleic acids encoding said antigen or a portion thereof.

### Generation of Bispecific PD-1/TIM-3 Antibodies of the Invention

The bispecific PD-1/TIM-3 antibodies of the invention (e.g. the bispecific antibodies comprising a first domain specifically binding PD-1 and a second domain specifically binding TIM-3) may be generated by combining PD-1 binding VH/VL domains with TIM-3 binding VH/VL domains isolated and characterized herein. Alternatively, the bispecific PD-1/TIM-3 antibodies may be engineered using VH/VL domains from publicly available monospecific anti-PD-1 and anti-TIM-3 antibodies, and/or by mix-matching the PD-1 or TIM-3 binding VH/VL domains identified herein with publicly available PD-1 or TIM-3 binding VH/VL domains.

Exemplary anti-PD-1 antibodies that may be used to engineer bispecific PD-1/TIM-3 molecules are for example those described in U.S. Pat. Nos. 5,897,862 and 7,488,802, and in Int. Patent Publ. Nos. WO2004/004771, WO2004/056875, WO2006/121168, WO2008/156712, WO2010/029435, WO2010/036959, WO2011/110604, WO2012/145493, WO2014/194302, WO2014/206107, WO2015/036394, WO2015/035606, WO2015/085847, WO2015/112900 and WO2015/112805. For example, the VH/VL domains of KEYTRUDA® (pembrolizumab) and OPDIVO® (nivolumab) may be used. These PD-1 VH/VL domains may be incorporated into bispecific antibodies comprising TIM-3 binding VH/VL domains described herein and in Table 3. For example, the VH/VL domains of the TIM-3 antibodies TM3B103, TM3B105, TM3B107, TM3B108, TM3B109, TM3B113, TM3B189, TM3B190 and TM3B196 described herein may be used to generate bispecific PD-1/TIM-3 antibodies.

Similarly, exemplary anti-TIM-3 antibodies that may be used to engineer bispecific PD-1/TIM-3 molecules are for example those described in Int. Patent Publ. Nos. WO2011/155607, WO2013/006490, and WO2015/117002. These TIM-3 VH/VL domains may be incorporated into bispecific antibodies comprising PD-1 binding VH/VL domains described herein and in Table 2. For example, the VH/VL domains of the PD-1 antibodies PD1B114, PD1B149, PD1B160, PD1B162, PD1B164, PD1B11, PD1B183, PD1B184, PD1B185, PD1B187, PD1B192, PD1B71, PD1B177, PD1B70, PD1B175, PD1B194, PD1B195, PD1B196, PD1B197, PD1B198, PD1B199, PD1B200, PD1B201, PD1B131 and PD1B132 described herein may be used to generate bispecific PD-1/TIM-3 antibodies.

The generated bispecific PD-1/TIM-3 antibodies may be tested for their binding to PD-1 and TIM-3, and for their desired functional characteristics, such as enhancement of activation of antigen specific CD4⁺ and CD4⁺ T cells using methods described herein.

Bispecific antibodies of the invention comprise antibodies having a full length antibody structure.

Full length bispecific antibodies may be generated for example using Fab arm exchange (e.g., half molecule exchange, exchanging on heavy chain—light chain pair) between two monospecific bivalent antibodies by introducing mutations at the heavy chain CH3 interface in each half-molecule to favor heterodimer formation of two antibody half-molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms may be engineered to favor heterodimerization over homodimerization. The resulting product is a bispecific antibody having two Fab arms r half molecules which each bind a distinct epitope. Mutations F405L in one heavy chain and K409R in the other heavy chain may be used in case of IgG1 antibodies. For IgG2 antibodies, a wild-type IgG2 and a IgG2 antibody with F405L and R409K substitutions may be used. To generate bispecific antibodies, first monospecific bivalent antibody and the second monospecific bivalent antibody are engineered to have a F405L or a K409R mutation in the Fc region, the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2 carboxyethyl)phosphine (TCEP), L-cysteine and beta-mercaptoethanol. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Bispecific antibodies may also be generated using designs such as the Knob-in-Hole (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody)(EMD Serono), and the Biclonic (Merus).

The “knob-in-hole” strategy (see, e.g., Intl. Publ. No. WO 2006/028936) may be used to generate full length bispecific antibodies of the invention. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

The CrossMAb technology may be used to generate full length bispecific antibodies of the invention. CrossMAbs, in addition to utilizing the “knob-in-hole” strategy to promoter Fab arm exchange, have in one of the half arms the CH1 and the CL domains exchanged to ensure correct light chain pairing of the resulting bispecific antibody (see e.g. U.S. Pat. No. 8,242,247).

Other cross-over strategies may be used to generate full length bispecific antibodies of the invention by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface may be used, as described in US Patent Publ. No. US2010/0015133; US Patent Publ. No. US2009/0182127; US Patent Publ. No. US2010/028637 or US Patent Publ. No. US2011/0123532. In other strategies, heterodimerization may be promoted by following substitutions (expressed as modified positions in the first CH3 domain of the first heavy chain/modified position in the second CH3 domain of the second heavy chain): L351Y_F405A_Y407V/T394W, T3661_K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V L351Y F405A Y407V/T350V T366L K392L T394W as described in U.S. Patent Publ. No. US2012/0149876 or U.S. Patent Publ. No. US2013/0195849.

LUZ-Y technology may be utilized to generate bispecific antibodies of the invention. In this technology, a leucine zipper is added into the C terminus of the CH3 domains to drive the heterodimer assembly from parental mAbs that is removed post-purification as described in Wranik et al., (2012) J Biol Chem 287(52): 42221-9.

SEEDbody technology may be utilized to generate bispecific antibodies of the invention. SEEDbodies have, in their constant domains, select IgG residues substituted with IgA residues to promote heterodimerization as described in U.S. Patent No. US20070287170.

Mutations are typically made at the DNA level to a molecule such as the constant domain of the antibody using standard methods.

The antibodies of the invention may be engineered into various well known antibody formats.

In some embodiments, the bispecific antibodies include recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

### Polynucleotides, Vectors and Host Cells

The invention also provides an antagonistic antibody that specifically binds PD-1, TIM-3 or PD-1 and TIM-3 having certain VH and VL sequences, wherein the antibody VH is encoded by a first polynucleotide and the antibody VL is encoded by a second polynucleotide. The polynucleotide may be a complementary deoxynucleic acid (cDNA), and may be codon optimized for expression in suitable host. Codon optimization is a well-known technology.

The invention also provides an isolated polynucleotide encoding the VH of the antibody of the invention, the VL of the antibody of the invention, the heavy chain of the antibody of the invention or the light chain of the antibody of the invention.

The invention also provides an isolated polynucleotide encoding the VH, the VL, or the VH and the VL of the antagonistic antibody specifically binding PD-1 of the invention.

The invention also provides an isolated polynucleotide encoding the VH of SEQ ID NOs: 41, 42, 43, 44, 45, 46, 47, 48, 63 or 64.

The invention also provides an isolated polynucleotide encoding the VL of SEQ ID NOs: 49, 50, 51, 52, 53, 54, 55, 56, 57, 58, 59, 60, 61, 62 or 65.

The invention also provides an isolated polynucleotide comprising the polynucleotide sequence of SEQ ID NOs: 196, 197, 198, 199, 200, 201, 202 or 203.

The invention also provides an isolated polynucleotide encoding the VH, the VL, or the VH and the VL of the antagonistic antibody specifically binding TIM-3 of the invention.

The invention also provides an isolated polynucleotide encoding the VH of SEQ ID NOs: 145, 146, 147, 148, 149, 150, 151, 152, 153, 154 or 172.

The invention also provides an isolated polynucleotide encoding the VL of SEQ ID NOs: 155, 156, 157, 158, 159, 160, 161, 162, 163 or 173.

The invention also provides an isolated polynucleotide comprising the polynucleotide sequence of SEQ ID NOs: 204, 205, 206, 207, 208, 209, 210 or 211.

The invention also provides an isolated polynucleotide encoding the HC1, the LC1, the HC2 or the LC2 of the antagonistic bispecific PD-1/TIM-3 antibody of the invention.

The invention also provides an isolated polynucleotide encoding the HC1 of SEQ ID NOs: 186, 187, 241, 242 or 243.

The invention also provides an isolated polynucleotide encoding the LC1 of SEQ ID NOs: 188 or 189.

The invention also provides an isolated polynucleotide encoding the HC2 of SEQ ID NOs: 190, 191, 192, 244, 245, 246, 247 or 248.

The invention also provides an isolated polynucleotide encoding the LC2 of SEQ ID NOs: 193, 194 or 195.

The invention also provides an isolated polynucleotide comprising the polynucleotide sequence of SEQ ID NOs: 253, 254, 255, 256, 257, 258, 259 and 260.

(PD1H170)
SEQ ID NO: 196
CAGGTGCAGCTGGTGCAGAGCGGCGCGGAAGTGAAAAAACCGGGCAGCAG
CGTGAAAGTGAGCTGCAAAGCGAGCGGCGGCACCTTTAGCAGCTATGCGA
TTAGCTGGGTGCGCCAGGCGCCGGGCCAGGGCCTGGAATGGATGGGCGGC
ATTATTCCGATTTTTGACACCGCGAACTATGCGCAGAAATTTCAGGGCCG
CGTGACCATTACCGCGGATGAAAGCACCAGCACCGCGTATATGGAACTGA
GCAGCCTGCGCAGCGAAGATACCGCGGTGTATTATTGCGCGCGCCCTGGT
CTCGCTGCGGCTTATGATACTGGTTCCTTGGACTATTGGGGCCAGGGCAC
CCTGGTGACCGTGAGCAGC
(PD1L148)
SEQ ID NO: 197
GAAATTGTGCTGACCCAGAGCCCGGCGACCCTGAGCCTGAGCCCGGGCGA
ACGCGCGACCCTGAGCTGCCGCGCGAGCCAGAGCGTTCGCTCCTACCTGG
CGTGGTATCAGCAGAAACCGGGCCAGGCGCCGCGCCTGCTGATCTACGAC
GCGAGCAATCGTGCGACCGGCATTCCGGCGCGCTTTAGCGGCTCCGGTAG
CGGCACCGATTTTACCCTGACCATTAGCAGCCTGGAACCGGAAGATTTTG
CGGTGTATTATTGCCAGCAACGTAATTATTGGCCGCTGACCTTTGGCCAG
GGCACCAAAGTGGAAATTAAA
(PD1H129)
SEQ ID NO: 198
GAAGTGCAGCTGGTGGAATCTGGCGGCGGACTGGTGCAGCCTGGCGGATC
TCTGAGACTGAGCTGTGCCGCCAGCGGCTTCGCCTTCAGCAGATACGACA
TGAGCTGGGTGCGCCAGGCCCCTGGCAAAGGACTGGAAAGCGTGGCCTAC
ATCTCTGGCGGAGGCGCCAACACCTACTACCTGGACAACGTGAAGGGCCG
GTTCACCATCAGCCGGGACAACGCCAAGAACAGCCTGTACCTGCAGATGA
ACTCCCTGCGGGCCGAGGACACCGCCGTGTACTATTGCGCCTCCCCCTAC
CTGAGCTACTTCGACGTGTGGGGCCAGGGCACACTCGTGACCGTGTCATC
T
(PD1L62)
SEQ ID NO: 199
GAGATCGTGATGACCCAGAGCCCTGCCACCCTGTCCGTGTCTCCAGGCGA
AAGAGCCACCCTGAGCTGCAGAGCCAGCCAGAGCCTGAGCGACTACCTGC
ACTGGTATCAGCAGAAGCCCGGCCAGGCCCCCAGACTGCTGATCAAGTCT
GCCAGCCAGTCCATCAGCGGCATCCCCGCCAGATTTTCTGGCAGCGGCTC
CGGCACCGAGTTCACCCTGACAATCAGCAGCCTGCAGAGCGAGGACTTCG
CCGTGTACTACTGCCAGAACGGCCACAGCTTCCCTTACACCTTCGGCCAG
GGCACCAAGCTGGAAATCAAG
(PD1H163)
SEQ ID NO: 200
CAGGTGCAGCTGGTGCAGAGCGGCGCGGAAGTGAAAAAACCGGGCAGCAG
CGTGAAAGTGAGCTGCAAAGCGAGCGGCGGCACCTTCAAGTCCTATGTGA
TTCATTGGGTGCGCCAGGCGCCGGGCCAGGGCCTGGAATGGATGGGCGGT
ATTATCCCAATTTTTGGCACCGCCAATTATGCGCAGAAATTTCAGGGCCG
CGTGACCATTACCGCTGATGAAAGCACCAGCACCGCGTATATGGAACTGA
GCAGCCTGCGCAGCGAAGATACCGCGGTGTATTATTGCGCGCGCGGTTAT
GTGCGGGCTACGGGCATGTTGGACTATTGGGGCCAGGGCACCCTGGTGAC
CGTGAGCAGC
(PD1L185)
SEQ ID NO: 201
GAAATTGTGCTGACCCAGAGCCCGGCGACCCTGAGCCTGAGCCCGGGCGA
ACGCGCGACCCTGAGCTGCCGCGCGAGCCAGAGCGTTAGCAATTATCTGG
CGTGGTATCAGCAGAAACCGGGCCAGGCGCCGCGCCTGCTGATCTACGAC
GCCAGCAATCGCGCGACCGGCATTCCGGCGCGCTTTAGCGGCTCCGGTAG
CGGCACCGATTTTACCCTGACCATTAGCAGCCTGGAACCGGAAGATTTTG
CGGTGTATTATTGCCAGCAACGTGCATATTGGCCGCTGACCTTTGGCCAG
GGCACCAAAGTGGAAATTAAA
(PD1H164)
SEQ ID NO: 202
CAGGTGCAGCTGGTGCAGAGCGGCGCGGAAGTGAAAAAACCGGGCAGCAG
CGTGAAAGTGAGCTGCAAAGCGAGCGGCGGCACCTTCAGCGATTATGTGA
TTTCCTGGGTGCGCCAGGCGCCGGGCCAGGGCCTGGAATGGATGGGCGGT
ATTATCCCGATTTACGGGACCGCTAACTATGCGCAGAAATTTCAGGGCCG
CGTGACCATTACCGCTGATGAAAGCACCAGCACCGCGTATATGGAACTGA
GCAGCCTGCGCAGCGAAGATACCGCGGTGTATTATTGCGCGCGCGGTACC
CTCGACCGGACCGGGCATTTGGACTATTGGGGCCAGGGCACCCTGGTGAC
CGTGAGCAGC
(PD1L86)
SEQ ID NO: 203
GAAATTGTGCTGACCCAGAGCCCGGCGACCCTGAGCCTGAGCCCGGGCGA
ACGCGCGACCCTGAGCTGCCGCGCGAGCCAGAGCGTCTCCTCCTACCTTG
CGTGGTATCAGCAGAAACCGGGCCAGGCGCCGCGCCTGCTGATCCACGAC
GCCTCTACGCGTGCGACCGGCATTCCGGCGCGCTTTAGCGGCTCCGGTAG
CGGCACCGATTTTACCCTGACCATTAGCAGCCTGGAACCGGAAGATTTTG
CGGTGTATTATTGCCAGCAACGTAATTATTGGCCGCTCACCTTTGGCCAG
GGCACCAAAGTGGAAATTAAA
(TM3H24)
SEQ ID NO: 204
GAAGTGCAGCTGCTGGAAAGCGGCGGCGGCCTGGTGCAGCCGGGCGGCAG
CCTGCGCCTGAGCTGCGCGGCAAGCGGCTTTACCTTTAGCAGCTATGCGA
TGAGCTGGGTGCGCCAGGCGCCGGGCAAAGGCCTGGAATGGGTGAGCGCG
ATTAGCGGCAGCGGCGGCAGCACCTATTATGCGGATAGCGTGAAAGGCCG
CTTTACCATTAGCCGCGATAACAGCAAAAACACCCTGTATCTGCAGATGA
ACAGCCTGCGCGCGGAAGATACCGCGGTGTATTATTGCGCGAAATCCCCG
TACGCGCCCTTGGACTATTGGGGCCAGGGCACCCTGGTGACCGTGAGCAG
C
(TM3L33)
SEQ ID NO: 205
GAAATTGTGCTGACCCAGAGCCCGGCGACCCTGAGCCTGAGCCCGGGCGA
ACGCGCGACCCTTAGCTGCCGTGCAAGTCAGAGTGTGAACGACTACCTGG
CGTGGTATCAGCAGAAACCGGGCCAGGCGCCGCGCCTGCTGATTTATGAT
GCGAGCAACCGCGCGACCGGCATTCCGGCGCGCTTTAGCGGCAGCGGCAG
CGGCACCGATTTTACCCTGACCATTAGCAGCCTGGAACCGGAAGATTTTG
CGGTGTATTATTGCCAGCAGGGTGGTCACGCGCCGATCACCTTTGGCCAG
GGCACCAAAGTGGAAATTAAA
(TM3H162)
SEQ ID NO: 206
GAAGTGCAGCTGGTGCAGTCTGGCGCCGAAGTGAAGAAGCCTGGCGAGAG
CCTGAAGATCAGCTGCAAGGGCAGCGGCTACAGCTTCACCAGCTACTGGA
TGCAGTGGGTGCGCCAGATGCCTGGCAAGGGCCTGGAATGGATGGGCGCC
ATCTATCCCGGCGACGGCGACATCAGATACACCCAGAACTTCAAGGGCCA
AGTGACCATCAGCGCCGACAAGAGCATCAGCACCGCCTACCTGCAGTGGT
CCAGCCTGAAGGCCAGCGACACCGCCATGTACTACTGTGCCAGATGGGAG
AAGTCCACCACCGTGGTGCAGCGGAACTACTTCGACTACTGGGGCCAGGG
CACCACAGTGACCGTGTCTAGT
(TM3L85)
SEQ ID NO: 207
GACATCCAGATGACCCAGAGCCCCAGCAGCCTGTCTGCCAGCGTGGGCGA
CAGAGTGACCATCACATGCAAGGCCAGCGAGAACGTGGGCACCTTCGTGT
CCTGGTATCAGCAGAAGCCCGGCAAGGCCCCCAAGCTGCTGATCTACGGC
GCCAGCAACAGATACACCGGCGTGCCCAGCAGATTCAGCGGCTCTGGCAG
CGGCACCGACTTCACCCTGACCATCTCTAGCCTGCAGCCCGAGGACTTCG
CCACCTACTACTGCGGCCAGAGCTACAGCTACCCCACCTTTGGCCAGGGC
ACCAAGCTGGAAATCAAG
(TM3H21)
SEQ ID NO: 208
GAAGTGCAGCTGCTGGAAAGCGGCGGCGGCCTGGTGCAGCCGGGCGGCAG
CCTGCGCCTGAGCTGCGCGGCGAGCGGCTTTACCTTTAGCAACTATTGGA
TGAGCTGGGTGCGCCAGGCGCCGGGCAAAGGCCTGGAATGGGTGAGCGCG
ATTAGCGGCAGCGGCGGCAGCACCTATTATGCGGATAGCGTGAAAGGCCG
CTTTACCATTAGCCGCGATAACAGCAAAAACACCCTGTATCTGCAGATGA
ACAGCCTGCGCGCGGAAGATACCGCGGTGTATTATTGCGCGAAAGATCAT
TGGGATCCCAATTTTTTGGACTATTGGGGCCAGGGCACCCTGGTGACCGT
GAGCAGC
(PH9L1)
SEQ ID NO: 209
GAAATTGTGCTGACCCAGAGCCCGGGCACCCTGAGCCTGAGCCCGGGCGA
ACGCGCGACCCTGAGCTGCCGCGCGAGCCAGAGCGTGAGCAGCAGCTATC
TGGCGTGGTATCAGCAGAAACCGGGCCAGGCGCCGCGCCTGCTGATTTAT
GGCGCGAGCAGCCGCGCGACCGGCATTCCGGATCGCTTTAGCGGCAGCGG
CAGCGGCACCGATTTTACCCTGACCATTAGCCGCCTGGAACCGGAAGATT
TTGCGGTGTATTATTGCCAGCAGTATGGCAGCAGCCCGCTGACCTTTGGC
CAGGGCACCAAAGTGGAAATTAAA
(TM3H65)
SEQ ID NO: 210
GAAGTGCAGCTGCTGGAAAGCGGCGGCGGCCTGGTGCAGCCGGGCGGCAG
CCTGCGCCTGAGCTGCGCGGCGAGCGGCTTTACCTTTAGCGACTATTGGA
TGAGCTGGGTGCGCCAGGCGCCGGGCAAAGGCCTGGAATGGGTGAGCGTG
ATCAAGTATAGCGGTGGCTCCAAATATTATGCGGATAGCGTGAAAGGCCG
CTTTACCATTAGCCGCGATAACAGCAAAAACACCCTGTATCTGCAGATGA
ACAGCCTGCGCGCGGAAGATACCGCGGTGTATTATTGCGCGAAAGAGCTG
GAGGGGGTGTTCGACTATTGGGGCCAGGGCACCCTGGTGACCGTGAGCAG
C
(TM3L12)
SEQ ID NO: 211
GAAATTGTGCTGACCCAGAGCCCGGGCACCCTGAGCCTGAGCCCGGGCGA
ACGCGCGACCCTGAGCTGCCGCGCGAGCCAGAGCGTTAGCAATAGCACTC
TGGCGTGGTATCAGCAGAAACCGGGCCAGGCGCCGCGCCTGCTGATTTAT
ACTGCGAGCAGCCGCGCGACCGGCATTCCGGATCGCTTTAGCGGCAGCGG
CAGCGGCACCGATTTTACCCTGACCATTAGCCGCCTGGAACCGGAAGATT
TTGCGGTGTATTATTGCCAGCAGTCTTACACATCTCCGTGGACTTTTGGC
CAGGGCACCAAAGTGGAAATTAAA

The polynucleotide sequences encoding the VH or the VL or an antigen-binding fragment thereof of the antibodies of the invention, or the heavy chain and the light chain of the antibodies of the invention may be operably linked to one or more regulatory elements, such as a promoter or enhancer, that allow expression of the nucleotide sequence in the intended host cell. The polynucleotide may be a cDNA.

The invention also provides a vector comprising the polynucleotide of the invention. Such vectors may be plasmid vectors, viral vectors, vectors for baculovirus expression, transposon based vectors or any other vector suitable for introduction of the synthetic polynucleotide of the invention into a given organism or genetic background by any means. For example, polynucleotides encoding light and/or heavy chain variable regions of the antibodies of the invention, optionally linked to constant regions, are inserted into expression vectors. The light and/or heavy chains may be cloned in the same or different expression vectors. The DNA segments encoding immunoglobulin chains may be operably linked to control sequences in the expression vector(s) that ensure the expression of immunoglobulin polypeptides. Such control sequences include signal sequences, promoters (e.g. naturally associated or heterologous promoters), enhancer elements, and transcription termination sequences, and are chosen to be compatible with the host cell chosen to express the antibody. Once the vector has been incorporated into the appropriate host, the host is maintained under conditions suitable for high level expression of the proteins encoded by the incorporated polynucleotides.

In some embodiments, the vector comprises the polynucleotide of SEQ ID NO: 196 and 197.

In some embodiments, the vector comprises the polynucleotide of SEQ ID NO: 198 and 199.

In some embodiments, the vector comprises the polynucleotide of SEQ ID NO: 200 and 201.

In some embodiments, the vector comprises the polynucleotide of SEQ ID NO: 202 and 203.

In some embodiments, the vector comprises the polynucleotide of SEQ ID NO: 204 and 205.

In some embodiments, the vector comprises the polynucleotide of SEQ ID NO: 206 and 207.

In some embodiments, the vector comprises the polynucleotide of SEQ ID NO: 208 and 209.

In some embodiments, the vector comprises the polynucleotide of SEQ ID NO: 210 and 211.

In some embodiments, the vector comprises the polynucleotide of SEQ ID NO: 253 and 254.

In some embodiments, the vector comprises the polynucleotide of SEQ ID NO: 255 and 256.

In some embodiments, the vector comprises the polynucleotide of SEQ ID NO: 257 and 258.

In some embodiments, the vector comprises the polynucleotide of SEQ ID NO: 259 and 260.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance or neomycin resistance to permit detection of those cells transformed with the desired DNA sequences.

Suitable promoter and enhancer elements are known in the art. For expression in a eukaryotic cell, exemplary promoters include light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various known tissue specific promoters. Selection of the appropriate vector and promoter is well within the level of ordinary skill in the art.

Exemplary vectors that may be used are Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia), pEE6.4 (Lonza) and pEE12.4 (Lonza).

The invention also provides a host cell comprising one or more vectors of the invention. “Host cell” refers to a cell into which a vector has been introduced. It is understood that the term host cell is intended to refer not only to the particular subject cell but to the progeny of such a cell, and also to a stable cell line generated from the particular subject cell. Because certain modifications may occur in succeeding generations due to either mutation or environmental influences, such progeny may not be identical to the parent cell, but are still included within the scope of the term “host cell” as used herein. Such host cells may be eukaryotic cells, prokaryotic cells, plant cells or archeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (for example, S. cerevisiae) and Pichia are examples of suitable yeast host cells. Exemplary eukaryotic cells may be of mammalian, insect, avian or other animal origins. Mammalian eukaryotic cells include immortalized cell lines such as hybridomas or myeloma cell lines such as SP2/0 (American Type Culture Collection (ATCC), Manassas, VA, CRL-1581), NSO (European Collection of Cell Cultures (ECACC), Salisbury, Wiltshire, UK, ECACC No. 85110503), FO (ATCC CRL-1646) and Ag653 (ATCC CRL-1580) murine cell lines. An exemplary human myeloma cell line is U266 (ATTC CRL-TIB-196). Other useful cell lines include those derived from Chinese Hamster Ovary (CHO) cells such as CHOK1SV (Lonza Biologics, Walkersville, MD), Potelligent® CHOK2SV (Lonza), CHO-K1 (ATCC CRL-61) or DG44.

The invention also provides a method of producing an antibody of the invention comprising culturing the host cell of the invention in conditions that the antibody is expressed, and recovering the antibody produced by the host cell. Methods of making antibodies and purifying them are well known in the art. Once synthesized (either chemically or recombinantly), the whole antibodies, their dimers, individual light and/or heavy chains, or other antibody fragments such as VH and/or VL, may be purified according to standard procedures, including ammonium sulfate precipitation, affinity columns, column chromatography, high performance liquid chromatography (HPLC) purification, gel electrophoresis, and the like (see generally Scopes, Protein Purification (Springer-Verlag, N.Y., (1982)). A subject antibody may be substantially pure, for example, at least about 80% to 85% pure, at least about 85% to 90% pure, at least about 90% to 95% pure, or at least about 98% to 99%, or more, pure, for example, free from contaminants such as cell debris, macromolecules, etc. other than the subject antibody.

The polynucleotide sequences of the invention may be incorporated into vectors using standard molecular biology methods. Host cell transformation, culture, antibody expression and purification are done using well known methods. Another embodiment of the invention is a method of producing the antagonistic antibody specifically binding PD-1 of the invention, comprising:
incorporating the first polynucleotide encoding the VH of the antibody and the second polynucleotide encoding the VL of the antibody into an expression vector; transforming a host cell with the expression vector;
culturing the host cell in culture medium under conditions wherein the VL and the VH are expressed and form the antibody; and
recovering the antibody from the host cell or culture medium.

Another embodiment of the invention described herein is a method of producing the antagonistic antibody specifically binding TIM-3 of the invention, comprising:
incorporating the first polynucleotide encoding the VH of the antibody and the second polynucleotide encoding the VL of the antibody into an expression vector; transforming a host cell with the expression vector;
culturing the host cell in culture medium under conditions wherein the VL and the VH are expressed and form the antibody; and
recovering the antibody from the host cell or culture medium.

The polynucleotides encoding certain VH or VL sequences of the invention described herein, and in some embodiments of each and every one of the numbered embodiments listed below, may be incorporated into vectors using standard molecular biology methods. Host cell transformation, culture, antibody expression and purification are done using well known methods.

### Pharmaceutical Compositions/Administration

The invention provides pharmaceutical compositions comprising the antibodies of the invention and a pharmaceutically acceptable carrier. For therapeutic use, the antibodies of the invention may be prepared as pharmaceutical compositions containing an effective amount of the antibody as an active ingredient in a pharmaceutically acceptable carrier. “Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the antibodies of the invention in such pharmaceutical formulation may vary, from less than about 0.5%, usually to at least about 1% to as much as 15 or 20% by weight and may be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21^{st} Edition, Troy, D. B. ed., Lipincott Williams and Wilkins, Philadelphia, PA 2006, Part 5, Pharmaceutical Manufacturing pp 691-1092, See especially pp. 958-989.

The mode of administration for therapeutic use of the antibodies of the invention may be any suitable route that delivers the antibody to the host, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intratumoral, intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

The antibodies of the invention may be administered to a subject by any suitable route, for example parentally by intravenous (i.v.) infusion or bolus injection, intramuscularly or subcutaneously or intraperitoneally. i.v. infusion may be given over for example 15, 30, 60, 90, 120, 180, or 240 minutes, or from 1, 2, 3, 4, 5, 6, 7, 8, 9, 10, 11 or 12 hours.

The dose given to a subject is sufficient to alleviate or at least partially arrest the disease being treated (“therapeutically effective amount”) and may be sometimes 0.005 mg to about 100 mg/kg, e.g. about 0.05 mg to about 30 mg/kg or about 5 mg to about 25 mg/kg, or about 4 mg/kg, about 8 mg/kg, about 16 mg/kg or about 24 mg/kg, or for example about 1, 2, 3, 4, 5, 6, 7, 8, 9 or 10 mg/kg, but may even higher, for example about 15, 16, 17, 18, 19, 20, 21, 22, 23, 24, 25, 30, 40, 50, 60, 70, 80, 90 or 100 mg/kg.

A fixed unit dose may also be given, for example, 50, 100, 200, 500 or 1000 mg, or the dose may be based on the patient's surface area, e.g., 500, 400, 300, 250, 200, or 100 mg/m². Usually between 1 and 8 doses, (e.g., 1, 2, 3, 4, 5, 6, 7 or 8) may be administered to treat the patient, but 9, 10, 11, 12, 13, 14, 15, 16, 17, 18, 19, 20 or more doses may be given.

The administration of the antibodies of the invention may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose. For example, the antibodies of the invention may be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion.

For example, the antibodies of the invention may be provided as a daily dosage in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

The antibodies of the invention, may also be administered prophylactically in order to reduce the risk of developing cancer, delay the onset of the occurrence of an event in cancer progression, and/or reduce the risk of recurrence when a cancer is in remission.

The antibodies of the invention may be lyophilized for storage and reconstituted in a suitable carrier prior to use. This technique has been shown to be effective with conventional protein preparations and well known lyophilization and reconstitution techniques can be employed.

### Methods and Uses

The antibodies of the invention have in vitro and in vivo diagnostic, as well as therapeutic and prophylactic utilities. For example, the antibodies of the invention may be administered to cells in culture, in vitro or ex vivo, or to a subject to treat, prevent, and/or diagnose a variety of disorders, such as cancers and infectious disorders.

The invention provides a method of modifying an immune response in a subject comprising administering to the subject the antibody of the invention for a time sufficient to modify the immune response.

In some embodiments, the immune response is enhanced, stimulated or up-regulated.

In some embodiments described herein, the subject is a human patient.

In some embodiments described herein, the subject is a human patient in need of enhancement of the immune response.

In some embodiments, the subject is immunocompromised.

In some embodiments, the subject is at risk of being immunocompromised.

Immunocompromised subject may be undergoing, or has undergone a chemotherapeutic or radiation therapy.

In some embodiment, the subject is or is at risk of being immunocompromised as a result of an infection.

The antibodies of the invention are suitable for treating a subject having a disorder that may be treated by augmenting T-cell mediated immune responses.

In some embodiments, the antagonistic antibody specifically binding PD-1 used in the methods of the invention described herein is PD1B114, PD1B149, PD1B160, PD1B162, PD1B164, PD1B11, PD1B183, PD1B184, PD1B185, PD1B187, PD1B71, PD1B177, PD1B70, PD1B175, PD1B194, PD1B195, PD1B196, PD1B197, PD1B198, PD1B199, PD1B200, PD1B201, PD1B243, PD1B244, PD1B131 or PD1B132. The VH and the VL amino acid sequences of these antibodies are shown in Table 2.

In some embodiments, the antagonistic antibody specifically binding TIM-3 used in the methods of the invention described herein is TM3B103, TM3B105, TM3B109, TM3B108, TM3B113, TM3B189, TM3B190, TM3B193, TM3B195, TM3B196 or TM3B291. The VH and the VL amino acid sequences of these antibodies are shown in Table 3.

In some embodiments, the bispecific PD-1/TIM-3 antibody used in the methods of the invention is PTBB14, PTBB15, PTBB16, PTBB17, PTBB24, PTBB30, PTBB27, PTBB28, PTBB18, PTBB20 or PTBB21. The HC1, the LC1, the HC2 and the LC2 amino acid sequences of these antibodies are shown in Table 41 and Table 42.

In some embodiments, the antagonistic antibody specifically binding PD-1 used in the methods of the invention comprises the VH of SEQ ID NO: 41 and the VL of SEQ ID NO: 49.

In some embodiments, the antagonistic antibody specifically binding PD-1 used in the methods of the invention comprises the VH of SEQ ID NO: 41 and the VL of SEQ ID NO: 50.

In some embodiments, the antagonistic antibody specifically binding PD-1 used in the methods of the invention comprises the VH of SEQ ID NO: 42 and the VL of SEQ ID NO: 51.

In some embodiments, the antagonistic antibody specifically binding PD-1 used in the methods of the invention comprises the VH of SEQ ID NO: 42 and the VL of SEQ ID NO: 52.

In some embodiments, the antagonistic antibody specifically binding PD-1 used in the methods of the invention comprises the VH of SEQ ID NO: 42 and the VL of SEQ ID NO: 53.

In some embodiments, the antagonistic antibody specifically binding PD-1 used in the methods of the invention comprises the VH of SEQ ID NO: 43 and the VL of SEQ ID NO: 49.

In some embodiments, the antagonistic antibody specifically binding PD-1 used in the methods of the invention comprises the VH of SEQ ID NO: 43 and the VL of SEQ ID NO: 54.

In some embodiments, the antagonistic antibody specifically binding PD-1 used in the methods of the invention comprises the VH of SEQ ID NO: 43 and the VL of SEQ ID NO: 50.

In some embodiments, the antagonistic antibody specifically binding PD-1 used in the methods of the invention comprises the VH of SEQ ID NO: 43 and the VL of SEQ ID NO: 55.

In some embodiments, the antagonistic antibody specifically binding PD-1 used in the methods of the invention comprises the VH of SEQ ID NO: 43 and the VL of SEQ ID NO: 56.

In some embodiments, the antagonistic antibody specifically binding PD-1 used in the methods of the invention comprises the VH of SEQ ID NO: 43 and the VL of SEQ ID NO: 57.

In some embodiments, the antagonistic antibody specifically binding PD-1 used in the methods of the invention comprises the VH of SEQ ID NO: 44 and the VL of SEQ ID NO: 49.

In some embodiments, the antagonistic antibody specifically binding PD-1 used in the methods of the invention comprises the VH of SEQ ID NO: 45 and the VL of SEQ ID NO: 49.

In some embodiments, the antagonistic antibody specifically binding PD-1 used in the methods of the invention comprises the VH of SEQ ID NO: 46 and the VL of SEQ ID NO: 49.

In some embodiments, the antagonistic antibody specifically binding PD-1 used in the methods of the invention comprises the VH of SEQ ID NO: 47 and the VL of SEQ ID NO: 49.

In some embodiments, the antagonistic antibody specifically binding PD-1 used in the methods of the invention comprises the VH of SEQ ID NO: 48 and the VL of SEQ ID NO: 53.

In some embodiments, the antagonistic antibody specifically binding PD-1 used in the methods of the invention comprises the VH of SEQ ID NO: 48 and the VL of SEQ ID NO: 52.

In some embodiments, the antagonistic antibody specifically binding PD-1 used in the methods of the invention comprises the VH of SEQ ID NO: 48 and the VL of SEQ ID NO: 56.

In some embodiments, the antagonistic antibody specifically binding PD-1 used in the methods of the invention comprises the VH of SEQ ID NO: 47 and the VL of SEQ ID NO: 58.

In some embodiments, the antagonistic antibody specifically binding PD-1 used in the methods of the invention comprises the VH of SEQ ID NO: 47 and the VL of SEQ ID NO: 59.

In some embodiments, the antagonistic antibody specifically binding PD-1 used in the methods of the invention comprises the VH of SEQ ID NO: 45 and the VL of SEQ ID NO: 60.

In some embodiments, the antagonistic antibody specifically binding PD-1 used in the methods of the invention comprises the VH of SEQ ID NO: 45 and the VL of SEQ ID NO: 61.

In some embodiments, the antagonistic antibody specifically binding PD-1 used in the methods of the invention comprises the VH of SEQ ID NO: 45 and the VL of SEQ ID NO: 62.

In some embodiments, the antagonistic antibody specifically binding TIM-3 used in the methods of the invention comprises the VH of SEQ ID NO: 145 and the VL of SEQ ID NO: 155.

In some embodiments, the antagonistic antibody specifically binding TIM-3 used in the methods of the invention comprises the VH of SEQ ID NO: 146 and the VL of SEQ ID NO: 156.

In some embodiments, the antagonistic antibody specifically binding TIM-3 used in the methods of the invention comprises the VH of SEQ ID NO: 148 and the VL of SEQ ID NO: 157.

In some embodiments, the antagonistic antibody specifically binding TIM-3 used in the methods of the invention comprises the VH of SEQ ID NO: 147 and the VL of SEQ ID NO: 155.

In some embodiments, the antagonistic antibody specifically binding TIM-3 used in the methods of the invention comprises the VH of SEQ ID NO: 149 and the VL of SEQ ID NO: 158.

In some embodiments, the antagonistic antibody specifically binding TIM-3 used in the methods of the invention comprises the VH of SEQ ID NO: 150 and the VL of SEQ ID NO: 159.

In some embodiments, the antagonistic antibody specifically binding TIM-3 used in the methods of the invention comprises the VH of SEQ ID NO: 151 and the VL of SEQ ID NO: 160.

In some embodiments, the antagonistic antibody specifically binding TIM-3 used in the methods of the invention comprises the VH of SEQ ID NO: 152 and the VL of SEQ ID NO: 161.

In some embodiments, the antagonistic antibody specifically binding TIM-3 used in the methods of the invention comprises the VH of SEQ ID NO: 153 and the VL of SEQ ID NO: 162.

In some embodiments, the antagonistic antibody specifically binding TIM-3 used in the methods of the invention comprises the VH of SEQ ID NO: 154 and the VL of SEQ ID NO: 163.

In some embodiments, the antagonistic antibody specifically binding TIM-3 used in the methods of the invention comprises the VH of SEQ ID NO: 172 and the VL of SEQ ID NO: 173.

In some embodiments, the antagonistic bispecific PD-1/TIM-3 antibody comprising a first domain specifically binding PD-1 and a second domain specifically binding TIM-3 used in the methods of the invention comprises the VH of SEQ ID NO: 48 and the VL of SEQ ID NO: 56 in the first domain, and the VH of SEQ ID NO: 153 and the VL of SEQ ID NO: 162 in the second domain.

In some embodiments, the antagonistic bispecific PD-1/TIM-3 antibody comprising a first domain specifically binding PD-1 and a second domain specifically binding TIM-3 used in the methods of the invention comprises the VH of SEQ ID NO: 48 and the VL of SEQ ID NO: 56 in the first domain, and the VH of SEQ ID NO: 146 and the VL of SEQ ID NO: 156 in the second domain.

In some embodiments, the antagonistic bispecific PD-1/TIM-3 antibody comprising a first domain specifically binding PD-1 and a second domain specifically binding TIM-3 used in the methods of the invention comprises the VH of SEQ ID NO: 64 and the VL of SEQ ID NO: 65 in the first domain, and the VH of SEQ ID NO: 153 and the VL of SEQ ID NO: 162 in the second domain.

In some embodiments, the antagonistic bispecific PD-1/TIM-3 antibody comprising a first domain specifically binding PD-1 and a second domain specifically binding TIM-3 used in the methods of the invention, comprises the VH of SEQ ID NO: 64 and the VL of SEQ ID NO: 65 in the first domain, and the VH of SEQ ID NO: 146 and the VL of SEQ ID NO: 156 in the second domain.

In some embodiments, the antagonistic bispecific PD-1/TIM-3 antibody comprising a first domain specifically binding PD-1 and a second domain specifically binding TIM-3 used in the methods of the invention comprises the VH of SEQ ID NO: 48 and the VL of SEQ ID NO: 56 in the first domain, and the VH of SEQ ID NO: 172 and the VL of SEQ ID NO: 173 in the second domain.

Cancer Blockade of PD-1 may enhance an immune response to cancerous cells in a subject. The ligand for PD-1, PD-L1, is abundantly expressed in a variety of human cancers (Dong et al., (2002) Nat Med 8:787-9). The interaction between PD-1 and PD-L1 can result in a decrease in tumor infiltrating lymphocytes, a decrease in T-cell receptor mediated proliferation, and/or immune evasion by the cancerous cells (Dong et al., (2003) J Mol Med 81:281-7; Blank et al., (2005) Cancer Immunol Immunother 54:307-314; Konishi et al., (2004) Clin Cancer Res 10:5094-100). Immune suppression may be reversed by inhibiting the local interaction of PD-1 to PD-Li; the effect is additive when the interaction of PD-1 to the second PD-1 ligand, PD-L2, is blocked as well (Iwai et al., (2002) PorcNatl Acad Sci 99:12293-7; Brown et al., (2003) J Immunol 170:1257-66). Thus, inhibition of PD-1 may result in augmenting an immune response.

TIM-3 is a coinhibitory protein expressed on activated T helper 1 (Th1) CD4⁺ and cytotoxic CD8⁺ T cells that secrete IFN-γ. TIM-3 is co-expressed on PD-1+ exhausted T cells as shown in preclinical models of cancer and viral exhaustion. Co-blockade of these pathways may restore effector T cell function (e.g., IFN-γ secretion, proliferation) in several models as well as human PBMCs derived from metastatic melanoma patients and patients with HIV or HCV. TIM-3 is also enriched on Foxp3+ regulatory T cells and Tregs co-expressing TIM-3, LAG3 and CTLA4 have been shown to be highly efficient suppressors of effector T cells (Teff) (Galuton et al., (2014) Eur J Immunol 44(9):2703-11). TIM-3 expression has been correlated with poorer prognosis in NSCLC (Zhuang et al., (2012) Am J Clin Pathol 137(6):978-85). Lymphocytes from tumor tissues of ovarian, colorectal, cervical and hepatocellular carcinoma patients exhibit higher proportion of TIM-3⁺ CD4 T cells, which cells have impaired capacity to produce ILF-γ (Yan et al., (2013) PLoS One 8(3):e58006).

The invention also provides a method of inhibiting growth of tumor cells in a subject, comprising administering to the subject a therapeutically effective amount of the antagonistic antibody specifically binding PD-1 of the invention for a time sufficient to inhibit growth of tumor cells . . . .

The invention also provides a method of inhibiting growth of tumor cells in a subject, comprising administering to the subject a therapeutically effective amount of the antagonistic antibody specifically binding TIM-3 of the invention for a time sufficient to inhibit growth of tumor cells.

The invention also provides a method of inhibiting growth of tumor cells in a subject, comprising administering to the subject a therapeutically effective amount of the antagonistic bispecific PD-1/TIM-3 antibody of the invention for a time sufficient to inhibit growth of tumor cells.

The invention also provides a method of treating a cancer by administering to the subject in need thereof a therapeutically effective amount of the antagonistic antibody specifically binding PD-1 of the invention for a time sufficient to treat the cancer.

The invention also provides a method of treating a cancer by administering to the subject in need thereof a therapeutically effective amount of the antagonistic antibody specifically binding TIM-3 of the invention for a time sufficient to treat the cancer.

The invention also provides a method of treating a cancer by administering to the subject in need thereof a therapeutically effective amount of the bispecific PD-1/TIM-3 antibody of the invention for a time sufficient to treat the cancer.

Exemplary antibodies that may be used are antagonistic antibodies specifically binding PD-1, antagonistic antibodies specifically binding TIM-3, and antagonistic bispecific PD-1/TIM-3 antibodies PD1B114, PD1B149, PD1B160, PD1B162, PD1B164, PD1B11, PD1B183, PD1B184, PD1B185, PD1B187, PD1B71, PD1B177, PD1B70, PD1B175, PD1B194, PD1B195, PD1B196, PD1B197, PD1B198, PD1B199, PD1B200, PD1B201, TM3B103, TM3B105, TM3B109, TM3B108, TM3B113, TM3B189, TM3B190, TM3B193, TM3B195, TM3B196, TM3B291, PTBB14, PTBB15, PTBB16, PTBB17, PTBB24, PTBB30, PTBB27, PTBB28, PTBB18, PTBB20 and PTBB21 having the VH and the VL amino acid sequence and characteristics as described herein.

Cancer may be a hyperproliferative condition or disorder, a solid tumor, a hematological malignancy, a soft tissue tumor, or a metastatic lesion.

“Cancer” is meant to include all types of cancerous growths or oncogenic processes, metastatic tissues or malignantly transformed cells, tissues, or organs, irrespective of histopathology type or stage of invasiveness. Examples of cancers include solid tumors, hematological malignancies, soft tissue tumors, and metastatic lesions. Exemplary solid tumors include malignancies, e.g., sarcomas, and carcinomas (including adenocarcinomas and squamous cell carcinomas) of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, a rectal cancer, a renal-cell carcinoma, a liver cancer, a non-small cell carcinoma of the lung, a cancer of the small intestine and a cancer of the esophagus. Squamous cell carcinomas include malignancies, e.g., in the lung, esophagus, skin, head and neck region, oral cavity, anus, and cervix.

In some embodiments, the cancer is a melanoma.

Metastatic lesions of the aforementioned cancers may also be treated or prevented using the methods and antibodies of the invention described herein.

Exemplary cancers whose growth may be inhibited or reduced using the antibodies of the invention include cancers that may be responsive to immunotherapy. Exemplary such cancers include a melanoma, a renal cancer, a prostate cancer, a breast cancer, a colon cancer, a gastrointestinal cancer, a stomach cancer, an esophageal cancer, a lung cancer, a metastatic malignant melanoma, a clear cell carcinoma, a hormone refractory prostate adenocarcinoma, a non-small cell lung cancer or cancer of the head and neck. Refractory or recurrent malignancies may be treated using the antibodies of the invention described herein.

Exemplary other cancers that may be treated with the antibodies of the invention ae an anal cancer, a basal cell carcinoma, a biliary tract cancer, a bladder cancer, a bone cancer, brain and CNS cancers, a carcinoma of the fallopian tubes, carcinoma of the vagina, a carcinoma of the vulva, a cutaneous or intraocular malignant melanoma, a astro-esophageal cancer, a testicular cancer, an ovarian cancer, a pancreatic cancer, a rectal cancer, an uterine cancer, a primary CNS lymphoma; a neoplasm of the central nervous system (CNS), a cervical cancer, a choriocarcinoma, a rectum cancer, a connective tissue cancer, a cancer of the digestive system, an endometrial cancer, an eye cancer; an intra-epithelial neoplasm, a kidney cancer, a larynx cancer, a liver cancer; a small cell lung cancer, a neuroblastoma, an oral cavity cancer (e.g., lip, tongue, mouth, and pharynx), a nasopharyngeal cancer, a retinoblastoma, a rhabdomyosarcoma, a cancer of the respiratory system, a sarcoma, a thyroid cancer, a cancer of the urinary system, a hepatocarcinoma, a cancer of the anal region, a carcinoma of the fallopian tubes, a carcinoma of the vagina, a carcinoma of the vulva, a cancer of the small intestine, a cancer of the endocrine system, a cancer of the parathyroid gland, a cancer of the adrenal gland, a sarcoma of soft tissue, a cancer of the urethra, a cancer of the penis, solid tumors of childhood, a tumor angiogenesis, a spinal axis tumor, a brain stem glioma, a pituitary adenoma, Kaposi's sarcoma, Merkel cell cancer, an epidermoid cancer, a squamous cell cancer, an environmentally induced cancers including those induced by asbestos, as well as other carcinomas and sarcomas, and combinations of said cancers.

Exemplary hematological malignancies that may be treated with the antibodies of the invention include leukemias, lymphomas and myeloma, such as a precursor B-cell lymphoblastic leukemia/lymphoma and a B-cell non-Hodgkin's lymphoma, an acute promyelocytic leukemia, an acute lymphoblastic leukemia (ALL), a B-cell chronic lymphocytic leukemia(CLL)/small lymphocytic lymphoma (SLL), a B-cell acute lymphocytic leukemia, a B-cell prolymphocytic leukemia, a lymphoplasmacytic lymphoma, a mantle cell lymphoma (MCL), a follicular lymphoma (FL), including low-grade, intermediate-grade and high-grade FL, a cutaneous follicle center lymphoma, a marginal zone B-cell lymphoma (MALT type, nodal and splenic type), a hairy cell leukemia, a diffuse large B-cell lymphoma (DLBCL), Burkitt's lymphoma (BL), a plasmacytoma, a multiple myeloma (MM), a plasma cell leukemia, a post-transplant lymphoproliferative disorder, Waldenstrom's macroglobulinemia, plasma cell disorders, an anaplastic large-cell lymphoma (ALCL), a T-cell acute lymphocytic leukemia, a primary systemic amyloidosis (e.g. light chain amyloidosis), a pro-lymphocytic/myelocytic leukemia, an acute myeloid leukemia (AML), a chronic myeloid leukemia (CML), a large granular lymphocytic (LGL) leukemia, a NK-cell leukemia and Hodgkin's lymphoma.

“Plasma cell disorder” refers to disorders characterized by clonal plasma cells, and includes a multiple myeloma, a light chain amyloidosis and Waldenstrom's macroglobulinemia. Light chain amyloidosis and Waldenstrom's macroglobulinemia can arise independently from multiple myeloma. They may also present simultaneously with multiple myeloma, and develop either before or after the development of multiple myeloma.

Exemplary B-cell non-Hodgkin's lymphomas are a lymphomatoid granulomatosis, a primary effusion lymphoma, an intravascular large B-cell lymphoma, a mediastinal large B-cell lymphoma, heavy chain diseases (including γ, μ, and a disease), lymphomas induced by therapy with immunosuppressive agents, such as cyclosporine-induced lymphoma, and methotrexate-induced lymphoma.

Patients having cancer including metastatic cancer that express PD-L1 may be treated with the antibodies of the invention. The cancer may be a melanoma, a renal cell carcinoma, a squamous non-small cell lung cancer (NSCLC), a non-squamous NSCLC, a colorectal cancer, a castration-resistant prostate cancer, an ovarian cancer, a gastric cancer, an adenocarcinoma (ACA), a squamous cell carcinoma (SCC), a hepatocellular carcinoma (HCC), a pancreatic carcinoma, a squamous cell carcinoma of the head and neck, carcinomas of the esophagus, gastrointestinal tract and breast.

Patients having cancer that expresses TIM-3 may be treated with the antibodies of the invention. TIM-3-expressing cancers include a cervical cancer, a lung cancer, a NSCLC, an acute myeloid leukemia (AML), a diffuse large B cell lymphoma (DLBCL), a melanoma, a renal cancer, a renal cell carcinoma (RCC), a kidney clear cell carcinoma, a kidney papillary cell carcinoma, a metastatic renal cell carcinoma, a squamous cell carcinoma, an esophageal squamous cell carcinoma, a nasopharyngeal carcinoma, a colorectal cancer, a breast cancer (e.g., a breast cancer that does not express one, two or all of estrogen receptor, progesterone receptor, or Her2/neu, e.g., a triple negative breast cancer), a mesothelioma, a hepatocellular carcinoma, and an ovarian cancer. The TIM-3-expressing cancer may be a metastatic cancer.

In some embodiments, the subject has a solid tumor.

In some embodiments, the subject has a hematological malignancy.

In some embodiments, the solid tumor is a melanoma.

In some embodiments, the solid tumor is a lung cancer.

In some embodiments, the solid tumor is a non-small cell lung cancer (NSCLC).

In some embodiments, the solid tumor is a squamous non-small cell lung cancer (NSCLC).

In some embodiments, the solid tumor is a non-squamous NSCLC.

In some embodiments, the solid tumor is a lung adenocarcinoma.

In some embodiments, the solid tumor is a renal cell carcinoma (RCC). In some embodiments, the solid tumor is a mesothelioma.

In some embodiments, the solid tumor is a nasopharyngeal carcinoma (NPC).

In some embodiments, the solid tumor is a colorectal cancer.

In some embodiments, the solid tumor is a prostate cancer.

In some embodiments, the solid tumor is castration-resistant prostate cancer.

In some embodiments, the solid tumor is a stomach cancer.

In some embodiments, the solid tumor is an ovarian cancer.

In some embodiments, the solid tumor is a gastric cancer.

In some embodiments, the solid tumor is a liver cancer.

In some embodiments, the solid tumor is pancreatic cancer.

In some embodiments, the solid tumor is a thyroid cancer.

In some embodiments, the solid tumor is a squamous cell carcinoma of the head and neck.

In some embodiments, the solid tumor is a carcinomas of the esophagus or gastrointestinal tract.

In some embodiments, the solid tumor is a breast cancer.

In some embodiments, the solid tumor is a fallopian tube cancer.

In some embodiments, the solid tumor is a brain cancer.

In some embodiments, the solid tumor is an urethral cancer.

In some embodiments, the solid tumor is a genitourinary cancer.

In some embodiments, the solid tumor is an endometriosis.

In some embodiments, the solid tumor is a cervical cancer.

In some embodiments, the solid tumor is a metastatic lesion of the cancer.

In some embodiments, the hematological malignancy is a lymphoma, a myeloma or a leukemia.

In some embodiments, the hematological malignancy is a B cell lymphoma.

In some embodiments, the hematological malignancy is Burkitt's lymphoma.

In some embodiments, the hematological malignancy is Hodgkin's lymphoma.

In some embodiments, the hematological malignancy is a non-Hodgkin's lymphoma.

In some embodiments, the hematological malignancy is a myelodysplastic syndrome.

In some embodiments, the hematological malignancy is an acute myeloid leukemia (AML).

In some embodiments, the hematological malignancy is a chronic myeloid leukemia (CML).

In some embodiments, the hematological malignancy is a chronic myelomoncytic leukemia (CMML).

In some embodiments, the hematological malignancy is a multiple myeloma (MM).

In some embodiments, the hematological malignancy is a plasmacytoma.

In some embodiments, the subject has a tumor that expresses PD-L1.

In some embodiments, the subject has tumor-infiltrating T lymphocytes (TILs) in the tumor tissue.

In some embodiments, the subject has PD-1⁺TIM-3⁺ TILs in the tumor tissue.

In some embodiments, the subject has increased number of PD-1⁺ TIM-3⁺ tumor-infiltrating T lymphocytes (TILs) in the tumor tissue.

“Increased number” refers to statistically significant increase in a subject when compared to a control. “Increased number” for example refers to statistically significant increase in the number of TILs in a subject (e.g. patient) pre- and post-treatment with a PD-1 antibody or other therapeutic.

In some embodiments, the subject has increased expression or activity of interferon-gamma (IFN-γ).

In some embodiments, the subject has been treated with an anti-PD-1 antibody.

In some embodiments, the subject is refractory to treatment with the anti-PD-1 antibody.

In some embodiments, the subject has a relapsed tumor after treatment with the anti-PD-1 antibody.

In some embodiments, the subject has been treated with the anti-PD-1 antibody comprising the VH of SEQ ID NO: 230 and the VL of SEQ ID NO: 231 (e.g. KEYTRUDA® (pembrolizumab)).

In some embodiments, the subject has been treated with the anti-PD-1 antibody comprising the VH of SEQ ID NO: 232 and the VL of SEQ ID NO: 233 (e.g. OPDIVO® (nivolumab)).

In some embodiments, the subject is refractory to treatment with the anti-PD-1 antibody comprising the VH of SEQ ID NO: 230 and the VL of SEQ ID NO: 231 (e.g. KEYTRUDA® (pembrolizumab)).

In some embodiments, the subject is refractory to treatment with the anti-PD-1 antibody comprising the VH of SEQ ID NO: 232 and the VL of SEQ ID NO: 233 (e.g. OPDIVO® (nivolumab)).

In some embodiments, the subject has a relapsed tumor after treatment with the anti-PD-1 antibody comprising the VH of SEQ ID NO: 230 and the VL of SEQ ID NO: 231 (e.g. KEYTRUDA® (pembrolizumab).

In some embodiments, the subject has a relapsed tumor after treatment with the anti-PD-1 antibody comprising the VH of SEQ ID NO: 232 and the VL of SEQ ID NO: 233 (e.g. OPDIVO® (nivolumab)).

SEQ ID NO: 230
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSS
SEQ ID NO: 231
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIK
SEQ ID NO: 232
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSS
SEQ ID NO: 233
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIK

In some embodiments, the subject has been treated or is being treated with a PD-L1 antibody.

In some embodiments, the subject is refractory to treatment with the PD-L1 antibody.

In some embodiments, the subject has a relapsed tumor after treatment with the PD-L1 antibody.

In some embodiments, the subject is refractory or relapsed after treatment with the PD-L1 antibody durvalumab (MEDI-4736). Durvalumab comprises the VH of SEQ ID NO: 234 and the VL of SEQ ID NO: 235.

SEQ ID NO: 234
EVQLVESGGGLVQPGGSLRLSCAASGFTFSRYWMSWVRQAPGKGLEWVAN
IKQDGSEKYYVDSVKGRFTISRDNAKNSLYLQMNSLRAEDTAVYYCAREG
GWFGELAFDYWGQGTLVTVSS
SEQ ID NO: 235
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK

In some embodiments, the subject is refractory or relapsed after treatment with the PD-L1 antibody atezolizumab.

Atezolizumab comprises the VH of SEQ ID NO: 236 and the VL of SEQ ID NO: 237.

SEQ ID NO: 236
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS
SEQ ID NO: 237
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIK

In some embodiments, the subject is refractory or relapsed after treatment with the PD-L1 antibody avelumab.

Avelumab comprises the VH of SEQ ID NO: 238 and the VL of SEQ ID NO: 239.

SEQ ID NO: 238
EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVTTVDYWGQGTLVTVSS
SEQ ID NO: 239
QSALTQPASVSGSPGQSITISCTGTSSDVGGYNYVSWYQQHPGKAPKLMI
YDVSNRPSGVSNRFSGSKSGNTASLTISGLQAEDEADYYCSSYTSSSTRV
FGTGTKVTVL

In some embodiments, the subject is refractory or relapsed after treatment with the PD-L1 antibody MDX-1105.

In some embodiments, the subject has been treated or is being treated with a PD-L2 antibody.

In some embodiments described herein, the subject is refractory to treatment with a PD-L2 antibody.

In some embodiments, the subject has a relapsed tumor after treatment with a PD-L2 antibody.

Various qualitative and/or quantitative methods may be used to determine relapse or refractory nature of the disease. Symptoms that may be associated with relapse or resistance are, for example, a decline or plateau of the well-being of the patient or re-establishment or worsening of various symptoms associated with solid tumors, and/or the spread of cancerous cells in the body from one location to other organs, tissues or cells.

TIM-3 expression was found herein to be elevated in CD8⁺ T cells isolated from tumors after anti-PD-1 antibody treatment. Therefore, therapeutic administration of antagonistic antibodies specifically binding TIM-3 or antagonistic bispecific PD-1/TIM-3 antibodies described herein to a subject who has already received or is receiving anti-PD-1 antibody therapy, is refractory to the anti-PD-1 antibody treatment or has relapsed after or during the anti-PD-1 antibody treatment may improve the clinical outcome of the patients.

The invention also provides a method of treating a cancer in a subject, comprising administering to the subject a therapeutically effective amount of the antagonistic antibody specifically binding TIM-3 of the invention, wherein the subject is being treated or has been treated with an anti-PD-1 antibody.

In some embodiments, the antagonistic antibody specifically binding TIM-3 comprises the VH of SEQ ID NO: 146 and the VL of SEQ ID NO: 156.

The invention also provides a method of treating a cancer in a subject, comprising administering to the subject a therapeutically effective amount of the antagonistic antibody specifically binding TIM-3 comprising the VH of SEQ ID NO: 146 and the VL of SEQ ID NO: 156, wherein the subject is being treat or has been treated with the anti-PD-1 antibody KEYTRUDA® (pembrolizumab) comprising the VH of SEQ ID NO: 230 and the VL of SEQ ID NO: 231.

The invention also provides a method of treating a cancer in a subject, comprising administering to the subject a therapeutically effective amount of the antagonistic antibody specifically binding TIM-3 comprising the VH of SEQ ID NO: 146 and the VL of SEQ ID NO: 156, wherein the subject is being treat or has been treated with the anti-PD-1 antibody OPDIVO® (nivolumab) comprising the VH of SEQ ID NO: 232 and the VL of SEQ ID NO: 233.

The invention also provides a method of treating a cancer in a subject, comprising administering to the subject a therapeutically effective amount of the antagonistic antibody specifically binding TIM-3 of the invention, wherein the subject is being treated or has been treated with an anti-PD-L1 antibody.

The invention also provides a method of treating a cancer in a subject, comprising administering to the subject a therapeutically effective amount of the antagonistic antibody specifically binding TIM-3 of the invention, wherein the subject is being treated or has been treated with an anti-PD-L2 antibody.

The invention also provides a method of treating a cancer in a subject, comprising administering to the subject a therapeutically effective amount of the antagonistic bispecific PD-1/TIM-3 antibody the invention, wherein the subject is being treated or has been treated with an anti-PD-1 antibody.

The invention also provides a method of treating a cancer in a subject, comprising administering to the subject a therapeutically effective amount of the antagonistic bispecific PD-1/TIM-3 antibody the invention, wherein the subject is being treated or has been treated with an anti-PD-L1 antibody.

The invention also provides a method of treating a cancer in a subject, comprising administering to the subject a therapeutically effective amount of the antagonistic bispecific PD-1/TIM-3 antibody the invention, wherein the subject is being treated or has been treated with an anti-PD-L2 antibody.

The invention also provides a method of treating a cancer in a subject, comprising administering to the subject a therapeutically effective amount of the antagonistic antibody specifically binding PD-1 comprising the VH of SEQ ID NO: 48 and the VL of SEQ ID NO: 56 for a time sufficient to treat the cancer.

The invention also provides a method of treating a cancer in a subject, comprising administering to the subject a therapeutically effective amount of the antagonistic antibody specifically binding PD-1 comprising the VH of SEQ ID NO: 64 and the VL of SEQ ID NO: 65 for a time sufficient to treat the cancer.

Any of the PD-1, TIM-3 or bispecific PD-1/TIM-3 antibodies of the invention described herein may be used in the methods of the invention.

“Treat” or “treatment” refers to therapeutic treatment wherein the object is to slow down (lessen) an undesired physiological change or disease, such as the development or spread of tumor or tumor cells, or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, lack of metastasis, amelioration or palliation of the disease state, and remission (whether partial or total), whether detectable or undetectable. “Treatment” may also mean prolonging survival as compared to expected survival if a subject was not receiving treatment. Those in need of treatment include those subjects already with the undesired physiological change or diseases well as those subjects prone to have the physiological change or disease.

A “therapeutically effective amount” refers to an amount effective, at dosages and for periods of time necessary, to achieve a desired therapeutic result. A therapeutically effective amount of the antibody of the invention may vary according to factors such as the disease state, age, sex, and weight of the individual, and the ability of the antibody of the invention to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body.

### Combination Therapies for Cancer Treatment

The antibodies of the invention may be administered in combination with a second therapeutic agent.

The antibodies of the invention may be administered in combination with one, two, three, four, five or six additional therapeutic agents.

Any of the antagonistic antibodies specifically binding PD-1, antagonistic antibodies specifically binding TIM-3 or antagonistic bispecific PD-1/TIM-3 antibodies of the invention may be used in combination with a second therapeutic agent.

Any of the antagonistic antibodies specifically binding PD-1, antagonistic antibodies specifically binding TIM-3 or antagonistic bispecific PD-1/TIM-3 antibodies of the invention may be used in combination with one, two, three, four, five or six additional therapeutic agents.

“In combination with” refers to administering of the antibodies of the invention and at least one second therapeutic agent concurrently as single agents or sequentially as single agents in any order. In general, each agent will be administered at a dose and/or on a time schedule determined for that agent.

In some embodiments, the second therapeutic agent modulates activity of a molecule involved in the cancer-immunity cycle, e.g. a molecule involved in stimulatory or inhibitory pathways functioning in release of cancer cell antigens, cancer antigen presentation, T cell priming and activation, trafficking of T cells to tumors, infiltration of T cells into tumors, recognition of cancer cells by T cells, and killing of cancer cells. The cancer-immunity cycle is described in Chen and Mellman (2013) Immunity 39:1-10. In some embodiments, the second therapeutic agend modulates activity of a molecule involved in regulation of activity of T regulatory cells (Treg), co-stimulatory or co-inhibitory ligands expressed on tumors, activating or inhibitory receptors on natural killer (NK) cells, or immunosuppressive factors in the tumor microenvironment. Combination cancer immunotherapies are described in Manoney et al., (2015) Nature Reviews 14:561-584.

The second therapeutic agent typically enhances the activity of stimulatory molecules and suppresses the activity of inhibitory molecules, as is well known. Thus, “modulate” refers to the enhancement of immune response by the second therapeutic agent, wheatear the agent itself is agonist or antagonist of a specific molecule.

In some embodiments, the antibodies of the invention are administered in combination with an inhibitor of a T cell inhibitory molecule.

In some embodiments, the antibodies of the invention are administered in combination with an inhibitor of a T cell inhibitory molecule PD-1, PD-L1, PD-L2, VISTA, BTNL2, B7-H3, B7-H4, HVEM, HHLA2, CTLA-4, LAG-3, TIM-3, BTLA, CD160, CEACAM-1, LAIR1, TGFβ, IL-10, Siglec family protein, KIR, CD96, TIGIT, NKG2A, CD 112, CD47, SIRPA or CD244.

In some embodiments, KIR is KIR2DLI, KIR2DL2 or KIR2DL3.

Inhibition of inhibitory molecules may be performed by inhibition at the DNA, RNA or protein level. In some embodiments, an inhibitory nucleic acid (e.g., a dsRNA, siRNA or shRNA) is used to inhibit expression of the inhibitory molecule.

In some embodiments, the inhibitor of the inhibitory molecule is a soluble ligand of the inhibitory molecule.

In some embodiments, the inhibitor of the inhibitory molecule is an antagonistic antibody specifically binding the inhibitory molecule.

In some embodiments, the inhibitor of the inhibitory molecule is CTLA-4-Fc or TIM-3-Fc fusion protein.

In some embodiments, the inhibitor of the inhibitory molecule is an antibody or an antibody fragment that binds PD-1, PD-L1, PD-L2, VISTA, BTNL2, B7-H3, B7-H4, HVEM, HHLA2, CTLA-4, LAG-3, TIM-3, BTLA, CD160, CEACAM-1, LAIR1, TGFβ, IL-10, Siglec family protein, KIR, CD96, TIGIT, NKG2A, CD112, CD47, SIRPA or CD244.

Exemplary anti-PD-1 antibodies that may be used in the methods of the invention are those described herein and in U.S. Pat. Nos. 5,897,862 and 7,488,802, and in Int. Patent Publ. Nos. WO2004/004771, WO2004/056875, WO2006/121168, WO2008/156712, WO2010/029435, WO2010/036959, WO2011/110604, WO2012/145493, WO2014/194302, WO2014/206107, WO2015/036394, WO2015/035606, WO2015/085847, WO2015/112900 and WO2015/112805. Exemplary anti-PD1 antibodies include KEYTRUDA®(pembrolizumab) and OPDIVO® (nivolumab).

In some embodiments, the antibodies of the invention are administered in combination with a soluble PD-1 ligand.

In some embodiments, the soluble PD-1 ligand is soluble PD-L1 or soluble PD-L2 fused to an Fc.

In some embodiments, the soluble PD-1 ligand is AMP-224.

In some embodiments, the antibodies of the invention are administered in combination with an anti-PD-L1 antibody, or antigen-binding fragments thereof.

Exemplary PD-L1 antibodies that may be used in the methods of the invention are antibodies MDPL3280A (Genentech/Roche) and other human monoclonal antibodies disclosed in U.S. Pat. No. 7,943,743 and U.S Patent Publ. No. 20120039906. Other anti-PD-L1 binding agents include YW243.55.S70 (heavy and light chain variable regions are shown in SEQ ID NOs 20 and 21 in WO2010/077634) and MDX-1105 (also referred to as BMS-936559, and, e.g., anti-PD-L1 binding agents disclosed in WO2007/005874). The VH and the VL sequences of anti-PD-L1 antibodies durvalumab, atezolimumab and avelumab that may be used are disclosed herein.

Exemplary PD-L2 antibodies that may be used in the methods of the invention are those described in U.S. Pat. Nos. 8,080,636, 8,188,238, U.S. Patent Publ. No. 20110271358 and Int. Patent Publ. No. WO2012145493.

Exemplary B7-H4 antibodies that may be used in the methods of the invention are those described in U.S. Pat. Nos. 7,888,477, 8,609,816, 7,931,896, European Patent No. 1817055, U.S. Patent Publ. No. US20140037551and US2014029486, and Int. Patent Publ. Nos. WO2014/100483 and WO2014/159835.

Exemplary anti-CTLA-4 antibodies that may be used in the methods of the invention are ipilimumab (MDX-010, CAS No. 477202-00-9) and tremelimumab (IgG2 monoclonal antibody available from Pfizer, formerly known as ticilimumab, CP-675,206).

Exemplary anti-LAG-3 antibodies that may be used in the methods of the invention are those described for example in Int. Patent Publ. Nos. WO2008/132601 and WO2010/019570.

Exemplary anti-CEACAM-1 antibodies that may be used in the methods of the invention are those described in U.S. Pat. No. 8,598,322 and in U.S. Patent Publ. Nos. US2004/0047858, US20140271618 and US20120100158. Without wishing to be bound by any particular theory, CEACAM-1 has been described as a ligand and partner of TIM-3 (see e.g., Int. Patent Publ. No. WO2014/022332). Synergistic in vivo effect of the combination of anti-TIM-3 and anti-CEACAM-1 antibodies have been detected in xenograft cancer models (see e.g., Int. Patent Publ. No. WO2014/022332). Tumors may use CEACAM-1 to inhibit the immune system. Therefore, anti-CEACAM-1 antibodies may be used in combination with the antibodies of the invention described herein.

Exemplary anti-LAIR1 antibodies that may be used in the methods of the invention are those described in U.S. Pat. No. 6,479,638 and Int. Patent Publ. No. WO2010/078580.

Exemplary anti-CD96 antibodies that may be used in the methods of the invention are those described in Int. Patent Publ. No. WO2015/024060.

Exemplary anti-TIM-3 antibodies that may be used in the methods of the invention are those described herein and in Int. Patent Publ. Nos. WO2011/155607, WO2013/006490 and WO2015/117002.

Exemplary anti-TIGIT antibodies that may be used in the methods of the invention are those described in U.S. Patent Publ. Nos. US20140056890 and US20150216970. An exemplary anti-TIGIT antibody is RG-6058 (MTIG-7192A).

TIGIT expression was found herein to be elevated in CD8⁺ T cells isolated from tumors after anti-TIM-3 antibody treatment in animal models of cancer. Therefore, therapeutic administration of antagonistic antibodies specifically binding TIGIT to a subject who has already received or is receiving anti-TIM-3 antibody therapy, is refractory to the anti-TIM-3 antibody treatment or has relapsed after or during the anti-TIM-3 antibody treatment may improve the clinical outcome of the patients.

The invention also provides a method of treating a cancer in a subject, comprising administering to the subject in need thereof a therapeutically effective amount of an antagonistic antibody that specifically binds TIM-3 and an antagonistic antibody that specifically binds TIGIT for a time sufficient to treat the cancer.

In some embodiments, the antagonistic antibody that specifically binds TIGIT is administered after administration of the antagonistic antibody specifically binding TIM-3.

In some embodiments, the antagonistic antibody that specifically binds TIGIT and the antagonistic antibody specifically binding TIM-3 are administered concurrently as single agents or sequentially as single agents in any order.

Exemplary anti-BTLA antibodies that may be used in the methods of the invention are those described in U.S. Pat. Nos. 8,546,541, 7,479,544, 8,188,232, 8,247,537, 8,563,694 and in Int. Patent Publ. No. WO2014184360.

Exemplary anti-HVEM antibodies that may be used in the methods of the invention are those described in U.S. Patent Publ. No. US20110280866.

Exemplary CD47 antibodies that may be used in the methods of the invention are those described in U.S. Pat. No. 8,101,719.

Exemplary CD244 antibodies that may be used in the methods of the invention include those described in U.S. Pat. No. 5,688,690.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-TIM-3 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-PD-L1 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-PD-L2 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-I/TIM-3 antibodies of the invention are administered in combination with an anti-VISTA antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-BTNL2 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-B7-H3 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-B7-H4 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-HVEM antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-HLA2 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CTLA-4 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-LAG-3 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-TIM-3 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-BTLA antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD160 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CEACAM-1 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-LAIR1 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-TGFβ antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-IL-10 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-TIGIT antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-KIR antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-NKG2A antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD112 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD47 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-SIRPA antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD244antibody or antigen-binding fragment thereof.

The immune inhibitory molecules may regulate or synergistically regulate T-cell functions to promote tumoral immune escape. Therefore, combination therapies with two or more inhibitors of the inhibitory molecules may provide an improved therapy to a patient when compared to monotherapy alone.

In some embodiments, the antibodies of the invention are administered in combination with an activator of an activating molecule.

In some embodiments, the antibodies of the invention are administered in combination with an activator of an activating molecule CD86, CD80, CD28, ICOS, ICOS ligand, TMIGD2, CD40, GITR ligand, 4-1BB ligand, OX40 ligand, CD70, CD40L, TNFRSF25, LIGHT, GITR, OX-40, CD27, CD137, NKG2D, CD48, CD226 or MICA.

Activation of activating molecules may be performed using for example soluble ligands or ligand derivatives of the activating molecules, peptides or agonistic antibodies.

In some embodiments, the activator of the activating molecule is a soluble ligand of the T cell activating molecule.

In some embodiments, the activator of the activating molecule is an agonistic antibody specifically binding the activating molecule.

Exemplary anti-CD40 antibodies that may be used in the methods of the invention include CP-870,893 and humanized S2C6 described in U.S. Pat. No. 7,288,251 (antibody 21.4.1) and U.S. Pat. No. 8,303,955, respectively, and anti-CD40 antibodies described in Int. Patent Publ. Nos. WO2001/056603, WO2001/083755, WO2013/034904 and WO2014/070934.

Exemplary GITR agonists include, e.g., GITR fusion proteins and anti-GITR antibodies (e.g., bivalent anti-GITR antibodies), such as, a GITR fusion protein described in U.S. Pat. No. 6,111,090, European Patent No. 090505B1, U.S. Pat. No. 8,586,023, Int. Patent. Publ. Nos. WO2010/003118 and WO2011/090754, or an anti-GITR antibody described in U.S. Pat. Nos. 7,025,962, 7,812,135, 8,388,967, 8,591,886 and 7,618,632, European Patent Nos. 1947183 and 1866339, or Int. Patent Publ. Nos. WO2011/028683, WO2013/039954, WO2005/007190, WO2007/133822, WO2005/055808, WO1999/40196, WO2001/03720, WO1999/20758, WO2006/083289, WO2005/115451 and WO2011/051726.

GITR expression was found herein to be elevated in CD8⁺ T cells isolated from tumors after anti-PD-1 antibody treatment in animal models of cancer. The restoration of GITR expression on TILs by anti-PD-1 treatment supports that combination therapy with anti-GITR and anti-PD-1 antibodies may improve the clinical outcome of the patients.

The invention also provides a method of treating a cancer in a subject, comprising administering to the subject in need thereof a therapeutically effective amount of an antagonistic antibody that specifically binds PD-1 and an agonistic antibody that specifically binds GITR for a time sufficient to treat the cancer.

In some embodiments, the agonistic antibody that specifically binds GITR is administered after administration of the antagonistic antibody specifically binding PD-1.

In some embodiments, the agonistic antibody that specifically binds GITR and the antagonistic antibody specifically binding PD-1 are administered concurrently as single agents or sequentially as single agents in any order.

Exemplary OX40 antibodies that may be used in the methods of the invention include those described in U.S. Pat. Nos. 8,133,983, 7,960,515, U.S. Patent Publ. No. 20130280275 and Int. Patent Publ. Nos. WO2013028231 and WO2014148895.

An exemplary OX40 antibody that may be used in the methods of the invention is an antibody comprising the VH of SEQ ID NO: 309 and the VL of SEQ ID NO: 310.

Another exemplary OX40 antibody that may be used in the methods of the invention is an antibody comprising the VH of SEQ ID NO: 311 and the VL of SEQ ID NO: 312.

OX40 expression was found herein to be elevated in CD8⁺ T cells isolated from tumors after anti-PD-1 antibody treatment in animal models of cancer. The restoration of OX40 expression on TILs by anti-PD-1 treatment supports that combination therapy with anti-OX40 and anti-PD-1 antibodies may improve the clinical outcome of the patients.

The invention also provides a method of treating a cancer in a subject, comprising administering to the subject in need thereof a therapeutically effective amount of an antagonistic antibody that specifically binds PD-1 and an agonistic antibody that specifically binds OX40 for a time sufficient to treat the cancer.

In some embodiments, the agonistic antibody that specifically binds OX40 is administered after administration of the antagonistic antibody specifically binding PD-1.

In some embodiments, the agonistic antibody that specifically binds OX40 and the antagonistic antibody specifically binding PD-1 are administered concurrently as single agents or sequentially as single agents in any order.

Exemplary CD70 antibodies that may be used in the methods of the invention include those described in U.S. Patent Publ. No. US20130336976.

Exemplary TNFRSF25 antibodies that may be used in the methods of the invention include those described in U.S. Pat. No. 7,708,996.

Exemplary CD27 antibodies that may be used in the methods of the invention include those described in U.S. Patent Publ. No. US20130336976.

Exemplary CD137 antibodies that may be used in the methods of the invention include those described in U.S. Pat. Nos. 6,974,863, 6,303,121, 7,138,500, 7,288,638, 8,716,452, 8,821,867 and in U.S. Patent Publ. No. US20130149301.

CD137 expression was found herein to be elevated in CD8⁺ T cells isolated from tumors after anti-PD-1 antibody treatment in animal models of cancer. The restoration of CD137 expression on TILs by anti-PD-1 treatment supports that combination therapy with anti-CD137 and anti-PD-1 antibodies may improve the clinical outcome of the patients.

The invention also provides a method of treating a cancer in a subject, comprising administering to the subject in need thereof a therapeutically effective amount of an antagonistic antibody that specifically binds PD-1 and an agonistic antibody that specifically binds CD137 for a time sufficient to treat the cancer.

In some embodiments, the agonistic antibody that specifically binds CD137 is administered after administration of the antagonistic antibody specifically binding PD-1.

In some embodiments, the agonistic antibody that specifically binds CD137 and the antagonistic antibody specifically binding PD-1 are administered concurrently as single agents or sequentially as single agents in any order.

Exemplary NKG2D antibodies that may be used in the methods of the invention include those described in U.S. Patent Publ. No. US20110150870.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD86 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD80 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD28 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-ICOS antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-ICOS ligand antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-TMIGD2 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD40 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-GITR ligand antibody or antigen-binding fragment thereof. In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-4-1BB ligand antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-OX40 ligand antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD70 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD40L antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-TNFRSF25 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-LIGHT antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-GITR antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-OX40 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD27 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD137 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-NKG2D antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD48 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-CD226 antibody or antigen-binding fragment thereof.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with an anti-MICA antibody or antigen-binding fragment thereof.

The combination of antibodies recited herein can be administered separately, e.g., as separate antibodies, or linked, e.g., as a bispecific or trispecific antibody molecule.

The efficacy of the combinations described herein may be tested in animal models known in the art.

Antibodies of the invention described herein may be administered in combination with a vaccine.

Exemplary vaccines are immunogenic agents, such as cancerous cells, purified tumor antigens (including recombinant proteins, antigen epitopes, peptides and carbohydrate molecules), tumor antigens delivered to a patient via gene therapy, cells, and cells transfected with genes encoding immune stimulating cytokines. Exemplary vaccines that may be used include peptides of melanoma antigens, such as peptides of gp100, MAGE antigens, Trp-2, MART1 and/or tyrosinase, or tumor cells transfected to express the cytokine GM-CSF, DNA-based vaccines, RNA-based vaccines, and viral transduction-based vaccines, peptides or prostate antigens or peptides of lung cancer antigens. The cancer vaccine may be prophylactic or therapeutic.

Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita, V. et al. (eds.), 1997, Cancer: Principles and Practice of Oncology. Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al., (1993) Proc Natl Acad Sci U.S.A. 90: 3539-43).

The antibodies of the invention described herein may be administered in combination with one or a collection of recombinant proteins and/or peptides expressed in or on a tumor in order to generate an immune response to these proteins. These proteins are normally viewed by the immune system as self-antigens and are therefore tolerant to them. The tumor antigen may also include the protein telomerase, which is required for the synthesis of telomeres of chromosomes and which is expressed in more than 85% of human cancers and in only a limited number of somatic tissues (Kim et al., (1994) Science 266: 2011-2013). Tumor antigens may also be “neo-antigens” expressed in or on cancer cells as a result of somatic mutations that alter protein sequence or create fusion proteins between two unrelated sequences (e.g., bcr-abl in the Philadelphia chromosome), or idiotype from B cell tumors. The tumor antigens may be antigen epitopes of prostate specific antigen (PSA), mesothelin, prostate-specific membrane antigen (PSMA), synovial sarcoma X2 (SSX2), NKX3.1, prostatic acidic phosphatase (PAP), or epidermal growth factor receptors, or peptides specific for variants of EGFR such as the well-known EGFRvIII overexpressed on tumor cells.

Other tumor vaccines may include the proteins from viruses implicated in human cancers such a Human Papilloma Viruses (HPV), Hepatitis Viruses (HBV and HCV) and Kaposi's Herpes Sarcoma Virus (KHSV), and Epstein-Barr virus (EBV). Another form of tumor specific antigens which may be used in combination with the antibodies of the invention described herein is purified heat shock proteins (HSP) isolated from the tumor tissue itself. HSP contain fragments of proteins from the tumor cells and are highly efficient at delivery to antigen presenting cells for eliciting tumor immunity (Suot and Srivastava (1995) Science 269:1585-1588; Tamura et al., (1997) Science 278:117-120).

Dendritic cells (DC) are potent antigen presenting cells that may be used to prime antigen-specific responses. DC's may be produced ex vivo and loaded with various protein and peptide antigens as well as tumor cell extracts (Nestle et al., (1998) Nature Medicine 4: 328-332). DCs may also be transduced by genetic means to express these tumor antigens. DCs have also been fused directly to tumor cells for the purposes of immunization (Kugler et al., (2000) Nature Medicine 6:332-336). As a method of vaccination, DC immunization may be effectively combined with the antibodies of the invention described herein to activate more potent anti-tumor responses.

In some embodiments, the vaccine is a polypeptide or a fragment thereof, or a DNA or a RNA encoding the polypeptide or fragment thereof expressed on tumor cells.

In some embodiments, the polypeptide or fragment thereof expressed on tumor cells is PSMA.

In some embodiments, the polypeptide or fragment thereof expressed on tumor cells is mesothelin.

In some embodiments, the polypeptide or fragment thereof expressed on tumor cells is EGFR or EGFR variant such as EGFRvIII.

In some embodiments, the polypeptide or fragment thereof expressed on tumor cells is PAP.

In some embodiments, the polypeptide or fragment thereof expressed on tumor cells is synovial sarcoma X2 (SSX2).

In some embodiments, the polypeptide or fragment thereof expressed on tumor cells is NKX3.1.

In some embodiments, the tumor cells are melanoma, lung cancer, squamous non-small cell lung cancer (NSCLC), non-squamous NSCLC, colorectal cancer, prostate cancer, castration-resistant prostate cancer, ovarian cancer, gastric cancer, liver cancer, pancreatic cancer, thyroid cancer, squamous cell carcinoma of the head and neck, carcinomas of the esophagus or gastrointestinal tract or breast cancer cells.

In some embodiments, the antibodies of the invention are administered in combination with a renal carcinoma (RCC) vaccine.

In some embodiments, the antibodies of the invention are administered in combination with a lung cancer vaccine.

In some embodiments, the antibodies of the invention are administered in combination with a prostate cancer vaccine.

In some embodiments, the antibodies of the invention are administered in combination with a lung cancer vaccine.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with a tumor vaccine comprising a peptide fragment of EGFR or EGFRvIII, or a vector encoding the peptide fragment of EGFR or EGFRvIII.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with a tumor vaccine comprising a peptide fragment of mesothelin, or a vector encoding the peptide fragment of mesothelin.

In some embodiments, the antagonistic antibodies specifically binding PD-1 of the invention, the antagonistic antibodies specifically binding TIM-3 or the invention, or the antagonistic bispecific PD-1/TIM-3 antibodies of the invention are administered in combination with a tumor vaccine comprising a peptide fragment of prostate specific antigen, or a vector encoding the peptide fragment of prostate specific antigen.

Suitable vectors that may be used in the methods of the invention are well known and include lentiviral vectors, adenoviral vectors, minimal nucleic acid vector (MNAV), vaccinia virus, flow pox virus, Alpha virus-derived VRP, Saccharomyces cerevisiae, MVA, Listeria moonocytogenes, pVAX-based plasmid, see e.g. Pol et al., (2014) Oncoimmunology 1(3):e28185.

The antibodies of the invention may be administered in combination with a standard of care cancer treatment.

The antibodies of the invention described herein may be administered in combination with a standard of care cancer chemotherapeutic regimes. In these instances, it may be possible to reduce the dose of chemotherapeutic reagent administered (Mokyr et al., (1998) Cancer Research 58: 5301-5304).

In some embodiments, the antibodies of the invention may be administered in combination with one or more of other antibody molecules, chemotherapy, other anti-cancer therapy (e.g., targeted anti-cancer therapies, or oncolytic drugs), cytotoxic agents, cytokines, surgical and/or radiation procedures.

Exemplary cytotoxic agents that may be administered in combination with the antibodies of the invention include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteosome inhibitors, and radiation (e.g., local or whole body irradiation).

Standard of care therapeutics include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), Ibrutinib, idelalisib, and brentuximab vedotin.

Exemplary alkylating agents include, nitrogen mustards, ethylenimine derivatives, alkyl sulfonates, nitrosoureas and triazenes: uracil mustard (Aminouracil Mustard®, Chlorethaminacil®, Demethyldopan®, Desmethyldopan®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), chlorambucil (Leukeran®), pipobroman (Amedel®, Vercyte®), triethylenemelamine (Hemel®, Hexalen®, Hexastat®), triethylenethiophosphoramine, temozolomide (Temodar®), thiotepa (Thioplex®), busulfan (Busilvex®, Myleran®), carmustine (BiCNU®), lomustine (CeeNU®) and streptozocin (Zanosar®). Additional exemplary alkylating agents include, oxaliplatin (Eloxatin®), temozolomide (Temodar® and Temodal®), dactinomycin (also known as actinomycin-D, Cosmegen®), altretamine (also known as hexamethylmelamine (HMM), Hexalen®), bendamustine (Treanda®), carboplatin (Paraplatin®), lomustine (also known as CCNU, CeeNU®), cisplatin (also known as CDDP, Platinol® and Platinol®-AQ), chlorambucil (Leukeran®), prednumustine, procarbazine (Matulane®),and thiotepa (also known as thiophosphoamide, TESPA and TSPA, Thioplex®).

Exemplary anthracyclines include, e.g., doxorubicin (Adriamycin® and Rubex®); bleomycin (Lenoxane®), daunorubicin (dauorubicin hydrochloride, daunomycin, and rubidomycin hydrochloride, Cerubidine®), daunorubicin liposomal (daunorubicin citrate liposome, DaunoXome®), mitoxantrone (DHAD, Novantrone®), epirubicin (Ellence™) idarubicin (Idamycin®, Idamycin PFS®), mitomycin C (Mutamycin®), geldanamycin, herbimycin, ravidomycin, and desacetylravidomycin.

Exemplary vinca alkaloids that may be used in combination with the antibodies of the invention include vinorelbine tartrate (Navelbine®), vincristine (Oncovin®), and vindesine (Eldisine®), vinblastine (also known as vinblastine sulfate, vincaleukoblastine and VLB, Alkaban-AQ® and Velban®) and vinorelbine (Navelbine®).

Exemplary proteosome inhibitors that may be used in combination with the antibodies of the invention are bortezomib (Velcade®); carfilzomib (Kyprolis®), ixazomib (Ninlaro®), marizomib (NPI-0052) and delanzomib (CEP-18770).

In some embodiments, the antibodies of the invention are administered in combination with a tyrosine kinase inhibitor (e.g., a receptor tyrosine kinase (RTK) inhibitor). Exemplary tyrosine kinase inhibitor include an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor), a platelet derived growth factor (PDGF) pathway inhibitor (e.g., a platelet derived growth factor receptor (PDGFR) inhibitor (e.g., a PDGFR-β inhibitor), a RAF-1 inhibitor, a KIT inhibitor and a RET inhibitor. In some embodiments, the second therapeutic is axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAMPATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, pazopanib hydrochloride, PD173074, Sorafenib Tosylate (Bay 43-9006), SU 5402, TSU-68 (SU6668), vatalanib, XL880 (GSK1363089, EXEL-2880). Selected tyrosine kinase inhibitors are chosen from sunitinib, erlotinib, gefitinib, or sorafenib. In some embodiments, the EGFR inhibitor is a bispecific EGFRc-Met antibody (EM-1 mAb) comprising the heavy and the light chains of SEQ ID NOs: 249, 250, 251 and 252 (US2014/0141000).

In some embodiments, the antibodies of the invention are administered in combination with Vascular Endothelial Growth Factor (VEGF) receptor inhibitors, including bevacizumab (Avastin®), axitinib (Inlyta®), brivanib alaninate (BMS-582664, (S)-((R)-1-(4-(4-Fluoro-2-methyl-1H-indol-5-yloxy)-5-methylpyrrolo[2,1-f][1,2,4]triazin-6-yloxy)propan-2-yl)2-aminopropanoate), sorafenib (Nexavar®); Pazopanib (Votrient®), sunitinib malate (Sutent®), cediranib (AZD2171, CAS 288383-20-1), vargatef (BIBF1120, CAS 928326-83-4), foretinib (GSK1363089), telatinib (BAY57-9352, CAS 332012-40-5), apatinib (YN968D1, CAS 811803-05-1), imatinib (Gleevec®), ponatinib (AP24534, CAS 943319-70-8), tivozanib (AV951, CAS 475108-18-0), regorafenib (BAY73-4506, CAS 755037-03-7), vatalanib dihydrochloride (PTK787, CAS 212141-51-0), brivanib (BMS-540215, CAS 649735-46-6), vandetanib (Caprelsa® or AZD6474), motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470), dovitinib dilactic acid (TKI258, CAS 852433-84-2), linfanib (ABT869, CAS 796967-16-3); Cabozantinib (XL184, CAS 849217-68-1), lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aa,5p,6aa)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and aflibercept (Eylea®).

In some embodiments, the antibodies of the invention are administered in combination with a PI3K inhibitor. In one embodiment, the PI3K inhibitor is an inhibitor of delta and gamma isoforms of PI3K. Exemplary PI3K inhibitors that may be used are described in, e.g., WO 2010/036380, WO 2010/006086, WO 09/114870, WO 05/113556, GSK 2126458, GDC-0980, GDC-0941, Sanofi XL147, XL756, XL147, PF-46915032, BKM 120, CAL-101, CAL 263, SF1126, PX-886, and a dual PI3K inhibitor (e.g., Novartis BEZ235).

In some embodiments, the antibodies of the invention are administered in combination with a mTOR inhibitor, e.g., one or more mTOR inhibitors chosen from one or more of rapamycin, temsirolimus (TORISEL®), AZD8055, BEZ235, BGT226, XL765, PF-4691502, GDC0980, SF1126, OSI-027, GSK1059615, KU-0063794, WYE-354, Palomid 529 (P529), PF-04691502, or PKI-587. ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.04,9]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-a-aspartylL-serine-(SEQ ID NO: 237), inner salt (SF1126, CAS 936487-67-1), and XL765.

In some embodiments, the antibodies of the invention are administered in combination with a BRAF inhibitor, e.g., GSK2118436, RG7204, PLX4032, GDC-0879, PLX4720, and sorafenib tosylate (Bay 43-9006).

In some embodiments, the antibodies of the invention are administered in combination with a MEK inhibitor.

In some embodiments, the antibodies of the invention are administered in combination with a JAK2 inhibitor, e.g., CEP-701, INCB18424, CP-690550 (tasocitinib).

In some embodiments, the antibodies of the invention are administered in combination with paclitaxel or a paclitaxel agent, e.g., TAXOL®, protein-bound paclitaxel (e.g., ABRAXANE®). Exemplary paclitaxel agents include nanoparticle albumin-bound paclitaxel (ABRAXANE, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate, see Liu et al., (2007) Bioorganic & Medicinal Chemistry Letters 17:617-620).

In some embodiments, the antibodies of the invention are administered in combination with a cellular immunotherapy (e.g., Provenge (e.g., Sipuleucel)), and optionally in combination with cyclophosphamide.

Exemplary therapeutic agents that may be used in combination with the antibodies of the invention for treatment of pancreatic cancer include a chemotherapeutic agent, e.g., paclitaxel or a paclitaxel agent (e.g., a paclitaxel formulation such as TAXOL, an albumin-stabilized nanoparticle paclitaxel formulation (e.g., ABRAXANE) or a liposomal paclitaxel formulation); gemcitabine (e.g., gemcitabine alone or in combination with AXP107-11); other chemotherapeutic agents such as oxaliplatin, 5-fluorouracil, capecitabine, rubitecan, epirubicin hydrochloride, NC-6004, cisplatin, docetaxel (e.g., TAXOTERE), mitomycin C, ifosfamide; interferon; tyrosine kinase inhibitor (e.g., EGFR inhibitor (e.g., erlotinib, panitumumab, cetuximab, nimotuzumab); HER2/neu receptor inhibitor (e.g., trastuzumab); dual kinase inhibitor (e.g., bosutinib, saracatinib, lapatinib, vandetanib); multikinase inhibitor (e.g., sorafenib, sunitinib, XL184, pazopanib); VEGF inhibitor (e.g., bevacizumab, AV-951, brivanib); radioimmunotherapy (e.g., XR303); cancer vaccine (e.g., GVAX, survivin peptide); COX-2 inhibitor (e.g., celecoxib); IGF-1 receptor inhibitor (e.g., AMG 479, MK-0646); mTOR inhibitor (e.g., everolimus, temsirolimus), IL-6 inhibitor (e.g., CNTO 328); cyclin-dependent kinase inhibitor (e.g., P276-00, UCN-01); Altered Energy Metabolism-Directed (AEMD) compound (e.g., CPI-613); HDAC inhibitor (e.g., vorinostat); TRAIL receptor 2 (TR-2) agonist (e.g., conatumumab); MEK inhibitor (e.g., AS703026, selumetinib, GSK1120212); Raf/MEK dual kinase inhibitor (e.g., RO5126766), Notch signaling inhibitor (e.g., MK0752), monoclonal antibody-antibody fusion protein (e.g., L191L2), curcumin; HSP90 inhibitor (e.g., tanespimycin, STA-9090), rIL-2; denileukin diftitox; topoisomerase 1 inhibitor (e.g., irinotecan, PEP02); statin (e.g., simvastatin), Factor VIIa inhibitor (e.g., PCI-27483), AKT inhibitor (e.g., RX-0201), hypoxia-activated prodrug (e.g., TH-302), metformin hydrochloride, gamma-secretase inhibitor (e.g., R04929097), ribonucleotide reductase inhibitor (e.g., 3-AP), immunotoxin (e.g., HuC242-DM4), PARP inhibitor (e.g., KU-0059436, veliparib), CTLA-4 inhbitor (e.g., CP-675,206, ipilimumab), AdV-tk therapy, proteasome inhibitor (e.g., bortezomib (Velcade), NPI-0052), thiazolidinedione (e.g., pioglitazone), NPC-1C; Aurora kinase inhibitor (e.g., R763/AS703569), CTGF inhibitor (e.g., FG-3019), siG12D LODER and radiation therapy (e.g., tomotherapy, stereotactic radiation, proton therapy), surgery, and a combination thereof. In certain embodiments, a combination of paclitaxel or a paclitaxel agent, and gemcitabine can be used with the antibodies of the invention.

Exemplary therapeutic agents that may be used in combination with the antibodies of the invention for treatment of small cell lung cancer (SCLC) include approved drugs for treatment of SCLC such as methotrexate (Folex®, Mexate®), everolimus (Afinitor®), doxorubicin hydrochloride, etoposide phosphate (Etopophos®), topotecan hydrochloride (Hycamtin®), mechlorethamine hydrochloride (Mustargen®), topotecan hydrochloride. Other therapeutic agents that may be used are carboplatin, cisplatin, oxaliplatin, irinotecan, gemcitabine, liposomal SN-38, bendamustine, temozolomide, belotecan, NK012, FR901228, flavopiridol), tyrosine kinase inhibitor (e.g., EGFR inhibitor (e.g., erlotinib, gefitinib, cetuximab, panitumumab), multikinase inhibitor (e.g., sorafenib, sunitinib), VEGF inhibitor (e.g., bevacizumab, vandetanib), cancer vaccine (e.g., GVAX); Bel-2 inhibitor (e.g., oblimersen sodium, ABT-263), proteasome inhibitor (e.g., bortezomib (Velcade), NPI-0052), paclitaxel or a paclitaxel agent; docetaxel, IGF-1 receptor inhibitor (e.g., AMG 479), HGF/SF inhibitor (e.g., AMG 102, MK-0646), chloroquine, Aurora kinase inhibitor (e.g., MLN8237), radioimmunotherapy (e.g., TF2), HSP90 inhibitor (e.g., tanespimycin, STA-9090), mTOR inhibitor (e.g., everolimus), Ep-CAM/CD3-bispecific antibody (e.g., MT110), CK-2 inhibitor (e.g., CX-4945), HDAC inhibitor (e.g., belinostat), SMO antagonist (e.g., BMS 833923), peptide cancer vaccine, and radiation therapy (e.g., intensity-modulated radiation therapy (IMRT), hypofractionated radiotherapy, hypoxia-guided radiotherapy), surgery, and combinations thereof.

Exemplary therapeutic agents that may be used in combination with the antibodies of the invention for treatment of non-small cell lung cancer include approved drugs for treatment of NSCLC including methotrexate (Folex®, Mexate®), paclitaxel (Abraxane®), afatinib (Gilotrif®), everolimus (Afmitor®), alectinib (Alecensa®), pemetrexed disodium (Alimta®), bevacizumab (Avastin®), carboplatin, ceritinib (Zykadia®), crizotinib (Xalkori®), ramucirumab (Cyramza®), docetaxel, everolimus (Afinitor®),gefitinib (Iressa®), afatinib dimaleate (Gilotrif®), gemcitabine hydrochloride (Gmezar®), pembrolizumab (Keytruda®), mechlorethamine hydrochloride (Mustargen®), vinorelbine tartrate (Navelbine®), necitumumab (Portrazza®), nivolumab (Opdivo®), osimertinib, paclitaxel (Taxol®), carboplatin, pemetrexed disodium, ramucirumab (Cyramza®), osimertinib (Tagrisso®). Other therapeutic agents that may be used are vinorelbine, cisplatin, docetaxel, pemetrexed disodium, etoposide, gemcitabine, carboplatin, liposomal SN-38, TLK286, temozolomide, topotecan, pemetrexed disodium, azacitidine, irinotecan, tegafur-gimeracil-oteracil potassium, sapacitabine), tyrosine kinase inhibitor (e.g., EGFR inhibitor (e.g., erlotinib, gefitinib, cetuximab, panitumumab, necitumumab, PF-00299804, nimotuzumab, R05083945), MET inhibitor (e.g., PF-02341066, ARQ 197), PI3K kinase inhibitor (e.g., XL147, GDC-0941), Raf/MEK dual kinase inhibitor (e.g., R05126766), PI3K/mTOR dual kinase inhibitor (e.g., XL765), SRC inhibitor (e.g., dasatinib), dual inhibitor (e.g., BIBW 2992, GSK1363089, ZD6474, AZD0530, AG-013736, lapatinib, MEHD7945A, linifanib), multikinase inhibitor (e.g., sorafenib, sunitinib, pazopanib, AMG 706, XL184, MGCD265, BMS-690514, R935788), VEGF inhibitor (e.g., endostar, endostatin, bevacizumab, cediranib, BIBF 1120, axitinib, tivozanib, AZD2171), cancer vaccine (e.g., BLP25 liposome vaccine, GVAX, recombinant DNA and adenovirus expressing L523S protein), Bel-2 inhibitor (e.g., oblimersen sodium), proteasome inhibitor (e.g., bortezomib, carfilzomib, NPI-0052, MLN9708), paclitaxel or a paclitaxel agent, docetaxel, IGF-1 receptor inhibitor (e.g., cixutumumab, MK-0646, OSI 906, CP-751,871, B11B022), hydroxychloroquine, HSP90 inhibitor (e.g., tanespimycin, STA-9090, AUY922, XL888), mTOR inhibitor (e.g., everolimus, temsirolimus, ridaforolimus), Ep-CAM/CD3-bispecific antibody (e.g., MT110), CK-2 inhibitor (e.g., CX-4945), HDAC inhibitor (e.g., MS 275, LBH589, vorinostat, valproic acid, FR901228), DHFR inhibitor (e.g., pralatrexate), retinoid (e.g., bexarotene, tretinoin), antibody-drug conjugate (e.g., SGN-15), bisphosphonate (e.g., zoledronic acid), cancer vaccine (e.g., belagenpumatucel-L), low molecular weight heparin (LMWH) (e.g., tinzaparin, enoxaparin), GSK1572932A, melatonin, talactoferrin, dimesna, topoisomerase inhibitor (e.g., amrubicin, etoposide, karenitecin), nelfinavir, cilengitide, ErbB3 inhibitor (e.g., MM-121, U3-1287), survivin inhibitor (e.g., YM155, LY2181308), eribulin mesylate, COX-2 inhibitor (e.g., celecoxib), pegfilgrastim, Polo-like kinase 1 inhibitor (e.g., BI 6727), TRAIL receptor 2 (TR-2) agonist (e.g., CS-1008), CNGRC peptide (SEQ ID NO: 225)-TNF alpha conjugate, dichloroacetate (DCA), HGF inhibitor (e.g., SCH 900105), SAR240550, PPAR-gamma agonist (e.g., CS-7017), gamma-secretase inhibitor (e.g., R04929097), epigenetic therapy (e.g., 5-azacitidine), nitroglycerin, MEK inhibitor (e.g., AZD6244), cyclin-dependent kinase inhibitor (e.g., UCN-01), cholesterol-Fusi, antitubulin agent (e.g., E7389), farnesyl-OH-transferase inhibitor (e.g., lonafarnib), immunotoxin (e.g., BB-10901, SS1 (dsFv) PE38), fondaparinux, vascular-disrupting agent (e.g., AVE8062), PD-L1 inhibitor (e.g., MDX-1105, MDX-1106), beta-glucan, NGR-hTNF, EMD 521873, MEK inhibitor (e.g., GSK1120212), epothilone analog (e.g., ixabepilone), kinesin-spindle inhibitor (e.g., 4SC-205), telomere targeting agent (e.g., KML-001), P70 pathway inhibitor (e.g., LY2584702), AKT inhibitor (e.g., MK-2206), angiogenesis inhibitor (e.g., lenalidomide), Notch signaling inhibitor (e.g., OMP-21M18), EGFR/c-Met bispecific antibody EM-1 as described in US2014/0141000A1, radiation therapy, surgery, and combinations thereof.

Exemplary therapeutic agents that may be used in combination with the antibodies of the invention for treatment of ovarian cancer include approved drugs for treatment of ovarian cancer, such as melphalan (Alkeran®), bevacizumab (Avastin®), carboplatin, cyclophosphamide (Clafen®, Cytoxan®), clisplatin, doxorubicin hydrochloride, gemcitabine hydrochloride (Gemzar®), topotecan hydrochloride (Hycamtin®), Olaparib (Lynparza®), carboplatin, cisplatin, paclitaxel (Taxol®), thiotepa and topotecan hydrochloride. Other therapeutic agents that may be used are, ifosfamide, olaparib, oxaliplatin, pemetrexed disodium, SJG-136, etoposide, decitabine; immunotherapy (e.g., APC8024, oregovomab, OPT-821), tyrosine kinase inhibitor (e.g., EGFR inhibitor (e.g., erlotinib), dual inhibitor (e.g., E7080), multikinase inhibitor (e.g., AZD0530, JI-101, sorafenib, sunitinib, pazopanib), VEGF inhibitor (e.g., bevacizumab, BIBF 1120, cediranib, AZD2171), PDGFR inhibitor (e.g., IMC-3G3), paclitaxel, topoisomerase inhibitor (e.g., karenitecin, Irinotecan), HDAC inhibitor (e.g., valproate, vorinostat), folate receptor inhibitor (e.g., farletuzumab), angiopoietin inhibitor (e.g., AMG 386), epothilone analog (e.g., ixabepilone), proteasome inhibitor (e.g., carfilzomib), IGF-1 receptor inhibitor (e.g., OSI 906, AMG 479), PARP inhibitor (e.g., veliparib, AG014699, iniparib, MK-4827), Aurora kinase inhibitor (e.g., MLN8237, ENMD-2076), angiogenesis inhibitor (e.g., lenalidomide), DHFR inhibitor (e.g., pralatrexate), radioimmunotherapeutic agent (e.g., Hu3S193), statin (e.g., lovastatin), topoisomerase 1 inhibitor (e.g., NKTR-102), cancer vaccine (e.g., p53 synthetic long peptides vaccine, autologous OC-DC vaccine), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), HGF/SF inhibitor (e.g., AMG 102), EGEN-001, Polo-like kinase 1 inhibitor (e.g., BI 6727), gamma-secretase inhibitor (e.g., R04929097), Wee-1 inhibitor (e.g., MK-1775), antitubulin agent (e.g., vinorelbine, E7389), immunotoxin (e.g., denileukin diftitox), SB-485232, vascular-disrupting agent (e.g., AVE8062), integrin inhibitor (e.g., EMD 525797), kinesin-spindle inhibitor (e.g., 4SC-205), revlimid, HER2 inhibitor (e.g., MGAH22), ErrB3 inhibitor (e.g., MM-121), radiation therapy, and combinations thereof.

Exemplary therapeutic agents that may be used in combination with the antibodies of the invention for treatment of a myeloma include one or more of chemotherapy or other anti-cancer agents (e.g., thalidomide analogs, e.g., lenalidomide), HSCT (Cook, (2008) J Manag Care Pharm. 14(7 Suppl):19-25), an anti-TIM-3 antibody (Hallett et al., (2011) J of American Society for Blood and Marrow Transplantation 17(8):1133-145), tumor antigen-pulsed dendritic cells, fusions (e.g., electrofusions) of tumor cells and dendritic cells, or vaccination with immunoglobulin idiotype produced by malignant plasma cells (reviewed in Yi (2009) Cancer J 15(6):502-10).

Exemplary therapeutics agents that may be used in combination with the antibodies of the invention for treatment of a renal cancer, e.g., a renal cell carcinoma (RCC) or metastatic RCC include drugs approved for treatment of RCC, including everolimus (Afinitor®), aldesleukin, bevacizumab (Avastin®), axitinib (Inlyta®), cabozantinib-S-Malate (Cabometyx®), aldesleukin (Proleukin®), lenvatinib mesylate (Lenvima®), sorafenib tosylate (Nexavar®), nivolumab (Opdivo®), pazopanib hydrochloride, sorafenib tosylate, sunitinib (Sutent®), temsirolimus (Torisel®) and pazopanib hydrochloride (Votrient®). Other therapeutics that may be used are a targeted agent (e.g., a VEGF inhibitor such as a monoclonal antibody to VEGF, e.g., bevacizumab, a VEGF tyrosine kinase inhibitor such as sorafenib, axitinib and pazopanib.

Exemplary therapeutic agents that may be used in combination with the antibodies of the invention for treatment of a chronic myelogenous leukemia (AML) include a chemotherapeutic (e.g., cytarabine, hydroxyurea, clofarabine, melphalan, thiotepa, fludarabine, busulfan, etoposide, cordycepin, pentostatin, capecitabine, azacitidine, cyclophosphamide, cladribine, topotecan), tyrosine kinase inhibitor (e.g., BCR/ABL inhibitor (e.g., imatinib, nilotinib), dual inhibitor (e.g., dasatinib, bosutinib), multikinase inhibitor (e.g., DCC-2036, ponatinib, sorafenib, sunitinib, RGB-286638), interferon alfa, steroids, apoptotic agent (e.g., omacetaxine mepesuccinat), immunotherapy (e.g., allogeneic CD4+ memory Th1-like T cells/microparticle-bound anti-CD3/anti-CD28, autologous cytokine induced killer cells (CIK), AHN-12), CD52 targeting agent (e.g., alemtuzumab), HSP90 inhibitor (e.g., tanespimycin, STA-9090, AUY922, XL888), mTOR inhibitor (e.g., everolimus), SMO antagonist (e.g., BMS 833923), ribonucleotide reductase inhibitor (e.g., 3-AP), JAK-2 inhibitor (e.g., INCB018424), hydroxychloroquine, retinoid (e.g., fenretinide), cyclin-dependent kinase inhibitor (e.g., UCN-01), HDAC inhibitor (e.g., belinostat, vorinostat, JNJ-26481585), PARP inhibitor (e.g., veliparib), MDM2 antagonist (e.g., R05045337), Aurora B kinase inhibitor (e.g., TAK-901), radioimmunotherapy (e.g., actinium-225-labeled anti-CD33 antibody HuM 195), Hedgehog inhibitor (e.g., PF-04449913), STAT3 inhibitor (e.g., OPB-31121), KB004, cancer vaccine (e.g., AG858), bone marrow transplantation, stem cell transplantation, radiation therapy, and combinations thereof.

Exemplary therapeutic agents that may be used in combination with the antibodies of the invention for treatment of a chronic lymphocytic leukemia (CLL) include a chemotherapeutic agent (e.g., fludarabine, cyclophosphamide, doxorubicin, vincristine, chlorambucil, bendamustine, chlorambucil, busulfan, gemcitabine, melphalan, pentostatin, mitoxantrone, 5-azacytidine, pemetrexed disodium), tyrosine kinase inhibitor (e.g., EGFR inhibitor (e.g., erlotinib), BTK inhibitor (e.g., PCI-32765 (ibrutinib), multikinase inhibitor (e.g., MGCD265, RGB-286638), CD-20 targeting agent (e.g., rituximab, ofatumumab, R05072759, LFB-R603), CD52 targeting agent (e.g., alemtuzumab), prednisolone, darbepoetin alfa, lenalidomide, Bel-2 inhibitor (e.g., ABT-263), immunotherapy (e.g., allogeneic CD4⁺ memory Th1-like T cells/microparticle-bound anti-CD3/anti-CD28, autologous cytokine induced killer cells (CIK), HDAC inhibitor (e.g., vorinostat, valproic acid, LBH589, JNJ-26481585, AR-42), XIAP inhibitor (e.g., AEG35156), CD-74 targeting agent (e.g., milatuzumab), mTOR inhibitor (e.g., everolimus), AT-101, immunotoxin (e.g., CAT-8015, anti-Tac(Fv)-PE38 (LMB-2)), CD37 targeting agent (e.g., TRU-016), radioimmunotherapy (e.g., 131-tositumomab), hydroxychloroquine, perifosine, SRC inhibitor (e.g., dasatinib), thalidomide, PI3K delta inhibitor (e.g., CAL-101), retinoid (e.g., fenretinide), MDM2 antagonist (e.g., R05045337), plerixafor, Aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), hypoxia-activated prodrug (e.g., TH-302), paclitaxel or a paclitaxel agent, HSP90 inhibitor, AKT inhibitor (e.g., MK2206), HMG-CoA inhibitor (e.g., simvastatin), GNKG186, radiation therapy, bone marrow transplantation, stem cell transplantation, and a combination thereof.

Exemplary therapeutic agents that may be used in combination with the antibodies of the invention for treatment of an acute lymphocytic leukemia (ALL) include a chemotherapeutic agent (e.g., prednisolone, dexamethasone, vincristine, asparaginase, daunorubicin, cyclophosphamide, cytarabine, etoposide, thioguanine, mercaptopurine, clofarabine, liposomal annamycin, busulfan, etoposide, capecitabine, decitabine, azacitidine, topotecan, temozolomide), tyrosine kinase inhibitor (e.g., BCR/ABL inhibitor (e.g., imatinib, nilotinib), ON 01910.Na, multikinase inhibitor (e.g., sorafenib), CD-20 targeting agent (e.g., rituximab), CD52 targeting agent (e.g., alemtuzumab), HSP90 inhibitor (e.g., STA-9090), mTOR inhibitor (e.g., everolimus, rapamycin), JAK-2 inhibitor (e.g., INCB018424), HER2/neu receptor inhibitor (e.g., trastuzumab), proteasome inhibitor (e.g., bortezomib), methotrexate, asparaginase, CD-22 targeting agent (e.g., epratuzumab, inotuzumab), immunotherapy (e.g., autologous cytokine induced killer cells (CIK), AHN-12), blinatumomab, cyclin-dependent kinase inhibitor (e.g., UCN-01), CD45 targeting agent (e.g., BC8), MDM2 antagonist (e.g., R05045337), immunotoxin (e.g., CAT-8015, DT2219ARL), HDAC inhibitor (e.g., JNJ-26481585), JVRS-100, paclitaxel or a paclitaxel agent, STAT3 inhibitor (e.g., OPB-31121), PARP inhibitor (e.g., veliparib), EZN-2285, radiation therapy, steroid, bone marrow transplantation, stem cell transplantation, or a combination thereof.

Exemplary therapeutic agents that may be used in combination with the antibodies of the invention for treatment of an acute myeloid leukemia (AML) include a chemotherapeutic agent (e.g., cytarabine, daunorubicin, idarubicin, clofarabine, decitabine, vosaroxin, azacitidine, clofarabine, ribavirin, CPX-351, treosulfan, elacytarabine, azacitidine), tyrosine kinase inhibitor (e.g., BCR/ABL inhibitor (e.g., imatinib, nilotinib), ON 01910.Na, multikinase inhibitor (e.g., midostaurin, SU 11248, quizartinib, sorafinib), immunotoxin (e.g., gemtuzumab ozogamicin), DT388IL3 fusion protein, HDAC inhibitor (e.g., vorinostat, LBH589), plerixafor, mTOR inhibitor (e.g., everolimus), SRC inhibitor (e.g., dasatinib), HSP90 inhbitor (e.g., STA-9090), retinoid (e.g., bexarotene, Aurora kinase inhibitor (e.g., BI 811283), JAK-2 inhibitor (e.g., INCB018424), Polo-like kinase inhibitor (e.g., BI 6727), cenersen, CD45 targeting agent (e.g., BC8), cyclin-dependent kinase inhibitor (e.g., UCN-01), MDM2 antagonist (e.g., R05045337), mTOR inhibitor (e.g., everolimus), LY573636-sodium, ZRx-101, MLN4924, lenalidomide, immunotherapy (e.g., AHN-12), histamine dihydrochloride, radiation therapy, bone marrow transplantation, stem cell transplantation, and a combination thereof.

Exemplary therapeutic agents that may be used in combination with the antibodies of the invention for treatment of a multiple myeloma (MM) include a chemotherapeutic agent (e.g., melphalan, amifostine, cyclophosphamide, doxorubicin, clofarabine, bendamustine, fludarabine, adriamycin, SyB L-0501), thalidomide, lenalidomide, dexamethasone, prednisone, pomalidomide, proteasome inhibitor (e.g., bortezomib, carfilzomib, MLN9708), cancer vaccine (e.g., GVAX), CD-40 targeting agent (e.g., SGN-40, CHIR-12.12), perifosine, zoledronic acid, Immunotherapy (e.g., MAGE-A3, NY-ESO-1, HuMax-CD38), HDAC inhibitor (e.g., vorinostat, LBH589, AR-42), aplidin, cycline-dependent kinase inhibitor (e.g., PD-0332991, dinaciclib), arsenic trioxide, CB3304, HSP90 inhibitor (e.g., KW-2478), tyrosine kinase inhibitor (e.g., EGFR inhibitor (e.g., cetuximab), multikinase inhibitor (e.g., AT9283), VEGF inhibitor (e.g., bevacizumab), plerixafor, MEK inhibitor (e.g., AZD6244), IPH2101, atorvastatin, immunotoxin (e.g., BB-10901), NPI-0052, radioimmunotherapeutic (e.g., yttrium Y 90 ibritumomab tiuxetan), STAT3 inhibitor (e.g., OPB-31121), MLN4924, Aurora kinase inhibitor (e.g., ENMD-2076), IMGN901, ACE-041, CK-2 inhibitor (e.g., CX-4945), an anti-CD38 antibody (e.g. DARZALEX® (daratumumab), radiation therapy, bone marrow transplantation, stem cell transplantation, and a combination thereof.

Exemplary therapeutic agents that may be used in combination with the antibodies of the invention for treatment of a prostate cancer are approved drugs for treatment of the prostate cancer, such as abiraterone acetate (Zytiga®), bicalutamide (Casodex®), cabazitaxel (Jevtana®), conjugated estrogens (Premarin®), stradiol (Estrace®), estradiol valerate (Delestrogen®), estrogens, esterified (Menest®), degarelix (Firmagon®), docetaxel (Taxotere®), enzalutamide (Xtandi®), flutamide, goserelin acetate (Zoladex®), Cabazitaxel (Jevtana®), leuprolide acetate (Lupron®), mitoxantrone hydrochloride, nilutamide (Nilandron®) Sipuleucel-T (Provenge®) and radium 223 dichloride (Xofigo®). Other drugs that may be used include a chemotherapeutic agent (e.g., carboplatin, fludarabine), hormonal therapy (e.g., cyproterone acetate, ketoconazole, aminoglutethimide, abarelix, degarelix, leuprolide, triptorelin, buserelin), tyrosine kinase inhibitor (e.g., dual kinase inhibitor (e.g., lapatanib), multikinase inhibitor (e.g., sorafenib, sunitinib), VEGF inhibitor (e.g., bevacizumab), TAK-700, cancer vaccine (e.g., BPX-101, PEP223), lenalidomide, TOK-001, IGF-1 receptor inhibitor (e.g., cixutumumab), TRC105, Aurora A kinase inhibitor (e.g., MLN8237), proteasome inhibitor (e.g., bortezomib), OGX-011, radioimmunotherapy (e.g., HuJ591-GS), HDAC inhibitor (e.g., valproic acid, SB939, LBH589), hydroxychloroquine, mTOR inhibitor (e.g., everolimus), dovitinib lactate, diindolylmethane, efavirenz, OGX-427, genistein, IMC-3G3, bafetinib, CP-675,206, radiation therapy, surgery, or a combination thereof.

Exemplary therapeutic agents that may be used in combination with the antibodies of the invention for treatment of a head and neck squamous cell carcinoma (HNSCC) include methotrexate (Folex®, Mexate®), bleomycin (Blenoxane®), docetaxel (Taxotere®), erbitux (Cetuximab®), hydroxyurea (Hydrea®) or pembrolizumab (Keytruda®), In some embodiments, the antibodies of the invention are administered in combination with a TLR agonist.

In some embodiments, the TLR3 agonist is TLR4 agonist.

In some embodiments, the TLR3 agonist is a TLR7/8 agonist.

Exemplary TLR agonists are Pam3Cys, a TLR-1/2 agonist; CFA, a TLR-2 agonist; MALP2, a TLR-2 agonist; Pam2Cys, a TLR-2 agonist; FSL-1, a TLR-2 agonist; Hib-OMPC, a TLR-2 agonist; polyribosinic:polyribocytidic acid (Poly J:C), a TLR-3 agonist; polyadenosine-polyuridylic acid (poly AU), a TLR-3 agonist; Polyinosinic-Polycytidylic acid stabilized with poly-L-lysine and carboxymethylcellulose (Hiltonol®), a TLR-3 agonist; monophosphoryl lipid A (MPL), a TLR-4 agonist; LPS, a TLR-4 agonist; bacterial flagellin, a TLR-5 agonist; sialyl-Tn (STn), a carbohydrate associated with the MUCI mucin on a number of human cancer cells and a TLR-4 agonist; imiquimod, a TLR-7 agonist; resiquimod, a TLR-7/8 agonist; loxoribine, a TLR-7/8 agonist; and unmethylated CpG dinucleotide (CpG-ODN), a TLR-9 agonist.

Exemplary TLR4 agonists are agonistic antibodies specifically binding TLR4.

In some embodiments described herein, the antibodies of the invention are administered in combination with an antibody that bids CSF-1R

Exemplary antibodies that bind CSF-1R are those described in Int. Patent Publ. No. WO2013132044.

In some embodiments described herein, the antibodies of the invention are administered in combination with LXRP agonist.

In some embodiments described herein, the antibodies of the invention are administered in combination with a DR4 agonist.

In some embodiments described herein, the antibodies of the invention are administered in combination with a DR5 agonist.

Suitable DR4 and DR5 agonists are described for example in Int. Patent Publ. No. WO2014159562.

In some embodiments described herein, the antibodies of the invention are administered in combination with an anti-galectin 1 antibody.

Exemplary anti-galectin 1 antibodies that may be used in combination with the antibodies of the invention are those described in Int. Patent Publ. No. WO2015013389.

In some embodiment described herein, the antibodies of the invention are administered in combination with a BTK inhibitor.

In some embodiments, the BTK inhibitor is IMBRUVICA® (ibrutinib).

In some embodiments described herein, the antibodies of the invention are administered in combination with an anti-HER2 antibody.

In some embodiments described herein, the antibodies of the invention are administered in combination with an anti-CD20 antibody.

In some embodiments, the antibodies of the invention are administered in conjunction with (e.g., before, simultaneously or following) bone marrow transplantation, T cell ablative therapy using chemotherapy agents such as, fludarabine, external-beam radiation therapy (XRT), cyclophosphamide, and/or antibodies such as OKT3 or CAMPATH. In some embodiments, the antibodies of the invention may be administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive the antibodies of the invention.

In some embodiments described herein, the antibodies of the invention are administered before or following surgery.

In some embodiments described herein, the antibodies of the invention are administered in combination with radiation therapy.

Radiation therapy may be administered using various methods, including external-beam therapy, internal radiation therapy, implant radiation, stereotactic radiosurgery, systemic radiation therapy, radiotherapy and permanent or temporary interstitial brachytherapy. External-beam therapy involves three dimensional, conformal radiation therapy where the field of radiation is designed, local radiation (e.g., radiation directed to a preselected target or organ), or focused radiation. Focused radiation may be selected from stereotactic radiosurgery, fractionated stereotactic radiosurgery or intensity-modulated radiation therapy. Focused radiation may have particle beam (proton), cobalt-60 (photon) linear accelerator (x-ray) as a radiation source (see e.g. WO 2012/177624).

“Brachytherapy,” refers to radiation therapy delivered by a spatially confined radioactive material inserted into the body at or near a tumor or other proliferative tissue disease site, and includes exposure to radioactive isotopes (e.g., At-211, I-131, I-125, Y-90, Re-186, Re-188, Sm-153, Bi-212, P-32, and radioactive isotopes of Lu). Suitable radiation sources for use as a cell conditioner include both solids and liquids. The radiation source can be a radionuclide, such as I-125, I-131, Yb-169, Ir-192 as a solid source, I-125 as a solid source, or other radionuclides that emit photons, beta particles, gamma radiation, or other therapeutic rays. The radioactive material may also be a fluid made from any solution of radionuclide(s), e.g., a solution of I-125 or I-131, or a radioactive fluid can be produced using a slurry of a suitable fluid containing small particles of solid radionuclides, such as Au-198, Y-90. The radionuclide(s) may be embodied in a gel or radioactive micro spheres.

In some embodiments, the antibodies of the invention are administered in combination with decarbazine for the treatment of melanoma. Without being bound by any particular theory, the combined use of PD-1 and/or TIM-3 blockade and chemotherapy is believed to be facilitated by cell death that is a consequence of the cytotoxic action of most chemotherapeutic compounds, which can result in increased levels of tumor antigen in the antigen presentation pathway. Other combination therapies that may result in synergy with PD-1 and/or TIM-3 blockade through cell death are radiation, surgery, and hormone deprivation. Each of these protocols creates a source of tumor antigen in the host. Angiogenesis inhibitors may also be combined with PD-1 and/or TIM-3 blockade. Inhibition of angiogenesis leads to tumor cell death which may feed tumor antigen into host antigen presentation pathways.

The monospecific PD-1 and/or TIM-3 antibodies of the invention may also be used in combination with bispecific antibodies. Bispecific antibodies may be used to target two separate antigens. For example anti-Fc receptor/anti-tumor antigen (e.g., Her-2/neu) bispecific antibodies have been used to target macrophages to sites of tumor. Bispecific targeting may more effectively activate tumor specific responses. The T cell arm of these responses would be augmented by the use of PD-1 and/or TIM-3 blockade. Alternatively, antigen may be delivered directly to DCs by the use of bispecific antibodies which bind to tumor antigen and a dendritic cell specific cell surface marker.

The antibodies of the invention may be used in unconjugated forms or conjugated to a second agent, e.g., a cytotoxic drug, radioisotope, or a protein, e.g., a protein toxin or a viral protein. The antibody molecules may be used to deliver a variety of therapeutic agents, e.g., a cytotoxic moiety, e.g., a therapeutic drug, a radioisotope, molecules of plant, fungal, or bacterial origin, or biological proteins (e.g., protein toxins) or particles (e.g., a recombinant viral particles, e.g.; via a viral coat protein), or mixtures thereof.

### Infectious Diseases

The invention also provides a method of treating a subject that has been exposed to particular toxins or pathogen with the antibodies of the invention for a time sufficient to treat the subject.

The invention also provides a method of treating a subject having an infectious disease, comprising administering a therapeutically efficient amount of the antibody of the invention to the subject in need thereof for a time sufficient to treat the infectious disease.

The invention also provides a method of treating a subject having a viral infection, comprising administering a therapeutically efficient amount of the antibody of the invention to the subject in need thereof for a time sufficient to treat the viral infection.

The invention also provides a method of treating a subject having a bacterial infection, comprising administering a therapeutically efficient amount of the antibody of the invention to the subject in need thereof for a time sufficient to treat the bacterial infection.

The invention also provides a method of treating a subject having a fungal infection, comprising administering a therapeutically efficient amount of the antibody of the invention to the subject in need thereof for a time sufficient to treat the fungal infection.

In the treatment of infection (e.g., acute and/or chronic), administration of the antibodies of the invention may be combined with conventional treatments in addition to or in lieu of stimulating natural host immune defenses to infection. Natural host immune defenses to infection include inflammation, fever, antibody-mediated host defense, T-lymphocyte-mediated host defenses, including lymphokine secretion and cytotoxic T-cells (especially during viral infection), complement mediated lysis and opsonization (facilitated phagocytosis), and phagocytosis. The ability of the antibodies of the invention to reactivate dysfunctional T-cells would be useful to treat chronic infections, in particular those in which cell-mediated immunity is important for complete recovery.

Similar to its application to tumors as discussed above, antibodies of the invention may be used alone, or as an adjuvant, in combination with vaccines, to stimulate the immune response to pathogens, toxins, and self-antigens. Examples of pathogens for which this therapeutic approach may be useful include pathogens for which there is currently no effective vaccine, or pathogens for which conventional vaccines are less than completely effective. These include HIV, Hepatitis (A, B, & C), Influenza, Herpes, Giardia, Malaria, Leishmania, Staphylococcus aureus and Pseudomonas Aeruginosa. PD-1 and/or TIM-3 blockade may be useful against established infections by agents such as HIV that present altered antigens over the course of the infections. These novel epitopes are recognized as foreign at the time of administration of the antibodies of the invention, thus provoking a strong T cell response that is not dampened by negative signals through PD-1 or TIM-3.

### Viruses

For infections resulting from viral causes, the antibodies of the invention may be combined with standard therapies for treating viral infections. Such standard therapies vary depending upon type of virus, although in almost all cases, administration of human serum containing antibodies (e.g., IgA, IgG) specific to the virus can be effective.

Exemplary pathogenic viruses causing infections that may be treatable by the antibodies of the invention include HIV, hepatitis (A, B, or C), herpes virus (e.g., VZV, HSV-1, HAV-6, HSV-JJ, and CMV, Epstein Barr virus), adenovirus, influenza virus, flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

In some embodiments, the virus infection is an influenza virus infection. Influenza infection can result in fever, cough, myalgia, headache and malaise, which often occur in seasonal epidemics. Influenza is also associated with a number of postinfectious disorders, such as encephalitis, myopericarditis, Goodpasture's syndrome, and Reye's syndrome. Influenza infection also suppresses normal pulmonary antibacterial defenses, such that patients recovering from influenza have an increased risk of developing bacterial pneumonia. Influenza viral surface proteins show marked antigenic variation, resulting from mutation and recombination. Thus, cytolytic T lymphocytes are the host's primary vehicle for the elimination of virus after infection. Influenza is classified into three primary types: A, B and C. Influenza A is unique in that it infects both humans and many other animals (e.g., pigs, horses, birds and seals) and is the principal cause of pandemic influenza. A cell can be infected by two different influenza A strains, the segmented RNA genomes of two parental virus types mix during replication to create a hybrid replicant, resulting in new epidemic strains. Influenza B does not replicate in animals and thus has less genetic variation and influenza C has only a single serotype.

Most conventional therapies are palliatives of the symptoms resulting from infection, while the host's immune response actually clears the disease. However, certain strains (e.g., influenza A) can cause more serious illness and death. Influenza A may be treated both clinically and prophylactically by the administration of the cyclic amines inhibitors amantadine and rimantadine, which inhibit viral replication. However, the clinical utility of these drugs is limited due to the relatively high incidence of adverse reactions, their narrow anti-viral spectrum (influenza A only), and the propensity of the virus to become resistant. The administration of serum IgG antibody to the major influenza surface proteins, hemagglutinin and neuraminidase can prevent pulmonary infection, whereas mucosal IgA is required to prevent infection of the upper respiratory tract and trachea. The most effective current treatment for influenza is vaccination with the administration of virus inactivated with formalin or P-propiolactone.

In some embodiments, the infection is a hepatitis infection, e.g., a Hepatitis B or C infection.

Hepatitis B virus (HB-V) is the most infectious known blood borne pathogen. It is a major cause of acute and chronic hepatitis and hepatic carcinoma, as well as life-long, chronic infection. Following infection, the virus replicates in hepatocytes, which also then shed the surface antigen HBsAg. The detection of excessive levels of HBsAg in serum is used as a standard method for diagnosing a hepatitis B infection. An acute infection may resolve or it can develop into a chronic persistent infection. Current treatments for chronic HBV include a-interferon, which increases the expression of class I human leukocyte antigen (HLA) on the surface of hepatocytes, thereby facilitating their recognition by cytotoxic T lymphocytes. Additionally, the nucleoside analogs ganciclovir, famciclovir and lamivudine have also shown some efficacy in the treatment of HBV infection in clinical trials. Additional treatments for HBV include pegylated a-interferon, adenfovir, entecavir and telbivudine. While passive immunity can be conferred through parental administration of anti-HBsAg serum antibodies, vaccination with inactivated or recombinant HBsAg also confers resistance to infection. The antibodies of the invention may be combined with conventional treatments for hepatitis B infections for therapeutic advantage.

Hepatitis C virus (HC-V) infection may lead to a chronic form of hepatitis, resulting in cirrosis. While symptoms are similar to infections resulting from Hepatitis B, in distinct contrast to HB-V, infected hosts can be asymptomatic for 10-20 years. The antibodies of the invention can be administered as a monotherapy, or combined with the standard of care for hepatitis C infection. For example, the antibodies of the invention can be administered with one or more of Sovaldi (sofosbuvir) Olysio (simeprevir), plus ribavirin or pegylated interferon. Although regimens that include Incivek (telaprevir) or Victrelis (boceprevir) plus ribavirin and pegylated interferon are also approved, they are associated with increased side effects and longer duration of treatment.

Conventional treatment for HC-V infection includes the administration of a combination of α-interferon and ribavirin. A promising potential therapy for HC-V infection is the protease inhibitor telaprevir (VX-960). Additional treatments include bavituximab (an antibody that binds anionic phospholipid phosphatidylserine in a B2-glycoprotein I dependent manner, Peregrine Pharmaceuticals), anti-HPV viral coat protein E2 antibod(y)(ies) (e.g., ATL 6865-Ab68+Ab65, XTL Pharmaceuticals) and Civacir® (polyclonal anti-HCV human immune globulin). The antibodies of the invention may be combined with one or more of these treatments for hepatitis C infections for therapeutic advantage. Protease, polymerase and NS5A inhibitors which may be used in combination with the antibodies of the invention to specifically treat Hepatitis C infection include those described in US 2013/0045202.

In another embodiment, the infection is a measles virus. After an incubation of 9-11 days, hosts infected with the measles virus develop fever, cough, coryza and conjunctivitis. Within 1-2 days, an erythematous, maculopapular rash develop, which quickly spreads over the entire body. Because infection also suppresses cellular immunity, the host is at greater risk for developing bacterial superinfections, including otitis media, pneumonia and postinfectious encephalomyelitis. Acute infection is associated with significant morbidity and mortality, especially in malnourished adolescents.

Treatment for measles includes the passive administration of pooled human IgG, which can prevent infection in non-immune subjects, even if given up to one week after exposure. However, prior immunization with live, attenuated virus is the most effective treatment and prevents disease in more than 95% of those immunized. As there is one serotype of this virus, a single immunization or infection typically results in protection for life from subsequent infection.

In a small proportion of infected hosts, measles can develop into SSPE, which is a chronic progressive neurologic disorder resulting from a persistent infection of the central nervous system. S SPE is caused by clonal variants of measles virus with defects that interfere with virion assembly and budding. For these patients, reactivation of T-cells with the antibodies of the invention so as to facilitate viral clearance would be desirable.

In another embodiment, the infection is HIV. HIV attacks CD4⁺ cells, including T-lymphocytes, monocyte-macrophages, follicular dendritic cells and Langerhan's cells, and CD4⁺ helper/inducer cells are depleted. As a result, the host acquires a severe defect in cell-mediated immunity. Infection with HIV results in AIDS in at least 50% of individuals, and is transmitted via sexual contact, administration of infected blood or blood products, artificial insemination with infected semen, exposure to blood-containing needles or syringes and transmission from an infected mother to infant during childbirth.

A host infected with HIV may be asymptomatic, or may develop an acute illness that resembling mononucleosis—fever, headache, sore throat, malaise and rash. Symptoms can progress to progressive immune dysfunction, including persistent fever, night sweats, weight loss, unexplained diarrhea, eczema, psoriasis, seborrheic dermatitis, herpes zoster, oral candidiasis and oral hairy leukoplakia. Opportunistic infections by a host of parasites are common in patients whose infections develop into AIDS.

Treatments for HIV include antiviral therapies including nucleoside analogs, zidovudine (AST) either alone or in combination with didanosine or zalcitabine, dideoxyinosine, dideoxycytidine, lamidvudine, stavudine; reverse transcriptive inhibitors such as delavirdine, nevirapine, loviride, and proteinase inhibitors such as saquinavir, ritonavir, indinavir and nelfinavir. Treatments for HIV include EDURANT® (rilpivirine). The antibodies of the invention may be combined with conventional treatments for HIV infections for therapeutic advantage.

In another embodiment, the infection is a Cytomegalovirus (CMV) infection. CMV infection is often associated with persistent, latent and recurrent infection. CMV infects and remains latent in monocytes and granulocyte-monocyte progenitor cells. The clinical symptoms of CMV include mononucleosis-like symptoms (i.e., fever, swollen glands, malaise), and a tendency to develop allergic skin rashes to antibiotics. The virus is spread by direct contact. The virus is shed in the urine, saliva, semen and to a lesser extent in other body fluids. Transmission can also occur from an infected mother to her fetus or newborn and by blood transfusion and organ transplants. CMV infection results in general impairment of cellular immunity, characterized by impaired blastogenic responses to nonspecific mitogens and specific CMV antigens and diminished cytotoxic ability.

Treatments of CMV infection include the anti-virals ganciclovir, foscarnet and cidovir, but these drugs are typically only prescribed in immunocompromised patients. The antibodies of the invention described herein may be combined with conventional treatments for cytomegalovirus infections for therapeutic advantage.

In another embodiment, the infection is Epstein-Barr virus (EBV) infection. EBV can establish persistent and latent infections and primarily attacks B cells. Infection with EBV results in the clinical condition of infectious mononucleosis, which includes fever, sore throat, often with exudate, generalized lymphadenopathy and splenomegaly. Hepatitis is also present, which can develop into jaundice.

While typical treatments for EBV infections are palliative of symptoms, EBV is associated with the development of certain cancers such as Burkitt's lymphoma and nasopharyngeal cancer. Thus, clearance of viral infection before the complications develop would be of great benefit. The antibodies of the invention may be combined with conventional treatments for Epstein-Barr virus infections for therapeutic advantage.

In another embodiment, the infection is Herpes simplex virus (HSV) infection. HSV is transmitted by direct contact with an infected host. A direct infection may be asymptomatic, but typically result in blisters containing infectious particles. The disease manifests as cycles of active periods of disease, in which lesions appear and disappear as the virus latently infects the nerve ganglion for subsequent outbreaks. Lesions may be on the face, genitals, eyes and/or hands. In some case, an infection can also cause encephalitis.

Treatments for herpes infections are directed primarily to resolving the symptomatic outbreaks, and include systemic antiviral medicines such as: acyclovir (e.g., Zovirax®), valaciclovir, famciclovir, penciclovir, and topical medications such as docosanol (Abreva®), tromantadine and zilactin. The clearance of latent infections of herpes would be of great clinical benefit. The antibodies of the invention may be combined with conventional treatments for herpes virus infections for therapeutic advantage.

In another embodiment, the infection is Human T-lymphotrophic virus (HTLV-1, HTLV-2). HTLV is transmitted via sexual contact, breast feeding or exposure to contaminated blood. The virus activates Th1 cells, resulting in their overproliferation and overproduction of Th1 related cytokines (e.g., IFN-γ and TNF-α). This in turn results in a suppression of Th2 lymphocytes and reduction of Th2 cytokine production (e.g., IL-4, IL-5, IL-10 and IL-13), causing a reduction in the ability of an infected host to mount an adequate immune response to invading organisms requiring a Th2-dependent response for clearance (e.g., parasitic infections, production of mucosal and huImoral antibodies).

HTLV infections lead to opportunistic infections resulting in bronchiectasis, dermatitis and superinfections with Staphylococcus spp. and Strongyloides spp. resulting in death from polymicrobial sepsis. HTLV infection can also lead directly to adult T-cell leukemia/lymphoma and progressive demyelinating upper motor neuron disease known as HAM/TSP. The clearance of HTLV latent infections would be of great clinical benefit. The antibodies of the invention may be combined with conventional treatments for HTLV infections for therapeutic advantage.

In another embodiment, the infection is Human papilloma virus (HPV). HPV primarily affects keratinocytes and occurs in two forms: cutaneous and genital. Transmission is believed to occur through direct contact and/or sexual activity. Both cutaneous and genital HPV infection can result in warts and latent infections and sometimes recurring infections, which are controlled by host immunity which controls the symptoms and blocks the appearance of warts, but leaves the host capable of transmitting the infection to others.

Infection with HPV can also lead to certain cancers, such as cervical, anal, vulvar, penile and oropharynial cancer. There are no known cures for HPV infection, but current treatment is topical application of Imiquimod, which stimulates the immune system to attack the affected area. The clearance of HPV latent infections would be of great clinical benefit. The antibodies of the invention may be combined with conventional treatments for HPV infections for therapeutic advantage.

### Bacterial Infections

Some examples of pathogenic bacteria causing infections that may be treated with the antibodies of the invention include syphilis, chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lymes disease bacteria. The antibodies of the invention can be used in combination with existing treatment modalities for the aforesaid infections. For example, treatments for syphilis include penicillin (e.g., penicillin G.), tetracycline, doxycycline, ceftriaxone and azithromycin.

Lyme disease, caused by Borrelia burgdorferi is transmitted into humans through tick bites. The disease manifests initially as a localized rash, followed by flu-like symptoms including malaise, fever, headache, stiff neck and arthralgias. Later manifestations can include migratory and polyarticular arthritis, neurologic and cardiac involvement with cranial nerve palsies and radiculopathy, myocarditis and arrhythmias. Some cases of Lyme disease become persistent, resulting in irreversible damage analogous to tertiary syphilis. Current therapy for Lyme disease includes primarily the administration of antibiotics. Antibiotic-resistant strains may be treated with hydroxychloroquine or methotrexate. Antibiotic refractory patients with neuropathic pain can be treated with gabapentin. Minocycline may be helpful in late/chronic Lyme disease with neurological or other inflammatory manifestations.

Other forms of borreliois, such as those resulting from B. recurentis, B. hermsii, B. turicatae, B. parikeri, B. hispanica, B. duttonii and B. persica, as well leptospirosis (E.g., L. interrogans), typically resolve spontaneously unless blood titers reach concentrations to cause intrahepatic obstruction.

### Fungi and Parasites

Some examples of pathogenic fungi causing infections that may be treated with the antibodies of the invention include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Some examples of pathogenic parasites causing infections treatable with the antibodies of the invention described herein include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

### Diagnostic Uses and Kits

### Kits

The invention also provides a kit comprising the antagonistic antibody specifically binding PD-1 of the invention.

The invention also provides a kit comprising the antagonistic antibody specifically binding TIM-3 of the invention.

The invention also provides a kit comprising the antagonistic bispecific PD-1/TIM-3 antibody comprising a first domain specifically binding PD-1 and a second domain specifically binding TIM-3 of the invention.

The kit may be used for therapeutic uses and as diagnostic kits.

The kit may be used to detect the presence of PD-1, TIM-3, or PD-1 and TIM-3 in a biological sample.

In some embodiments, the kit comprises the antibody of the invention described herein and reagents for detecting the antibody. The kit can include one or more other elements including: instructions for use; other reagents, e.g., a label, a therapeutic agent, or an agent useful for chelating, or otherwise coupling, an antibody to a label or therapeutic agent, or a radioprotective composition; devices or other materials for preparing the antibody for administration; pharmaceutically acceptable carriers; and devices or other materials for administration to a subject.

In some embodiments, the kit comprises the antibody of the invention in a container and instructions for use of the kit.

In some embodiments, the antibody in the kit is labeled.

In some embodiments, the kit comprises the antagonistic antibody specifically binding PD-1, comprising
the VH of SEQ ID NO: 41 and the VL of SEQ ID NO: 49;
the VH of SEQ ID NO: 41 and the VL of SEQ ID NO: 50;
the VH of SEQ ID NO: 42 and the VL of SEQ ID NO: 51;
the VH of SEQ ID NO: 42 and the VL of SEQ ID NO: 52;
the VH of SEQ ID NO: 42 and the VL of SEQ ID NO: 53;
the VH of SEQ ID NO: 43 and the VL of SEQ ID NO: 49;
the VH of SEQ ID NO: 43 and the VL of SEQ ID NO: 54;
the VH of SEQ ID NO: 43 and the VL of SEQ ID NO: 50;
the VH of SEQ ID NO: 43 and the VL of SEQ ID NO: 55;
the VH of SEQ ID NO: 43 and the VL of SEQ ID NO: 56;
the VH of SEQ ID NO: 43 and the VL of SEQ ID NO: 57;
the VH of SEQ ID NO: 44 and the VL of SEQ ID NO: 49;
the VH of SEQ ID NO: 45 and the VL of SEQ ID NO: 49;
the VH of SEQ ID NO: 46 and the VL of SEQ ID NO: 49;
the VH of SEQ ID NO: 47 and the VL of SEQ ID NO: 49;
the VH of SEQ ID NO: 48 and the VL of SEQ ID NO: 53;
the VH of SEQ ID NO: 48 and the VL of SEQ ID NO: 52;
the VH of SEQ ID NO: 48 and the VL of SEQ ID NO: 56;
the VH of SEQ ID NO: 47 and the VL of SEQ ID NO: 58;
the VH of SEQ ID NO: 47 and the VL of SEQ ID NO: 59;
the VH of SEQ ID NO: 45 and the VL of SEQ ID NO: 60;
the VH of SEQ ID NO: 45 and the VL of SEQ ID NO: 61;
the VH of SEQ ID NO: 45 and the VL of SEQ ID NO: 62;
the VH of SEQ ID NO: 63 and the VL of SEQ ID NO: 65; or
the VH of SEQ ID NO: 64 and the VL of SEQ ID NO: 65.

In some embodiments, the kit comprises the antagonistic antibody specifically binding PD-1 comprising the VH of SEQ ID NO: 48 and the VL of SEQ ID NO: 56.

In some embodiments, the kit comprises the antagonistic antibody specifically binding PD-1 comprising the VH of SEQ ID NO: 64 and the VL of SEQ ID NO: 65.

In some embodiments, the kit comprises the antagonistic antibody specifically binding TIM-3, comprising
the VH of SEQ ID NO: 145 and the VL of SEQ ID NO: 155;
the VH of SEQ ID NO: 146 and the VL of SEQ ID NO: 156;
the VH of SEQ ID NO: 148 and the VL of SEQ ID NO: 157;
the VH of SEQ ID NO: 147 and the VL of SEQ ID NO: 155;
the VH of SEQ ID NO: 149 and the VL of SEQ ID NO: 158;
the VH of SEQ ID NO: 150 and the VL of SEQ ID NO: 159;
the VH of SEQ ID NO: 151 and the VL of SEQ ID NO: 160;
the VH of SEQ ID NO: 152 and the VL of SEQ ID NO: 161;
the VH of SEQ ID NO: 153 and the VL of SEQ ID NO: 162;
the VH of SEQ ID NO: 154 and the VL of SEQ ID NO: 163; or
the VH of SEQ ID NO: 172 and the VL of SEQ ID NO: 173.

In some embodiments, the kit comprises the antagonistic antibody specifically binding TIM-3 comprising the VH of SEQ ID NO: 146 and the VL of SEQ ID NO: 156.

In some embodiments, the kit comprises the antagonistic antibody specifically binding TIM-3 comprising the VH of SEQ ID NO: 172 and the VL of SEQ ID NO: 173.

In some embodiments, the kit comprises the antagonistic bispecific PD-1/TIM-3 antibody comprising the HC1, the LC1, the HC2 and the LC2 of
SEQ ID NOs: 186, 188, 190 and 193, respectively;
SEQ ID NOs: 186, 188, 191 and 194, respectively;
SEQ ID NOs: 187, 189, 190 and 193, respectively;
SEQ ID NOs: 187, 189, 191, 194, respectively;
SEQ ID NOs: 186, 188, 192 and 195, respectively;
SEQ ID NOs: 186, 188, 248 and 194, respectively;
SEQ ID NOs: 241, 188, 244, 195, respectively;
SEQ ID NOs: 241, 188, 245, 194, respectively;
SEQ ID NOs: 242, 189, 246, 194, respectively;
SEQ ID NOs: 243, 188, 246, 194, respectively; or
SEQ ID NOs: 243, 188, 247, 195, respectively.

### Methods of Detecting PD-1, TIM-3 or PD-1 and TIM-3

The invention also provides a method of detecting PD-1 in a sample, comprising obtaining the sample, contacting the sample with the antagonistic antibody specifically binding PD-1 of the invention, and detecting the antibody bound to PD-1 in the sample.

The invention also provides a method of detecting TIM-3 in a sample, comprising obtaining the sample, contacting the sample with the antagonistic antibody specifically binding TIM-3 of the invention, and detecting the antibody bound to TIM-3 in the sample.

The invention also provides a method of detecting PD-1 and TIM-3 in a sample, comprising obtaining the sample, contacting the sample with the antagonistic bispecific PD-1/TIM-3 antibody comprising a first domain specifically binding PD-1 and a second domain specifically binding TIM-3 of the invention, and detecting the antibody bound to PD-1 and TIM-3 in the sample.

In some embodiments, the sample may be derived from urine, blood, serum, plasma, saliva, ascites, circulating cells, circulating tumor cells, cells that are not tissue associated (i.e., free cells), tissues (e.g., surgically resected tumor tissue, biopsies, including fine needle aspiration), histological preparations, and the like.

The antibodies of the invention bound to PD-1, TIM-3 or PD-1 and TIM-3 may be detected using known methods. Exemplary methods include direct labeling of the antibodies using fluorescent or chemiluminescent labels, or radiolabels, or attaching to the antibodies of the invention a moiety which is readily detectable, such as biotin, enzymes or epitope tags. Exemplary labels and moieties are ruthenium, ¹¹In-DOTA, ¹¹¹In-diethylenetriaminepentaacetic acid (DTPA), horseradish peroxidase, alkaline phosphatase and beta-galactosidase, poly-histidine (HIS tag), acridine dyes, cyanine dyes, fluorone dyes, oxazin dyes, phenanthridine dyes, rhodamine dyes and Alexafluor® dyes.

The antibodies of the invention may be used in a variety of assays to detect PD-1, TIM-3 or PD-1 and TIM-3 in the sample. Exemplary assays are western blot analysis, radioimmunoassay, surface plasmon resonance, immunoprecipitation, equilibrium dialysis, immunodiffusion, electrochemiluminescence (ECL) immunoassay, immunohistochemistry, fluorescence-activated cell sorting (FACS) or ELISA assay.

The present invention will now be described with reference to the following specific, non-limiting examples.

### Example 1. General Methods

Purified human mixed lymphocyte reaction (MLR)

A purified human mixed lymphocyte reaction (MLR assay) was used to measure changes in cytokine production induced by addition of test antibodies to co-cultures of CD4⁺ T cells and dendritic cells.

Peripheral blood mononuclear cells (PBMCs) were isolated from a leukopak (Biological Specialty Corporation) using a Ficoll gradient. CD4 T cells were then freshly isolated by negative selection from PBMCs using the Miltenyi AutoMACS and CD4⁺ T cell isolation beads per manufacturer's instructions or were commercially purchased as frozen CD4⁺ T cells (Hemacare Corporation). One dendritic cell donor (Hemacare Corporation) was used. Post-isolation or thaw, CD4⁺ T cells and dendritic cells were washed and resuspended in assay media (RMPI1640 media supplemented with 10% fetal bovine serum, 1% penicillin/streptomycin, 1× non-essential amino acids, and 1× sodium pyruvate-Invitrogen). The purified human CD4⁺ T cells were diluted to 1×10⁶ cells/mL and seeded at 100,000 cells/100 μL/well. Dendritic cells were diluted to 0.1×10⁶cells/mL and seeded at 5,000 cells/50 μL/well in U-bottom plates. Test antibodies or control antibodies were prepared at a 4× concentration in assay media yielding 1× when 50 μL of antibody was added to 150 μL of cells.

10-point serial dilutions of test or control antibodies were added to the wells at a final concentration of: 30, 10, 3.33, 1.11, 0.37, 0.12, 0.04, 0.01, 0.0046 and 00015 nM. CD4⁺ T cells plus dendritic cells and dendritic cells alone were included as controls to measure basal cytokine secretion. Cells were maintained at 37° C., 5% C02 for 5 days. On Day 5, 100 μL of tissue culture supernatant was removed from culture plates and transferred to V-bottom plates. Supernatant was frozen at least overnight at −80° C. Cumulative cytokine production was measured in tissue culture supernatant using Meso Scale Discovery (MSD) Th1/Th2 human cytokine 10-plex plates following manufacturer's protocol. Briefly, MSD plates were blocked with 1% blocker B overnight at 4° C. The following day, blocker was removed and plates were washed using the Biotek 406 plate washer. An 8-point standard curve were prepared and added in duplicate to the plates. Thawed tissue culture supernatant was added at 25 μL/well, plates were sealed and shaken vigorously for 1.5 hours. Without removing standards or supernatant, 25 μL of detection antibody was added to each well. Plates were sealed, and shaken vigorously for 1.5 hours. Plates were washed, read buffer was added and plates were read using Meso Scale Discovery's plate reader.

Cytokine concentrations were calculated by MSD software. The concentration of cytokine in unknown samples is calculated by comparing the unknown's output signal to the output signal and known cytokine concentrations in the standard curve. Calculated concentrations were uploaded in Spotfire TIBCO software for visualization. After a visual inspection of the data, MAD-median outlier procedure with a threshold of 3.5 was used to identify and exclude outliers on log-transformed data. Robust analysis of the half-maximal effective concentration (Robust EC50) was carried out on each cytokine for each antibody.

### CMV Assay

A cytomegalovirus antigen recall assay (CMV assay) was used to measure changes in cytokine production induced by addition of test antibodies to cultures of peripheral blood mononuclear cells (PBMCs) with CMV whole antigen (for PD-1 antibodies) or with a pool of 138 15-mer peptides that overlap through the 65 kd phosphoprotein (pp65) (for TIM-3 mAbs and PD1/TIM-3 bispecific mAbs).

Post-thaw, PBMCs (Astarte Biologics and Hemcare Corporation) were washed and resuspended in assay media (RMPI1640 media supplemented with 10% fetal bovine serum, 1% penicillin/streptomycin, 1× non-essential amino acids, and 1× sodium pyruvate-Invitrogen). The PBMCs were diluted tol.5×106 cells/mL and seeded at 150,000 cells/100 μL/well. CMV antigen (Astarte Biologics) was prepared at a 4× concentration of 0.4 μg/mL in assay media yielding 0.1 μg/mL when 50 μL of antigen was added to 100 μL of cells and 50 μL of antibody. Antibodies were prepared at a 4× concentration in assay media yielding 1× when 50 μL of antibody was added to cells and peptide.

Serial dilutions of test antibodies were added to the wells at a final concentration between 150-0.001 nM. Cells plus CMV antigen or pp65 pool, cells alone, and isotype control prepared at a final concentration of 50 or 30 nM were included as controls to measure basal cytokine secretion. Cells were maintained at 37° C., 5% C02 for 6 days. For MSD analysis, on Day 6, 100 μL of tissue culture supernatant was removed from culture plates and transferred to V-bottom plates. Supernatant was frozen at least overnight at −80° C. Cumulative cytokine production was measured in tissue culture supernatant using Meso Scale Discovery (MSD) Th1/Th2 human cytokine 10-plex plates following manufacturer's protocol. Briefly, MSD plates were blocked with 1% blocker B overnight at 4° C. The following day, blocker was removed and plates were washed using the Biotek 406 plate washer. An 8-point standard curve was prepared and added in duplicate to the plates. Thawed tissue culture supernatant was added at 25 μL/well, plates were sealed and shaken vigorously for 1.5 hours. Without removing standards or supernatant, 25 μL of detection antibody was added to each well. Plates were sealed, and shaken vigorously for 1.5 hours. Plates were washed, read buffer was added and plates were read using Meso Scale Discovery's plate reader.

Cytokine concentrations were calculated by MSD software. The concentration of cytokine in unknown samples is calculated by comparing the unknown's output signal to the output signal and known cytokine concentrations in the standard curve. Calculated concentrations were uploaded in Spotfire TIBCO software for visualization. After a visual inspection of the data, MAD-median outlier procedure with a threshold of 3.5 was used to identify and exclude outliers on log-transformed data. Robust analysis of the half-maximal effective concentration (Robust EC50) was carried out on each cytokine for each antibody.

For TIM-3 antibodies and PD1/TIM-3 bispecific antibodies, at day 6, after supernatant was collected for MSD analysis, cells were washed once with PBS and subsequently stained for Live/Dead discrimination and the following cell surface markers: CD3, CD4, CD8, CD137, PD-1 and TIM-3. Flow cytometry was performed on a LSR Fortessa (BD). Data was analyzed using the Flow Jo software. CD137+ cells were identified based on Fluorescence Minus One (FMO) method on viable CMV-treated CD8+ and CD4+ cells.

For the sequential treatment experiments, CMV recall assays were carried out as above with pp65 peptide pool stimulation for six days. On day six, supernatant was removed and cells were restimulated with pp65 pool in the presence of anti-TIM-3 antibodies. Twenty-four hours later, supernatant was removed and IFN-γ levels were measured by MSD, as described above.

### PD-1 Ligand Inhibition Assay

The ligand inhibition assay design was MSD (Mescoscale Discovery) based. A MSD plate was directly coated with ligand (cynoPDLI-ECD, huPDLI-ECD or huPDL2-ECD) and incubated overnight at 4° C. The following day, the coating solution was removed and the plate was blocked. A fixed concentration of biotinylated PD-1 (huPD1-ECD) was pre incubated with antibodies or with an isotype control antibody as a negative control. Depending on the panel of antibodies to be tested, the antibodies were tested as titrations or at a fixed concentration. The MSD plate was washed and the biotinylated PD-1/antibody mixture was added to the ligand coated MSD plate. The plate was washed and biotinylated PD-1 bound to ligand was detected by ruthenylated streptavidin. Inhibition of PD-1 binding by an antibody resulted in decreased signal in the MSD assay. Maximal biotinylated PD-1 binding in the absence of inhibitor was determined and sometimes used to normalize the data to a percentage of maximal biotinylated PD-1 signal. The mAbs that were positive for inhibition of ligand binding at one concentration were also tested in dose responses for inhibition of various PD-1 ligands.

### Jurkat Cell Binding

Jurkat cells were stimulated overnight with 20 ng/ml of PHA, harvested, washed, and checked for viability. The cells were then incubated at 6-10° C. for 45-60 minutes with various concentrations of test antibodies, washed and incubated at 6-10° C. for 45-60 minutes with FITC-labeled goat anti-human IgG. The cells were washed and fixed with BD Cytofix, refrigerated overnight and analyzed on a MACSQuant flow cytometer. The percentage of PD-1 positive cells at each antibody concentration was plotted vs log of the antibody concentration and EC₅₀ values were generated in Prism.

### Affinity Measurements

PD-1 mAbs

Anti-PD-1 mAbs were tested for binding affinity to huPD1-ECD and cynoPD-1-ECD. Affinity measurements using Surface Plasmon Resonance (SPR) were performed using a ProteOn XPR36 system. A biosensor surface was prepared by coupling a mixture of anti-IgG Fc modified alginate polymer layer surface of a GLC chip using the manufacturer instructions for amine-coupling chemistry. Test mAbs were captured and their interactions with analytes (huPD1-ECD or cynoPD1-ECD) were monitored in PBS-based buffer at 25° C. The collected data were processed and fitted to a Langmuir 1:1 binding model. The result for each mAb was reported in the format of kₒₙ (On-rate), k_{off} (Off-rate) and K_{D} (equilibrium dissociation constant).

### TIM-3 Ligand Inhibition Assay

TIM-3/galectin-9 competition ELISAs were done by binding 1 μg/ml recombinant human Fc-TIM-3 chimera (R&D Systems-cat #: 2365-TM-05) in PBS per well of a 96-well White Maxisorp plate (Nunc). The plates were washed and blocked with StartingBlock T20 (Pierce) and inhibitor at a 10 μg/ml concentration was added to the wells. Without washing, 7.5 μg/ml galectin-9 at was added to the wells and incubated for 30 min. Anti-galectin-9-biotin antibody polyclonal antibody (R&D Systems) at 0.5 μg/mL was then added and incubated for 30 minutes. The plates were washed and neutravidin-HRP-conjugated (Pierce) was added and the plates incubated for an additional 45 minutes. The plates were washed and POD Chemiluminescence substrate (Roche) was added immediately prior to reading plates and the luminescence was read on a luminometer.

### Generation of Antigens Used in the Study

Cloning, expression and purification of the antigens was done using standard methods. Various protein fragments were expressed as hexahistidine tag or Fc fusion proteins. The amino acid sequences of the used proteins without the tag sequences are shown in SEQ ID NOs: 1-9, 138 and 89.

Full length human PD1 (huPD1);
SEQ ID NO: 1
PGWFLDSPDRPWNPPTFSPALLVVTEGDNATFTCSFSNTSESFVLNWYRM
SPSNQTDKLAAFPEDRSQPGQDCRFRVTQLPNGRDFHMSVVRARRNDSGT
YLCGAISLAPKAQIKESLRAELRVTERRAEVPTAHPSPSPRPAGQFQTLV
VGVVGGLLGSLVLLVWVLAVICSRAARGTIGARRTGQPLKEDPSAVPVFS
VDYGELDFQWREKTPEPPVPCVPEQTEYATIVFPSGMGTSSPARRGSADG
PRSAQPLRPE DGHCSWPL
Extracellular domain of human PD1 (huPD1-ECD);
SEQ ID NO: 2
PGWFLDSPDRPWNPPTFSPALLVVTEGDNATFTCSFSNTSESFVLNWYRM
SPSNQTDKLAAFPEDRSQPGQDCRFRVTQLPNGRDFHMSVVRARRNDSGT
YLCGAISLAPKAQIKESLRAELRVTERRAEVPTAHPSPSPRPAGQFQTL
Macaca fascicularis (cynomolgous, herein
referred to as cyno) PD1 (cPD1);
SEQ ID NO: 3)
PGWFLESPDRPWNAPTFSPALLLVTEGDNATFTCSFSNASESFVLNWYRM
SPSNQTDKLAAFPEDRSQPGQDCRFRVTRLPNGRDFHMSVVRARRNDSGT
YLCGAISLAPKAQIKESLRAELRVTERRAEVPTAHPSPSPRPAGQFQALV
VGVVGGLLGSLVLLVWVLAVICSRAAQGTIEARRTGQPLKEDPSAVPVFS
VDYGELDFQWREKTPEPPAPCVPEQTEYATIVFPSGLGTSSPARRGSADG
PRSPRPLRPEDGHCSWPL
Extracellular domain of cyno PD1 (cPD1-ECD);
SEQ ID NO: 4
PGWFLESPDRPWNAPTFSPALLLVTEGDNATFTCSFSNASESFVLNWYRM
SPSNQTDKLAAFPEDRSQPGQDCRFRVTRLPNGRDFHMSVVRARRNDSGT
YLCGAISLAPKAQIKESLRAELRVTERRAEVPTAHPSPSPRPAGQFQAL
Full length human PD-L1 (huPD-L1);
SEQ ID NO: 5
FTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISY
GGADYKRITVKVNAPYNKINQRILVVDPVTSEHELTCQAEGYPKAEVIWT
SSDHQVLSGKTTTTNSKREEKLFNVTSTLRINTTTNEIFYCTFRRLDPEE
NHTAELVIPELPLAHPPNER
Extracellular domain of human PD-L1 (huPDL1-ECD)
SEQ ID NO: 6
FTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISY
GGADYKRITVKVNAPYNKINQRILVVDPVTSEHELTCQAEGYPKAEVIWT
SSDHQVLSGKTTTTNSKREEKLFNVTSTLRINTTTNEIFYCTFRRLDPEE
NHTAELVIPELPLAHPPNERT
Extracellular domain of cynomolgus PD-L1
(cynoPDL1-ECD)
SEQ ID NO: 7
AFTVTVPKDLYVVEYGSNMTIECKFPVEKQLDLTSLIVYWEMEDKNIIQF
VHGEEDLKVQHSNYRQRAQLLKDQLSLGNAALRITDVKLQDAGVYRCMIS
YGGADYKRITVKVNAPYNKINQRILVVDPVTSEHELTCQAEGYPKAEVIW
TSSDHQVLSGKTTTTNSKREEKLLNVTSTLRINTTANEIFYCIFRRLDPE
ENHTAELVIPELPLALPPNERT
Extracellular domain of human PD-L2
(huPDL2-ECD)
SEQ ID NO: 8
LFTVTVPKELYIIEHGSNVTLECNFDTGSHVNLGAITASLQKVENDTSPH
RERATLLEEQLPLGKASFHIPQVQVRDEGQYQCIIIYGVAWDYKYLTLKV
KASYRKINTHILKVPETDEVELTCQATGYPLAEVSWPNVSVPANTSHSRT
PEGLYQVTSVLRLKPPPGRNFSCVFWNTHVRELTLASIDLQSQMEPRTHP
T
Extracellular domain of mouse PD1 (musPD1-ECD)
SEQ ID NO: 9
LEVPNGPWRSLTFYPAWLTVSEGANATFTCSLSNWSEDLMLNWNRLSPSN
QTEKQAAFCNGLSQPVQDARFQIIQLPNRHDFHMNILDTRRNDSGIYLCG
AISLHPKAKIEESPGAELVVTERILETSTRYPSPSPKPEGRFQ
Full length human TIM-3,
SEQ ID NO: 138
SEVEYRAEVGQNAYLPCFYTPAAPGNLVPVCWGKGACPVFECGNVVLRTD
ERDVNYWTSRYWLNGDFRKGDVSLTIENVTLADSGIYCCRIQIPGIMNDE
KFNLKLVIKPAKVTPAPTRQRDFTAAFPRMLTTRGHGPAETQTLGSLPDI
NLTQISTLANELRDSRLANDLRDSGATIRIGIYIGAGICAGLALALIFGA
LIFKWYSHSKEKIQNLSLISLANLPPSGLANAVAEGIRSEENIYTIEENV
YEVEEPNEYYCYVSSRQQPSQPLGCRFAMP
Extracellular domain of human TIM-3
(huTIM-3-ECD)
SEQ ID NO: 89
SEVEYRAEVGQNAYLPCFYTPAAPGNLVPVCWGKGACPVFECGNVVLRTD
ERDVNYWTSRYWLNGDFRKGDVSLTIENVTLADSGIYCCRIQIPGIMNDE
KFNLKLVIKPAKVTPAPTRQRDFTAAFPRMLTTRGHGPAETQTLGSLPDI
NLTQISTLANELRDSRLANDLRDSGATIR

### Example 2. Selection of Human Anti-PD-1 Antibodies from Phage Display Libraries

PD-1 binding Fabs were selected from de novo pIX phage display libraries as described in Shi et al., J Mol Biol 397:385-96, 2010, Int. Patent Publ. No. WO2009/085462 and U.S. Patent Publ. No. US2010/0021477. Briefly, the libraries were generated by diversifying human scaffolds where germline VH genes IGHV1-69*01, IGHV3-23*01, and IGHV5-51*01 were recombined with the human IGHJ-4 minigene via the H3 loop, and human germline VL kappa genes 012 (IGKV1-39*01), L6 (IGKV3-11*01), A27 (IGKV3-20*01), and B3 (IGKV4-1*01) were recombined with the IGKJ-1 minigene to assemble complete VH and VL domains. The positions in the heavy and light chain variable regions around H1, H2, L1, L2 and L3 loops corresponding to positions identified to be frequently in contact with protein and peptide antigens were chosen for diversification. Sequence diversity at selected positions was limited to residues occurring at each position in the IGHV or IGLV germline gene families of the respective IGHV or IGLV genes. Diversity at the H3 loop was generated by utilizing short to mid-sized synthetic loops of lengths 7-14 amino acids. The amino acid distribution at H3 was designed to mimic the observed variation of amino acids in human antibodies. Library design is detailed in Shi et al., (2010) J Mol Biol 397:385-96. The scaffolds utilized to generate libraries were named according to their human VH and VL germline gene origin. The three heavy chain libraries were combined with the four germline light chains or combined with the diversified light chain libraries to generate 12 unique VH:VL combinations. These libraries were later combined further based on library versions to generate additional libraries for panning experiments against PD-1.

The libraries were panned against huPD1-ECD, cynoPD1-ECD, musPD1-ECD, huPD1-Fc and/or musPD1-Fc. The recombinant proteins were biotinylated (bt) and captured on streptavidin magnetic beads (Dynal), then exposed to the de novo pIX Fab libraries at a final concentration of 100 nM or IOnM. Non-specific phages were washed away in PBS-Tween and bound phages were recovered by infection of MC106IF′ E. coli cells. Phages were amplified from these cells overnight and panning was repeated for a total of three or four rounds. Following the final round of biopanning, monoclonal Fab was screened for binding to huPD1-ECD, huPD1-Fc, musPD1-Fc and/or cynoPD1-Fc in two ELISA formats. In Format 1, Fab was captured on an ELISA plate by anti-Fd antibody and the various forms of btPD1's were added to captured Fab, followed by detection of bt-PD1's with Streptavidin:HRP. In Format 2, the various forms of btPD1's were captured on ELISA plates by Streptavidin and secreted Fab was added to the captured antigen, followed by detection of the Fab with GoatAntiFab′2HRP. Clones that demonstrated binding to the proteins were sequenced in the heavy and light chain variable regions.

Fabs from the human PD-1 or mouse PD-1 selections were then tested for cross-reactivity to cynoPD1-Fc secreted in mammalian cell supernatant. Fab was captured on an ELISA plate by anti-Fd antibody and the cynoPD1-Fc supernatant was added to the captured Fab, followed by detection of cynoPD1-Fc with GoatAntiHumanFc:HRP. Based on binding characteristics to cynoPD1-Fc, select antibodies were chosen for further characterization.

Select Fabs were chosen for further characterization and were cloned as IgG2sigma/κ. IgG2sigma has abolished effector functions and has V234A, G237A, P238S, H268A, V309L, A330S and P331S substitutions when compared to the wild type IgG2. IgG2sigma is described in U.S. Pat. No. 8,961,967. The antibodies were evaluated for their ability to block human PD-1 binding to cynomolgus PD-L1, affinity to human and cynomolgus PD-1 proteins, and their ability to bind to cells endogenously expressing human PD-1 (Jurkat cells). The antibodies were subsequently evaluated for their ability to block human PD-L1 and human PD-L2 binding to huPD1.

Based on the results, several antibodies were chosen for affinity maturation. Characteristics of select antibodies chosen for affinity maturation are shown in Table 7.

TABLE 7

| | Ligand inhibition; IC₅₀ (μg/ml) | | | | ProteOn SPR affinity | | |
|---|---|---|---|---|---|---|---|
| | cynoPD- | huPD- | huPD- | Jurkat binding; | kₒₙ | k_{off} | K_{D} |
| mAb | L1 | L1 | L2 | EC₅₀ μg/ml | (1/Ms) | (1/s) | (nM) |
| PD1B11 | 0.017-0.018 | 0.019 | 0.029 | 0.03-0.24 | 4.68E+05 | 8.96E−03 | 19.2 |
| PD1B70 | 0.010-0.021 | 0.040 | 0.059 | 0.69-1.32 | 1.84E+05 | 3.04E−02 | 166 |
| PD1B71 | 0.014-0.015 | 0.024 | 0.035 | 0.13-0.47 | 2.31E+05 | 2.77E−02 | 120 |

Hu: human
Cyno: cynomolgus

### Example 3. Affinity-Maturation of Human Anti-PD-1 Antibodies

Antibodies PD1B70, PD1B71 and PD1B114 (close homolog to PD1B11), were affinity matured in Fab format using phage display libraries with diversity at select VL positions and at HCDR1 and HCDR2. The design of affinity-maturation libraries for each Fab is shown in Table 8. Residue numbering is according to PD1B114 VH SEQ ID NO: 41 in Table 8.

TABLE 8
Diversification of PD1B114, PD1B70 and PD1B71 VH

| Position | Parent amino acid | Residues used for diversification |
|---|---|---|
| 30 | S | D, K, S |
| 31 | S | D, N, S, T |
| 32 | Y | A, D, S, Y |
| 33 | A | A, D, G, S, W, Y |
| 35 | S | H, N, S |
| 50 | G | A, E, G, N, R, T, W, Y |
| 52 | I | A, D, I, N, R, S |
| 54 | I | E, I, N, S, Y |
| 55 | F | E, F, Q, S, Y |
| 57 | T | D, N, R, S, T, Y |
| 59 | N | E, G, N, Q, R, Y |

Diversification of PD1B114, PD1B70 and PD1B71 VL

| Position | Parent amino acid | Residues used for diversification |
|---|---|---|
| 30 | S | D, N, R, S |
| 31 | S | N, S, T |
| 32 | Y | D, N, R, S, Y |
| 49 | Y | E, H, K, Y |
| 50 | D | D, G, S, W, Y |
| 53 | N | D, N, S, T, Y |
| 91 | R | A, D, E, G, H, N, R, S, W, Y |
| 92 | S | A, D, E, G, H, N, R, S, W, Y |
| 93 | N | A, D, E, G, H, N, R, S, W, Y |
| 94 | W | A, D, E, G, H, N, R, S, W, Y |
| 96 | L | F, I, L, N, R, W, Y |

The libraries were constructed and phage was generated. The VH and the VL phage libraries were then used for phage panning against huPD1-ECD and cynoPD1-ECD biotinylated recombinant proteins. Following phage panning, soluble Fabs were screened for binding to both human and cyno PD-1. Select Fabs were cloned as IgG2sigma isotype and characterized for their Jurkat cell binding and cynomolgus PD-L1 ligand inhibition at concentrations 1 μg/ml and 10 μg/ml.

Table 9 shows the characterization results of the parental and affinity-matured antibodies.

TABLE 9

| | Ligand inhibition | | Jurkat Cell |
|---|---|---|---|
| | at indicated | | binding; |
| | concentration* | | EC₅₀ |
| mAb | 1 μg/ml | 10 μg/ml | (μg/ml) |
| PD1B11 | 5% | 5% | 0.05 |
| PD1B114 | 8% | 13% | 0.47 |
| PD1B149 | 7% | 7% | 0.08 |
| PD1B160 | 4% | 3% | 0.08 |
| PD1B162 | 7% | 6% | 0.05 |
| PD1B164 | 6% | 3% | 0.06 |
| PD1B183 | 5% | 5% | 0.08 |
| PD1B184 | 4% | 4% | 0.08 |
| PD1B185 | 8% | 5% | 0.09 |
| PD1B187 | 7% | 5% | 0.09 |
| PD1B192 | 5% | 5% | 0.06 |
| PD1B70 | 6% | 6% | 0.69 |
| PD1B175 | 6% | 5% | 0.09 |
| PD1B71 | 6% | 9% | 0.13 |
| PD1B177 | 7% | 8% | 0.05 |

*value indicates percentage ligand not blocked

The affinity matured antibodies were assessed in affinity experiments as described above using ProteOn SPR analyses for binding to huPD1-ECD and cynoPD1-ECD. The binding characteristics of the mAbs to cyno PD-1 are shown in Table 10 and to human PD-1 in Table 11. STDEV were calculated for 3 or more replicates generated for human and cyno proteins. If less than 3 replicates were calculated, RANGE was indicated. RANGE is defined as the low and high values for the replicates tested. For samples in the Table 10 or Table 11 without value indicated in RANGE or STDEV, only one experiment was performed. The best affinity matured variants had affinities for human and cyno PD-1 in the single digit nM range following ˜4-20 fold gains in affinity compared to their parental mAbs.

TABLE 10

| | antigen: cyno PD-1 | | | | | |
|---|---|---|---|---|---|---|
| | | | | STDEV. | | STDEV. |
| | kₒₙ | STDEV. kon | k_{off} | koff | K_{D} | K_{D} |
| Sample | (1/Ms) | or RANGE | (1/s) | or RANGE | (nM) | or RANGE |
| PD1B70 | 2.10E+05 | (1.99-2.25)E+05 | 2.58E−02 | (2.45-2.75)E−02 | 123 | 109-138 |
| PD1B175 | 2.14E+05 | (1.98-2.30)E+05 | 6.40E−03 | (6.06-6.73)E−03 | 30 | 26-34 |
| PD1B71 | 3.04E+05 | 2.35E+04 | 2.03E−02 | 7.27E−04 | 66.8 | 5.68 |
| PD1B177 | 2.92E+05 | (2.80-3.04)E+05 | 1.89E−03 | (1.84-1.93)E−03 | 6.47 | 6.1-6.9 |
| PD1B114 | 2.94E+05 | 1.69E+04 | 2.39E−02 | 1.45E−03 | 81.5 | 6.8 |
| PD1B149 | 3.20E+05 | (3.04-3.36)E+05 | 3.57E−03 | (3.48-3.65)E−03 | 11.2 | (10.9-11.4) |
| PD1B160 | 3.17E+05 | (3.16-3.17)E+05 | 1.66E−03 | (1.63-1.68)E−03 | 5.23 | 5.1-5.3 |
| PD1B162 | 3.87E+05 | (3.84-3.89)E+05 | 9.79E−04 | (9.59-9.98)E−04 | 2.53 | 2.5-2.6 |
| PD1B164 | 2.67E+05 | (2.67-2.67)E+05 | 2.87E−04 | (2.82-2.91)E−04 | 1.07 | 1.06-1.09 |
| PD1B11 | 2.93E+05 | (2.85-3.01)E+05 | 9.17E−03 | (0.8-1.00)E−02 | 31.3 | (27.7-35.1) |
| PD1B183 | 3.20E+05 | (3.04-3.37)E+05 | 8.39E−03 | (8.01-8.76)E−03 | 26.3 | 23.9-28.8 |
| PD1B184 | 2.38E+05 | (2.08-2.68)E+05 | 2.74E−03 | (2.55-2.92)E−03 | 11.5 | 9.5-14.1 |
| PD1B185 | 3.11E+05 | (2.80-3.43)E+05 | 9.47E−03 | (9.38-9.55)E−03 | 30.5 | 27.5-34.1 |
| PD1B187 | 2.94E+05 | (2.20-3.70)E+05 | 1.57E−03 | (1.28-1.85)E−03 | 5.32 | 3.5-8.4 |
| PD1B192 | 3.07E+05 | (2.90-3.24)E+05 | 5.04E−03 | (4.86-5.22)E−03 | 16.4 | 15.0-18.0 |

TABLE 11

| | Antigen: human PD-1 | | |
|---|---|---|---|
| | kₒₙ | k_{off} | K_{D} |
| Sample | (1/Ms) | (1/s) | (nM) |
| PD1B70 | 4.15E+05 | 4.18E−02 | 101 |
| PD1B175 | 4.22E+05 | 9.72E−03 | 23 |
| PD1B71 | 5.48E+05 | 2.73E−02 | 49.9 |
| PD1B177 | 5.15E+05 | 2.57E−03 | 5 |
| PD1B114 | 5.17E+05 | 2.79E−02 | 54.1 |
| PD1B149 | 5.32E+05 | 6.20E−03 | ~12* |
| PD1B160 | 5.40E+05 | 3.71E−03 | 6.87 |
| PD1B162 | 6.49E+05 | 3.86E−03 | 5.95 |
| PD1B164 | 4.48E+05 | 1.31E−03 | 2.92 |
| PD1B11 | 5.16E+05 | 8.52E−03 | ~17* |
| PD1B183 | 5.27E+05 | 8.44E−03 | 16 |
| PD1B184 | 4.45E+05 | 5.09E−03 | 11.4 |
| PD1B185 | 5.85E+05 | 7.65E−03 | 13.1 |
| PD1B187 | 5.35E+05 | 2.78E−03 | 5.2 |
| PD1B192 | 5.41E+05 | 1.17E−02 | ~228 |

*Values did not pass the data acceptance criteria (chi2 > 20%) and were therefore considered approximations.

### Example 4. Combinatorial Variant PD-1 mAb Production

Following the analysis of the affinity results, combinatorial sequences were considered.

PD1B11 and PD1B114 have very similar sequences. Because PD1B11 had approximately a 3-fold tighter affinity to human PD-1 and a 2-fold tighter affinity to cyno PD-1 compared to PD1B114, antibodies having combinations of their various CDRs were made. The HCDR3 of PD1B11 was placed into PD1B164 and PD1B162 (affinity-matured variants of PD1B114), using site directed mutagenesis while the HCDR2 of PD1B164 (affinity matured variant of PD1B114) was placed into PD1B187 (affinity matured variant of PD1B11). The resulting heavy chains were paired with parental light chains resulting in new antibodies PD1B194, PD1B195 and PD1B196, respectively.

PD1B175 and PD1B177 both contained the parental light chain even though the antibodies were generated using diversified VL libraries during affinity maturation. In an attempt to increase antibody affinities, PD1B175 heavy chain was paired with PD1L185 or PD1L187 affinity matured light chains, and PD1B177 heavy chain was paired with PD1L86, PD1L168 or PD1L190 affinity matured light chains, resulting in antibodies PD1B197, PD1B198, PD1B199, PD1B200 and PD1B201. VH and VL pairing of the antibodies is shown in Table 20 in Example 5.

The HCDR, LCDR, VH and VL sequences of these antibodies are shown in Tables 14, 15, 16, 17, 18, 19, 21 and 22 in Example 5. The antibodies were cloned as IgG2sigma/κmAbs and transiently expressed in HEK293 expi cells for affinity measurements.

Affinities of the resulting antibodies were determined as described above. Table 12 shows the measured affinities of the combinatorial mAb variants to cyno PD-1 and Table 13 shows the affinities to human PD-1. STDEV were calculated for 3 or more replicates generated for human and cyno proteins. If less than 3 replicates were calculated, RANGE is indicated. RANGE is defined as the low and high values for the replicates tested. For samples without RANGE or STDEV, only one experiment was performed

TABLE 12

| | binding to cyno PD-1 | | | | | |
|---|---|---|---|---|---|---|
| | kₒₙ | STDEV. kon | k_{off} | STDEV. koff | K_{D} | STDEV. KD |
| Sample | (1/Ms) | or RANGE | (1/s) | or RANGE | (nM) | or RANGE |
| PD1B70 | 2.50E+05 | (2.25-2.74)E+05 | 2.22E−02 | (2.18-2.26)E−02 | 88.98 | (79.6-100) |
| (Parent) | | | | | | |
| PD1B197 | 2.75E+05 | 1.27E+04 | 1.26E−03 | 4.04E−05 | 4.6 | 0.3 |
| PD1B198 | 3.72E+05 | 1.61E+04 | 4.16E−03 | 9.29E−05 | 11.18 | 0.54 |
| PD1B11 | 3.50E+05 | (3.49-3.50)E+05 | 9.42E−03 | (9.38-9.46)E−03 | 26.95 | (26.8-27.1) |
| (Parent) | | | | | | |
| PD1B194 | 3.22E+05 | 2.86E+04 | 1.93E−04 | 5.86E−06 | 0.6 | 0.06 |
| PD1B195 | 4.32E+05 | (4.30-4.34)E+05 | 4.08E−04 | (3.96-4.19)E−04 | 0.94 | (0.91-0.97) |
| PD1B196 | 3.03E+05 | 6.66E+03 | 1.76E−04 | 9.85E−06 | 0.58 | 0.03 |
| PD1B71 | 3.77E+05 | (3.37-4.17)E+05 | 1.96E−02 | (1.85-2.07)E−02 | 51.99 | (44.4-61.4) |
| (Parent) | | | | | | |
| PD1B199 | 3.40E+05 | 7.94E+03 | 1.77E−04 | 1.55E−05 | 0.52 | 0.05 |
| PD1B200 | 3.80E+05 | 2.21E+04 | 4.22E−04 | 1.99E−05 | 1.11 | 0.08 |
| PD1B201 | 3.05E+05 | 1.80E+04 | 2.93E−04 | 2.35E−05 | 0.96 | 0.1 |

TABLE 13

| | binding to human PD-1 | | | | | |
|---|---|---|---|---|---|---|
| | | STDEV. | | STDEV. | | |
| | kₒₙ | kon | k_{off} | koff | K_{D} | STDEV. KD |
| Sample | (1/Ms) | or RANGE | (1/s) | or RANGE | (nM) | or RANGE |
| PD1B70 | 7.69E+05 | (7.37-8.00)E+05 | 3.49E−02 | (3.41-3.56)E−02 | 45.35 | (42.6-43.8) |
| (Parent) | | | | | | |
| PD1B197 | 6.58E+05 | 2.26E+04 | 3.24E−03 | 1.74E−04 | 4.9 | 0.3 |
| PD1B198 | 8.95E+05 | 6.44E+04 | 9.34E−03 | 9.90E−04 | 10.43 | 1.34 |
| PD1B11 | 9.33E+05 | (8.84-9.82)E+05 | 9.05E−03 | (8.67-9.43)E−03 | 9.7 | (9.6-9.81) |
| (Parent) | | | | | | |
| PD1B194 | 8.97E+05 | 1.45E+05 | 9.60E−04 | 2.78E−05 | 1.07 | 0.18 |
| PD1B195 | 1.23E+06 | 1.79E+05 | 1.52E−03 | 6.51E−05 | 1.23 | 0.19 |
| PD1B196 | 8.83E+05 | 6.39E+04 | 3.66E−04 | 2.01E−05 | 0.41 | 0.04 |
| PD1B71 | 9.55E+05 | (9.33-9.76)E+05 | 2.25E−02 | (2.19-2.30)E−02 | 23.52 | (22.4-24.7) |
| (Parent) | | | | | | |
| PD1B199 | 9.33E+05 | 6.92E+04 | 5.64E−04 | 1.98E−05 | 0.6 | 0.05 |
| PD1B200 | 1.05E+06 | 1.40E+05 | 1.22E−03 | 3.21E−05 | 1.17 | 0.16 |
| PD1B201 | 8.58E+05 | 8.22E+04 | 9.57E−04 | 3.06E−05 | 1.12 | 0.11 |

### Example 5. Structural Characterization of an-PD1 Antibodies Derived from Phage Display Libraries

The cDNA sequences and amino acid translations of the antibodies were obtained using standard techniques throughout the generation of the antibodies using various campaigns. After polypeptide sequence determination, some antibody cDNAs encoding the variable regions or full length antibodies were codon optimized using standard methods for scale-up expression.

Table 14 shows the HCDR1 sequences of select PD-1 antibodies.

Table 15 shows the HCDR2 sequences of select PD-1 antibodies.

Table 16 shows the HCDR3 sequences of select PD-1 antibodies.

Table 17 shows the LCDR1 sequences of select PD-1 antibodies.

Table 18 shows the LCDR2 sequences of select PD-1 antibodies.

Table 19 shows the LCDR3 sequences of select PD-1 antibodies.

Table 20 shows the VH and the VL pairing of select PD-1 antibodies.

Table 21 shows the VH sequences of select PD-1 antibodies.

Table 22 shows the VL sequences of select PD-1 antibodies.

TABLE 14

| | HCDR1 | | | | | |
|---|---|---|---|---|---|---|
| | | | | | | SEQ ID |
| Antibody | Sequence | | | | | NO: |
| PD1B114 | S | Y | A | I | S | 10 |
| PD1B149 | S | Y | A | I | S | 10 |
| PD1B160 | S | Y | A | I | S | 10 |
| PD1B162 | S | Y | A | I | S | 10 |
| PD1B164 | S | Y | A | I | S | 10 |
| PD1B11 | S | Y | A | I | S | 10 |
| PD1B183 | S | Y | A | I | S | 10 |
| PD1B184 | S | Y | A | I | S | 10 |
| PD1B185 | S | Y | A | I | S | 10 |
| PD1B187 | S | Y | A | I | S | 10 |
| PD1B192 | S | Y | A | I | S | 10 |
| PD1B71 | S | Y | A | I | S | 10 |
| PD1B177 | D | Y | V | I | S | 11 |
| PD1B70 | S | Y | A | I | S | 10 |
| PD1B175 | S | Y | V | I | H | 12 |
| PD1B194 | S | Y | A | I | S | 10 |
| PD1B195 | S | Y | A | I | S | 10 |
| PD1B196 | S | Y | A | I | S | 10 |
| PD1B197 | S | Y | V | I | H | 12 |
| PD1B198 | S | Y | V | I | H | 12 |
| PD1B199 | D | Y | V | I | S | 11 |
| PD1B200 | D | Y | V | I | S | 11 |
| PD1B201 | D | Y | V | I | S | 11 |

TABLE 15

| | HCDR2 | | | | | | | | | | | | | | | | | |
|---|---|---|---|---|---|---|---|---|---|---|---|---|---|---|---|---|---|---|
| Antibody | Sequence | | | | | | | | | | | | | | | | | SEQ ID NO: |
| PD1B114 | G | I | I | P | I | F | G | T | A | N | Y | A | Q | K | F | Q | G | 13 |
| PD1B149 | G | I | I | P | I | F | G | T | A | N | Y | A | Q | K | F | Q | G | 13 |
| PD1B160 | G | I | I | P | I | F | D | T | A | N | Y | A | Q | K | F | Q | G | 14 |
| PD1B162 | G | I | I | P | I | F | D | T | A | N | Y | A | Q | K | F | Q | G | 14 |
| PD1B164 | G | I | I | P | I | F | D | T | A | N | Y | A | Q | K | F | Q | G | 14 |
| PD1B11 | G | I | I | P | I | F | G | T | A | N | Y | A | Q | K | F | Q | G | 13 |
| PD1B183 | G | I | I | P | I | F | G | T | A | N | Y | A | Q | K | F | Q | G | 13 |
| PD1B184 | G | I | I | P | I | F | G | T | A | N | Y | A | Q | K | F | Q | G | 13 |
| PD1B185 | G | I | I | P | I | F | G | T | A | N | Y | A | Q | K | F | Q | G | 13 |
| PD1B187 | G | I | I | P | I | F | G | T | A | N | Y | A | Q | K | F | Q | G | 13 |
| PD1B192 | G | I | I | P | I | F | G | T | A | N | Y | A | Q | K | F | Q | G | 13 |
| PD1B71 | G | I | I | P | I | F | G | T | A | N | Y | A | Q | K | F | Q | G | 13 |
| PD1B177 | G | I | I | P | I | Y | G | T | A | N | Y | A | Q | K | F | Q | G | 15 |
| PD1B70 | G | I | I | P | I | F | G | T | A | N | Y | A | Q | K | F | Q | G | 13 |
| PD1B175 | G | I | I | P | I | F | G | T | A | N | Y | A | Q | K | F | Q | G | 13 |
| PD1B194 | G | I | I | P | I | F | D | T | A | N | Y | A | Q | K | F | Q | G | 14 |
| PD1B195 | G | I | I | P | I | F | D | T | A | N | Y | A | Q | K | F | Q | G | 14 |
| PD1B196 | G | I | I | P | I | F | D | T | A | N | Y | A | Q | K | F | Q | G | 14 |
| PD1B197 | G | I | I | P | I | F | G | T | A | N | Y | A | Q | K | F | Q | G | 13 |
| PD1B198 | G | I | I | P | I | F | G | T | A | N | Y | A | Q | K | F | Q | G | 13 |
| PD1B199 | G | I | I | P | I | Y | G | T | A | N | Y | A | Q | K | F | Q | G | 15 |
| PD1B200 | G | I | I | P | I | Y | G | T | A | N | Y | A | Q | K | F | Q | G | 15 |
| PD1B201 | G | I | I | P | I | Y | G | T | A | N | Y | A | Q | K | F | Q | G | 15 |

TABLE 16

| | HCDR3 | | | | | | | | | | | | | | |
|---|---|---|---|---|---|---|---|---|---|---|---|---|---|---|---|
| | | | | | | | | | | | | | | | SEQ |
| | | | | | | | | | | | | | | | ID |
| Antibody | Sequence | | | | | | | | | | | | | | NO: |
| PD1B114 | P | G | L | A | A | A | Y | D | T | G | N | L | D | Y | 16 |
| PD1B149 | P | G | L | A | A | A | Y | D | T | G | N | L | D | Y | 16 |
| PD1B160 | P | G | L | A | A | A | Y | D | T | G | N | L | D | Y | 16 |
| PD1B162 | P | G | L | A | A | A | Y | D | T | G | N | L | D | Y | 16 |
| PD1B164 | P | G | L | A | A | A | Y | D | T | G | N | L | D | Y | 16 |
| PD1B11 | P | G | L | A | A | A | Y | D | T | G | S | L | D | Y | 17 |
| PD1B183 | P | G | L | A | A | A | Y | D | T | G | S | L | D | Y | 17 |
| PD1B184 | P | G | L | A | A | A | Y | D | T | G | S | L | D | Y | 17 |
| PD1B185 | P | G | L | A | A | A | Y | D | T | G | S | L | D | Y | 17 |
| PD1B187 | P | G | L | A | A | A | Y | D | T | G | S | L | D | Y | 17 |
| PD1B192 | P | G | L | A | A | A | Y | D | T | G | S | L | D | Y | 17 |
| PD1B71 | G | T | L | D | R | T | G | H | L | D | Y | | | | 18 |
| PD1B177 | G | T | L | D | R | T | G | H | L | D | Y | | | | 18 |
| PD1B70 | G | Y | V | R | A | T | G | M | L | D | Y | | | | 19 |
| PD1B175 | G | Y | V | R | A | T | G | M | L | D | Y | | | | 19 |
| PD1B194 | P | G | L | A | A | A | Y | D | T | G | S | L | D | Y | 17 |
| PD1B195 | P | G | L | A | A | A | Y | D | T | G | S | L | D | Y | 17 |
| PD1B196 | P | G | L | A | A | A | Y | D | T | G | S | L | D | Y | 17 |
| PD1B197 | G | Y | V | R | A | T | G | M | L | D | Y | | | | 19 |
| PD1B198 | G | Y | V | R | A | T | G | M | L | D | Y | | | | 19 |
| PD1B199 | G | T | L | D | R | T | G | H | L | D | Y | | | | 18 |
| PD1B200 | G | T | L | D | R | T | G | H | L | D | Y | | | | 18 |
| PD1B201 | G | T | L | D | R | T | G | H | L | D | Y | | | | 18 |

TABLE 17

| | LCDR1 | | | | | | | | | | | |
|---|---|---|---|---|---|---|---|---|---|---|---|---|
| | | | | | | | | | | | | SEQ |
| | | | | | | | | | | | | ID |
| Antibody | Sequence | | | | | | | | | | | NO: |
| PD1B114 | R | A | S | Q | S | V | S | S | Y | L | A | 20 |
| PD1B149 | R | A | S | Q | S | V | R | N | Y | L | A | 21 |
| PD1B160 | R | A | S | Q | S | V | D | S | Y | L | A | 22 |
| PD1B162 | R | A | S | Q | S | V | D | S | Y | L | A | 22 |
| PD1B164 | R | A | S | Q | S | V | R | S | Y | L | A | 23 |
| PD1B11 | R | A | S | Q | S | V | S | S | Y | L | A | 20 |
| PD1B183 | R | A | S | Q | S | V | S | S | Y | L | A | 20 |
| PD1B184 | R | A | S | Q | S | V | R | N | Y | L | A | 21 |
| PD1B185 | R | A | S | Q | S | V | R | N | Y | L | A | 21 |
| PD1B187 | R | A | S | Q | S | V | R | S | Y | L | A | 23 |
| PD1B192 | R | A | S | Q | S | V | D | S | Y | L | A | 22 |
| PD1B71 | R | A | S | Q | S | V | S | S | Y | L | A | 20 |
| PD1B177 | R | A | S | Q | S | V | S | S | Y | L | A | 20 |
| PD1B70 | R | A | S | Q | S | V | S | S | Y | L | A | 20 |
| PD1B175 | R | A | S | Q | S | V | S | S | Y | L | A | 20 |
| PD1B194 | R | A | S | Q | S | V | R | S | Y | L | A | 23 |
| PD1B195 | R | A | S | Q | S | V | D | S | Y | L | A | 22 |
| PD1B196 | R | A | S | Q | S | V | R | S | Y | L | A | 23 |
| PD1B197 | R | A | S | Q | S | V | S | N | Y | L | A | 24 |
| PD1B198 | R | A | S | Q | S | V | S | S | Y | L | A | 20 |
| PD1B199 | R | A | S | Q | S | V | S | S | Y | L | A | 20 |
| PD1B200 | R | A | S | Q | S | V | D | N | Y | L | A | 25 |
| PD1B201 | R | A | S | Q | S | V | S | N | Y | L | A | 24 |

TABLE 18

| | LCDR2 | | | | | | | |
|---|---|---|---|---|---|---|---|---|
| | | | | | | | | SEQ |
| | | | | | | | | ID |
| Antibody | Sequence | | | | | | | NO: |
| PD1B114 | D | A | S | N | R | A | T | 26 |
| PD1B149 | D | A | S | N | R | A | T | 26 |
| PD1B160 | D | A | S | D | R | A | T | 27 |
| PD1B162 | D | A | S | N | R | A | T | 26 |
| PD1B164 | D | A | S | Y | R | A | T | 28 |
| PD1B11 | D | A | S | N | R | A | T | 26 |
| PD1B183 | D | A | S | N | R | A | T | 26 |
| PD1B184 | D | A | S | N | R | A | T | 26 |
| PD1B185 | D | A | S | D | R | A | T | 27 |
| PD1B187 | D | A | S | N | R | A | T | 26 |
| PD1B192 | D | A | S | N | R | A | T | 26 |
| PD1B71 | D | A | S | N | R | A | T | 26 |
| PD1B177 | D | A | S | N | R | A | T | 26 |
| PD1B70 | D | A | S | N | R | A | T | 26 |
| PD1B175 | D | A | S | N | R | A | T | 26 |
| PD1B194 | D | A | S | Y | R | A | T | 28 |
| PD1B195 | D | A | S | N | R | A | T | 26 |
| PD1B196 | D | A | S | N | R | A | T | 26 |
| PD1B197 | D | A | S | N | R | A | T | 26 |
| PD1B198 | D | A | S | S | R | A | T | 29 |
| PD1B199 | D | A | S | T | R | A | T | 30 |
| PD1B200 | D | A | S | N | R | A | T | 26 |
| PD1B201 | D | A | S | N | R | A | T | 26 |

TABLE 19

| | LCDR3 | | | | | | | | | |
|---|---|---|---|---|---|---|---|---|---|---|
| | | | | | | | | | | SEQ |
| | | | | | | | | | | ID |
| Antibody | Sequence | | | | | | | | | NO: |
| PD1B114 | Q | Q | R | S | N | W | P | L | T | 31 |
| PD1B149 | Q | Q | R | N | Y | W | P | L | T | 32 |
| PD1B160 | Q | Q | R | G | N | W | P | L | T | 33 |
| PD1B162 | Q | Q | R | E | Y | W | P | L | T | 34 |
| PD1B164 | Q | Q | R | D | Y | W | P | L | T | 35 |
| PD1B11 | Q | Q | R | S | N | W | P | L | T | 31 |
| PD1B183 | Q | Q | R | G | Y | W | P | L | T | 36 |
| PD1B184 | Q | Q | R | N | Y | W | P | L | T | 32 |
| PD1B185 | Q | Q | R | W | N | W | P | L | T | 37 |
| PD1B187 | Q | Q | R | N | Y | W | P | L | T | 32 |
| PD1B192 | Q | Q | R | N | Y | W | P | L | T | 32 |
| PD1B71 | Q | Q | R | S | N | W | P | L | T | 31 |
| PD1B177 | Q | Q | R | S | N | W | P | L | T | 31 |
| PD1B70 | Q | Q | R | S | N | W | P | L | T | 31 |
| PD1B175 | Q | Q | R | S | N | W | P | L | T | 31 |
| PD1B194 | Q | Q | R | D | Y | W | P | L | T | 35 |
| PD1B195 | Q | Q | R | E | Y | W | P | L | T | 34 |
| PD1B196 | Q | Q | R | N | Y | W | P | L | T | 32 |
| PD1B197 | Q | Q | R | A | Y | W | P | L | T | 38 |
| PD1B198 | Q | Q | R | A | E | W | P | L | T | 39 |
| PD1B199 | Q | Q | R | N | Y | W | P | L | T | 32 |
| PD1B200 | Q | Q | R | S | A | W | P | L | T | 40 |
| PD1B201 | Q | Q | R | N | Y | W | P | L | T | 32 |

TABLE 20

| | VH | | VL | |
|---|---|---|---|---|
| | peptide | VH SEQ | peptide | VL SEQ |
| Antibody | ID | ID NO: | ID | ID NO: |
| PD1B114 | PD1H24 | 41 | PH9L3 | 49 |
| PD1B149 | PD1H24 | 41 | PD1L128 | 50 |
| PD1B160 | PD1H131 | 42 | PD1L101 | 51 |
| PD1B162 | PD1H131 | 42 | PD1L67 | 52 |
| PD1B164 | PD1H131 | 42 | PD1L71 | 53 |
| PD1B11 | PD1H3 | 43 | PH9L3 | 49 |
| PD1B183 | PD1H3 | 43 | PD1L109 | 54 |
| PD1B184 | PD1H3 | 43 | PD1L128 | 50 |
| PD1B185 | PD1H3 | 43 | PD1L132 | 55 |
| PD1B187 | PD1H3 | 43 | PD1L148 | 56 |
| PD1B192 | PD1H3 | 43 | PD1L133 | 57 |
| PD1B71 | PD1H108 | 44 | PH9L3 | 49 |
| PD1B177 | PD1H164 | 45 | PH9L3 | 49 |
| PD1B70 | PD1H107 | 46 | PH9L3 | 49 |
| PD1B175 | PD1H163 | 47 | PH9L3 | 49 |
| PD1B194 | PD1H170 | 48 | PD1L71 | 53 |
| PD1B195 | PD1H170 | 48 | PD1L67 | 52 |
| PD1B196 | PD1H170 | 48 | PD1L148 | 56 |
| PD1B197 | PD1H163 | 47 | PD1L185 | 58 |
| PD1B198 | PD1H163 | 47 | PD1L187 | 59 |
| PD1B199 | PD1H164 | 45 | PD1L86 | 60 |
| PD1B200 | PD1H164 | 45 | PD1L168 | 61 |
| PD1B201 | PD1H164 | 45 | PD1L190 | 62 |

TABLE 21

| VH | VH | |
|---|---|---|
| peptide | SEQ | |
| ID | ID NO: | VH sequence |
| PD1H24 | 41 | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARPGLAAAY |
| | | DTGNLDYWGQGTLVTVSS |
| PD1H131 | 42 | QVQLVQSGAEVKKPGSSVKVSCKASGGTFSSYAIS |
| | | WVRQAPGQGLEWMGGIIPIFDTANYAQKFQGRVTI |
| | | TADESTSTAYMELSSLRSEDTAVYYCARPGLAAAY |
| | | DTGNLDYWGQGTLVTVSS |
| PD1H3 | 43 | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARPGLAAAY |
| | | DTGSLDYWGQGTLVTVSS |
| PD1H108 | 44 | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARGTLDRTG |
| | | HLDYWGQGTLVTVSS |
| PD1H164 | 45 | QVQLVQSGAEVKKPGSSVKVSCKASGGTFSDYVIS |
| | | WVRQAPGQGLEWMGGIIPIYGTANYAQKFQGRVTI |
| | | TADESTSTAYMELSSLRSEDTAVYYCARGTLDRTG |
| | | HLDYWGQGTLVTVSS |
| PD1H107 | 46 | QVQLVQSGAEVKKPGSSVKVSCKASGGTFSSYAIS |
| | | WVRQAPGQGLEWMGGIIPIFGTANYAQKFQGRVTI |
| | | TADESTSTAYMELSSLRSEDTAVYYCARGYVRATG |
| | | MLDYWGQGTLVTVSS |
| PD1H163 | 47 | QVQLVQSGAEVKKPGSSVKVSCKASGGTFKSYVIH |
| | | WVRQAPGQGLEWMGGIIPIFGTANYAQKFQGRVTI |
| | | TADESTSTAYMELSSLRSEDTAVYYCARGYVRATG |
| | | MLDYWGQGTLVTVSS |
| PD1H170 | 48 | QVQLVQSGAEVKKPGSSVKVSCKASGGTFSSYAIS |
| | | WVRQAPGQGLEWMGGIIPIFDTANYAQKFQGRVTI |
| | | TADESTSTAYMELSSLRSEDTAVYYCARPGLAAAY |
| | | DTGSLDYWGQGTLVTVSS |

TABLE 22

| VL peptide | VL SEQ | |
|---|---|---|
| ID | ID NO: | VL sequence |
| PH9L3 | 49 | EIVLTQSPATLSLSPGERATLSCRASQSVSSYLAWYQQKPGQAPR |
| | | LLIYDASNRATGIPARFSGSGSGTDFTLTISSLEPEDFAVYYCQQ |
| | | RSNWPLTFGQGTKVEIK |
| PD1L128 | 50 | EIVLTQSPATLSLSPGERATLSCRASQSVRNYLAWYQQKPGQAPR |
| | | LLIHDASNRATGIPARFSGSGSGTDFTLTISSLEPEDFAVYYCQQ |
| | | RNYWPLTFGQGTKVEIK |
| PD1L101 | 51 | EIVLTQSPATLSLSPGERATLSCRASQSVDSYLAWYQQKPGQAPR |
| | | LLIKDASDRATGIPARFSGSGSGTDFTLTISSLEPEDFAVYYCQQ |
| | | RGNWPLTFGQGTKVEIK |
| PD1L67 | 52 | EIVLTQSPATLSLSPGERATLSCRASQSVDSYLAWYQQKPGQAPR |
| | | LLIYDASNRATGIPARFSGSGSGTDFTLTISSLEPEDFAVYYCQQ |
| | | REYWPLTFGQGTKVEIK |
| PD1L71 | 53 | EIVLTQSPATLSLSPGERATLSCRASQSVRSYLAWYQQKPGQAPR |
| | | LLIYDASYRATGIPARFSGSGSGTDFTLTISSLEPEDFAVYYCQQ |
| | | RDYWPLTFGQGTKVEIK |
| PD1L109 | 54 | EIVLTQSPATLSLSPGERATLSCRASQSVSSYLAWYQQKPGQAPR |
| | | LLIKDASNRATGIPARFSGSGSGTDFTLTISSLEPEDFAVYYCQQ |
| | | RGYWPLTFGQGTKVEIK |
| PD1L132 | 55 | EIVLTQSPATLSLSPGERATLSCRASQSVRNYLAWYQQKPGQAPR |
| | | LLIYDASDRATGIPARFSGSGSGTDFTLTISSLEPEDFAVYYCQQ |
| | | RWNWPLTFGQGTKVEIK |
| PD1L148 | 56 | EIVLTQSPATLSLSPGERATLSCRASQSVRSYLAWYQQKPGQAPR |
| | | LLIYDASNRATGIPARFSGSGSGTDFTLTISSLEPEDFAVYYCQQ |
| | | RNYWPLTFGQGTKVEIK |
| PD1L133 | 57 | EIVLTQSPATLSLSPGERATLSCRASQSVDSYLAWYQQKPGQAPR |
| | | LLIHDASNRATGIPARFSGSGSGTDFTLTISSLEPEDFAVYYCQQ |
| | | RNYWPLTFGQGTKVEIK |
| PD1L185 | 58 | EIVLTQSPATLSLSPGERATLSCRASQSVSNYLAWYQQKPGQAPR |
| | | LLIYDASNRATGIPARFSGSGSGTDFTLTISSLEPEDFAVYYCQQ |
| | | RAYWPLTFGQGTKVEIK |
| PD1L187 | 59 | EIVLTQSPATLSLSPGERATLSCRASQSVSSYLAWYQQKPGQAPR |
| | | LLIEDASSRATGIPARFSGSGSGTDFTLTISSLEPEDFAVYYCQQ |
| | | RAEWPLTFGQGTKVEIK |
| PD1L86 | 60 | EIVLTQSPATLSLSPGERATLSCRASQSVSSYLAWYQQKPGQAPR |
| | | LLIHDASTRATGIPARFSGSGSGTDFTLTISSLEPEDFAVYYCQQ |
| | | RNYWPLTFGQGTKVEIK |
| PD1L168 | 61 | EIVLTQSPATLSLSPGERATLSCRASQSVDNYLAWYQQKPGQAPR |
| | | LLIHDASNRATGIPARFSGSGSGTDFTLTISSLEPEDFAVYYCQQ |
| | | RSAWPLTFGQGTKVEIK |
| PD1L190 | 62 | EIVLTQSPATLSLSPGERATLSCRASQSVSNYLAWYQQKPGQAPR |
| | | LLIYDASNRATGIPARFSGSGSGTDFTLTISSLEPEDFAVYYCQQ |
| | | RNYWPLTFGQGTKVEIK |

All anti-PD-1 antibodies were identified to have VH1-69 (SEQ ID NO: 170) and IGKV3-11 (L6) (SEQ ID NO: 171) frameworks.

SEQ ID NO: 170
QVQLVQSGAEVKKPGSSVKVSCKASGGTFS SYAIS
WVRQAPGQGLEWMG GIIPIFGTANYAQKFQG
RVTITADESTSTAYMELSSLRSEDTAVYYCAR
SEQ ID NO: 171
EIVLTQSPATLSLSPGERATLSCRASQSVSSYLAWYQQKPGQAPRLLIYD
ASNRATGIPARFSGSGSGTDFTLTISSLEPEDFAVYYCQQRSNWP

### Example 6. Generation and Characterization of PD-1 Antibodies in Mice

BALB/c were immunized intraperitoneally with huPD1-ECD and assessed for specific IgG titers. Once sufficient titers were obtained, splenocytes were isolated and fused with FO cells. The resulting hybridomas were plated in 96 well plates and cultured for 10 days. Antigen specific clones were identified by standard capture ELISA for binding to huPD1-ECD. Human PD-1-specific hybridomas were further tested for their affinity to human and cyno PD-1, binding to Jurkat cells and cyno PD-L1 inhibition. Based on the results, clone PD1B28 was selected for humanization using framework adaptation.

Framework adaptation process was done as essentially described in U.S. Patent Publ. No. 2009/0118127 and Fransson et al., (2010) J Mol Biol 398:214-231. Briefly, the heavy and light chain sequences were compared with the human germline sequences (only the “01” alleles as of Oct. 1, 2007) using BLAST search against the IMGT database (Kaas, et al., (2004) Nucl Acids Res 32, D208-D210; Lefranc et al., (2005) Nucl Acid Res 33, D593-D597). From this set of human germline genes, redundant genes (100% identical at amino acid level) and those with unpaired cysteine residues were removed. The remaining closest matching human germline genes in both the framework and CDR regions were chosen as the acceptor human frameworks. Several VL and VH germline human frameworks were selected based upon overall sequence homology and CDR lengths as well as CDR similarity. FR-4 was selected based on sequence similarity of the IGHJ/IGJK germline genes. Then, the CDRs of PD1B28 were transferred into the selected acceptor human frameworks to generate the HFA variants, except in the region corresponding to the HCDR1 of V_{H}. For this region a combination of CDR and HV, or a shorter HCDR2 (referred to as Kabat-7, see U.S. Patent Publ. No. 2009/0118127) were transferred from the non-human antibody into the human FRs because the remaining HCDR2 residues have not been found in contact in antigen-antibody complexes of known structures (Almagro, (2004) J Mol Recognit. 17:132). Backumtations were introduced into certain residue positions in the humanized antibodies. PD1B131 backmutations: VH: V37I_Q39L_W47S_R98S, VL: Y49K. PD1B132: VH W47S_R98S, VL: Y49K (residue numbering according to Chothia). Select antibodies were expressed as IgG2sigma/κ. The resulting antibodies were characterized for their binding to recombinant PD-1 and PD-1 expressed on cells (Jurkat cells), and their ligand inhibition (cyno PD-L1 and human PD-L1). Characteristics of select humanized antibodies are shown in Table 23. The VH and the VL sequences of the generated antibodies are shown in Table 24 and Table 25, respectively.

TABLE 23

| | Jurkat | | | | | |
|---|---|---|---|---|---|---|
| | cell | | | | PD-L1 | |
| | binding | Human PD-1 | | | Inhibition, | |
| | relative | Affinity | | | IC₅₀ (ng/ml) | |
| | to | kon | koff | K_{D} | Human | Cyno |
| mAb | PD1B28 | (1/Ms) | (1/s) | (pM) | PD-L1 | PD-L1 |
| PD1B28 | 100% | 9.70E+05 | 1.18E−04 | 122 | 67 | 96 |
| PD1B131 | 100% | 8.27E+05 | 1.05E−04 | 127 | 79 | 96 |
| PD1B132 | 100% | 9.14E+05 | 8.80E−05 | 96 | 55 | 79 |

TABLE 24

| | VH | VL | | VH SEQ |
|---|---|---|---|---|
| mAb | ID | ID | VH sequence | ID NO: |
| PD1B131 | PD1H130 | PD1L62 | EVQLVESGGGLVQPGG | 63 |
| | | | SLRLSCAASGFAFSRY | |
| | | | DMSWIRLAPGKGLESV | |
| | | | AYISGGGANTYYLDNV | |
| | | | KGRFTISRDNAKNSLY | |
| | | | LQMNSLRAEDTAVYYC | |
| | | | ASPYLSYFDVWGQGTL | |
| | | | VTVSS | |
| PD1B132 | PD1H129 | PD1L62 | EVQLVESGGGLVQPGG | 64 |
| | | | SLRLSCAASGFAFSRY | |
| | | | DMSWVRQAPGKGLESV | |
| | | | AYISGGGANTYYLDNV | |
| | | | KGRFTISRDNAKNSLY | |
| | | | LQMNSLRAEDTAVYYC | |
| | | | ASPYLSYFDVWGQGTL | |
| | | | VTVSS | |

TABLE 25

| | VH | VL | | VL SEQ |
|---|---|---|---|---|
| mAb | ID | ID | VL sequence | ID NO: |
| PD1B131 | PD1H130 | PD1L62 | EIVMTQSPATLSVSPG | 65 |
| | | | ERATLSCRASQSLSDY | |
| | | | LHWYQQKPGQAPRLLI | |
| | | | KSASQSISGIPARFSG | |
| | | | SGSGTEFTLTISSLQS | |
| | | | EDFAVYYCQNGHSFPY | |
| | | | TFGQGTKLEIK | |
| PD1B132 | PD1H129 | PD1L62 | EIVMTQSPATLSVSPG | 65 |
| | | | ERATLSCRASQSLSDY | |
| | | | LHWYQQKPGQAPRLLI | |
| | | | KSASQSISGIPARFSG | |
| | | | SGSGTEFTLTISSLQS | |
| | | | EDFAVYYCQNGHSFPY | |
| | | | TFGQGTKLEIK | |

HCDR1
(SEQ ID NO: 66)
RYDMS
HCDR2
(SEQ ID NO: 67)
YISGGGANTYYLDNVKG
HCDR3
(SEQ ID NO: 68)
PYLSYFDV
LCDR1
(SEQ ID NO: 69)
RASQSLSDYLH
LCDR2
(SEQ ID NO: 70)
SASQSIS
LCDR3
(SEQ ID NO: 71)
QNGHSFPYT

### Example 7. Effect of Isotype Switching on Anti-PD-1 Antibody Properties

Variable regions of antibodies PD1B196 and PD1B199 (of IgG2sigma/κisotype) were cloned as IgG4 S228P isotypes and variable regions from antibody PD1B132 (of IgG2) into IgG2sigma isotype to assess possible differences in functionality and developability.

The antibodies were named PD1B244 (PD1B196 VH/VL on IgG4 S228P) PD1B245 (PD1B199 VH/VL on IgG4 S228P) AND PD1B243 (PD1B132 VH/VL on IgG2sigma).

Isotype switch had no consistent effect on the antibody properties however, for some of the antibodies, some change in EC₅₀ values were seen in the CMV assay.

Exemplified below are heavy chain and light chain amino acid sequences of various antibodies. Table 26 shows the summary of the VH, VL, heavy chain and light chain SEQ ID NOs: for select antibodies.

TABLE 26

| | VH | VH | VL | VL | HC | LC |
|---|---|---|---|---|---|---|
| | peptide | SEQ | peptide | SEQ | SEQ | SEQ |
| Antibody | ID | ID NO: | ID | ID NO: | ID NO | ID NO: |
| PD1B114 | PD1H24 | 41 | PH9L3 | 49 | 212 | 213 |
| PD1B149 | PD1H24 | 41 | PD1L128 | 50 | 214 | 215 |
| PD1B160 | PD1H131 | 42 | PD1L101 | 51 | 216 | 217 |
| PD1B162 | PD1H131 | 42 | PD1L67 | 52 | 218 | 219 |
| PD1B164 | PD1H131 | 42 | PD1L71 | 53 | 220 | 221 |
| PD1B183 | PD1H3 | 43 | PD1L109 | 54 | 222 | 223 |
| PD1B184 | PD1H3 | 43 | PD1L128 | 50 | 224 | 225 |
| PD1B185 | PD1H3 | 43 | PD1L132 | 55 | 226 | 227 |
| PD1B192 | PD1H3 | 43 | PD1L133 | 57 | 228 | 229 |
| PD1B243 | PD1H129 | 64 | PD1L62 | 65 | 74 | 75 |
| PD1B244 | PD1H170 | 48 | PD1L148 | 56 | 72 | 73 |
| PD1B245 | PD1H164 | 45 | PD1L86 | 60 | 76 | 77 |

HC of PD1B244
SEQ ID NO: 72
QVQLVQSGAEVKKPGSSVKVSCKASGGTFSSYAISWVRQAPGQGLEW
MGGIIPIFDTANYAQKFQGRVTITADESTSTAYMELSSLRSEDTAVY
YCARPGLAAAYDTGSLDYWGQGTLVTVSSASTKGPSVFPLAPCSRST
SESTAALGCLVKDYFPEPVTVSWNSGALTSGVHTFPAVLQSSGLYSL
SSVVTVPSSSLGTKTYTCNVDHKPSNTKVDKRVESKYGPPCPPCPAP
EFLGGPSVFLFPPKPKDTLMISRTPEVTCVVVDVSQEDPEVQFNWYV
DGVEVHNAKTKPREEQFNSTYRVVSVLTVLHQDWLNGKEYKCKVSNK
GLPSSIEKTISKAKGQPREPQVYTLPPSQEEMTKNQVSLTCLVKGFY
PSDIAVEWESNGQPENNYKTTPPVLDSDGSFFLYSRLTVDKSRWQEG
NVFSCSVMHEALHNHYTQKSLSLSLGK
LC of PD1B244
SEQ ID NO: 73
EIVLTQSPATLSLSPGERATLSCRASQSVRSYLAWYQQKPGQAPRLL
IYDASNRATGIPARFSGSGSGTDFTLTISSLEPEDFAVYYCQQRNYW
PLTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
HC of PD1B243
SEQ ID NO: 74
EVQLVESGGGLVQPGGSLRLSCAASGFAFSRYDMSWVRQAPGKGLES
VAYISGGGANTYYLDNVKGRFTISRDNAKNSLYLQMNSLRAEDTAVY
YCASPYLSYFDVWGQGTLVTVSSASTKGPSVFPLAPCSRSTSESTAA
LGCLVKDYFPEPVTVSWNSGALTSGVHTFPAVLQSSGLYSLSSVVTV
PSSSLGTKTYTCNVDHKPSNTKVDKRVESKYGPPCPPCPAPEFLGGP
SVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAV
EWESNGQPENNYKTTPPVLDSDGSFFLYSRLTVDKSRWQEGNVFSCS
VMHEALHNHYTQKSLSLSLGK
LC of PD1B243
SEQ ID NO: 75
EIVMTQSPATLSVSPGERATLSCRASQSLSDYLHWYQQKPGQAPRLL
IKSASQSISGIPARFSGSGSGTEFTLTISSLQSEDFAVYYCQNGHSF
PYTFGQGTKLEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
HC of PD1B245
SEQ ID NO: 76
QVQLVQSGAEVKKPGSSVKVSCKASGGTFSDYVISWVRQAPGQGLEW
MGGIIPIYGTANYAQKFQGRVTITADESTSTAYMELSSLRSEDTAVY
YCARGTLDRTGHLDYWGQGTLVTVSSASTKGPSVFPLAPCSRSTSES
TAALGCLVKDYFPEPVTVSWNSGALTSGVHTFPAVLQSSGLYSLSSV
VTVPSSSLGTKTYTCNVDHKPSNTKVDKRVESKYGPPCPPCPAPEFL
GGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLP
SSIEKTISKAKGQPREPQVYTLPPSQEEMTKNQVSLTCLVKGFYPSD
IAVEWESNGQPENNYKTTPPVLDSDGSFFLYSRLTVDKSRWQEGNVF
SCSVMHEALHNHYTQKSLSLSLGK
LC of PD1B245
SEQ ID NO: 77
EIVLTQSPATLSLSPGERATLSCRASQSVSSYLAWYQQKPGQAPRLL
IHDASTRATGIPARFSGSGSGTDFTLTISSLEPEDFAVYYCQQRNYW
PLTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
HC of PD1B114
SEQ ID NO: 212
QVQLVQSGAEVKKPGSSVKVSCKASGGTFSSYAISWVRQAPGQGLEW
MGGIIPIFGTANYAQKFQGRVTITADESTSTAYMELSSLRSEDTAVY
YCARPGLAAAYDTGNLDYWGQGTLVTVSSASTKGPSVFPLAPCSRST
SESTAALGCLVKDYFPEPVTVSWNSGALTSGVHTFPAVLQSSGLYSL
SSVVTVTSSNFGTQTYTCNVDHKPSNTKVDKTVERKCCVECPPCPAP
PAAASSVFLFPPKPKDTLMISRTPEVTCVVVDVSAEDPEVQFNWYVD
GVEVHNAKTKPREEQFNSTFRVVSVLTVLHQDWLNGKEYKCKVSNKG
LPSSIEKTISKTKGQPREPQVYTLPPSREEMTKNQVSLTCLVKGFYP
SDIAVEWESNGQPENNYKTTPPMLDSDGSFFLYSKLTVDKSRWQQGN
VFSCSVMHEALHNHYTQKSLSLSPGK
LC of PD1B114
SEQ ID NO: 213
EIVLTQSPATLSLSPGERATLSCRASQSVSSYLAWYQQKPGQAPRLL
IYDASNRATGIPARFSGSGSGTDFTLTISSLEPEDFAVYYCQQRSNW
PLTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
HC of PD1B149
SEQ ID NO 214
QVQLVQSGAEVKKPGSSVKVSCKASGGTFSSYAISWVRQAPGQGLEW
MGGIIPIFGTANYAQKFQGRVTITADESTSTAYMELSSLRSEDTAVY
YCARPGLAAAYDTGNLDYWGQGTLVTVSSASTKGPSVFPLAPCSRST
SESTAALGCLVKDYFPEPVTVSWNSGALTSGVHTFPAVLQSSGLYSL
SSVVTVTSSNFGTQTYTCNVDHKPSNTKVDKTVERKCCVECPPCPAP
PAAASSVFLFPPKPKDTLMISRTPEVTCVVVDVSAEDPEVQFNWYVD
GVEVHNAKTKPREEQFNSTFRVVSVLTVLHQDWLNGKEYKCKVSNKG
LPSSIEKTISKTKGQPREPQVYTLPPSREEMTKNQVSLTCLVKGFYP
SDIAVEWESNGQPENNYKTTPPMLDSDGSFFLYSKLTVDKSRWQQGN
VFSCSVMHEALHNHYTQKSLSLSPGK
LC of PD1B149
SEQ ID NO: 215
EIVLTQSPATLSLSPGERATLSCRASQSVRNYLAWYQQKPGQAPRLL
IHDASNRATGIPARFSGSGSGTDFTLTISSLEPEDFAVYYCQQRNYW
PLTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
HC of PD1B160
SEQ ID NO: 216
QVQLVQSGAEVKKPGSSVKVSCKASGGTFSSYAISWVRQAPGQGLEW
MGGIIPIFDTANYAQKFQGRVTITADESTSTAYMELSSLRSEDTAVY
YCARPGLAAAYDTGNLDYWGQGTLVTVSSASTKGPSVFPLAPCSRST
SESTAALGCLVKDYFPEPVTVSWNSGALTSGVHTFPAVLQSSGLYSL
SSVVTVTSSNFGTQTYTCNVDHKPSNTKVDKTVERKCCVECPPCPAP
PAAASSVFLFPPKPKDTLMISRTPEVTCVVVDVSAEDPEVQFNWYVD
GVEVHNAKTKPREEQFNSTFRVVSVLTVLHQDWLNGKEYKCKVSNKG
LPSSIEKTISKTKGQPREPQVYTLPPSREEMTKNQVSLTCLVKGFYP
SDIAVEWESNGQPENNYKTTPPMLDSDGSFFLYSKLTVDKSRWQQGN
VFSCSVMHEALHNHYTQKSLSLSPGK
LC of PD1B160
SEQ ID NO: 217
EIVLTQSPATLSLSPGERATLSCRASQSVDSYLAWYQQKPGQAPRLL
IKDASDRATGIPARFSGSGSGTDFTLTISSLEPEDFAVYYCQQRGNW
PLTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
HC of PD1B162
SEQ ID NO: 218
QVQLVQSGAEVKKPGSSVKVSCKASGGTFSSYAISWVRQAPGQGLEW
MGGIIPIFDTANYAQKFQGRVTITADESTSTAYMELSSLRSEDTAVY
YCARPGLAAAYDTGNLDYWGQGTLVTVSSASTKGPSVFPLAPCSRST
SESTAALGCLVKDYFPEPVTVSWNSGALTSGVHTFPAVLQSSGLYSL
SSVVTVTSSNFGTQTYTCNVDHKPSNTKVDKTVERKCCVECPPCPAP
PAAASSVFLFPPKPKDTLMISRTPEVTCVVVDVSAEDPEVQFNWYVD
GVEVHNAKTKPREEQFNSTFRVVSVLTVLHQDWLNGKEYKCKVSNKG
LPSSIEKTISKTKGQPREPQVYTLPPSREEMTKNQVSLTCLVKGFYP
SDIAVEWESNGQPENNYKTTPPMLDSDGSFFLYSKLTVDKSRWQQGN
VFSCSVMHEALHNHYTQKSLSLSPGK
LC of PD1B162
SEQ ID NO: 219
EIVLTQSPATLSLSPGERATLSCRASQSVDSYLAWYQQKPGQAPRLL
IYDASNRATGIPARFSGSGSGTDFTLTISSLEPEDFAVYYCQQREYW
PLTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
HC of PD1B164
SEQ ID NO: 220
QVQLVQSGAEVKKPGSSVKVSCKASGGTFSSYAISWVRQAPGQGLEW
MGGIIPIFDTANYAQKFQGRVTITADESTSTAYMELSSLRSEDTAVY
YCARPGLAAAYDTGNLDYWGQGTLVTVSSASTKGPSVFPLAPCSRST
SESTAALGCLVKDYFPEPVTVSWNSGALTSGVHTFPAVLQSSGLYSL
SSVVTVTSSNFGTQTYTCNVDHKPSNTKVDKTVERKCCVECPPCPAP
PAAASSVFLFPPKPKDTLMISRTPEVTCVVVDVSAEDPEVQFNWYVD
GVEVHNAKTKPREEQFNSTFRVVSVLTVLHQDWLNGKEYKCKVSNKG
LPSSIEKTISKTKGQPREPQVYTLPPSREEMTKNQVSLTCLVKGFYP
SDIAVEWESNGQPENNYKTTPPMLDSDGSFFLYSKLTVDKSRWQQGN
VFSCSVMHEALHNHYTQKSLSLSPGK
LC of PD1B164
SEQ ID NO: 221
EIVLTQSPATLSLSPGERATLSCRASQSVRSYLAWYQQKPGQAPRLL
IYDASYRATGIPARFSGSGSGTDFTLTISSLEPEDFAVYYCQQRDYW
PLTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
HC of PD1B183
SEQ ID NO: 222
QVQLVQSGAEVKKPGSSVKVSCKASGGTFSSYAISWVRQAPGQGLEW
MGGIIPIFGTANYAQKFQGRVTITADESTSTAYMELSSLRSEDTAVY
YCARPGLAAAYDTGSLDYWGQGTLVTVSSASTKGPSVFPLAPCSRST
SESTAALGCLVKDYFPEPVTVSWNSGALTSGVHTFPAVLQSSGLYSL
SSVVTVTSSNFGTQTYTCNVDHKPSNTKVDKTVERKCCVECPPCPAP
PAAASSVFLFPPKPKDTLMISRTPEVTCVVVDVSAEDPEVQFNWYVD
GVEVHNAKTKPREEQFNSTFRVVSVLTVLHQDWLNGKEYKCKVSNKG
LPSSIEKTISKTKGQPREPQVYTLPPSREEMTKNQVSLTCLVKGFYP
SDIAVEWESNGQPENNYKTTPPMLDSDGSFFLYSKLTVDKSRWQQGN
VFSCSVMHEALHNHYTQKSLSLSPGK
LC of PD1B183
SEQ ID NO: 223
EIVLTQSPATLSLSPGERATLSCRASQSVSSYLAWYQQKPGQAPRLL
IKDASNRATGIPARFSGSGSGTDFTLTISSLEPEDFAVYYCQQRGYW
PLTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
HC of PD1B184
SEQ ID NO: 224
QVQLVQSGAEVKKPGSSVKVSCKASGGTFSSYAISWVRQAPGQGLEW
MGGIIPIFGTANYAQKFQGRVTITADESTSTAYMELSSLRSEDTAVY
YCARPGLAAAYDTGSLDYWGQGTLVTVSSASTKGPSVFPLAPCSRST
SESTAALGCLVKDYFPEPVTVSWNSGALTSGVHTFPAVLQSSGLYSL
SSVVTVTSSNFGTQTYTCNVDHKPSNTKVDKTVERKCCVECPPCPAP
PAAASSVFLFPPKPKDTLMISRTPEVTCVVVDVSAEDPEVQFNWYVD
GVEVHNAKTKPREEQFNSTFRVVSVLTVLHQDWLNGKEYKCKVSNKG
LPSSIEKTISKTKGQPREPQVYTLPPSREEMTKNQVSLTCLVKGFYP
SDIAVEWESNGQPENNYKTTPPMLDSDGSFFLYSKLTVDKSRWQQGN
VFSCSVMHEALHNHYTQKSLSLSPGK
LC of PD1B184
SEQ ID NO: 225
EIVLTQSPATLSLSPGERATLSCRASQSVRNYLAWYQQKPGQAPRLL
IHDASNRATGIPARFSGSGSGTDFTLTISSLEPEDFAVYYCQQRNYW
PLTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
HC of PD1B185
SEQ ID NO: 226
QVQLVQSGAEVKKPGSSVKVSCKASGGTFSSYAISWVRQAPGQGLEW
MGGIIPIFGTANYAQKFQGRVTITADESTSTAYMELSSLRSEDTAVY
YCARPGLAAAYDTGSLDYWGQGTLVTVSSASTKGPSVFPLAPCSRST
SESTAALGCLVKDYFPEPVTVSWNSGALTSGVHTFPAVLQSSGLYSL
SSVVTVTSSNFGTQTYTCNVDHKPSNTKVDKTVERKCCVECPPCPAP
PAAASSVFLFPPKPKDTLMISRTPEVTCVVVDVSAEDPEVQFNWYVD
GVEVHNAKTKPREEQFNSTFRVVSVLTVLHQDWLNGKEYKCKVSNKG
LPSSIEKTISKTKGQPREPQVYTLPPSREEMTKNQVSLTCLVKGFYP
SDIAVEWESNGQPENNYKTTPPMLDSDGSFFLYSKLTVDKSRWQQGN
VFSCSVMHEALHNHYTQKSLSLSPGK
LC of PD1B185
SEQ ID NO: 227
EIVLTQSPATLSLSPGERATLSCRASQSVRNYLAWYQQKPGQAPRLL
IYDASDRATGIPARFSGSGSGTDFTLTISSLEPEDFAVYYCQQRWNW
PLTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC
HC of PD1B192
SEQ ID NO: 228
QVQLVQSGAEVKKPGSSVKVSCKASGGTFSSYAISWVRQAPGQGLEW
MGGIIPIFGTANYAQKFQGRVTITADESTSTAYMELSSLRSEDTAVY
YCARPGLAAAYDTGSLDYWGQGTLVTVSSASTKGPSVFPLAPCSRST
SESTAALGCLVKDYFPEPVTVSWNSGALTSGVHTFPAVLQSSGLYSL
SSVVTVTSSNFGTQTYTCNVDHKPSNTKVDKTVERKCCVECPPCPAP
PAAASSVFLFPPKPKDTLMISRTPEVTCVVVDVSAEDPEVQFNWYVD
GVEVHNAKTKPREEQFNSTFRVVSVLTVLHQDWLNGKEYKCKVSNKG
LPSSIEKTISKTKGQPREPQVYTLPPSREEMTKNQVSLTCLVKGFYP
SDIAVEWESNGQPENNYKTTPPMLDSDGSFFLYSKLTVDKSRWQQGN
VFSCSVMHEALHNHYTQKSLSLSPGK
LC or PD1B192
SEQ ID NO: 229
EIVLTQSPATLSLSPGERATLSCRASQSVDSYLAWYQQKPGQAPRLL
IHDASNRATGIPARFSGSGSGTDFTLTISSLEPEDFAVYYCQQRNYW
PLTFGQGTKVEIKRTVAAPSVFIFPPSDEQLKSGTASVVCLLNNFYP
REAKVQWKVDNALQSGNSQESVTEQDSKDSTYSLSSTLTLSKADYEK
HKVYACEVTHQGLSSPVTKSFNRGEC

### Example 8. Characterization of PD-1 Antibodies in Cell-Based Assays

Select antibodies were characterized in MLR and CMV assays using protocols described in Example 1. The EC₅₀ values for IFN-γ induction from MLR and CMV assays are shown in Table 27. In most cases, anti-PD-1 antibodies showed a dose-dependent increase in IFN-γ levels in both MLR and CMV assays.

TABLE 27

| Origin | mAb | MLR EC₅₀, nM | CMV EC₅₀, nM |
|---|---|---|---|
| Phage display | PD1B3 | 0.29 | 0.06 |
| | PD1B91 | 0.05 | 0.03 |
| | PD1B194 | NT | NC |
| | PD1B195 | NT | 1.64 |
| | PD1B196 | 0.14 | 0.31 |
| | PD1B199 | 0.63 | NC |
| | PD1B200 | NT | 3.81 |
| | PD1B201 | NT | 2.60 |
| | PD1B244 | 0.08 | 0.03 |
| HFA | PD1B132 | NT | 0.07 |
| | PD1B243 | 0.07 | 0.02 |

NT: not tested
NC: no convergence
HFA: human framework adaptation

In addition to IFN-γ, secreted levels of additional cytokines were also affected by PD-1 blockade in the two assays. Upon CMV stimulation, anti-PD-1 antibodies led to a dose-dependent induction of TNF-α and IL-4, whereas in the MLR assay they increased TNF-α and IL-2 levels.

### Example 9. Generation of Human Anti-TIM-3 Antibodies Using Phage Display Libraries

The de novo pIX Fab libraries described in Example 2 were panned against the extracellular domain of recombinant human TIM-3-Fc fusion protein (R&D Systems, #2365-TM; residues Ser22-Arg200 of full length TIM-3) (huTIM-3-Fc).

The recombinant protein was biotinylated (bt) and captured on streptavidin magnetic beads (Dynal), then exposed to the de novo pIX Fab libraries at a final concentration of 100 nM. Non-specific phages were washed away in PBS-Tween and bound phages were recovered by infection of MCI061F′ E. coli cells. Phages were amplified from these cells overnight and panning was repeated for a total of three rounds. Following the final round of biopanning, monoclonal Fab was screened for binding to biotinylated human TIM-3-Fc captured on ELISA plates by Streptavidin and secreted Fab was added to the captured antigen, followed by detection of the Fab with Goat Anti human kappa:HRP. Select antibodies were expressed and cloned on various IgG isotypes as indicated below, and characterized further.

### Example 10. Generation of Anti-TIM-3 Antibodies in Mice

Balb/c mice were immunized with recombinant human TIM-3-Fc fusion protein (R&D Systems, catalog #2365-TM) over the course of 18 days. Spleens were harvested, and a B cell enriched population was fused with FO mouse myeloma cells to generate mAb secreting hybridomas. The hybridoma supernatants were screened for binding by ELISA to TIM-3-Fc protein and an irrelevant human IgG1 Fc. TIM-3 specific supernatants were then assayed for the ability to bind to TIM-3 expressing THP-1 cells.

Select mAb HC and LC v-genes were cloned from the TIM-3 positive hybridomas using standard molecular biology techniques (RT-PCR followed by PCR fragment ligation into plasmid expression vectors). mAbs were expressed recombinantly, and the ELISA was repeated to confirm TIM-3 specific binding. Molecular models for murine antibody sequences to be human framework adapted were constructed using MOE (CCG, Montreal) and visually inspected. Potential problem positions that might influence antigen binding, VL/VH packing and/or core residues that might affect domain stabilities were identified. For both VL and VH, multiple human frameworks were proposed with or without back mutations to mouse framework sequences if problem positions were identified. The designed sequences were cloned into heavy and light chain plasmids and expressed in Expi293F cells. Expressed antibody in the culture supernatants were quantified and assessed for binding to HEK293 cells transfected with recombinant human TIM-3.

### Example 11. Isotypes of Anti-TIM-3 Antibodies

The VH and VL of isolated anti-TIM-3 antibodies were cloned onto various heavy chain isotypes, optionally with various Fc substitutions, and allotypes with κ light chains during the course of antibody characterization to evaluate the effect, if any, of isotype switch on functionality or developability of the antibodies. The various isotypes used are shown in Table 28.

TABLE 28

| | Substitution when | |
|---|---|---|
| | compared to wild | |
| Isotype | type* | Purpose of substitution |
| IgG2sigma | V234A, G237A, | Abolishing effector functions |
| | P238S, H268A, | |
| | V309L, A330S, | |
| | P331S | |
| IgG2sigma_K409R | V234A, G237A, | Abolishing effector functions, |
| | P238S, H268A, | improving heterodimer formation in |
| | V309L, A330S, | bispecific antibody |
| | P331S, K409R | |
| IgG2sigma_F405L | V234A, G237A, | Abolishing effector functions, |
| | P238S, H268A, | improving heterodimer formation in |
| | V309L, A330S, | bispecific antibody |
| | P331S, F405L | |
| IgG4_PAA | S228P, F234A, | Antibody stability, abolishing effector |
| | L235A | functions |
| IgG4_PAA_ | S228P, F234A, | Antibody stability, abolishing effector |
| F405L_R409K | L235A, F450L, | functions, improving heterodimer |
| | R409K | formation in bispecific antibody |
| IgG4_S228P | S228P | Antibody stability |
| IgG1 | Wild type | |
| IgG1sigma | L234A, L235A, | Abolishing effector functions |
| | G237A, P238S, | |
| | H268A, A330S, | |
| | P331S | |
| IgG1sigma_K409R | L234A, L235A, | Abolishing effector functions, |
| | G237A, P238S, | improving heterodimer formation in |
| | H268A, A330S, | bispecific antibody |
| | P331S, K409R | |
| IgG1sigma_F405L | L234A, L235A, | Abolishing effector functions, |
| | G237A, P238S, | improving heterodimer formation in |
| | H268A, A330S, | bispecific antibody |
| | P331S, F405L | |
| IgG1_AA | L234A, L235A | Abolishing effector functions |

*Residue numbering according to the EU Index

The various allotypes used in the generated antibodies are shown in Table 29. Some of the antibodies had chimeric allotypes. Antibodies TM3B105 and TM3B403 for example differ by one amino acid substitution in a constant region at position 189. TM3B105 heavy and light chains SEQ ID NOs: 240 and 79, respectively; TM3B403 heavy and light chains SEQ ID NOs: 78 and 79, respectively. The two antibodies are expected to have the same characteristics.

TABLE 29
Isotype/Allotype/Substitutions
IgG2sigma_G2m(n-)/(n)_K409R
IgG2sigma_G2m(n-)_K409R
IgG2sigma_G2m(n-)/(n)
IgG2sigma_F405L
IgG2_K409R
IgG2sigma_G2m(n-)
IgG2
IgG4_S228P
IgG4_S228P_F405L_R409K
IgG4_nG4m(a)_PAA_F405L_R409K
IgG4_PAA
IgG1sigma
IgG1_G1m(17)
IgG1_ Glm(17,1)_AA

In general, anti-TIM-3 antibodies with IgG2sigma Fc had greater activity in the CMV assay than anti-TIM-3 antibodies with huIgG4 Fc. In addition, antibodies with huIgG2 Fe demonstrated functionality that was intermediate between IgG2sigma and IgG4. Allotype had no effect on antibody activity.

### Example 12. Structural Characterization of Anti-TIM-3 Antibodies

The cDNA sequences and amino acid translations of the antibodies were obtained using standard techniques throughout the generation of the antibodies using various campaigns. After polypeptide sequence determination, some antibody cDNAs encoding the variable regions or full length antibodies were codon optimized using standard methods for scale-up expression. Antibodies TM3B103, TM3B105, M3B108, TM3B109 and TM3B1113 were isolated from phage display libraries. Antibodies TM3B189, TM3B190, TM3B193, TM3B195 and TM3B196 were generated by immunizing mice.
Table 30 shows the HCDR1 sequences of select anti-TIM-3 antibodies.
Table 31 shows the HCDR2 sequences of select anti-TIM-3 antibodies.
Table 32 shows the HCDR3 sequences of select anti-TIM-3 antibodies.
Table 33 shows the LCDR1 sequences of select anti-TIM-3 antibodies.
Table 34 shows the LCDR2 sequences of select anti-TIM-3 antibodies.
Table 35 shows the LCDR3 sequences of select anti-TIM-3 antibodies.
Table 36 shows the VH sequences of select anti-TIM-3 antibodies.
Table 37 shows the VL sequences of select anti-TIM-3 antibodies.
Table 38 shows the frameworks of select anti-TIM-3 antibodies.

TABLE 30

| | HCDR1 | | | | | | |
|---|---|---|---|---|---|---|---|
| mAb | | | | | | | SEQ |
| name | Sequence | | | | | | ID NO: |
| TM3B103 | N | Y | W | M | S | | 90 |
| TM3B105 | S | Y | A | M | S | | 91 |
| TM3B109 | S | Y | A | M | S | | 91 |
| TM3B108 | G | Y | W | M | H | | 92 |
| TM3B113 | D | Y | W | M | S | | 93 |
| TM3B189 | S | Y | V | M | Y | | 94 |
| TM3B190 | S | D | Y | A | W | N | 95 |
| TM3B193 | D | T | Y | L | H | | 96 |
| TM3B195 | S | Y | W | M | Q | | 97 |
| TM3B196 | S | Y | G | V | H | | 98 |
| TM3B291 | S | Y | W | M | Q | | 97 |

TABLE 31

| | HCDR2 | | | | | | | | | | | | | | | | | |
|---|---|---|---|---|---|---|---|---|---|---|---|---|---|---|---|---|---|---|
| | | | | | | | | | | | | | | | | | | SEQ |
| | | | | | | | | | | | | | | | | | | ID |
| mAb | Sequence | | | | | | | | | | | | | | | | | NO: |
| TM3B103 | A | I | S | G | S | G | G | S | T | Y | Y | A | D | S | V | K | G | 99 |
| TM3B105 | A | I | S | G | S | G | G | S | T | Y | Y | A | D | S | V | K | G | 99 |
| TM3B109 | A | I | S | G | S | G | G | S | T | Y | Y | A | D | S | V | K | G | 99 |
| TM3B108 | A | I | S | Y | S | G | S | S | T | Y | Y | A | D | S | V | K | G | 100 |
| TM3B113 | V | I | K | Y | S | G | G | S | K | Y | Y | A | D | S | V | K | G | 101 |
| TM3B189 | Y | I | N | P | Y | N | D | G | T | K | Y | N | E | K | F | K | G | 102 |
| TM3B190 | Y | I | N | Y | S | G | R | T | S | Y | N | P | S | L | K | S | | 103 |
| TM3B193 | R | I | D | P | T | N | G | N | I | K | Y | D | P | K | F | Q | G | 104 |
| TM3B195 | A | I | Y | P | G | D | G | D | I | R | Y | T | Q | N | F | K | G | 105 |
| TM3B196 | V | I | W | S | D | G | S | T | T | Y | N | S | A | L | K | S | | 106 |
| TM3B291 | A | I | Y | P | G | D | G | D | I | R | Y | T | Q | N | F | K | G | 105 |

TABLE 32

| | HCDR3 | | | | | | | | | | | | | | | |
|---|---|---|---|---|---|---|---|---|---|---|---|---|---|---|---|---|
| | | | | | | | | | | | | | | | | SEQ |
| | | | | | | | | | | | | | | | | ID |
| mAb | Sequence | | | | | | | | | | | | | | | NO: |
| TM3B103 | D | H | W | D | P | N | F | L | D | Y | | | | | | 107 |
| TM3B105 | S | P | Y | A | P | L | D | Y | | | | | | | | 108 |
| TM3B109 | N | E | E | P | D | D | R | L | D | Y | | | | | | 109 |
| TM3B108 | G | T | N | W | L | D | Y | | | | | | | | | 110 |
| TM3B113 | E | L | E | G | V | F | D | Y | | | | | | | | 111 |
| TM3B189 | D | D | Y | D | V | A | P | F | A | Y | | | | | | 112 |
| TM3B190 | G | G | N | F | D | Y | | | | | | | | | | 113 |
| TM3B193 | P | Y | Y | G | F | F | D | Y | | | | | | | | 114 |
| TM3B195 | W | E | K | S | T | T | V | V | Q | R | N | Y | F | D | Y | 115 |
| TM3B196 | Q | A | N | Y | R | Y | D | S | A | M | D | Y | | | | 116 |
| TM3B291 | W | E | K | S | T | T | V | V | Q | R | N | Y | F | D | Y | 115 |

TABLE 33

| | LCDR1 | | | | | | | | | | | | | | | | | |
|---|---|---|---|---|---|---|---|---|---|---|---|---|---|---|---|---|---|---|
| | | | | | | | | | | | | | | | | | | SEQ |
| | | | | | | | | | | | | | | | | | | ID |
| mAb | Sequence | | | | | | | | | | | | | | | | | NO: |
| TM3B103 | R | A | S | Q | S | V | S | S | S | Y | L | A | | | | | | 117 |
| TM3B105 | R | A | S | Q | S | V | N | D | Y | L | A | | | | | | | 118 |
| TM3B109 | K | S | S | Q | S | V | L | A | S | S | N | N | K | N | Y | L | A | 119 |
| TM3B108 | R | A | S | Q | S | V | S | S | S | Y | L | A | | | | | | 117 |
| TM3B113 | R | A | S | Q | S | V | S | N | S | T | L | A | | | | | | 120 |
| TM3B189 | R | A | S | E | S | L | D | S | Y | G | N | S | Y | I | H | | | 121 |
| TM3B190 | Q | A | T | Q | D | I | V | K | N | L | N | | | | | | | 122 |
| TM3B193 | K | A | S | Q | D | V | N | T | A | V | A | | | | | | | 123 |
| TM3B195 | K | A | S | E | N | V | G | T | F | V | S | | | | | | | 124 |
| TM3B196 | K | A | S | Q | S | V | D | Y | D | G | D | S | Y | M | N | | | 125 |
| TM3B291 | K | A | S | E | N | V | G | T | F | V | S | | | | | | | 124 |

TABLE 34

| | LCDR2 | |
|---|---|---|
| | | SEQ |
| | | ID |
| mAb | Sequence | NO: |

| | | | | | | | | |
|---|---|---|---|---|---|---|---|---|
| TM3B103 | G | A | S | S | R | A | T | 126 |
| TM3B105 | D | A | S | N | R | A | T | 127 |
| TM3B109 | W | A | S | T | R | E | S | 128 |
| TM3B108 | G | A | S | S | R | A | T | 126 |
| TM3B113 | T | A | S | S | R | A | T | 129 |
| TM3B189 | L | A | S | N | L | E | S | 130 |
| TM3B190 | Y | V | T | E | L | A | E | 131 |
| TM3B193 | S | A | T | Y | R | Y | T | 132 |
| TM3B195 | G | A | S | N | R | Y | T | 133 |
| TM3B196 | T | A | A | N | L | Q | S | 134 |
| TM3B291 | G | A | S | N | R | Y | T | 133 |

TABLE 35

| | LCDR3 | | | | | | | | | |
|---|---|---|---|---|---|---|---|---|---|---|
| | | | | | | | | | | SEQ |
| | | | | | | | | | | ID |
| mAb | Sequence | | | | | | | | | NO: |
| TM3B103 | Q | Q | Y | G | S | S | P | L | T | 135 |
| TM3B105 | Q | Q | G | G | H | A | P | I | T | 136 |
| TM3B109 | Q | Q | Y | Y | S | T | P | L | T | 137 |
| TM3B108 | Q | Q | Y | G | S | S | P | L | T | 135 |
| TM3B113 | Q | Q | S | Y | T | S | P | W | T | 139 |
| TM3B189 | Q | Q | N | N | E | D | P | F | T | 140 |
| TM3B190 | L | Q | F | Y | E | F | P | L | T | 141 |
| TM3B193 | Q | Q | H | Y | S | T | P | Y | T | 142 |
| TM3B195 | G | Q | S | Y | S | Y | P | T | | 143 |
| TM3B196 | Q | Q | S | N | E | D | P | F | T | 144 |
| TM3B291 | G | Q | S | Y | S | Y | P | T | | 143 |

TABLE 36

| mAb | VH | | SEQ |
|---|---|---|---|
| name | name | VH sequence | ID NO: |
| TM3B103 | TM3H21 | EVQLLESGGGLVQPGGSLRLS | 145 |
| | | CAASGFTFSNYWMSWVRQAPG | |
| | | KGLEWVSAISGSGGSTYYADS | |
| | | VKGRFTISRDNSKNTLYLQMN | |
| | | SLRAEDTAVYYCAKDHWDPNF | |
| | | LDYWGQGTLVTVSS | |
| TM3B105 | TM3H24 | EVQLLESGGGLVQPGGSLRLS | 146 |
| | | CAASGFTFSSYAMSWVRQAPG | |
| | | KGLEWVSAISGSGGSTYYADS | |
| | | VKGRFTISRDNSKNTLYLQMN | |
| | | SLRAEDTAVYYCAKSPYAPLD | |
| | | YWGQGTLVTVSS | |
| TM3B108 | TM3H30 | EVQLLESGGGLVQPGGSLRLS | 147 |
| | | CAASGFTFSGYWMHWVRQAPG | |
| | | KGLEWVSAISYSGSSTYYADS | |
| | | VKGRFTISRDNSKNTLYLQMN | |
| | | SLRAEDTAVYYCAKGTNWLDY | |
| | | WGQGTLVTVSS | |
| TM3B109 | TM3H31 | EVQLLESGGGLVQPGGSLRLS | 148 |
| | | CAASGFTFSSYAMSWVRQAPG | |
| | | KGLEWVSAISGSGGSTYYADS | |
| | | VKGRFTISRDNSKNTLYLQMN | |
| | | SLRAEDTAVYYCAKNEEPDDR | |
| | | LDYWGQGTLVTVSS | |
| TM3B113 | TM3H65 | EVQLLESGGGLVQPGGSLRLS | 149 |
| | | CAASGFTFSDYWMSWVRQAPG | |
| | | KGLEWVSVIKYSGGSKYYADS | |
| | | VKGRFTISRDNSKNTLYLQMN | |
| | | SLRAEDTAVYYCAKELEGVFD | |
| | | YWGQGTLVTVSS | |
| TM3B189 | TM3H141 | EVQLQQSGPELLKPGASVKMS | 150 |
| | | CKASGYTFTSYVMYWVKQKPG | |
| | | QGLEWIGYINPYNDGTKYNEK | |
| | | FKGKATLTSDKSSSTAYMELS | |
| | | RLTSEDSAVYYCTRDDYDVAP | |
| | | FAYWGQGTLVTVSA | |
| TM3B190 | TM3H96 | DVQLQESGPGLVKPSQSLSLT | 151 |
| | | CTVTGYSITSDYAWNWIRQFP | |
| | | GNKLEWMGYINYSGRTSYNPS | |
| | | LKSRISITRDTSKNQFFLQLN | |
| | | SVTTEDTATYYCTSGGNFDYW | |
| | | GQGTTLTVSS | |
| TM3B193 | TM3H99 | EVQLQQSGAELVKPGASVKLS | 152 |
| | | CTASGFHIKDTYLHWVKQRPE | |
| | | QGLEWIGRIDPTNGNIKYDPK | |
| | | FQGKATITSDTSSNTAYLQLS | |
| | | SLTSEDTAVYYCARPYYGFFD | |
| | | YWGQGTTLTVSS | |
| TM3B195 | TM3H144 | EVQLQQSGAELARPGASVKLS | 153 |
| | | CKASGYTFTSYWMQWVKQRPG | |
| | | QGLEWIGAIYPGDGDIRYTQN | |
| | | FKGKATLTADKSSSTAYMQLS | |
| | | SLASEDSAVYYCARWEKSTTV | |
| | | VQRNYFDYWGQGTTLTVSS | |
| | | CORRECT? | |
| TM3B196 | TM3H102 | QVQLKESGPGLVAPSQSLSIT | 154 |
| | | CTISGFSLTSYGVHWVRQPPG | |
| | | KGLEWLVVIWSDGSTTYNSAL | |
| | | KSRLSISKDNSKSQVFLKMNS | |
| | | LQTDDTAMYYCARQANYRYDS | |
| | | AMDYWGQGTSVTVSS | |
| TM3B291 | TM3H162 | EVQLVQSGAEVKKPGESLKIS | 172 |
| | | CKGSGYSFTSYWMQWVRQMPG | |
| | | KGLEWMGAIYPGDGDIRYTQN | |
| | | FKGQVTISADKSISTAYLQWS | |
| | | SLKASDTAMYYCARWEKSTTV | |
| | | VQRNYFDYWGQGTTVTVSS | |

TABLE 37

| mAb | VL | | SEQ |
|---|---|---|---|
| name | name | VL sequence | ID NO: |
| TM3B103 | PH9L1 | EIVLTQSPGTLSLSPGERATL | 155 |
| | | SCRASQSVSSSYLAWYQQKPG | |
| | | QAPRLLIYGASSRATGIPDRF | |
| | | SGSGSGTDFTLTISRLEPEDF | |
| | | AVYYCQQYGSSPLTFGQGTKV | |
| | | EIK | |
| TM3B105 | TM3L33 | EIVLTQSPATLSLSPGERATL | 156 |
| | | SCRASQSVNDYLAWYQQKPGQ | |
| | | APRLLIYDASNRATGIPARFS | |
| | | GSGSGTDFTLTISSLEPEDFA | |
| | | VYYCQQGGHAPITFGQGTKVE | |
| | | IK | |
| TM3B108 | PH9L1 | EIVLTQSPGTLSLSPGERATL | 155 |
| | | SCRASQSVSSSYLAWYQQKPG | |
| | | QAPRLLIYGASSRATGIPDRF | |
| | | SGSGSGTDFTLTISRLEPEDF | |
| | | AVYYCQQYGSSPLTFGQGTKV | |
| | | EIK | |
| TM3B109 | PYYL6 | DIVMTQSPDSLAVSLGERATI | 157 |
| | | NCKSSQSVLASSNNKNYLAWY | |
| | | QQKPGQPPKLLIYWASTRESG | |
| | | VPDRFSGSGSGTDFTLTISSL | |
| | | QAEDVAVYYCQQYYSTPLTFG | |
| | | QGTKVEIK | |
| TM3B113 | TM3L12 | EIVLTQSPGTLSLSPGERATL | 158 |
| | | SCRASQSVSNSTLAWYQQKPG | |
| | | QAPRLLIYTASSRATGIPDRF | |
| | | SGSGSGTDFTLTISRLEPEDF | |
| | | AVYYCQQSYTSPWTFGQGTKV | |
| | | EIK | |
| TM3B189 | TM3L61 | DIVLTQSPASLAVSLGQRATI | 159 |
| | | SCRASESLDSYGNSYIHWYQQ | |
| | | KPGQPPKLLIYLASNLESGVP | |
| | | ARFSGSGSKTDFTLTIDPVEA | |
| | | DDPATYYCQQNNEDPFTFGSG | |
| | | TKLEIK | |
| TM3B190 | TM3L62 | DIVMTQSPSSMSASLGDRITI | 160 |
| | | TCQATQDIVKNLNWYQQKPGK | |
| | | PPSFLIHYVTELAEGVPSRFS | |
| | | GSGSGSDYSLTISNLESEDFA | |
| | | DYYCLQFYEFPLTFGAGTKLE | |
| | | LK | |
| TM3B193 | TM3L52 | DIVMTQSHKFMSTSVGDRVSI | 161 |
| | | TCKASQDVNTAVAWYQQKPGQ | |
| | | SPKLLIYSATYRYTGVPDRFT | |
| | | GSGSGTDFTFTISSVQAEDLA | |
| | | VYYCQQHYSTPYTFGSGTKLE | |
| | | IK | |
| TM3B195 | TM3L67 | DVQMIQSPKSMSMSVGERVTL | 162 |
| | | SCKASENVGTFVSWYQQKPDQ | |
| | | SPKLLIYGASNRYTGVPDRFT | |
| | | GSGSATDFTLTISSVQAEDLA | |
| | | DYHCGQSYSYPTFGSGTKLEM | |
| TM3B196 | TM3L64 | DIQMTQSPASLAVSLGQRATI | 163 |
| | | SCKASQSVDYDGDSYMNWYQQ | |
| | | KPGQPPKLLIYTAANLQSGIP | |
| | | ARFSGSGSGTDFTLNIHPVEE | |
| | | EDAATYYCQQSNEDPFTFGSG | |
| | | TKLEIK | |
| TM3B291 | TM3L85 | DIQMTQSPSSLSASVGDRVTI | 173 |
| | | TCKASENVGTFVSWYQQKPGK | |
| | | APKLLIYGASNRYTGVPSRFS | |
| | | GSGSGTDFTLTISSLQPEDFA | |
| | | TYYCGQSYSYPTFGQGTKLEI | |
| | | K | |

TABLE 38

| | | VH framework | | | VL framework | |
|---|---|---|---|---|---|---|
| | | | SEQ | | | SEQ |
| mAb | VH | | ID | VL | | ID |
| name | name | Name | NO: | name | Name | NO: |
| TM3B103 | TM3H21 | IGHV3-23 | 174 | PH9L1 | IGKV3-20 | 180 |
| TM3B105 | TM3H24 | IGHV3-23 | 174 | TM3L33 | IGKV3-11 | 171 |
| TM3B108 | TM3H30 | IGHV3-23 | 174 | PH9L1 | IGKV3-20 | 180 |
| TM3B109 | TM3H31 | IGHV3-23 | 174 | PYYL6 | IGKV4-1 | 181 |
| TM3B113 | TM3H65 | IGHV3-23 | 174 | TM3L12 | IGKV3-20 | 180 |
| TM3B189 | TM3H141 | IGHV1-02 | 175 | TM3L61 | IGKV4-1 | 181 |
| TM3B190 | TM3H96 | IGHV4-30 | 176 | TM3L62 | IGKV1-39 | 182 |
| TM3B193 | TM3H99 | IGHV1-03 | 177 | TM3L52 | IGKV1-33 | 183 |
| TM3B195 | TM3H144 | IGHV1-03 | 177 | TM3L67 | IGKV1-39 | 182 |
| TM3B196 | TM3H102 | IGHV2-26 | 178 | TM3L64 | IGKV4-1 | 181 |
| TMB291 | TM3H162 | IGHV5-51 | 179 | TM3L85 | IGKV1-39 | 182 |

IGHV3-23
SEQ ID NO: 174
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
IGHV1-02
SEQ ID NO: 175
QVQLVQSGAEVKKPGASVKVSCKASGYTFTGYYMHWVRQAPGQGLEWMG
RINPNSGGTNYAQKFQGRVTSTRDTSISTAYMELSRLRSDDTVVYYCAR
IGHV4-30
SEQ ID NO: 176
QVQLQESGPGLVKPSQTLSLTCTVSGGSISSGDYYWSWIRQPPGKGLEW
IGYIYYSGSTYYNPSLKSRVTISVDTSKNQFSLKLSSVTAADTAVYYCA
R
IGHV1-03
SEQ ID NO: 177
QVQLVQSGAEVKKPGASVKVSCKASGYTFTSYAMHWVRQAPGQRLEWMG
WINAGNGNTKYSQKFQGRVTITRDTSASTAYMELSSLRSEDTAVYYCAR
IGHV2-26
SEQ ID NO: 178
QVTLKESGPVLVKPTETLTLTCTVSGFSLSNARMGVSWIRQPPGKALEW
LAHIFSNDEKSYSTSLKSRLTISKDTSKSQVVLTMTNMDPVDTATYYCA
RI
IGHV5-51
SEQ ID NO: 179
EVQLVQSGAEVKKPGESLKISCKGSGYSFTSYWIGWVRQMPGKGLEWMG
IIYPGDSDTRYSPSFQGQVTISADKSISTAYLQWSSLKASDTAMYYCAR
IGKV3-20
SEQ ID NO: 180
EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSP
IGKV3-11
SEQ ID NO: 171
EIVLTQSPATLSLSPGERATLSCRASQSVSSYLAWYQQKPGQAPRLLIY
DASNRATGIPARFSGSGSGTDFTLTISSLEPEDFAVYYCQQRSNWP
IGKV4-1
SEQ ID NO: 181
EIVLTQSPGTLSLSPGERATLSCRASQSVSSSYLAWYQQKPGQAPRLLI
YGASSRATGIPDRFSGSGSGTDFTLTISRLEPEDFAVYYCQQYGSSP
IGKV1-39
SEQ ID NO: 182
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPI
GKV1-33
SEQ ID NO: 183
DIQMTQSPSSLSASVGDRVTITCQASQDISNYLNWYQQKPGKAPKLLIY
DASNLETGVPSRFSGSGSGTDFTFTISSLQPEDIATYYCQQYDNLP

### Example 13. Characterization of Anti-TIM-3 Antibodies

Select antibodies were characterized for their binding to human or cyno cells, and their ability to block ligand galectin 9 binding. Table 39 shows the characteristics of select antibodies in these assays. The cell binding data represents the calculated EC₅₀ values of the antibodies binding to cells transfected with the indicated TIM-3 recombinant protein expressed in μg/ml units. The galectin-9 inhibition represents the maximal level of inhibition of galectin-9 binding to human TIM-3 seen with the indicated antibodies. The tested antibodies were tested as IgG2sigma isotypes.

Epitope mapping assays were performed by coating recombinant huTIM-3-Fc protein on MSD plates. Plates were blocked and washed, followed by the addition of the mixture of the MSD-tag-labeled anti-TIM-3 mAbs incubated with increasing concentrations of unlabeled anti-TIM-3 mABs. After incubation with gentle shaking at room temperature, plates were washed and analyzed with a SECTOR Imager 6000. Antibodies that competed with each other for binding to human TIM-3 were considered to bind to similar epitopes. Positive inhibition was noted if >75% of the binding was inhibited. Partial inhibition was 40-75% inhibition. <40% inhibition was denoted as negative.

TABLE 39

| | Cell binding | | Galectin 9 | |
|---|---|---|---|---|
| | EC₅₀, μg/ml | | Inhibition, | |
| | Human | Cyno | % | Epitope |
| mAb | cells | cells | inhibition | Bin |
| TM3B103 | 0.71 | 0.09 | 71.2 | 1 |
| TM3B105 | 0.46 | 0.03 | 69.8 | 1 |
| TM3B107 | | | 74.8 | 2 |
| TM3B108 | 0.42 | 0.03 | 64.2 | 1 |
| TM3B109 | | | 77.0 | 1 |
| TM3B113 | | | 75.6 | 2 |
| TM3B189 | 0.74 | 0.19 | 76.4 | 3 |
| TM3B190 | 0.35 | 0.08 | 60.7 | 1 |
| TM3B193 | | | 47.4 | 3 |
| TM3B219 | 0.60 | 0.10 | 38.0 | 3 |
| TM3B196 | | | 57.0 | 4 |

### Example 14. Development of a Functional In Vitro Assay to Characterize Anti-TIM-3 Antibodies

Functional assessment of inhibitory receptors such as PD-1 can be done using T cells from normal donor that are stimulated by allogeneic dendritic cells or specific antigens, such as Tetanus toxoid or CMV. In this setting, changes in T cell function with antibody treatment can be detected by measuring supernatant cytokine levels or markers of T cell activation. Effects of anti-TIM-3 antibodies can be very variable in these types of assays, with little overall change in the state of activation or functionality of bulk T cell (non-antigen-specific). On the other hand, using tetramer approaches to follow single T cell sub-populations/clones in these assays does not provide the resolution needed to detect functional effects of anti-TIM-3 antibodies, due to the low frequency and heterogeneous functional profile of these T cell clones. In addition, this approach necessitates the prior identification of the epitopes recognized by CMV-specific T cells in each donor.

CD137 was recently described as a surrogate marker for activated antigen-specific T cells (Wolf et al., (2007) Blood 110(1):201-210; Klinger et al., (2013) PLoS One 8(9): e74231). In our assays, using CD137 enabled the identification of antigen specific CD8+ and CD4⁺ T cells that expand in response to CMV antigen stimulation and allowed the detection of the functional effects of anti-TIM-3 antibodies. In addition to CD137 expression, cytokine secretion by MSD was also evaluated in these assays.

The activity of select anti-TIM-3 antibodies was tested in CMV pp65-stimulated PBMCs. In these assays, anti-TIM-3 antibodies augmented T cell activation, as evidenced by increased CD137 expression on both CD8⁺ and CD4⁺ T cells. In addition, selected anti-TIM-3 antibodies also enhanced secretion of IFN-γ and TNF-α in this assay.

Table 40 shows the results of the CMV assay where enhanced surface expression of CD137 was evaluated on CD8⁺ or CD4⁺ cells for select TIM-3 antibodies. The table shows the p values generated using the Two-tailed T-test (unequal variance).

TABLE 40

| | CD8⁺ CD137⁺, p values | | | CD4⁺ CD137⁺, p values | | |
|---|---|---|---|---|---|---|
| | Mean | Std Dev | n | Mean | Std Dev | n |
| TM3B103 | 0.043 | 0.025 | 5 | 0.071 | 0.112 | 3 |
| TM3B105 | 0.029 | 0.036 | 6 | 0.01 | 0.017 | 3 |
| TM3B107 | 0.182 | 0.188 | 5 | 0.157 | 0.125 | 3 |
| TM3B108 | 0.022 | 0.018 | 5 | 0.01 | 0.01 | 3 |
| TM3B109 | 0.035 | 0.041 | 5 | 0.017 | 0.015 | 3 |
| TM3B113 | 0.082 | 0.064 | 6 | 0.05 | 0.026 | 3 |
| TM3B189 | 0.027 | 0.026 | 6 | 0.007 | 0.011 | 3 |
| TM3B190 | 0.078 | 0.159 | 6 | 0.004 | 0.005 | 3 |
| TM3B193 | 0.467 | 0.252 | 3 | 0.1 | NA | 1 |
| TM3B195 | 0.035 | 0.043 | 7 | 0.01 | 0.01 | 3 |
| TM3B196 | 0.328 | 0.183 | 6 | 0.733 | 0.058 | 3 |
| TM3B197 | 0.473 | 0.303 | 4 | 0.3 | NA | 1 |

### Example 15. Generation of Bispecific PD-1/TIM-3 Antibodies

Select monospecific PD-1 and TIM-3 antibodies were expressed as IgG1/κ, IgG2/κ or IgG4/κ. Substitutions were made at positions 405 and 409 (EU numbering) in the monospecific antibodies to promote subsequent in vitro arm exchange and formation of the bispecific antibodies. The IgG1 and IgG2 anti-PD-1 and anti-TIM-3 antibodies were engineered to have a F405L and a K409R substitution, respectively, to promote arm exchange and generation the bispecific antibodies. On IgG4, the 409 WT position is R, hence the IgG4 anti-PD-1 antibody was not engineered and the IgG4 anti-TIM-3 antibody was engineered to have F405L and R409K substitutions. In addition to position 405 and 409 substitutions, the IgG4 mAbs were engineered to have S228P substitution and the IgG2 antibodies were optionally engineered to include IgG2sigma substitution (V234A, G237A, P238S, H268A, V309L, A330S and P331S).

The monospecific antibodies were expressed and purified using standard methods using a Protein A column (HiTrap MabSelect SuRe column). After elution, the pools were dialyzed into D-PBS, pH 7.2

Bispecific PD-1/TIM-3 antibodies were generated by combining a monospecific PD-1 mAb and a monospecific TIM-3 mAb in in vitro Fab arm exchange as described in Int. Patent Publ. No. WO2011/131746. Briefly, at about 1-20 mg/ml at a molar ratio of 1:1 of each antibody in PBS, pH 7-7.4 and 75 mM 2-mercaptoethanolamine (2-MEA) was mixed together and incubated at 25-37° C. for 2-6 h, followed by removal of the 2-MEA via dialysis, diafiltration, tangential flow filtration and/or spinned cell filtration using standard methods.

The bispecific antibodies were further purified after the in vitro Fab-arm exchange using hydrophobic interaction chromatography to minimize residual parental PD-1 and TIM-3 antibodies using standard methods.

Select monospecific anti-PD-1 antibodies and anti-TIM-3 antibodies were combined in matrix in in vitro Fab arm exchange to generate bispecific antibodies. Table 41, Table 42 and Table 43 show the VH, the VL, the HC and the LC sequences of the generated bispecific antibodies and their isotypes. The G2 antibody allotypes were G2m(n)/(n-) or G2m(n-).

In some experiments, control antibodies were used that were monovalent for either PD-1 or TIM-3 with the second arm being inert binding to gp120. The gp120 binding arm had a VH of SEQ ID NO: 184 and the VL of SEQ ID NO: 185. Table 44 shows the generated control antibodies.

VH of gp120 binding mAb
SEQ ID NO: 184
QVQLVQSGAEVKKPGASVKVSCQASGYRFSNFVIHWVRQAPGQRFEWMG
WINPYNGNKEFSAKFQDRVTFTADTSANTAYMELRSLRSADTAVYYCAR
VGPYSWDDSPQDNYYMDVWGKGTTVIVSS
VL of gp120 binding mAb
SEQ ID NO: 185
EIVLTQSPGTLSLSPGERATFSCRSSHSIRSRRVAWYQHKPGQAPRLVI
HGVSNRASGISDRFSGSGSGTDFTLTITRVEPEDFALYYCQVYGASSYT
FGQGTKLERK

TABLE 41

| | PD-1 binding arm | | | | |
|---|---|---|---|---|---|
| | | VH1 | | VL1 | |
| | | SEQ | | SEQ | |
| mAb | VH1 | ID NO: | VL1 | ID NO: | Isotype |
| PTBB14 | PD1H170 | 48 | PD1L148 | 56 | IgG2sigma |
| PTBB15 | PD1H170 | 48 | PD1L148 | 56 | IgG2sigma |
| PTBB16 | PD1H129 | 64 | PD1L62 | 65 | IgG2sigma |
| PTBB17 | PD1H129 | 64 | PD1L62 | 65 | IgG2sigma |
| PTBB24 | PD1H170 | 48 | PD1L148 | 56 | IgG2sigma |
| PTBB30 | PD1H170 | 48 | PD1L148 | 56 | IgG2sigma |
| PTBB27 | PD1H170 | 48 | PD1L148 | 56 | IgG2 |
| PTBB28 | PD1H170 | 48 | PD1L148 | 56 | IgG2 |
| PTBB18 | PD1H129 | 64 | PD1L62 | 65 | IgG4_S228P |
| PTBB20 | PD1H170 | 48 | PD1L148 | 56 | IgG4_S228P |
| PTBB21 | PD1H170 | 48 | PD1L148 | 56 | IgG4_S228P |

TABLE 42

| | TIM-3 binding arm | | | | |
|---|---|---|---|---|---|
| | | VH2 | | VL2 | |
| | | SEQ | | SEQ | |
| mAb | VH2 | ID NO: | VL2 | ID NO: | Isotype |
| PTBB14 | TM3H144 | 153 | TM3L67 | 162 | IgG2sigma |
| PTBB15 | TM3H24 | 146 | TM3L33 | 156 | IgG2sigma |
| PTBB16 | TM3H144 | 153 | TM3L67 | 162 | IgG2sigma |
| PTBB17 | TM3H24 | 146 | TM3L33 | 156 | IgG2sigma |
| PTBB24 | TM3H162 | 172 | TM3L85 | 173 | IgG2sigma |
| PTBB30 | TM3H24 | 146 | TM3L33 | 156 | IgG2sigma |
| PTBB27 | TM3H162 | 172 | TM3L85 | 173 | IgG2 |
| PTBB28 | TM3H24 | 146 | TM3L33 | 156 | IgG2 |
| PTBB18 | TM3H24 | 146 | TM3L33 | 156 | IgG4_S228 |
| PTBB20 | TM3H24 | 146 | TM3L33 | 156 | IgG4_S228 |
| PTBB21 | TM3H162 | 172 | TM3L85 | 173 | IgG4_S228 |

TABLE 43

| SEQ ID NO: | |
|---|---|
| PD-1 binding arm | TIM-3 binding arm |

| mAb | HC1 | LC1 | HC2 | LC2 |
|---|---|---|---|---|
| PTBB14 | 186 | 188 | 190 | 193 |
| PTBB15 | 186 | 188 | 191 | 194 |
| PTBB16 | 187 | 189 | 190 | 193 |
| PTBB17 | 187 | 189 | 191 | 194 |
| PTBB24 | 186 | 188 | 192 | 195 |
| PTBB30 | 186 | 188 | 248 | 194 |
| PTBB27 | 241 | 188 | 244 | 195 |
| PTBB28 | 241 | 188 | 245 | 194 |
| PTBB18 | 242 | 189 | 246 | 194 |
| PTBB20 | 243 | 188 | 246 | 194 |
| PTBB21 | 243 | 188 | 247 | 195 |

SEQ ID NO: 186
QVQLVQSGAEVKKPGSSVKVSCKASGGTFSSYAISWVRQAPGQGLEWMG
GIIPIFDTANYAQKFQGRVTITADESTSTAYMELSSLRSEDTAVYYCAR
PGLAAAYDTGSLDYWGQGTLVTVSSASTKGPSVFPLAPCSRSTSESTAA
LGCLVKDYFPEPVTVSWNSGALTSGVHTFPAVLQSSGLYSLSSVVTVTS
SNFGTQTYTCNVDHKPSNTKVDKTVERKCCVECPPCPAPPAAASSVFLF
PPKPKDTLMISRTPEVTCVVVDVSAEDPEVQFNWYVDGVEVHNAKTKPR
EEQFNSTFRVVSVLTVLHQDWLNGKEYKCKVSNKGLPSSIEKTISKTKG
QPREPQVYTLPPSREEMTKNQVSLTCLVKGFYPSDIAVEWESNGQPENN
YKTTPPMLDSDGSFLLYSKLTVDKSRWQQGNVFSCSVMHEALHNHYTQK
SLSLSPGK
SEQ ID NO: 187
EVQLVESGGGLVQPGGSLRLSCAASGFAFSRYDMSWVRQAPGKGLESVA
YISGGGANTYYLDNVKGRFTISRDNAKNSLYLQMNSLRAEDTAVYYCAS
PYLSYFDVWGQGTLVTVSSASTKGPSVFPLAPCSRSTSESTAALGCLVK
DYFPEPVTVSWNSGALTSGVHTFPAVLQSSGLYSLSSVVTVTSSNFGTQ
TYTCNVDHKPSNTKVDKTVERKCCVECPPCPAPPAAASSVFLFPPKPKD
TLMISRTPEVTCVVVDVSAEDPEVQFNWYVDGVEVHNAKTKPREEQFNS
TFRVVSVLTVLHQDWLNGKEYKCKVSNKGLPSSIEKTISKTKGQPREPQ
VYTLPPSREEMTKNQVSLTCLVKGFYPSDIAVEWESNGQPENNYKTTPP
MLDSDGSFLLYSKLTVDKSRWQQGNVFSCSVMHEALHNHYTQKSLSLSP
GK
SEQ ID NO: 188
EIVLTQSPATLSLSPGERATLSCRASQSVRSYLAWYQQKPGQAPRLLIY
DASNRATGIPARFSGSGSGTDFTLTISSLEPEDFAVYYCQQRNYWPLTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
SEQ ID NO: 189
EIVMTQSPATLSVSPGERATLSCRASQSLSDYLHWYQQKPGQAPRLLIK
SASQSISGIPARFSGSGSGTEFTLTISSLQSEDFAVYYCQNGHSFPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
SEQ ID NO: 190
EVQLQQSGAELARPGASVKLSCKASGYTFTSYWMQWVKQRPGQGLEWIG
AIYPGDGDIRYTQNFKGKATLTADKSSSTAYMQLSSLASEDSAVYYCAR
WEKSTTVVQRNYFDYWGQGTTLTVSSASTKGPSVFPLAPCSRSTSESTA
ALGCLVKDYFPEPVTVSWNSGALTSGVHTFPAVLQSSGLYSLSSVVTVT
SSNFGTQTYTCNVDHKPSNTKVDKTVERKCCVECPPCPAPPAAASSVFL
FPPKPKDTLMISRTPEVTCVVVDVSAEDPEVQFNWYVDGVEVHNAKTKP
REEQFNSTFRVVSVLTVLHQDWLNGKEYKCKVSNKGLPSSIEKTISKTK
GQPREPQVYTLPPSREEMTKNQVSLTCLVKGFYPSDIAVEWESNGQPEN
NYKTTPPMLDSDGSFFLYSRLTVDKSRWQQGNVFSCSVMHEALHNHYTQ
KSLSLSPGK
SEQ ID NO: 191
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
SPYAPLDYWGQGTLVTVSSASTKGPSVFPLAPCSRSTSESTAALGCLVK
DYFPEPVTVSWNSGALTSGVHTFPAVLQSSGLYSLSSVVTVTSSNFGTQ
TYTCNVDHKPSNTKVDKTVERKCCVECPPCPAPPAAASSVFLFPPKPKD
TLMISRTPEVTCVVVDVSAEDPEVQFNWYVDGVEVHNAKTKPREEQFNS
TFRVVSVLTVLHQDWLNGKEYKCKVSNKGLPSSIEKTISKTKGQPREPQ
VYTLPPSREEMTKNQVSLTCLVKGFYPSDIAVEWESNGQPENNYKTTPP
MLDSDGSFFLYSRLTVDKSRWQQGNVFSCSVMHEALHNHYTQKSLSLSP
GK
SEQ ID NO: 192
EVQLVQSGAEVKKPGESLKISCKGSGYSFTSYWMQWVRQMPGKGLEWMG
AIYPGDGDIRYTQNFKGQVTISADKSISTAYLQWSSLKASDTAMYYCAR
WEKSTTVVQRNYFDYWGQGTTVTVSSASTKGPSVFPLAPCSRSTSESTA
ALGCLVKDYFPEPVTVSWNSGALTSGVHTFPAVLQSSGLYSLSSVVTVT
SSNFGTQTYTCNVDHKPSNTKVDKTVERKCCVECPPCPAPPAAASSVFL
FPPKPKDTLMISRTPEVTCVVVDVSAEDPEVQFNWYVDGVEVHNAKTKP
REEQFNSTFRVVSVLTVLHQDWLNGKEYKCKVSNKGLPSSIEKTISKTK
GQPREPQVYTLPPSREEMTKNQVSLTCLVKGFYPSDIAVEWESNGQPEN
NYKTTPPMLDSDGSFFLYSRLTVDKSRWQQGNVFSCSVMHEALHNHYTQ
KSLSLSPGK
SEQ ID NO: 193
DVQMIQSPKSMSMSVGERVTLSCKASENVGTFVSWYQQKPDQSPKLLIY
GASNRYTGVPDRFTGSGSATDFTLTISSVQAEDLADYHCGQSYSYPTFG
SGTKLEMKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC
SEQ ID NO: 194
EIVLTQSPATLSLSPGERATLSCRASQSVNDYLAWYQQKPGQAPRLLIY
DASNRATGIPARFSGSGSGTDFTLTISSLEPEDFAVYYCQQGGHAPITF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
SEQ ID NO: 195
DIQMTQSPSSLSASVGDRVTITCKASENVGTFVSWYQQKPGKAPKLLIY
GASNRYTGVPSRFSGSGSGTDFTLTISSLQPEDFATYYCGQSYSYPTFG
QGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC
SEQ ID NO: 241
QVQLVQSGAEVKKPGSSVKVSCKASGGTFSSYAISWVRQAPGQGLEWMG
GIIPIFDTANYAQKFQGRVTITADESTSTAYMELSSLRSEDTAVYYCAR
PGLAAAYDTGSLDYWGQGTLVTVSSASTKGPSVFPLAPCSRSTSESTAA
LGCLVKDYFPEPVTVSWNSGALTSGVHTFPAVLQSSGLYSLSSVVTVPS
SNFGTQTYTCNVDHKPSNTKVDKTVERKCCVECPPCPAPPVAGPSVFLF
PPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKG
QPREPQVYTLPPSREEMTKNQVSLTCLVKGFYPSDIAVEWESNGQPENN
YKTTPPMLDSDGSFLLYSKLTVDKSRWQQGNVFSCSVMHEALHNHYTQK
SLSLSPGK
SEQ ID NO: 242
EVQLVESGGGLVQPGGSLRLSCAASGFAFSRYDMSWVRQAPGKGLESVA
YISGGGANTYYLDNVKGRFTISRDNAKNSLYLQMNSLRAEDTAVYYCAS
PYLSYFDVWGQGTLVTVSSASTKGPSVFPLAPCSRSTSESTAALGCLVK
DYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPK
DTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLS
LGK
SEQ ID NO: 243
QVQLVQSGAEVKKPGSSVKVSCKASGGTFSSYAISWVRQAPGQGLEWMG
GIIPIFDTANYAQKFQGRVTITADESTSTAYMELSSLRSEDTAVYYCAR
PGLAAAYDTGSLDYWGQGTLVTVSSASTKGPSVFPLAPCSRSTSESTAA
LGCLVKDYFPEPVTVSWNSGALTSGVHTFPAVLQSSGLYSLSSVVTVPS
SSLGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQ
KSLSLSLGK
SEQ ID NO: 244
EVQLVQSGAEVKKPGESLKISCKGSGYSFTSYWMQWVRQMPGKGLEWMG
AIYPGDGDIRYTQNFKGQVTISADKSISTAYLQWSSLKASDTAMYYCAR
WEKSTTVVQRNYFDYWGQGTTVTVSSASTKGPSVFPLAPCSRSTSESTA
ALGCLVKDYFPEPVTVSWNSGALTSGVHTFPAVLQSSGLYSLSSVVTVP
SSNFGTQTYTCNVDHKPSNTKVDKTVERKCCVECPPCPAPPVAGPSVFL
FPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTK
GQPREPQVYTLPPSREEMTKNQVSLTCLVKGFYPSDIAVEWESNGQPEN
NYKTTPPMLDSDGSFFLYSRLTVDKSRWQQGNVFSCSVMHEALHNHYTQ
KSLSLSPGK
SEQ ID NO: 245
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
SPYAPLDYWGQGTLVTVSSASTKGPSVFPLAPCSRSTSESTAALGCLVK
DYFPEPVTVSWNSGALTSGVHTFPAVLQSSGLYSLSSVVTVPSSNFGTQ
TYTCNVDHKPSNTKVDKTVERKCCVECPPCPAPPVAGPSVFLFPPKPKD
TLMISRTPEVTCVVVDVSHEDPEVQFNWYVDGVEVHNAKTKPREEQFNS
TFRVVSVLTVVHQDWLNGKEYKCKVSNKGLPAPIEKTISKTKGQPREPQ
VYTLPPSREEMTKNQVSLTCLVKGFYPSDIAVEWESNGQPENNYKTTPP
MLDSDGSFFLYSRLTVDKSRWQQGNVFSCSVMHEALHNHYTQKSLSLSP
GK
SEQ ID NO: 246
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
SPYAPLDYWGQGTLVTVSSASTKGPSVFPLAPCSRSTSESTAALGCLVK
DYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPK
DTLMISRTPEVTCVVVDVSQEDPEVQFNWYVDGVEVHNAKTKPREEQFN
STYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTP
PVLDSDGSFLLYSKLTVDKSRWQEGNVFSCSVMHEALHNHYTQKSLSLS
LGK
SEQ ID NO: 247
EVQLVQSGAEVKKPGESLKISCKGSGYSFTSYWMQWVRQMPGKGLEWMG
AIYPGDGDIRYTQNFKGQVTISADKSISTAYLQWSSLKASDTAMYYCAR
WEKSTTVVQRNYFDYWGQGTTVTVSSASTKGPSVFPLAPCSRSTSESTA
ALGCLVKDYFPEPVTVSWNSGALTSGVHTFPAVLQSSGLYSLSSVVTVP
SSSLGTKTYTCNVDHKPSNTKVDKRVESKYGPPCPPCPAPEFLGGPSVF
LFPPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPE
NNYKTTPPVLDSDGSFLLYSKLTVDKSRWQEGNVFSCSVMHEALHNHYT
QKSLSLSLGK
SEQ ID NO: 248
EVQLLESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
SPYAPLDYWGQGTLVTVSSASTKGPSVFPLAPCSRSTSESTAALGCLVK
DYFPEPVTVSWNSGALTSGVHTFPAVLQSSGLYSLSSVVTVPSSNFGTQ
TYTCNVDHKPSNTKVDKTVERKCCVECPPCPAPPAAASSVFLFPPKPKD
TLMISRTPEVTCVVVDVSAEDPEVQFNWYVDGVEVHNAKTKPREEQFNS
TFRVVSVLTVLHQDWLNGKEYKCKVSNKGLPSSIEKTISKTKGQPREPQ
VYTLPPSREEMTKNQVSLTCLVKGFYPSDIAVEWESNGQPENNYKTTPP
MLDSDGSFFLYSRLTVDKSRWQQGNVFSCSVMHEALHNHYTQKSLSLSP
GK

TABLE 44

| | Arm 1 VH/VL | Arm 2 VH/VL | |
|---|---|---|---|
| Control | with F405L | with K409R | |
| mAb | substitution | substitution | Isotype |
| TM3B342 | gp120 | TM3B195 | IgG2sigma |
| TM3B343 | gp120 | TM3B299 | IgG2sigma |
| B23B74 | gp120 | B23B32 | IgG2sigma |
| PTBB23 | gp120 | TM3B291 | IgG2sigma |
| PD1B355 | PD1B246 | gp120 | IgG2sigma |
| PD1B356 | PD1B248 | gp120 | IgG2sigma |

### Example 16. Characterization of Bispecific PD-1/TIM-3 Antibodies

The generated antagonistic bispecific antibodies were tested in the CMV assay for their ability to enhance antigen-specific T cell responses. Functionality was measured by assessing CD137 expression on both CD4⁺ and CD⁺ T cells and by IFN-γ and TNF-α levels in the culture supernatants as described in Example 14. Table 45 and Table 46 summarize the activity of bispecific PD-1/TIM-3 antibodies in this assay for the different readouts. As shown in this table, select bispecific molecules led to significant increases in CD137 expression on CD4⁺ and CD8⁺ T cells and in levels of secreted IFN-γ and TNF-α. Overall, the PD-1/TIM-3 bispecifics with huIgG2sigma Fc had the most robust activity, followed by those molecules with huIgG2 and then huIgG4.

TABLE 45

| | | Statistical Significance | | | |
|---|---|---|---|---|---|
| | | CD4⁺CD137⁺ | | CD8⁺CD137⁺ | |
| mAb | | Avg p | | Avg p | |
| Isotype | name | value | St Dev | value | St Dev |
| IgG2sigma | PTBB14 | 0.1144 | 0.1591 | 0.0002 | 0.0001 |
| IgG2sigma | PTBB15 | 0.0467 | 0.0988 | 0.0001 | 0.0000 |
| IgG2sigma | PTBB16 | 0.0017 | 0.0023 | 0.0001 | 0.0000 |
| IgG2sigma | PTBB17 | 0.4148 | 0.5051 | 0.0001 | 0.0001 |
| IgG2sigma | PTBB24 | 0.0031 | 0.0051 | 0.0001 | 0.0000 |
| IgG2 | PTBB27 | 0.0009 | 0.0011 | 0.0001 | 0.0000 |
| IgG2 | PTBB28 | 0.0003 | 0.0002 | 0.0001 | 0.0000 |
| IgG4 | PTBB18* | 0.0353 | | 0.0071 | |
| IgG4 | PTBB20 | 0.6025 | 0.1710 | 0.0004 | 0.0004 |
| IgG4 | PTBB21 | 0.1071 | 0.1372 | 0.0059 | 0.0081 |

*one p value reported

TABLE 46

| | | Statistical significance | | | |
|---|---|---|---|---|---|
| | | IFN-γ | | TNF-α | |
| mAb | | Avg p | | Avg p | |
| Isotype | name | value | St Dev | value | St Dev |
| IgG2sigma | PTBB14 | 0.0001 | 0.0000 | 0.0112 | 0.0157 |
| IgG2sigma | PTBB15 | 0.0001 | 0.0000 | 0.0005 | 0.0008 |
| IgG2sigma | PTBB16 | 0.0001 | 0.0000 | 0.0012 | 0.0016 |
| IgG2sigma | PTBB17 | 0.0001 | 0.0000 | 0.0001 | 0.0000 |
| IgG2sigma | PTBB24 | 0.0001 | 0.0001 | 0.0008 | 0.0008 |
| IgG2 | PTBB27 | 0.0026 | 0.0030 | 0.3406 | 0.4757 |
| IgG2 | PTBB28 | 0.0001 | 0.0000 | 0.1437 | 0.1229 |
| IgG4 | PTBB18 | 0.0001 | #DIV/0! | 0.0008 | #DIV/0! |
| IgG4 | PTBB20 | 0.0544 | 0.0768 | 0.1754 | 0.2140 |
| IgG4 | PTBB21 | 0.0174 | 0.0245 | 0.2685 | 0.1103 |

*one p value reported

### Example 17. Anti-PD1 Antibodies Upregulate TIM-3 Expression on Tumors

Effect of anti-PD-1 antibody treatment in expression of TIM-3 on tumors were evaluated in CT26 or MC38 colon carcinoma mouse model.

Balb/c mice were implanted subcutaneously with 1×10⁶ CT26 colon carcinoma tumors. Seven days after tumor cell implant, tumors were measured and mice were randomized by tumor size. Treatment with PBS or 10 mg/kg anti-mouse PD-1 antibodies (clone RMP1-14, BioXCell) began on day 7 after tumor cell implant and continued biweekly for the remainder of the study. To analyze T cell expression of TIM-3, tumors were harvested at day 22 and dissociated using GentleMACS (Miltenyi). Staining for flow cytometry was carried out with Live/Dead and markers for CD3, CD4, CD8 and TIM-3. Flow cytometry was performed on a LSR Fortessa (BD). Data was analyzed using the Flow Jo software.

Wild-type C57Bl/6 female mice were implanted subcutaneously with 5×10⁵ MC-38 colon carcinoma cells suspended in PBS. Tumors were measured and mice were randomized by tumor size (50-100 mm³). Treatment with PBS or 10 mg/kg anti-mouse PD-1 (clone RMP1-14, BioXCell) began after randomization and continued biweekly for the remainder of the study. To profile tumor infiltrating T cells, tumors were harvested and dissociated using GentleMACS (Miltenyi) 12, 15, 19, or 22 days after implant.

Staining for flow cytometry was carried out with Live/Dead and markers for CD45, Thy1, CD3, CD4, CD8, TIM-3, CD137, OX40, GITR, TIGIT. Flow cytometry data was collected on a LSR Fortessa (BD). Data was analyzed using the FlowJo software (v9.9.4) and visualized with GraphPad Prism. Statistics were generated by GraphPad Prism.

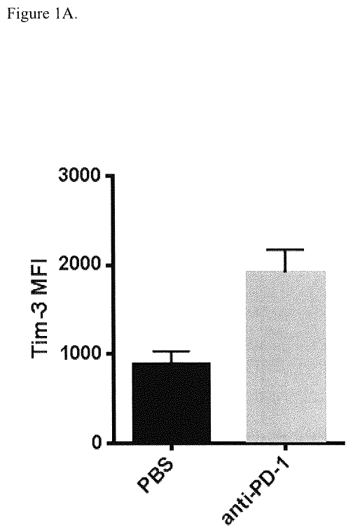

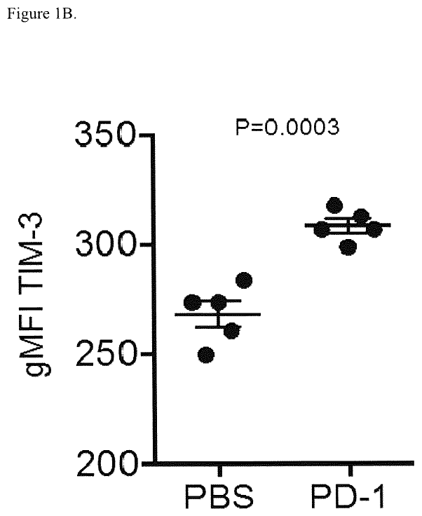

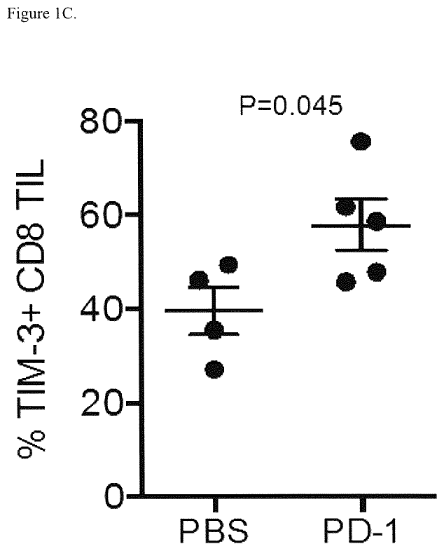

Analysis of TIM-3 expression on CD8⁺ T cells isolated from CT26 tumors at day 22 revealed an increase of TIM-3 expression in the PD-1 treated samples, compared to PBS control. FIG. 1A shows the mean fluorescent intensity of TIM-3 expression in the two treatment groups.

TIM-3 expression was also increased in MC-38 tumors in the anti-PD-1 mAb treated samples when compared to PBS control. FIG. 1B shows the geometric mean fluorescent intensity of TIM-3 expression in the CD8⁺ TIL population. FIG. 1C shows the percentage (%) relative frequency of TIM-3⁺ CD8⁺ cells of total CD8⁺ TILs.

These data show that TIM-3 is upregulated in response to anti-PD-1 treatment, supporting the rational for targeting TIM-3 in PD-1 treated subjects.

CD137, OX40 and GITR expression was also analyzed on CD8⁺ T cells infiltrating MC38 tumors isolated from mice treated with anti-mouse PD-1 antibodies.

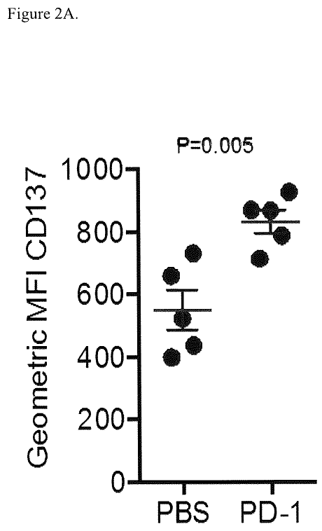

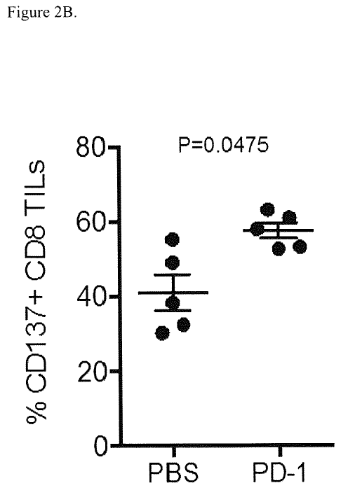

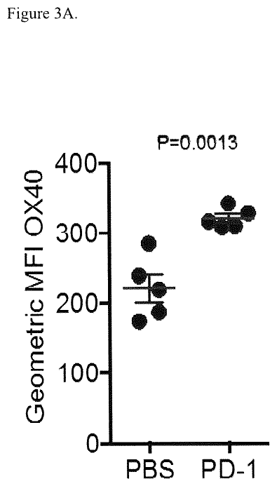

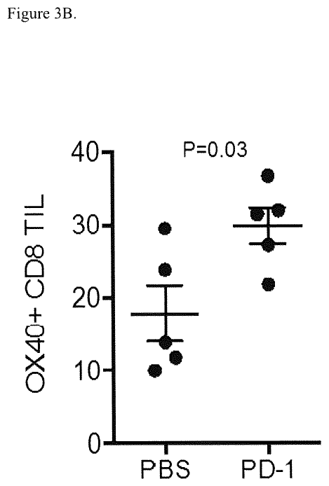

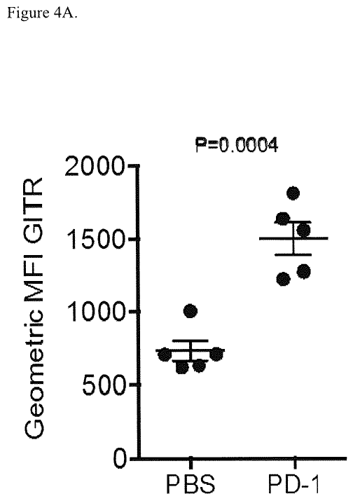

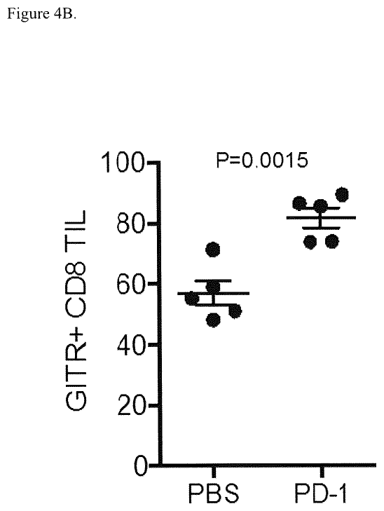

These results showed that both the frequency and level (gMFJ) of TNF family costimulatory receptors CD137, OX40 and GITR expression was increased following PD-1 blockade. FIG. 2A and FIG. 2B show the gMFI and relative frequency of CD137 expression on CD8 TILs, respectively. FIG. 3A and FIG. 3B show the gMFI and relative frequency of OX40 expression on CD8 TILs, respectively, and FIG. 4A and FIG. 4B show the gMFI and relative expression of GITR on CD8 TILs, respectively.

These data support the rational for targeting CD137, OX40 and/or GITR in PD-1 treated subjects.

### Example 18. Activity of Anti-TIM-3 Antibodies Following PD-1 Blockade

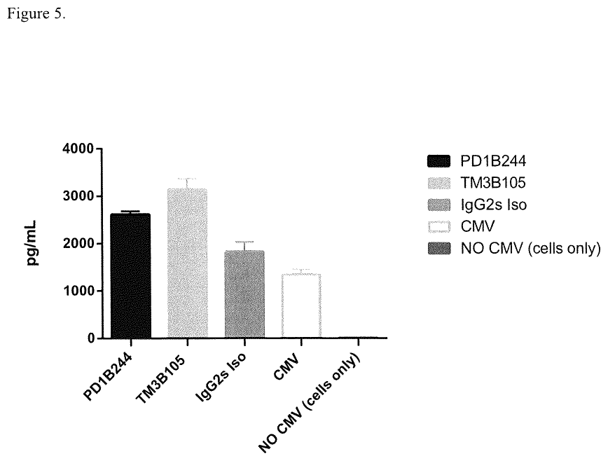

The activity of anti-TIM-3 antibodies was also tested following anti-PD-1 antibody blockade in the CMV assay. In these experiments, PBMCs from one normal donor (CMV-sera positive) were incubated with pp65 peptide pools and anti-PD-1 antibodies for 5 days. On day 5, supernatants were harvested and cells were re-stimulated with pp65 peptide pool in the presence of either anti-TIM-3 or anti-PD-1 antibody. IFN-γ levels in the supernatant were measured 24 hours later. Treatment with anti-TIM-3 antibodies after 5 days of anti-PD-1 blockade resulted in a significant increase of IFN-γ levels. This effect was significant (p=0.0183) compared to continued anti-PD-1 treatment. In the experiment, anti-TIM-3 antibody TM3B403 and anti-PD-1 antibody PD1B244 were used. FIG. 5 shows the increased IFN-γ levels in the CMV assay, where PBMCs were treated with anti-TIM-3 antibody TM3B105 following 5 days of treatment with anti-PD-1 PD1B244. Values represent average of six biological replicates used for each condition.

### Example 19. Epitope Mapping of Anti-TIM-3 Antibodies

Solution hydrogen/deuterium exchange-mass spectrometry (HDX-MS) was performed to identify the binding epitopes of TMB403 and TMB291. For the experiments, the VH and the VL of TM3B403 and TM3B291 were cloned as IgG1 Fabs with a hexahistidine tag in the C-terminus. The Fabs, were generated from transient transfections of HEK293 Expi cells in suspension shake flasks. TIM-3 IgG1 Fc Chimera, Ser22-Arg200 (Accession #Q8TDQ0), produced in Mouse myeloma cell line (NSO derived) from R&D Systems (Catalog #2365-TM) was used.

For H/D exchange, the procedures used to analyze the Fab perturbation were similar to those described previously (Hamuro et al., Biomolecular Techniques 14: 171-182, 2003; Horn et al., Biochemistry 45: 8488-8498, 2006) with some modifications. Briefly, deglycosylated human TIM-3/Fc fusion protein or deglycosylated human TIM-3-Fc plus Fab mixture was incubated with deuterium oxide labeling buffer at 0° C. for various times up to 2 hours. Deuterium exchange was quenched by adding guanidine hydrochloride and the quenched sample was subjected to on-column pepsin digestion and LC-MS analysis. The mass spectra were recorded in MS only mode. For the calculation of deuterium incorporation, the mass spectra for a given peptide were combined across the extracted ion chromatogram peak and the weighted average m/z was calculated. The mass increase from the mass of the native peptide (0 min) to the weighted averaged mass corresponds to the level of deuterium incorporation. About 98.4% of the protein could be mapped to specific peptides.

The deuterium levels at the identified peptides were monitored from the mass shift on LC-MS. The selected deuterium buildup curves, which show significant difference in deuterium levels and/or slopes, over exchange time for the peptides were plotted.

Deglycosylated human Tim-3/Fc fusion protein showed significant reduction in deuterium uptakes upon binding to TM3B403 at sequences ₃₂WGKGACPVFECGNVVL₄₇, (SEQ ID NO: 261) and upon binding to TM3B291 at sequences ₉₀RIQIPGIMNDEKF₁₀₂.(SEQ ID NO: 262). These regions with significant reduction in deuterium uptakes upon binding to Fabs can thus be regarded as main epitopes of the mAbs.

A segment, ₅₀DERDVNY₅₆, (SEQ ID NO: 263) demonstrated modest reduction in deuterium exchange upon binding to TM3B403 or TM3B291. This region may be also considered as a potential epitope for both antibodies.

The major binding epitopes for TM3B403 or TM3B291 are different. However, they may share the similar modest protection region, ₅₀DERDVNY₅₆, (SEQ ID NO: 263) based on the HDX mapping results. To help assess if this region contributes to common binding epitope region for both Fab molecules, competition ELISA was performed. Recombinant human Tim-3/Fc protein was directly coated on plates which were then blocked and washed. A mixture of Ruthenium (Ru)-labeled TM3B291 Fab which was pre-incubated with different concentrations of unlabeled TM3B105 or TM3B291. Plates were incubated, washed and MSD Read Buffer T was dispensed into each well followed by reading with a SECTOR Imager 6000 (Meso Scale Discovery, Gaithersburg, MD).

The competition analysis demonstrated that that TM3B403 competed for binding to TIM-3 with TM3B291. This result could indicate that the modestly protected region, DERDVNY (SEQ ID NO: 263) is part of the epitope for both antibodies or that the antibodies may be sterically blocking each other's binding due to the close proximity of their epitopes.

### Example 20. TIM-3 Blockade Increases TIGIT Expression on CD8⁺ TILs

Effect of anti-TIM-3 antibody treatment on expression of TIGIT in tumors was evaluated in CT26 and MC38 colon carcinoma mouse models. The studies were conducted as described in Example 17 except that 10 mg/ml anti-TIM-3 antibody RMT3-23 was used.

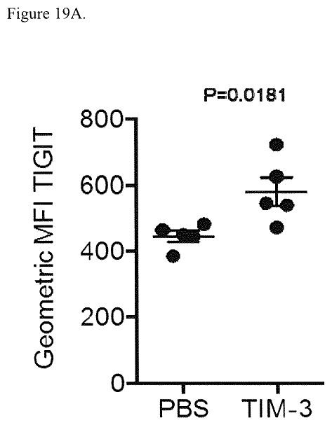

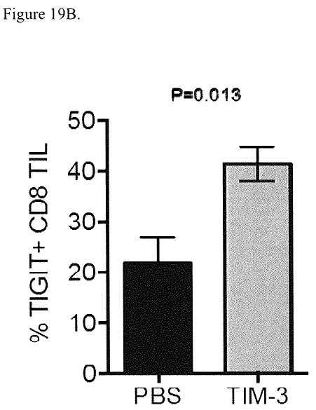

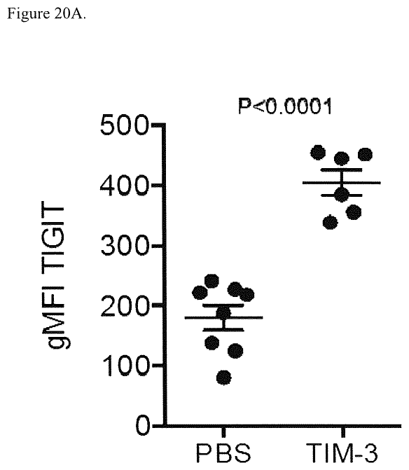

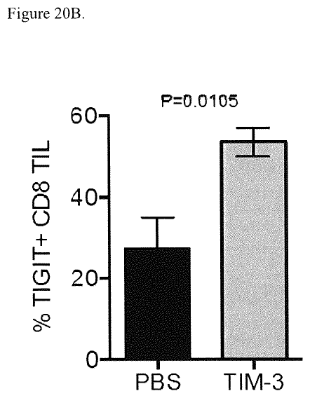

TIGIT expression on CD8+ TILs (FIG. 19A, FIG. 20A) and relative frequency of TIGIT+ TILs f(FIG. 19B, FIG. 20B) were elevated in both CT26 (FIG. 19A, FIG. 19B) and MC38 (FIG. 20A, FIG. 20B) tumor models following TIM-3 blockage.

### Example 21. TIM-3 Expression is Increased after Ex Vivo PD-1 Blockade in Melanoma Patient PBMC

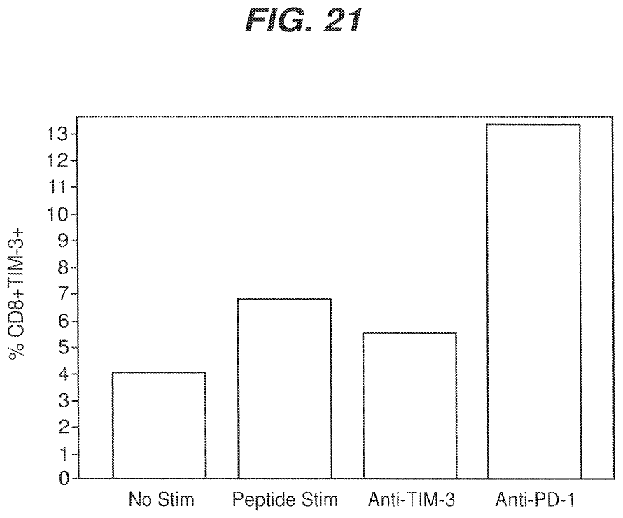

PBMCs from treatment naïve melanoma patients were stimulated with melanoma antigen peptide pools (NY-ESO, gp100, MART-1) in the presence of anti-PD-1 or anti-TIM-3 function blocking antibodies. Expression of TIM-3 was evaluated on peptide-restimulated cells on day 6. Results showed significant increases in the frequency of TIM-3+ CD8+ T cells in the anti-PD-1 treated samples compared to controls or TIM-3 treated PBMCs (FIG. 21).

On day 0, frozen PBMCs from treatment naïve melanoma patients were rapidly thawed in a 37° C. water bath. Cells were thawed, washed and counted in complete RPMI media (RPMI+10% FBS+1% sodium pyruvate+1% NEAA+1% pen/strep). Cells were plates at 200,000 cells per well in a 96 well, U-bottom plate in the presence or absence of anti-PD-1 or anti-TIM-3 function blocking antibodies (PD1B244 and TM3B403, respectively) and 1 g/mL of melanoma antigen peptide pools (NY-ESO, gp100, MART-1) for 6 days at 37 C. Cells were restimulated with the peptide pool at day 6 and analyzed by flow cytometry for expression of PD-1 and TIM-3 as well as T cell activation and proliferation markers.

### Example 22. Anti-TIM-3 Antibodies Increase the Frequency of Activated NK Cells in IL-2 Stimulated PBMCs

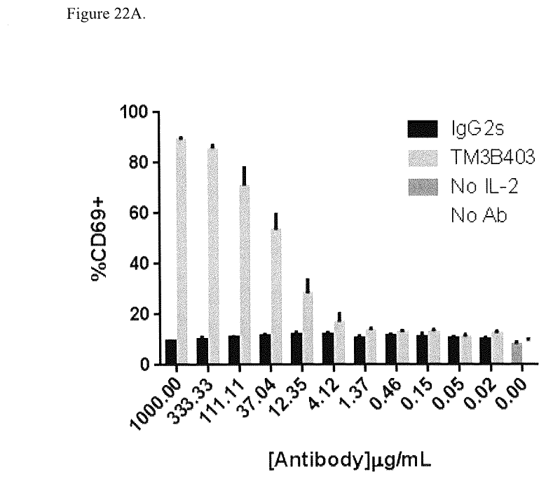

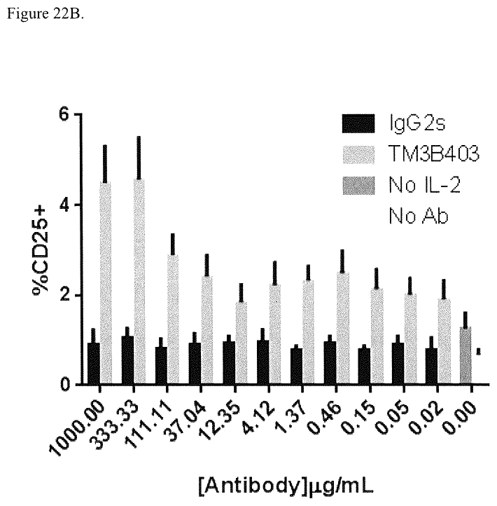

The effects of anti-TIM-3 antibody TM3B403 on the frequency of activated NK cells was determined in assays where human PBMCs were stimulated with IL-2 (20U). Frequency of CD69 and CD25, markers of NK cell activation, were evaluated by flow cytometry 48 hours post-treatment at a range of mAb concentrations. TM3B403 increased the frequency of activated NK cells when the activation was assessed by percentage of CD69 positive cells (FIG. 22A) or percentage of CD25 positive cells (FIG. 22B).

1-89. (canceled)

90. An isolated antagonistic antibody or an antigen-binding portion thereof specifically binding PD-1, comprising a heavy chain complementarity determining region (HCDR) 1 a HCDR2 and a HCDR3, and a light chain complementarity determining region (LCDR) 1, a LCDR2 and a LCDR3, of SEQ ID NOs:
a. 10, 13, 16, 20, 26 and 31, respectively;
b. 10, 13, 16, 21, 26 and 32, respectively;
c. 10, 14, 16, 22, 27 and 33, respectively;
d. 10, 14, 16, 22, 26 and 34, respectively;
e. 10, 14, 16, 23, 28 and 35, respectively;
f. 10, 13, 17, 20, 26 and 31, respectively;
g. 10, 13, 17, 20, 26 and 36, respectively;
h. 10, 13, 17, 21, 26 and 32, respectively;
i. 10, 13, 17, 21, 27 and 37, respectively;
j. 10, 13, 17, 23, 26 and 32, respectively;
k. 10, 14, 17, 23, 28 and 35, respectively; or
l. 10, 14, 17, 22, 26 and 34, respectively.

91. The antibody or the antigen-binding portion thereof of claim 90, comprising a heavy chain variable region (VH) and a light chain variable region (VL) of SEQ ID NOs:
a. 41 and 49, respectively;
b. 41 and 50, respectively;
c. 42 and 51, respectively;
d. 42 and 52, respectively;
e. 42 and 53, respectively;
f. 43 and 49, respectively;
g. 43 and 54, respectively;
h. 43 and 50, respectively;
i. 43 and 55, respectively;
j. 43 and 56, respectively;
k. 48 and 53, respectively; or
l. 48 and 52, respectively.

92. The antibody or the antigen-binding portion thereof of claim 90, wherein the antibody is human or humanized.

93. The antibody or the antigen-binding portion thereof of claim 90, wherein the antibody is
a. an IgG1 isotype, optionally comprising one, two, three, four, five, six, seven, eight, nine or ten substitutions in an Fc region;
b. an IgG2 isotype, optionally comprising one, two, three, four, five, six, seven, eight, nine or ten substitutions in the Fc region;
c. an IgG3 isotype, optionally comprising one, two, three, four, five, six, seven, eight, nine or ten substitutions in the Fc region;
d. an IgG4 isotype, optionally comprising one, two, three, four, five, six, seven, eight, nine or ten substitutions in the Fc region;
e. an IgG1 isotype comprising L234A, L235A, G237A, P238S, H268A, A330S and P331 S substitutions;
f. an IgG2 isotype comprising V234A, G237A, P238S, H268A, V309L, A330S and P331 S substitutions;
g. an IgG4 isotype comprising F234A, L235A, G237A, P238S and Q268A substitutions;
h. an IgG1 isotype comprising L234A, L235A or L234A and L235A substitutions;
i. an IgG4 isotype comprising F234A, L235A or F234A and L235A substitutions;
j. an IgG2 isotype comprising a V234A substitution;
k. an IgG4 isotype comprising a S228P substitution; or
l. an IgG4 isotype comprising S228P, F234A and L235A substitutions, wherein residue numbering is according to the EU Index.

94. The antibody or the antigen-binding portion thereof of claim 91, comprising a heavy chain (HC) and a light chain (LC) of SEQ ID NOs:
a. 212 and 213, respectively;
b. 214 and 215, respectively;
c. 216 and 217, respectively;
d. 218 and 219, respectively;
e. 220 and 221, respectively;
f. 222 and 223, respectively;
g. 224 and 225, respectively; or
h. 226 and 227, respectively;

95. A pharmaceutical composition comprising the antibody or the antigen-binding portion thereof of claim 90 and a pharmaceutically accepted carrier.

96. A kit comprising the antibody of claim 90.


SEQUENCE LISTING (312 sequences)

SEQ ID NO: 1 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 149 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTL

SEQ ID NO: 3 - protein, 268 aa, Macaca fascicularis
PGWFLESPDRPWNAPTFSPALLLVTEGDNATFTCSFSNASESFVLNWYRMSPSNQTDKLAAFPEDRSQPGQDCRFRVTRLPNGRDFHMSVVRARRNDSGTYLCGAISLAPKAQIKESLRAELRVTERRAEVPTAHPSPSPRPAGQFQALVVGVVGGLLGSLVLLVWVLAVICSRAAQGTIEARRTGQPLKEDPSAVPVFSVDYGELDFQWREKTPEPPAPCVPEQTEYATIVFPSGLGTSSPARRGSADGPRSPRPLRPEDGHCSWPL

SEQ ID NO: 4 - protein, 149 aa, Macaca fascicularis
PGWFLESPDRPWNAPTFSPALLLVTEGDNATFTCSFSNASESFVLNWYRMSPSNQTDKLAAFPEDRSQPGQDCRFRVTRLPNGRDFHMSVVRARRNDSGTYLCGAISLAPKAQIKESLRAELRVTERRAEVPTAHPSPSPRPAGQFQAL

SEQ ID NO: 5 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 6 - protein, 221 aa, Homo sapiens [sequence omitted]

SEQ ID NO: 7 - protein, 222 aa, Macaca fascicularis
AFTVTVPKDLYVVEYGSNMTIECKFPVEKQLDLTSLIVYWEMEDKNIIQFVHGEEDLKVQHSNYRQRAQLLKDQLSLGNAALRITDVKLQDAGVYRCMISYGGADYKRITVKVNAPYNKINQRILVVDPVTSEHELTCQAEGYPKAEVIWTSSDHQVLSGKTTTTNSKREEKLLNVTSTLRINTTANEIFYCIFRRLDPEENHTAELVIPELPLALPPNERT

SEQ ID NO: 8 - protein, 201 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 9 - protein, 143 aa, Mus musculus
LEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQ

SEQ ID NO: 10 - protein, 5 aa, synthetic construct
  REGION 1..5: note = PD-1 antibody HCDR1
SYAIS

SEQ ID NO: 11 - protein, 5 aa, synthetic construct
  REGION 1..5: note = PD-1 antibody HCDR1
DYVIS

SEQ ID NO: 12 - protein, 5 aa, synthetic construct
  REGION 1..5: note = PD-1 antibody HCDR1
SYVIH

SEQ ID NO: 13 - protein, 17 aa, synthetic construct
  REGION 1..17: note = PD-1 antibody HCDR2
GIIPIFGTANYAQKFQG

SEQ ID NO: 14 - protein, 17 aa, synthetic construct
  REGION 1..17: note = PD-1 antibody HCDR2
GIIPIFDTANYAQKFQG

SEQ ID NO: 15 - protein, 17 aa, synthetic construct
  REGION 1..17: note = PD-1 antibody HCDR2
GIIPIYGTANYAQKFQG

SEQ ID NO: 16 - protein, 14 aa, synthetic construct
  REGION 1..14: note = PD-1 antibody HCDR3
PGLAAAYDTGNLDY

SEQ ID NO: 17 - protein, 14 aa, synthetic construct
  REGION 1..14: note = PD-1 antibody HCDR3
PGLAAAYDTGSLDY

SEQ ID NO: 18 - protein, 11 aa, synthetic construct
  REGION 1..11: note = PD-1 antibody HCDR3
GTLDRTGHLDY

SEQ ID NO: 19 - protein, 11 aa, synthetic construct
  REGION 1..11: note = PD-1 antibody HCDR3
GYVRATGMLDY

SEQ ID NO: 20 - protein, 11 aa, synthetic construct
  REGION 1..11: note = PD-1 antibody LCDR1
RASQSVSSYLA

SEQ ID NO: 21 - protein, 11 aa, synthetic construct
  REGION 1..11: note = PD-1 antibody LCDR1
RASQSVRNYLA

SEQ ID NO: 22 - protein, 11 aa, synthetic construct
  REGION 1..11: note = PD-1 antibody LCDR1
RASQSVDSYLA

SEQ ID NO: 23 - protein, 11 aa, synthetic construct
  REGION 1..11: note = PD-1 antibody LCDR1
RASQSVRSYLA

SEQ ID NO: 24 - protein, 11 aa, synthetic construct
  REGION 1..11: note = PD-1 antibody LCDR1
RASQSVSNYLA

SEQ ID NO: 25 - protein, 11 aa, synthetic construct
  REGION 1..11: note = PD-1 antibody LCDR1
RASQSVDNYLA

SEQ ID NO: 26 - protein, 7 aa, synthetic construct
  REGION 1..7: note = PD-1 antibody LCDR2
DASNRAT

SEQ ID NO: 27 - protein, 7 aa, synthetic construct
  REGION 1..7: note = PD-1 antibody LCDR2
DASDRAT

SEQ ID NO: 28 - protein, 7 aa, synthetic construct
  REGION 1..7: note = PD-1 antibody LCDR2
DASYRAT

SEQ ID NO: 29 - protein, 7 aa, synthetic construct
  REGION 1..7: note = PD-1 antibody LCDR2
DASSRAT

SEQ ID NO: 30 - protein, 7 aa, synthetic construct
  REGION 1..7: note = PD-1 antibody LCDR2
DASTRAT

SEQ ID NO: 31 - protein, 9 aa, synthetic construct
  REGION 1..9: note = PD-1 antibody LCDR3
QQRSNWPLT

SEQ ID NO: 32 - protein, 9 aa, synthetic construct
  REGION 1..9: note = PD-1 antibody LCDR3
QQRNYWPLT

SEQ ID NO: 33 - protein, 9 aa, synthetic construct
  REGION 1..9: note = PD-1 antibody LCDR3
QQRGNWPLT

SEQ ID NO: 34 - protein, 9 aa, synthetic construct
  REGION 1..9: note = PD-1 antibody LCDR3
QQREYWPLT

SEQ ID NO: 35 - protein, 9 aa, synthetic construct
  REGION 1..9: note = PD-1 antibody LCDR3
QQRDYWPLT

SEQ ID NO: 36 - protein, 9 aa, synthetic construct
  REGION 1..9: note = PD-1 antibody LCDR3
QQRGYWPLT

SEQ ID NO: 37 - protein, 9 aa, synthetic construct
  REGION 1..9: note = PD-1 antibody LCDR3
QQRWNWPLT

SEQ ID NO: 38 - protein, 9 aa, synthetic construct
  REGION 1..9: note = PD-1 antibody LCDR3
QQRAYWPLT

SEQ ID NO: 39 - protein, 9 aa, synthetic construct
  REGION 1..9: note = PD-1 antibody LCDR3
QQRAEWPLT

SEQ ID NO: 40 - protein, 9 aa, synthetic construct
  REGION 1..9: note = PD-1 antibody LCDR3
QQRSAWPLT

SEQ ID NO: 41 - protein, 123 aa, synthetic construct
  REGION 1..123: note = PD1H24
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARPGLAAAYDTGNLDYWGQGTLVTVSS

SEQ ID NO: 42 - protein, 123 aa, synthetic construct
  REGION 1..123: note = PD1H131
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGNLDYWGQGTLVTVSS

SEQ ID NO: 43 - protein, 123 aa, synthetic construct
  REGION 1..123: note = PD1H3
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARPGLAAAYDTGSLDYWGQGTLVTVSS

SEQ ID NO: 44 - protein, 120 aa, synthetic construct
  REGION 1..120: note = PD1H108
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGTLDRTGHLDYWGQGTLVTVSS

SEQ ID NO: 45 - protein, 120 aa, synthetic construct
  REGION 1..120: note = PD1H164
QVQLVQSGAEVKKPGSSVKVSCKASGGTFSDYVISWVRQAPGQGLEWMGGIIPIYGTANYAQKFQGRVTITADESTSTAYMELSSLRSEDTAVYYCARGTLDRTGHLDYWGQGTLVTVSS

SEQ ID NO: 46 - protein, 120 aa, synthetic construct
  REGION 1..120: note = PD1H107
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYVRATGMLDYWGQGTLVTVSS

SEQ ID NO: 47 - protein, 120 aa, synthetic construct
  REGION 1..120: note = PD1H163
QVQLVQSGAEVKKPGSSVKVSCKASGGTFKSYVIHWVRQAPGQGLEWMGGIIPIFGTANYAQKFQGRVTITADESTSTAYMELSSLRSEDTAVYYCARGYVRATGMLDYWGQGTLVTVSS

SEQ ID NO: 48 - protein, 123 aa, synthetic construct
  REGION 1..123: note = PD1H170
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSS

SEQ ID NO: 49 - protein, 107 aa, synthetic construct
  REGION 1..107: note = PH9L3
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIK

SEQ ID NO: 50 - protein, 107 aa, synthetic construct
  REGION 1..107: note = PD1L128
EIVLTQSPATLSLSPGERATLSCRASQSVRNYLAWYQQKPGQAPRLLIHDASNRATGIPARFSGSGSGTDFTLTISSLEPEDFAVYYCQQRNYWPLTFGQGTKVEIK

SEQ ID NO: 51 - protein, 107 aa, synthetic construct
  REGION 1..107: note = PD1L101
EIVLTQSPATLSLSPGERATLSCRASQSVDSYLAWYQQKPGQAPRLLIKDASDRATGIPARFSGSGSGTDFTLTISSLEPEDFAVYYCQQRGNWPLTFGQGTKVEIK

SEQ ID NO: 52 - protein, 107 aa, synthetic construct
  REGION 1..107: note = PD1L67
EIVLTQSPATLSLSPGERATLSCRASQSVDSYLAWYQQKPGQAPRLLIYDASNRATGIPARFSGSGSGTDFTLTISSLEPEDFAVYYCQQREYWPLTFGQGTKVEIK

SEQ ID NO: 53 - protein, 107 aa, synthetic construct
  REGION 1..107: note = PD1L71
EIVLTQSPATLSLSPGERATLSCRASQSVRSYLAWYQQKPGQAPRLLIYDASYRATGIPARFSGSGSGTDFTLTISSLEPEDFAVYYCQQRDYWPLTFGQGTKVEIK

SEQ ID NO: 54 - protein, 107 aa, synthetic construct
  REGION 1..107: note = PD1L109
EIVLTQSPATLSLSPGERATLSCRASQSVSSYLAWYQQKPGQAPRLLIKDASNRATGIPARFSGSGSGTDFTLTISSLEPEDFAVYYCQQRGYWPLTFGQGTKVEIK

SEQ ID NO: 55 - protein, 107 aa, synthetic construct
  REGION 1..107: note = PD1L132
EIVLTQSPATLSLSPGERATLSCRASQSVRNYLAWYQQKPGQAPRLLIYDASDRATGIPARFSGSGSGTDFTLTISSLEPEDFAVYYCQQRWNWPLTFGQGTKVEIK

SEQ ID NO: 56 - protein, 107 aa, synthetic construct
  REGION 1..107: note = PD1L148
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIK

SEQ ID NO: 57 - protein, 107 aa, synthetic construct
  REGION 1..107: note = PD1L133
EIVLTQSPATLSLSPGERATLSCRASQSVDSYLAWYQQKPGQAPRLLIHDASNRATGIPARFSGSGSGTDFTLTISSLEPEDFAVYYCQQRNYWPLTFGQGTKVEIK

SEQ ID NO: 58 - protein, 107 aa, synthetic construct
  REGION 1..107: note = PD1L185
EIVLTQSPATLSLSPGERATLSCRASQSVSNYLAWYQQKPGQAPRLLIYDASNRATGIPARFSGSGSGTDFTLTISSLEPEDFAVYYCQQRAYWPLTFGQGTKVEIK

SEQ ID NO: 59 - protein, 107 aa, synthetic construct
  REGION 1..107: note = PD1L187
EIVLTQSPATLSLSPGERATLSCRASQSVSSYLAWYQQKPGQAPRLLIEDASSRATGIPARFSGSGSGTDFTLTISSLEPEDFAVYYCQQRAEWPLTFGQGTKVEIK

SEQ ID NO: 60 - protein, 107 aa, synthetic construct
  REGION 1..107: note = PD1L86
EIVLTQSPATLSLSPGERATLSCRASQSVSSYLAWYQQKPGQAPRLLIHDASTRATGIPARFSGSGSGTDFTLTISSLEPEDFAVYYCQQRNYWPLTFGQGTKVEIK

SEQ ID NO: 61 - protein, 107 aa, synthetic construct
  REGION 1..107: note = PD1L168
EIVLTQSPATLSLSPGERATLSCRASQSVDNYLAWYQQKPGQAPRLLIHDASNRATGIPARFSGSGSGTDFTLTISSLEPEDFAVYYCQQRSAWPLTFGQGTKVEIK

SEQ ID NO: 62 - protein, 107 aa, synthetic construct
  REGION 1..107: note = PD1L190
EIVLTQSPATLSLSPGERATLSCRASQSVSNYLAWYQQKPGQAPRLLIYDASNRATGIPARFSGSGSGTDFTLTISSLEPEDFAVYYCQQRNYWPLTFGQGTKVEIK

SEQ ID NO: 63 - protein, 117 aa, synthetic construct
  REGION 1..117: note = PD1H130
EVQLVESGGGLVQPGGSLRLSCAASGFAFSRYDMSWIRLAPGKGLESVAYISGGGANTYYLDNVKGRFTISRDNAKNSLYLQMNSLRAEDTAVYYCASPYLSYFDVWGQGTLVTVSS

SEQ ID NO: 64 - protein, 117 aa, synthetic construct
  REGION 1..117: note = PD1H129
EVQLVESGGGLVQPGGSLRLSCAASGFAFSRYDMSWVRQAPGKGLESVAYISGGGANTYYLDNVKGRFTISRDNAKNSLYLQMNSLRAEDTAVYYCASPYLSYFDVWGQGTLVTVSS

SEQ ID NO: 65 - protein, 107 aa, synthetic construct
  REGION 1..107: note = PD1L62
EIVMTQSPATLSVSPGERATLSCRASQSLSDYLHWYQQKPGQAPRLLIKSASQSISGIPARFSGSGSGTEFTLTISSLQSEDFAVYYCQNGHSFPYTFGQGTKLEIK

SEQ ID NO: 66 - protein, 5 aa, synthetic construct
  REGION 1..5: note = PD1 antibody HCDR1
RYDMS

SEQ ID NO: 67 - protein, 17 aa, synthetic construct
  REGION 1..17: note = PD1 antibody HCDR2
YISGGGANTYYLDNVKG

SEQ ID NO: 68 - protein, 8 aa, synthetic construct
  REGION 1..8: note = PD1 antibody HCDR3
PYLSYFDV

SEQ ID NO: 69 - protein, 11 aa, synthetic construct
  REGION 1..11: note = PD1 antibody LCDR1
RASQSLSDYLH

SEQ ID NO: 70 - protein, 7 aa, synthetic construct
  REGION 1..7: note = PD1 antibody LCDR2
SASQSIS

SEQ ID NO: 71 - protein, 9 aa, synthetic construct
  REGION 1..9: note = PD1 antibody LCDR3
QNGHSFPYT

SEQ ID NO: 72 - protein, 450 aa, synthetic construct
  REGION 1..450: note = PD1B244 HC
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 73 - protein, 214 aa, synthetic construct
  REGION 1..214: note = PD1B244 LC
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 444 aa, synthetic construct
  REGION 1..444: note = PD1B243 HC
EVQLVESGGGLVQPGGSLRLSCAASGFAFSRYDMSWVRQAPGKGLESVAYISGGGANTYYLDNVKGRFTISRDNAKNSLYLQMNSLRAEDTAVYYCASPYLSYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - protein, 214 aa, synthetic construct
  REGION 1..214: note = PD1B243 LC
EIVMTQSPATLSVSPGERATLSCRASQSLSDYLHWYQQKPGQAPRLLIKSASQSISGIPARFSGSGSGTEFTLTISSLQSEDFAVYYCQNGHSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 447 aa, synthetic construct
  REGION 1..447: note = PD1B245 HC
QVQLVQSGAEVKKPGSSVKVSCKASGGTFSDYVISWVRQAPGQGLEWMGGIIPIYGTANYAQKFQGRVTITADESTSTAYMELSSLRSEDTAVYYCARGTLDRTGHLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 214 aa, synthetic construct
  REGION 1..214: note = PD1B245 LC
EIVLTQSPATLSLSPGERATLSCRASQSVSSYLAWYQQKPGQAPRLLIHDASTRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 443 aa, synthetic construct
  REGION 1..443: note = TIM3 antibody heavy chain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPYAPLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 79 - protein, 214 aa, synthetic construct
  REGION 1..214: note = TIM-3 antibody light chain
EIVLTQSPATLSLSPGERATLSCRASQSVNDYLAWYQQKPGQAPRLLIYDASNRATGIPARFSGSGSGTDFTLTISSLEPEDFAVYYCQQGGHAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 450 aa, synthetic construct
  REGION 1..450: note = TIM-3 antibody heavy chain
EVQLVQSGAEVKKPGESLKISCKGSGYSFTSYWMQWVRQMPGKGLEWMGAIYPGDGDIRYTQNFKGQVTISADKSISTAYLQWSSLKASDTAMYYCARWEKSTTVVQRNYFDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 81 - protein, 213 aa, synthetic construct
  REGION 1..213: note = TIM-3 antibody light chain
DIQMTQSPSSLSASVGDRVTITCKASENVGTFVSWYQQKPGKAPKLLIYGASNRYTGVPSRFSGSGSGTDFTLTISSLQPEDFATYYCGQSYSYPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - skipped

SEQ ID NO: 83 - protein, 17 aa, synthetic construct
  REGION 1..17: note = PD-1 HCDR2 genus
  VARIANT 6: note = MISC_FEATURE - Xaa may be Tyr or Phe
  VARIANT 7: note = MISC_FEATURE - Xaa may be Gly or Asp
GIIPIXXTANYAQKFQG

SEQ ID NO: 84 - protein, 14 aa, synthetic construct
  REGION 1..14: note = PD-1 HCDR3 genus 1
  VARIANT 11: note = MISC_FEATURE - Xaa is Asn or Ser
PGLAAAYDTGXLDY

SEQ ID NO: 85 - protein, 11 aa, synthetic construct
  REGION 1..11: note = PD-1 HCDR3 genus 2
  VARIANT 2: note = MISC_FEATURE - Xaa is Thr or Tyr
  VARIANT 3: note = MISC_FEATURE - Xaa is Leu or Val
  VARIANT 4: note = MISC_FEATURE - Xaa is Asp or Arg
  VARIANT 5: note = MISC_FEATURE - Xaa is Arg or Ala
  VARIANT 8: note = MISC_FEATURE - Xaa is His or Met
GXXXXTGXLDY

SEQ ID NO: 86 - protein, 11 aa, synthetic construct
  REGION 1..11: note = PD-1 LCDR1 genus
  VARIANT 7: note = MISC_FEATURE - Xaa is Ser, Arg or Asp
  VARIANT 8: note = MISC_FEATURE - Xaa is Ser or Asn
RASQSVXXYLA

SEQ ID NO: 87 - protein, 7 aa, synthetic construct
  REGION 1..7: note = PD-1 LCDR2 genus
  VARIANT 4: note = MISC_FEATURE - Xaa is Asn, Asp, Tyr, Ser or Thr
DASXRAT

SEQ ID NO: 88 - protein, 9 aa, synthetic construct
  REGION 1..9: note = PD-1 LCDR3 genus
  VARIANT 4: note = MISC_FEATURE - Xaa is Ser, Asn, Gly, Glu, Asp, Trp or Ala
  VARIANT 5: note = MISC_FEATURE - Xaa is Asn, Tyr, Glu or Ala
QQRXXWPLT

SEQ ID NO: 89 - protein, 179 aa, Homo sapiens
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIR

SEQ ID NO: 90 - protein, 5 aa, synthetic construct
  REGION 1..5: note = TIM-3 antibody HCDR1
NYWMS

SEQ ID NO: 91 - protein, 5 aa, synthetic construct
  REGION 1..5: note = TIM-3 antibody HCDR1
SYAMS

SEQ ID NO: 92 - protein, 5 aa, synthetic construct
  REGION 1..5: note = TIM-3 antibody HCDR1
GYWMH

SEQ ID NO: 93 - protein, 5 aa, synthetic construct
  REGION 1..5: note = TIM-3 antibody HCDR1
DYWMS

SEQ ID NO: 94 - protein, 5 aa, synthetic construct
  REGION 1..5: note = TIM-3 antibody HCDR1
SYVMY

SEQ ID NO: 95 - protein, 6 aa, synthetic construct
  REGION 1..6: note = TIM-3 antibody HCDR1
SDYAWN

SEQ ID NO: 96 - protein, 5 aa, synthetic construct
  REGION 1..5: note = TIM-3 antibody HCDR1
DTYLH

SEQ ID NO: 97 - protein, 5 aa, synthetic construct
  REGION 1..5: note = TIM-3 antibody HCDR1
SYWMQ

SEQ ID NO: 98 - protein, 5 aa, synthetic construct
  REGION 1..5: note = TIM-3 antibody HCDR1
SYGVH

SEQ ID NO: 99 - protein, 17 aa, synthetic construct
  REGION 1..17: note = TIM-3 antibody HCDR2
AISGSGGSTYYADSVKG

SEQ ID NO: 100 - protein, 17 aa, synthetic construct
  REGION 1..17: note = TIM-3 antibody HCDR2
AISYSGSSTYYADSVKG

SEQ ID NO: 101 - protein, 17 aa, synthetic construct
  REGION 1..17: note = TIM-3 antibody HCDR2
VIKYSGGSKYYADSVKG

SEQ ID NO: 102 - protein, 17 aa, synthetic construct
  REGION 1..17: note = TIM-3 antibody HCDR2
YINPYNDGTKYNEKFKG

SEQ ID NO: 103 - protein, 16 aa, synthetic construct
  REGION 1..16: note = TIM-3 antibody HCDR2
YINYSGRTSYNPSLKS

SEQ ID NO: 104 - protein, 17 aa, synthetic construct
  REGION 1..17: note = TIM-3 antibody HCDR2
RIDPTNGNIKYDPKFQG

SEQ ID NO: 105 - protein, 17 aa, synthetic construct
  REGION 1..17: note = TIM-3 antibody HCDR2
AIYPGDGDIRYTQNFKG

SEQ ID NO: 106 - protein, 16 aa, synthetic construct
  REGION 1..16: note = TIM-3 antibody HCDR2
VIWSDGSTTYNSALKS

SEQ ID NO: 107 - protein, 10 aa, synthetic construct
  REGION 1..10: note = TIM-3 antibody HCDR2
DHWDPNFLDY

SEQ ID NO: 108 - protein, 8 aa, synthetic construct
  REGION 1..8: note = TIM-3 antibody HCDR3
SPYAPLDY

SEQ ID NO: 109 - protein, 10 aa, synthetic construct
  REGION 1..10: note = TIM-3 antibody HCDR3
NEEPDDRLDY

SEQ ID NO: 110 - protein, 7 aa, synthetic construct
  REGION 1..7: note = TIM-3 antibody HCDR3
GTNWLDY

SEQ ID NO: 111 - protein, 8 aa, synthetic construct
  REGION 1..8: note = TIM-3 antibody HCDR3
ELEGVFDY

SEQ ID NO: 112 - protein, 10 aa, synthetic construct
  REGION 1..10: note = TIM-3 antibody HCDR3
DDYDVAPFAY

SEQ ID NO: 113 - protein, 6 aa, synthetic construct
  REGION 1..6: note = TIM-3 antibody HCDR3
GGNFDY

SEQ ID NO: 114 - protein, 8 aa, synthetic construct
  REGION 1..8: note = TIM-3 antibody HCDR3
PYYGFFDY

SEQ ID NO: 115 - protein, 15 aa, synthetic construct
  REGION 1..15: note = TIM-3 antibody HCDR3
WEKSTTVVQRNYFDY

SEQ ID NO: 116 - protein, 12 aa, synthetic construct
  REGION 1..12: note = TIM-3 antibody HCDR3
QANYRYDSAMDY

SEQ ID NO: 117 - protein, 12 aa, synthetic construct
  REGION 1..12: note = TIM-3 antibody LCDR1
RASQSVSSSYLA

SEQ ID NO: 118 - protein, 11 aa, synthetic construct
  REGION 1..11: note = TIM-3 antibody LCDR1
RASQSVNDYLA

SEQ ID NO: 119 - protein, 17 aa, synthetic construct
  REGION 1..17: note = TIM-3 antibody LCDR1
KSSQSVLASSNNKNYLA

SEQ ID NO: 120 - protein, 12 aa, synthetic construct
  REGION 1..12: note = TIM-3 antibody LCDR1
RASQSVSNSTLA

SEQ ID NO: 121 - protein, 15 aa, synthetic construct
  REGION 1..15: note = TIM-3 antibody LCDR1
RASESLDSYGNSYIH

SEQ ID NO: 122 - protein, 11 aa, synthetic construct
  REGION 1..11: note = TIM-3 antibody LCDR1
QATQDIVKNLN

SEQ ID NO: 123 - protein, 11 aa, synthetic construct
  REGION 1..11: note = TIM-3 antibody LCDR1
KASQDVNTAVA

SEQ ID NO: 124 - protein, 11 aa, synthetic construct
  REGION 1..11: note = TIM-3 antibody LCDR1
KASENVGTFVS

SEQ ID NO: 125 - protein, 15 aa, synthetic construct
  REGION 1..15: note = TIM-3 antibody LCDR1
KASQSVDYDGDSYMN

SEQ ID NO: 126 - protein, 7 aa, synthetic construct
  REGION 1..7: note = TIM-3 antibody LCDR2
GASSRAT

SEQ ID NO: 127 - protein, 7 aa, synthetic construct
  REGION 1..7: note = TIM-3 antibody LCDR2
DASNRAT

SEQ ID NO: 128 - protein, 7 aa, synthetic construct
  REGION 1..7: note = TIM-3 antibody LCDR2
WASTRES

SEQ ID NO: 129 - protein, 7 aa, synthetic construct
  REGION 1..7: note = TIM-3 antibody LCDR2
TASSRAT

SEQ ID NO: 130 - protein, 7 aa, synthetic construct
  REGION 1..7: note = TIM-3 antibody LCDR2
LASNLES

SEQ ID NO: 131 - protein, 7 aa, synthetic construct
  REGION 1..7: note = TIM-3 antibody LCDR2
YVTELAE

SEQ ID NO: 132 - protein, 7 aa, synthetic construct
  REGION 1..7: note = TIM-3 antibody LCDR2
SATYRYT

SEQ ID NO: 133 - protein, 7 aa, synthetic construct
  REGION 1..7: note = TIM-3 antibody LCDR2
GASNRYT

SEQ ID NO: 134 - protein, 7 aa, synthetic construct
  REGION 1..7: note = TIM-3 antibody LCDR2
TAANLQS

SEQ ID NO: 135 - protein, 9 aa, synthetic construct
  REGION 1..9: note = TIM-3 antibody LCDR3
QQYGSSPLT

SEQ ID NO: 136 - protein, 9 aa, synthetic construct
  REGION 1..9: note = TIM-3 antibody LCDR3
QQGGHAPIT

SEQ ID NO: 137 - protein, 9 aa, synthetic construct
  REGION 1..9: note = TIM-3 antibody LCDR3
QQYYSTPLT

SEQ ID NO: 138 - protein, 280 aa, Homo sapiens
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 139 - protein, 9 aa, synthetic construct
  REGION 1..9: note = TIM-3 antibody LCDR3
QQSYTSPWT

SEQ ID NO: 140 - protein, 9 aa, synthetic construct
  REGION 1..9: note = TIM-3 antibody LCDR3
QQNNEDPFT

SEQ ID NO: 141 - protein, 9 aa, synthetic construct
  REGION 1..9: note = TIM-3 antibody LCDR3
LQFYEFPLT

SEQ ID NO: 142 - protein, 9 aa, synthetic construct
  REGION 1..9: note = TIM-3 antibody LCDR3
QQHYSTPYT

SEQ ID NO: 143 - protein, 8 aa, synthetic construct
  REGION 1..8: note = TIM-3 antibody LCDR3
GQSYSYPT

SEQ ID NO: 144 - protein, 8 aa, synthetic construct
  REGION 1..8: note = TIM-3 antibody LCDR3
GQSYSYPT

SEQ ID NO: 145 - protein, 119 aa, synthetic construct
  REGION 1..119: note = TM3H21
EVQLLESGGGLVQPGGSLRLSCAASGFTFSNYWMSWVRQAPGKGLEWVSAISGSGGSTYYADSVKGRFTISRDNSKNTLYLQMNSLRAEDTAVYYCAKDHWDPNFLDYWGQGTLVTVSS

SEQ ID NO: 146 - protein, 117 aa, synthetic construct
  REGION 1..117: note = TM3H24
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPYAPLDYWGQGTLVTVSS

SEQ ID NO: 147 - protein, 116 aa, synthetic construct
  REGION 1..116: note = TM3H30
EVQLLESGGGLVQPGGSLRLSCAASGFTFSGYWMHWVRQAPGKGLEWVSAISYSGSSTYYADSVKGRFTISRDNSKNTLYLQMNSLRAEDTAVYYCAKGTNWLDYWGQGTLVTVSS

SEQ ID NO: 148 - protein, 119 aa, synthetic construct
  REGION 1..119: note = TM3H31
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKNEEPDDRLDYWGQGTLVTVSS

SEQ ID NO: 149 - protein, 117 aa, synthetic construct
  REGION 1..117: note = TM3H65
EVQLLESGGGLVQPGGSLRLSCAASGFTFSDYWMSWVRQAPGKGLEWVSVIKYSGGSKYYADSVKGRFTISRDNSKNTLYLQMNSLRAEDTAVYYCAKELEGVFDYWGQGTLVTVSS

SEQ ID NO: 150 - protein, 119 aa, synthetic construct
  REGION 1..119: note = TM3H141
EVQLQQSGPELLKPGASVKMSCKASGYTFTSYVMYWVKQKPGQGLEWIGYINPYNDGTKYNEKFKGKATLTSDKSSSTAYMELSRLTSEDSAVYYCTRDDYDVAPFAYWGQGTLVTVSA

SEQ ID NO: 151 - protein, 115 aa, synthetic construct
  REGION 1..115: note = TM3H96
DVQLQESGPGLVKPSQSLSLTCTVTGYSITSDYAWNWIRQFPGNKLEWMGYINYSGRTSYNPSLKSRISITRDTSKNQFFLQLNSVTTEDTATYYCTSGGNFDYWGQGTTLTVSS

SEQ ID NO: 152 - protein, 117 aa, synthetic construct
  REGION 1..117: note = TM3H99
EVQLQQSGAELVKPGASVKLSCTASGFHIKDTYLHWVKQRPEQGLEWIGRIDPTNGNIKYDPKFQGKATITSDTSSNTAYLQLSSLTSEDTAVYYCARPYYGFFDYWGQGTTLTVSS

SEQ ID NO: 153 - protein, 130 aa, synthetic construct
  REGION 1..130: note = TM3H144
EVQLQQSGAELARPGASVKLSCKASGYTFTSYWMQWVKQRPGQGLEWIGAIYPGDGDIRYTQNFKGKATLTADKSSSTAYMQLSSLASEDSAVYYCARWEKSTTVVQRNYFDYWGQGTTLTVSSCRRECT

SEQ ID NO: 154 - protein, 120 aa, synthetic construct
  REGION 1..120: note = TM3H102
QVQLKESGPGLVAPSQSLSITCTISGFSLTSYGVHWVRQPPGKGLEWLVVIWSDGSTTYNSALKSRLSISKDNSKSQVFLKMNSLQTDDTAMYYCARQANYRYDSAMDYWGQGTSVTVSS

SEQ ID NO: 155 - protein, 108 aa, synthetic construct
  REGION 1..108: note = PH9L1
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 156 - protein, 107 aa, synthetic construct
  REGION 1..107: note = TM3L33
EIVLTQSPATLSLSPGERATLSCRASQSVNDYLAWYQQKPGQAPRLLIYDASNRATGIPARFSGSGSGTDFTLTISSLEPEDFAVYYCQQGGHAPITFGQGTKVEIK

SEQ ID NO: 157 - protein, 113 aa, synthetic construct
  REGION 1..113: note = PYYL6
DIVMTQSPDSLAVSLGERATINCKSSQSVLASSNNKNYLAWYQQKPGQPPKLLIYWASTRESGVPDRFSGSGSGTDFTLTISSLQAEDVAVYYCQQYYSTPLTFGQGTKVEIK

SEQ ID NO: 158 - protein, 108 aa, synthetic construct
  REGION 1..108: note = TM3L12
EIVLTQSPGTLSLSPGERATLSCRASQSVSNSTLAWYQQKPGQAPRLLIYTASSRATGIPDRFSGSGSGTDFTLTISRLEPEDFAVYYCQQSYTSPWTFGQGTKVEIK

SEQ ID NO: 159 - protein, 111 aa, synthetic construct
  REGION 1..111: note = TM3L61
DIVLTQSPASLAVSLGQRATISCRASESLDSYGNSYIHWYQQKPGQPPKLLIYLASNLESGVPARFSGSGSKTDFTLTIDPVEADDPATYYCQQNNEDPFTFGSGTKLEIK

SEQ ID NO: 160 - protein, 107 aa, synthetic construct
  REGION 1..107: note = TM3L62
DIVMTQSPSSMSASLGDRITITCQATQDIVKNLNWYQQKPGKPPSFLIHYVTELAEGVPSRFSGSGSGSDYSLTISNLESEDFADYYCLQFYEFPLTFGAGTKLELK

SEQ ID NO: 161 - protein, 107 aa, synthetic construct
  REGION 1..107: note = TM3L52
DIVMTQSHKFMSTSVGDRVSITCKASQDVNTAVAWYQQKPGQSPKLLIYSATYRYTGVPDRFTGSGSGTDFTFTISSVQAEDLAVYYCQQHYSTPYTFGSGTKLEIK

SEQ ID NO: 162 - protein, 105 aa, synthetic construct
  REGION 1..105: note = TM3L67
DVQMIQSPKSMSMSVGERVTLSCKASENVGTFVSWYQQKPDQSPKLLIYGASNRYTGVPDRFTGSGSATDFTLTISSVQAEDLADYHCGQSYSYPTFGSGTKLEM

SEQ ID NO: 163 - protein, 111 aa, synthetic construct
  REGION 1..111: note = TM3L64
DIQMTQSPASLAVSLGQRATISCKASQSVDYDGDSYMNWYQQKPGQPPKLLIYTAANLQSGIPARFSGSGSGTDFTLNIHPVEEEDAATYYCQQSNEDPFTFGSGTKLEIK

SEQ ID NO: 164 - skipped

SEQ ID NO: 165 - protein, 17 aa, synthetic construct
  REGION 1..17: note = TIM-3 HCDR2 genus
  VARIANT 1: note = MISC_FEATURE - Xaa is Ala or Val
  VARIANT 3: note = MISC_FEATURE - Xaa is Ser or Lys
  VARIANT 4: note = MISC_FEATURE - Xaa is Gly or Tyr
  VARIANT 9: note = MISC_FEATURE - Xaa is Thr or Lys
XIXXSGGSXYYADSVKG

SEQ ID NO: 166 - skipped

SEQ ID NO: 167 - protein, 17 aa, synthetic construct
  REGION 1..17: note = TIM-3 LCDR1 genus
  VARIANT 1: note = MISC_FEATURE - Xaa is Arg or Lys
  VARIANT 2: note = MISC_FEATURE - Xaa is Ala or Ser
  VARIANT 7: note = MISC_FEATURE - Xaa is Ser, Asn or Leu
  VARIANT 8: note = MISC_FEATURE - Xaa is Ser, Ala, Asn or deleted
  VARIANT 9: note = MISC_FEATURE - Xaa is Ser or deleted
  VARIANT 10: note = MISC_FEATURE - Xaa is Ser or deleted
  VARIANT 11: note = MISC_FEATURE - Xaa is Asn or deleted
  VARIANT 12: note = MISC_FEATURE - Xaa is Asn or deleted
  VARIANT 13: note = MISC_FEATURE - Xaa is Lys or deleted
  VARIANT 14: note = MISC_FEATURE - Xaa is Ser, Asn or Asp
  VARIANT 15: note = MISC_FEATURE - Xaa is Tyr or Thr
XXSQSVXXXXXXXXXLA

SEQ ID NO: 168 - skipped

SEQ ID NO: 169 - protein, 9 aa, synthetic construct
  REGION 1..9: note = TIM-3 LCDR3 genus
  VARIANT 3: note = MISC_FEATURE - Xaa is Tyr, Gly or Ser
  VARIANT 4: note = MISC_FEATURE - Xaa is Gly or Tyr
  VARIANT 5: note = MISC_FEATURE - Xaa is Ser, His or Thr
  VARIANT 6: note = MISC_FEATURE - Xaa is Ser, Ala or Thr
  VARIANT 8: note = MISC_FEATURE - Xaa is Leu, Ile or Trp
QQXXXXPXT

SEQ ID NO: 170 - protein, 98 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 171 - protein, 95 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 172 - protein, 124 aa, synthetic construct
  REGION 1..124: note = TM3H162
EVQLVQSGAEVKKPGESLKISCKGSGYSFTSYWMQWVRQMPGKGLEWMGAIYPGDGDIRYTQNFKGQVTISADKSISTAYLQWSSLKASDTAMYYCARWEKSTTVVQRNYFDYWGQGTTVTVSS

SEQ ID NO: 173 - protein, 106 aa, synthetic construct
  REGION 1..106: note = TM3L85
DIQMTQSPSSLSASVGDRVTITCKASENVGTFVSWYQQKPGKAPKLLIYGASNRYTGVPSRFSGSGSGTDFTLTISSLQPEDFATYYCGQSYSYPTFGQGTKLEIK

SEQ ID NO: 174 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 175 - protein, 98 aa, Homo sapiens
QVQLVQSGAEVKKPGASVKVSCKASGYTFTGYYMHWVRQAPGQGLEWMGRINPNSGGTNYAQKFQGRVTSTRDTSISTAYMELSRLRSDDTVVYYCAR

SEQ ID NO: 176 - protein, 99 aa, Homo sapiens
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAR

SEQ ID NO: 177 - protein, 98 aa, Homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 178 - protein, 100 aa, Homo sapiens
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCARI

SEQ ID NO: 179 - protein, 98 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 180 - protein, 96 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 181 - protein, 96 aa, Homo sapiens; identical to SEQ ID NO: 180

SEQ ID NO: 182 - protein, 96 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPI

SEQ ID NO: 183 - protein, 95 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 184 - protein, 127 aa, synthetic construct
  REGION 1..127: note = VH of gp120 binding mAb
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSS

SEQ ID NO: 185 - protein, 108 aa, synthetic construct
  REGION 1..108: note = VL of gp120 binding mAb
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERK

SEQ ID NO: 186 - protein, 449 aa, synthetic construct
  REGION 1..449: note = Bispecific mAb HC1
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFLLYSKLTVDKSRWQQGNVFSCSVMHEALHNHYTQKSLSLSPGK

SEQ ID NO: 187 - protein, 443 aa, synthetic construct
  REGION 1..443: note = Bispecific mAb HC1
EVQLVESGGGLVQPGGSLRLSCAASGFAFSRYDMSWVRQAPGKGLESVAYISGGGANTYYLDNVKGRFTISRDNAKNSLYLQMNSLRAEDTAVYYCASPYLSYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFLLYSKLTVDKSRWQQGNVFSCSVMHEALHNHYTQKSLSLSPGK

SEQ ID NO: 188 - protein, 214 aa, synthetic construct; identical to SEQ ID NO: 73
  REGION 1..214: note = Bispecific mAb LC1

SEQ ID NO: 189 - protein, 214 aa, synthetic construct; identical to SEQ ID NO: 75
  REGION 1..214: note = Bispecific mAb LC1

SEQ ID NO: 190 - protein, 450 aa, synthetic construct
  REGION 1..450: note = Bispecific mAb HC2
EVQLQQSGAELARPGASVKLSCKASGYTFTSYWMQWVKQRPGQGLEWIGAIYPGDGDIRYTQNFKGKATLTADKSSSTAYMQLSSLASEDSAVYYCARWEKSTTVVQRNYFDYWGQGTTLTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 191 - protein, 443 aa, synthetic construct
  REGION 1..443: note = Bispecific mAb HC2
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPYAPLDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 192 - protein, 450 aa, synthetic construct
  REGION 1..450: note = Bispecific mAb HC2
EVQLVQSGAEVKKPGESLKISCKGSGYSFTSYWMQWVRQMPGKGLEWMGAIYPGDGDIRYTQNFKGQVTISADKSISTAYLQWSSLKASDTAMYYCARWEKSTTVVQRNYFDYWGQGTTVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 193 - protein, 213 aa, synthetic construct
  REGION 1..213: note = Bispecific mAb LC2
DVQMIQSPKSMSMSVGERVTLSCKASENVGTFVSWYQQKPDQSPKLLIYGASNRYTGVPDRFTGSGSATDFTLTISSVQAEDLADYHCGQSYSYPTFGSGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 214 aa, synthetic construct; identical to SEQ ID NO: 79
  REGION 1..214: note = Bispecific mAb LC2

SEQ ID NO: 195 - protein, 213 aa, synthetic construct; identical to SEQ ID NO: 81
  REGION 1..213: note = Bispecific mAb LC2

SEQ ID NO: 196 - DNA, 369 bp, synthetic construct
  misc_feature 1..369: note = DNA of PD1H170
  source 1..369: mol_type = other DNA
caggtgcagctggtgcagagcggcgcggaagtgaaaaaaccgggcagcagcgtgaaagtgagctgcaaagcgagcggcggcacctttagcagctatgcgattagctgggtgcgccaggcgccgggccagggcctggaatggatgggcggcattattccgatttttgacaccgcgaactatgcgcagaaatttcagggccgcgtgaccattaccgcggatgaaagcaccagcaccgcgtatatggaactgagcagcctgcgcagcgaagataccgcggtgtattattgcgcgcgccctggtctcgctgcggcttatgatactggttccttggactattggggccagggcaccctggtgaccgtgagcagc

SEQ ID NO: 197 - DNA, 321 bp, synthetic construct
  misc_feature 1..321: note = DNA of PD1L148
  source 1..321: mol_type = other DNA
gaaattgtgctgacccagagcccggcgaccctgagcctgagcccgggcgaacgcgcgaccctgagctgccgcgcgagccagagcgttcgctcctacctggcgtggtatcagcagaaaccgggccaggcgccgcgcctgctgatctacgacgcgagcaatcgtgcgaccggcattccggcgcgctttagcggctccggtagcggcaccgattttaccctgaccattagcagcctggaaccggaagattttgcggtgtattattgccagcaacgtaattattggccgctgacctttggccagggcaccaaagtggaaattaaa

SEQ ID NO: 198 - DNA, 351 bp, synthetic construct
  misc_feature 1..351: note = DNA of PD1H129
  source 1..351: mol_type = other DNA
gaagtgcagctggtggaatctggcggcggactggtgcagcctggcggatctctgagactgagctgtgccgccagcggcttcgccttcagcagatacgacatgagctgggtgcgccaggcccctggcaaaggactggaaagcgtggcctacatctctggcggaggcgccaacacctactacctggacaacgtgaagggccggttcaccatcagccgggacaacgccaagaacagcctgtacctgcagatgaactccctgcgggccgaggacaccgccgtgtactattgcgcctccccctacctgagctacttcgacgtgtggggccagggcacactcgtgaccgtgtcatct

SEQ ID NO: 199 - DNA, 321 bp, synthetic construct
  misc_feature 1..321: note = DNA of PD1L62
  source 1..321: mol_type = other DNA
gagatcgtgatgacccagagccctgccaccctgtccgtgtctccaggcgaaagagccaccctgagctgcagagccagccagagcctgagcgactacctgcactggtatcagcagaagcccggccaggcccccagactgctgatcaagtctgccagccagtccatcagcggcatccccgccagattttctggcagcggctccggcaccgagttcaccctgacaatcagcagcctgcagagcgaggacttcgccgtgtactactgccagaacggccacagcttcccttacaccttcggccagggcaccaagctggaaatcaag

SEQ ID NO: 200 - DNA, 360 bp, synthetic construct
  misc_feature 1..360: note = DNA of PD1H163
  source 1..360: mol_type = other DNA
caggtgcagctggtgcagagcggcgcggaagtgaaaaaaccgggcagcagcgtgaaagtgagctgcaaagcgagcggcggcaccttcaagtcctatgtgattcattgggtgcgccaggcgccgggccagggcctggaatggatgggcggtattatcccaatttttggcaccgccaattatgcgcagaaatttcagggccgcgtgaccattaccgctgatgaaagcaccagcaccgcgtatatggaactgagcagcctgcgcagcgaagataccgcggtgtattattgcgcgcgcggttatgtgcgggctacgggcatgttggactattggggccagggcaccctggtgaccgtgagcagc

SEQ ID NO: 201 - DNA, 321 bp, synthetic construct
  misc_feature 1..321: note = DNA of PD1L185
  source 1..321: mol_type = other DNA
gaaattgtgctgacccagagcccggcgaccctgagcctgagcccgggcgaacgcgcgaccctgagctgccgcgcgagccagagcgttagcaattatctggcgtggtatcagcagaaaccgggccaggcgccgcgcctgctgatctacgacgccagcaatcgcgcgaccggcattccggcgcgctttagcggctccggtagcggcaccgattttaccctgaccattagcagcctggaaccggaagattttgcggtgtattattgccagcaacgtgcatattggccgctgacctttggccagggcaccaaagtggaaattaaa

SEQ ID NO: 202 - DNA, 360 bp, synthetic construct
  misc_feature 1..360: note = DNA of PD1H164
  source 1..360: mol_type = other DNA
caggtgcagctggtgcagagcggcgcggaagtgaaaaaaccgggcagcagcgtgaaagtgagctgcaaagcgagcggcggcaccttcagcgattatgtgatttcctgggtgcgccaggcgccgggccagggcctggaatggatgggcggtattatcccgatttacgggaccgctaactatgcgcagaaatttcagggccgcgtgaccattaccgctgatgaaagcaccagcaccgcgtatatggaactgagcagcctgcgcagcgaagataccgcggtgtattattgcgcgcgcggtaccctcgaccggaccgggcatttggactattggggccagggcaccctggtgaccgtgagcagc

SEQ ID NO: 203 - DNA, 321 bp, synthetic construct
  misc_feature 1..321: note = DNA of PD1L86
  source 1..321: mol_type = other DNA
gaaattgtgctgacccagagcccggcgaccctgagcctgagcccgggcgaacgcgcgaccctgagctgccgcgcgagccagagcgtctcctcctaccttgcgtggtatcagcagaaaccgggccaggcgccgcgcctgctgatccacgacgcctctacgcgtgcgaccggcattccggcgcgctttagcggctccggtagcggcaccgattttaccctgaccattagcagcctggaaccggaagattttgcggtgtattattgccagcaacgtaattattggccgctcacctttggccagggcaccaaagtggaaattaaa

SEQ ID NO: 204 - DNA, 351 bp, synthetic construct
  misc_feature 1..351: note = DNA of TM3H24
  source 1..351: mol_type = other DNA
gaagtgcagctgctggaaagcggcggcggcctggtgcagccgggcggcagcctgcgcctgagctgcgcggcaagcggctttacctttagcagctatgcgatgagctgggtgcgccaggcgccgggcaaaggcctggaatgggtgagcgcgattagcggcagcggcggcagcacctattatgcggatagcgtgaaaggccgctttaccattagccgcgataacagcaaaaacaccctgtatctgcagatgaacagcctgcgcgcggaagataccgcggtgtattattgcgcgaaatccccgtacgcgcccttggactattggggccagggcaccctggtgaccgtgagcagc

SEQ ID NO: 205 - DNA, 321 bp, synthetic construct
  misc_feature 1..321: note = DNA of TM3L33
  source 1..321: mol_type = other DNA
gaaattgtgctgacccagagcccggcgaccctgagcctgagcccgggcgaacgcgcgacccttagctgccgtgcaagtcagagtgtgaacgactacctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagggtggtcacgcgccgatcacctttggccagggcaccaaagtggaaattaaa

SEQ ID NO: 206 - DNA, 372 bp, synthetic construct
  misc_feature 1..372: note = DNA of TM3H162
  source 1..372: mol_type = other DNA
gaagtgcagctggtgcagtctggcgccgaagtgaagaagcctggcgagagcctgaagatcagctgcaagggcagcggctacagcttcaccagctactggatgcagtgggtgcgccagatgcctggcaagggcctggaatggatgggcgccatctatcccggcgacggcgacatcagatacacccagaacttcaagggccaagtgaccatcagcgccgacaagagcatcagcaccgcctacctgcagtggtccagcctgaaggccagcgacaccgccatgtactactgtgccagatgggagaagtccaccaccgtggtgcagcggaactacttcgactactggggccagggcaccacagtgaccgtgtctagt

SEQ ID NO: 207 - DNA, 318 bp, synthetic construct
  misc_feature 1..318: note = DNA of TM3L85
  source 1..318: mol_type = other DNA
gacatccagatgacccagagccccagcagcctgtctgccagcgtgggcgacagagtgaccatcacatgcaaggccagcgagaacgtgggcaccttcgtgtcctggtatcagcagaagcccggcaaggcccccaagctgctgatctacggcgccagcaacagatacaccggcgtgcccagcagattcagcggctctggcagcggcaccgacttcaccctgaccatctctagcctgcagcccgaggacttcgccacctactactgcggccagagctacagctaccccacctttggccagggcaccaagctggaaatcaag

SEQ ID NO: 208 - DNA, 357 bp, synthetic construct
  misc_feature 1..357: note = DNA of TM3H21
  source 1..357: mol_type = other DNA
gaagtgcagctgctggaaagcggcggcggcctggtgcagccgggcggcagcctgcgcctgagctgcgcggcgagcggctttacctttagcaactattggatgagctgggtgcgccaggcgccgggcaaaggcctggaatgggtgagcgcgattagcggcagcggcggcagcacctattatgcggatagcgtgaaaggccgctttaccattagccgcgataacagcaaaaacaccctgtatctgcagatgaacagcctgcgcgcggaagataccgcggtgtattattgcgcgaaagatcattgggatcccaattttttggactattggggccagggcaccctggtgaccgtgagcagc

SEQ ID NO: 209 - DNA, 324 bp, synthetic construct
  misc_feature 1..324: note = DNA of PH9L1
  source 1..324: mol_type = other DNA
gaaattgtgctgacccagagcccgggcaccctgagcctgagcccgggcgaacgcgcgaccctgagctgccgcgcgagccagagcgtgagcagcagctatctggcgtggtatcagcagaaaccgggccaggcgccgcgcctgctgatttatggcgcgagcagccgcgcgaccggcattccggatcgctttagcggcagcggcagcggcaccgattttaccctgaccattagccgcctggaaccggaagattttgcggtgtattattgccagcagtatggcagcagcccgctgacctttggccagggcaccaaagtggaaattaaa

SEQ ID NO: 210 - DNA, 351 bp, synthetic construct
  misc_feature 1..351: note = DNA of TM3H65
  source 1..351: mol_type = other DNA
gaagtgcagctgctggaaagcggcggcggcctggtgcagccgggcggcagcctgcgcctgagctgcgcggcgagcggctttacctttagcgactattggatgagctgggtgcgccaggcgccgggcaaaggcctggaatgggtgagcgtgatcaagtatagcggtggctccaaatattatgcggatagcgtgaaaggccgctttaccattagccgcgataacagcaaaaacaccctgtatctgcagatgaacagcctgcgcgcggaagataccgcggtgtattattgcgcgaaagagctggagggggtgttcgactattggggccagggcaccctggtgaccgtgagcagc

SEQ ID NO: 211 - DNA, 324 bp, synthetic construct
  misc_feature 1..324: note = DNA of TM3L12
  source 1..324: mol_type = other DNA
gaaattgtgctgacccagagcccgggcaccctgagcctgagcccgggcgaacgcgcgaccctgagctgccgcgcgagccagagcgttagcaatagcactctggcgtggtatcagcagaaaccgggccaggcgccgcgcctgctgatttatactgcgagcagccgcgcgaccggcattccggatcgctttagcggcagcggcagcggcaccgattttaccctgaccattagccgcctggaaccggaagattttgcggtgtattattgccagcagtcttacacatctccgtggacttttggccagggcaccaaagtggaaattaaa

SEQ ID NO: 212 - protein, 449 aa, synthetic construct
  REGION 1..449: note = HC of PD1B114
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARPGLAAAYDTGNLDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 213 - protein, 214 aa, synthetic construct
  REGION 1..214: note = LC of PD1B114
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 449 aa, synthetic construct; identical to SEQ ID NO: 212
  REGION 1..449: note = HC of PD1B149

SEQ ID NO: 215 - protein, 214 aa, synthetic construct
  REGION 1..214: note = LC of PD1B149
EIVLTQSPATLSLSPGERATLSCRASQSVRNYLAWYQQKPGQAPRLLIHDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 216 - protein, 449 aa, synthetic construct
  REGION 1..449: note = HC of PD1B160
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGNLDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 217 - protein, 214 aa, synthetic construct
  REGION 1..214: note = LC of PD1B160
EIVLTQSPATLSLSPGERATLSCRASQSVDSYLAWYQQKPGQAPRLLIKDASDRATGIPARFSGSGSGTDFTLTISSLEPEDFAVYYCQQRGNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 449 aa, synthetic construct; identical to SEQ ID NO: 216
  REGION 1..449: note = HC of PD1B162

SEQ ID NO: 219 - protein, 214 aa, synthetic construct
  REGION 1..214: note = LC of PD1B162
EIVLTQSPATLSLSPGERATLSCRASQSVDSYLAWYQQKPGQAPRLLIYDASNRATGIPARFSGSGSGTDFTLTISSLEPEDFAVYYCQQREYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 449 aa, synthetic construct; identical to SEQ ID NO: 216
  REGION 1..449: note = HC of PD1B164

SEQ ID NO: 221 - protein, 214 aa, synthetic construct
  REGION 1..214: note = LC of PD1B164
EIVLTQSPATLSLSPGERATLSCRASQSVRSYLAWYQQKPGQAPRLLIYDASYRATGIPARFSGSGSGTDFTLTISSLEPEDFAVYYCQQRDYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - protein, 449 aa, synthetic construct
  REGION 1..449: note = HC of PD1B183
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 223 - protein, 214 aa, synthetic construct
  REGION 1..214: note = LC of PD1B183
EIVLTQSPATLSLSPGERATLSCRASQSVSSYLAWYQQKPGQAPRLLIKDASNRATGIPARFSGSGSGTDFTLTISSLEPEDFAVYYCQQRGYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 224 - protein, 449 aa, synthetic construct; identical to SEQ ID NO: 222
  REGION 1..449: note = HC of PD1B184

SEQ ID NO: 225 - protein, 214 aa, synthetic construct; identical to SEQ ID NO: 215
  REGION 1..214: note = LC of PD1B184

SEQ ID NO: 226 - protein, 449 aa, synthetic construct; identical to SEQ ID NO: 222
  REGION 1..449: note = HC of PD1B185

SEQ ID NO: 227 - protein, 214 aa, synthetic construct
  REGION 1..214: note = LC of PD1B185
EIVLTQSPATLSLSPGERATLSCRASQSVRNYLAWYQQKPGQAPRLLIYDASDRATGIPARFSGSGSGTDFTLTISSLEPEDFAVYYCQQRWNWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - protein, 449 aa, synthetic construct; identical to SEQ ID NO: 222
  REGION 1..449: note = HC of PD1B192

SEQ ID NO: 229 - protein, 214 aa, synthetic construct
  REGION 1..214: note = LC or PD1B192
EIVLTQSPATLSLSPGERATLSCRASQSVDSYLAWYQQKPGQAPRLLIHDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 120 aa, synthetic construct [sequence omitted]

SEQ ID NO: 231 - protein, 111 aa, synthetic construct [sequence omitted]

SEQ ID NO: 232 - protein, 113 aa, synthetic construct [sequence omitted]

SEQ ID NO: 233 - protein, 107 aa, synthetic construct [sequence omitted]

SEQ ID NO: 234 - protein, 121 aa, synthetic construct
  REGION 1..121: note = Durvalumab VH
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 235 - protein, 108 aa, synthetic construct
  REGION 1..108: note = Durvalumab VL
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 236 - protein, 118 aa, synthetic construct [sequence omitted]

SEQ ID NO: 237 - protein, 107 aa, synthetic construct [sequence omitted]

SEQ ID NO: 238 - protein, 120 aa, synthetic construct
  REGION 1..120: note = Avelumab VH
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 239 - protein, 110 aa, synthetic construct
  REGION 1..110: note = Avelumab VL
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 240 - protein, 443 aa, synthetic construct
  REGION 1..443: note = TM3B105 Heavy chain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPYAPLDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 241 - protein, 449 aa, synthetic construct
  REGION 1..449: note = Bispecific mAb HC1
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFLLYSKLTVDKSRWQQGNVFSCSVMHEALHNHYTQKSLSLSPGK

SEQ ID NO: 242 - protein, 444 aa, synthetic construct; identical to SEQ ID NO: 74
  REGION 1..444: note = Bispecific mAb HC1

SEQ ID NO: 243 - protein, 450 aa, synthetic construct; identical to SEQ ID NO: 72
  REGION 1..450: note = Bispecific mAb HC1

SEQ ID NO: 244 - protein, 450 aa, synthetic construct
  REGION 1..450: note = Bispecific mAb HC2
EVQLVQSGAEVKKPGESLKISCKGSGYSFTSYWMQWVRQMPGKGLEWMGAIYPGDGDIRYTQNFKGQVTISADKSISTAYLQWSSLKASDTAMYYCARWEKSTTVVQRNYFDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 245 - protein, 443 aa, synthetic construct
  REGION 1..443: note = Bispecific mAb HC2
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPYAPLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 246 - protein, 444 aa, synthetic construct
  REGION 1..444: note = Bispecific mAb HC2
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPYAPLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 247 - protein, 451 aa, synthetic construct
  REGION 1..451: note = Bispecific mAb HC2
EVQLVQSGAEVKKPGESLKISCKGSGYSFTSYWMQWVRQMPGKGLEWMGAIYPGDGDIRYTQNFKGQVTISADKSISTAYLQWSSLKASDTAMYYCARWEKSTTVVQRNYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 248 - protein, 443 aa, synthetic construct
  REGION 1..443: note = Bispecific mAb HC2
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKSPYAPLDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSRLTVDKSRWQQGNVFSCSVMHEALHNHYTQKSLSLSPGK

SEQ ID NO: 249 - protein, 455 aa, synthetic construct
  REGION 1..455: note = EM1 mAb HC1
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 250 - protein, 214 aa, synthetic construct
  REGION 1..214: note = EM1 mAb LC1
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 251 - protein, 449 aa, synthetic construct
  REGION 1..449: note = EM1 mAb HC2
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 252 - protein, 214 aa, synthetic construct
  REGION 1..214: note = EM1 mAb LC2
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 253 - DNA, 1807 bp, synthetic construct
  misc_feature 1..1807: note = PTBB28 HC1 DNA
  source 1..1807: mol_type = other DNA
cagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgccgccaccatggcttgggtgtggaccttgctattcctgatggcagctgcccaaagtatacaggcccaggtgcagctggtgcagagcggcgcggaagtgaaaaaaccgggcagcagcgtgaaagtgagctgcaaagcgagcggcggcacctttagcagctatgcgattagctgggtgcgccaggcgccgggccagggcctggaatggatgggcggcattattccgatttttgacaccgcgaactatgcgcagaaatttcagggccgcgtgaccattaccgcggatgaaagcaccagcaccgcgtatatggaactgagcagcctgcgcagcgaagataccgcggtgtattattgcgcgcgccctggtctcgctgcggcttatgatactggttccttggactattggggccagggcaccctggtgaccgtgagcagcgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcctgctctacagcaagctcaccgtggacaagagcaggtggcagcaggggaacgtcttctcatgctccgtgatgcatgaggctctgcacaaccactacacgcagaagagcctctccctgtctccgggtaaatgatagttcgaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcac

SEQ ID NO: 254 - DNA, 1105 bp, synthetic construct
  misc_feature 1..1105: note = PTBB28 LC1 DNA
  source 1..1105: mol_type = other DNA
cagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgccgccaccatggagacacattctcaggtctttgtatacatgttgctgtggttgtctggtgtcgagggagaaattgtgctgacccagagcccggcgaccctgagcctgagcccgggcgaacgcgcgaccctgagctgccgcgcgagccagagcgttcgctcctacctggcgtggtatcagcagaaaccgggccaggcgccgcgcctgctgatctacgacgcgagcaatcgtgcgaccggcattccggcgcgctttagcggctccggtagcggcaccgattttaccctgaccattagcagcctggaaccggaagattttgcggtgtattattgccagcaacgtaattattggccgctgacctttggccagggcaccaaagtggaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtgattcgaatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcat

SEQ ID NO: 255 - DNA, 1789 bp, synthetic construct
  misc_feature 1..1789: note = PTBB28 HC2 DNA
  source 1..1789: mol_type = other DNA
cagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgccgccaccatggcttgggtgtggaccttgctattcctgatggcagctgcccaaagtatacaggccgaagtgcagctgctggaaagcggcggcggcctggtgcagccgggcggcagcctgcgcctgagctgcgcggcaagcggctttacctttagcagctatgcgatgagctgggtgcgccaggcgccgggcaaaggcctggaatgggtgagcgcgattagcggcagcggcggcagcacctattatgcggatagcgtgaaaggccgctttaccattagccgcgataacagcaaaaacaccctgtatctgcagatgaacagcctgcgcgcggaagataccgcggtgtattattgcgcgaaatccccgtacgcgcccttggactattggggccagggcaccctggtgaccgtgagcagcgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagccggctcaccgtggacaagagcaggtggcagcaggggaacgtcttctcatgctccgtgatgcatgaggctctgcacaaccactacacgcagaagagcctctccctgtctccgggtaaatgatagttcgaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcac

SEQ ID NO: 256 - DNA, 1105 bp, synthetic construct
  misc_feature 1..1105: note = PTBB28 LC2 DNA
  source 1..1105: mol_type = other DNA
cagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgccgccaccatggagacacattctcaggtctttgtatacatgttgctgtggttgtctggtgtcgagggagaaattgtgctgacccagagcccggcgaccctgagcctgagcccgggcgaacgcgcgacccttagctgccgtgcaagtcagagtgtgaacgactacctggcgtggtatcagcagaaaccgggccaggcgccgcgcctgctgatttatgatgcgagcaaccgcgcgaccggcattccggcgcgctttagcggcagcggcagcggcaccgattttaccctgaccattagcagcctggaaccggaagattttgcggtgtattattgccagcagggtggtcacgcgccgatcacctttggccagggcaccaaagtggaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtgattcgaatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcat

SEQ ID NO: 257 - DNA, 1807 bp, synthetic construct
  misc_feature 1..1807: note = PTBB30 HC1 DNA
  source 1..1807: mol_type = other DNA
cagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgccgccaccatggcttgggtgtggaccttgctattcctgatggcagctgcccaaagtatacaggcccaggtgcagctggtgcagagcggcgcggaagtgaaaaaaccgggcagcagcgtgaaagtgagctgcaaagcgagcggcggcacctttagcagctatgcgattagctgggtgcgccaggcgccgggccagggcctggaatggatgggcggcattattccgatttttgacaccgcgaactatgcgcagaaatttcagggccgcgtgaccattaccgcggatgaaagcaccagcaccgcgtatatggaactgagcagcctgcgcagcgaagataccgcggtgtattattgcgcgcgccctggtctcgctgcggcttatgatactggttccttggactattggggccagggcaccctggtgaccgtgagcagcgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgccgcagccagctcagtcttcctcttccccccaaaacccaaggacaccctcatgatctcccggacccctgaggtcacgtgcgtggtggtggacgtgagcgccgaagaccccgaggtccagttcaactggtacgtggacggcgtggaggtgcataatgccaagacaaagccacgggaggagcagttcaacagcacgttccgtgtggtcagcgtcctcaccgttctgcaccaggactggctgaacggcaaggagtacaagtgcaaggtctccaacaaaggcctcccatcctccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcctgctctacagcaagctcaccgtggacaagagcaggtggcagcaggggaacgtcttctcatgctccgtgatgcatgaggctctgcacaaccactacacgcagaagagcctctccctgtctccgggtaaatgatagttcgaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcac

SEQ ID NO: 258 - DNA, 1105 bp, synthetic construct; identical to SEQ ID NO: 254
  misc_feature 1..1105: note = PTBB30 LC1 DNA
  source 1..1105: mol_type = other DNA

SEQ ID NO: 259 - DNA, 1789 bp, synthetic construct
  misc_feature 1..1789: note = PTBB30 HC2 DNA
  source 1..1789: mol_type = other DNA
cagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgccgccaccatggcttgggtgtggaccttgctattcctgatggcagctgcccaaagtatacaggccgaagtgcagctgctggaaagcggcggcggcctggtgcagccgggcggcagcctgcgcctgagctgcgcggcaagcggctttacctttagcagctatgcgatgagctgggtgcgccaggcgccgggcaaaggcctggaatgggtgagcgcgattagcggcagcggcggcagcacctattatgcggatagcgtgaaaggccgctttaccattagccgcgataacagcaaaaacaccctgtatctgcagatgaacagcctgcgcgcggaagataccgcggtgtattattgcgcgaaatccccgtacgcgcccttggactattggggccagggcaccctggtgaccgtgagcagcgcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgccgcagccagctcagtcttcctcttccccccaaaacccaaggacaccctcatgatctcccggacccctgaggtcacgtgcgtggtggtggacgtgagcgccgaagaccccgaggtccagttcaactggtacgtggacggcgtggaggtgcataatgccaagacaaagccacgggaggagcagttcaacagcacgttccgtgtggtcagcgtcctcaccgttctgcaccaggactggctgaacggcaaggagtacaagtgcaaggtctccaacaaaggcctcccatcctccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagccggctcaccgtggacaagagcaggtggcagcaggggaacgtcttctcatgctccgtgatgcatgaggctctgcacaaccactacacgcagaagagcctctccctgtctccgggtaaatgatagttcgaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcac

SEQ ID NO: 260 - DNA, 1105 bp, synthetic construct; identical to SEQ ID NO: 256
  misc_feature 1..1105: note = PTBB30 LC2 DNA
  source 1..1105: mol_type = other DNA

SEQ ID NO: 261 - protein, 16 aa, Homo sapiens
WGKGACPVFECGNVVL

SEQ ID NO: 262 - protein, 7 aa, Homo sapiens
DERDVNY

SEQ ID NO: 263 - protein, 13 aa, Homo sapiens
RIQIPGIMNDEKF

SEQ ID NO: 264 - protein, 306 aa, Homo sapiens
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 265 - protein, 254 aa, Homo sapiens
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 266 - protein, 202 aa, Homo sapiens
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 267 - protein, 179 aa, Homo sapiens
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 268 - protein, 284 aa, Homo sapiens
DTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 269 - protein, 260 aa, Homo sapiens
LSVQQGPNLLQVRQGSQATLVCQVDQATAWERLRVKWTKDGAILCQPYITNGSLSLGVCGPQGRLSWQAPSHLTLQLDPVSLNHSGAYVCWAAVEIPELEEAEGNITRLFVDPDDPTQNRNRIASFPGFLFVLLGVGSMGVAAIVWGAWFWGRRSCQQRDSGNSPGNAFYSNVLYRPRGAPKKSEDCSGEGKDQRGQSIYSTSFPQPAPRQPHLASRPCPSPRPCPSPRPGHPVSMVRVSPRPSPTQQPRPKGFPKVGEE

SEQ ID NO: 270 - protein, 257 aa, Homo sapiens
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 271 - protein, 216 aa, Homo sapiens
QRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 272 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 273 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 274 - protein, 155 aa, Homo sapiens
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 275 - protein, 240 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 276 - protein, 393 aa, Homo sapiens
QGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 277 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 278 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 279 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 280 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 281 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 282 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 283 - protein, 194 aa, Homo sapiens
QGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARS

SEQ ID NO: 284 - protein, 318 aa, Homo sapiens
EEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 285 - protein, 360 aa, Homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQTFHVSAVAAAAIFVIIIFYVRCCKKKTSAAEGPELVSLQVLDQHPVGTSDHRDATQLGFQPLMSDLGSTGSTEGA

SEQ ID NO: 286 - protein, 279 aa, Homo sapiens
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 287 - protein, 432 aa, Homo sapiens
KQSEDFRVIGPAHPILAGVGEDALLTCQLLPKRTTMHVEVRWYRSEPSTPVFVHRDGVEVTEMQMEEYRGWVEWIENGIAKGNVALKIHNIQPSDNGQYWCHFQDGNYCGETSLLLKVAGLGSAPSIHMEGPGESGVQLVCTARGWFPEPQVYWEDIRGEKLLAVSEHRIQDKDGLFYAEATLVVRNASAESVSCLVHNPVLTEEKGSVISLPEKLQTELASLKVNGPSQPILVRVGEDIQLTCYLSPKANAQSMEVRWDRSHRYPAVHVYMDGDHVAGEQMAEYRGRTVLVSDAIDEGRLTLQILSARPSDDGQYRCLFEKDDVYQEASLDLKVVSLGSSPLITVEGQEDGEMQPMCSSDGWFPQPHVPWRDMEGKTIPSSSQALTQGSHGLFHVQTLLRVTNISAVDVTCSISIPFLGEEKIATFSLSGW

SEQ ID NO: 288 - protein, 506 aa, Homo sapiens
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 289 - protein, 258 aa, Homo sapiens
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 290 - protein, 245 aa, Homo sapiens
LPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 291 - protein, 392 aa, Homo sapiens
IFPLAFFIYVPMNEQIVIGRLDEDIILPSSFERGSEVVIHWKYQDSYKVHSYYKGSDHLESQDPRYANRTSLFYNEIQNGNASLFFRRVSLLDEGIYTCYVGTAIQVITNKVVLKVGVFLTPVMKYEKRNTNSFLICSVLSVYPRPIITWKMDNTPISENNMEETGSLDSFSINSPLNITGSNSSYECTIENSLLKQTWTGRWTMKDGLHKMQSEHVSLSCQPVNDYFSPNQDFKVTWSRMKSGTFSVLAYYLSSSQNTIINESRFSWNKELINQSDFSMNLMDLNLSDSGEYLCNISSDEYTLLTIHTVHVEPSQETASHNKGLWILVPSAILAAFLLIWSVKCCRAQLEARRSRHPADGAQQERCCVPPGERCPSAPDNGEENVPLSGKV

SEQ ID NO: 292 - protein, 188 aa, Homo sapiens
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 293 - protein, 497 aa, Homo sapiens
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 294 - protein, 259 aa, Homo sapiens
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 295 - protein, 133 aa, Homo sapiens
INITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLS

SEQ ID NO: 296 - protein, 492 aa, Homo sapiens
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 297 - protein, 266 aa, Homo sapiens
QEEDLPRPSISAEPGTVIPLGSHVTFVCRGPVGVQTFRLERESRSTYNDTEDVSQASPSESEARFRIDSVSEGNAGPYRCIYYKPPKWSEQSDYLELLVKETSGGPDSPDTEPGSSAGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 298 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 299 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 300 - protein, 564 aa, Homo sapiens
VWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMSWPVIVAALLFCCMILFGLGVRKWCQYQKEIMERPPPFKPPPPPIKYTCIQEPNESDLPYHEMETL

SEQ ID NO: 301 - protein, 223 aa, Homo sapiens [sequence omitted]

SEQ ID NO: 302 - protein, 233 aa, Homo sapiens
MDNQGVIYSDLNLPPNPKRQQRKPKGNKNSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 303 - protein, 507 aa, Homo sapiens
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 304 - protein, 305 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 305 - protein, 474 aa, Homo sapiens
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 306 - protein, 349 aa, Homo sapiens
CQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 307 - protein, 651 aa, Homo sapiens
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSA

SEQ ID NO: 308 - protein, 355 aa, Homo sapiens
MAFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQNPRTVPVQPAFSTVPFSQPVCFPPRPRGRRQKPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 309 - protein, 119 aa, synthetic construct
  REGION 1..119: note = OX40 antiboyd VH
QVQLVQSGAEVKKPGASVKVSCKASGYTFTSYVMHWVRQAPGQRLEWIGYINPYNDGTKYNEKFKGRATLTSDKSASTAYMELSSLRSEDTAVYYCANYYGSSLSMDYWGQGTLVTVSS

SEQ ID NO: 310 - protein, 107 aa, synthetic construct
  REGION 1..107: note = OX40 antibody VL
DIQMTQSPSSLSASVGDRVTITCRASQDISNYLNWYQQKPGKAVKLLIYYTSRLHSGVPSRFSGSGSGTDYTLTISSLQPEDFATYFCQQGNTLPWTFGQGTKVEIK

SEQ ID NO: 311 - protein, 121 aa, synthetic construct
  REGION 1..121: note = OX40 antibody VH
QVQLVQSGAEVKKPGSSVKVSCKASGYTFKDYTMHWVRQAPGQGLEWMGGIYPNNGGSTYNQNFKDRVTITADKSTSTAYMELSSLRSEDTAVYYCARMGYHGPHLDFDVWGQGTTVTVSS

SEQ ID NO: 312 - protein, 107 aa, synthetic construct
  REGION 1..107: note = OX40 antibody VL
DIQMTQSPSSLSASVGDRVTITCKASQDVGAAVAWYQQKPGKAPKLLIYWASTRHTGVPSRFSGSGSGTDFTLTISSLQPEDFATYYCQQYINYPLTFGGGTKVEIK